(19) United States
(12) Patent Application Publication
(10) Pub. No.: US 2026/0174749 A1
(43) Pub. Date: Jun. 25, 2026

(54) COMBINATION THERAPY WITH SUBSTITUTED 3-((3-AMINOPHENYL)AMINO)PIPERIDINE-2,6- DIONE COMPOUNDS

(71) Applicant: Bristol-Myers Squibb Company, Princeton, NJ (US)

(72) Inventors: Raju KANDIMALLA, San Diego, CA (US); Debbie LIAO, San Diego, CA (US)

(73) Assignee: Bristol-Myers Squibb Company, Princeton, NJ (US)

(21) Appl. No.: 19/128,536

(22) PCT Filed: Nov. 7, 2023
(86) PCT No.: PCT/US2023/078908

§ 371 Date: May 8, 2025

Related U.S. Application Data
(60) Provisional application No. 63/424,014, filed on Nov. 9, 2022.

(51) Int. Cl. A61K 31/496; A61K 31/44; A61K 31/517; A61K 31/519; A61K 31/5377; A61K 31/551; A61P 35/00

(52) U.S. Cl. CPC A61K 31/496; A61K 31/44; A61K 31/517; A61K 31/519; A61K 31/5377; A61K 31/551; A61P 35/00

(57) ABSTRACT

Provided herein are methods of treating, preventing, managing, and/or ameliorating androgen receptor mediated diseases with a combination of 3-((3-aminophenyl)amino)piperidine-2, 6-dione compounds and a second agent.

### RELATED APPLICATIONS

This application claims the benefit of U.S. Provisional Application No. 63/424,014, filed Nov. 9, 2022, the disclosure of which is incorporated herein by reference in its entirety.

### FIELD

Provided herein are methods of treating, preventing, managing, and/or ameliorating androgen receptor mediated diseases with a combination of 3-((3-aminophenyl)amino)piperidine-2,6-dione compounds and a second agent. Also provided herein is a combination of a 3-((3-aminophenyl)amino)piperidine-2,6-dione compound and a second agent for use in such methods.

### BACKGROUND

Androgen receptor signaling is known to play a crucial role in the pathogenesis of prostate cancer and is involved in the development of other androgen receptor positive cancers (Chen Y et al., Lancet Oncol, 2009, 10:981-91; Mills I G, Nat Rev Cancer, 2014, 14:187-98; Taplin M E, Nat Clin Pract Oncol, 2007, 4:236-44; Wirth M P et al., Eur Urol, 2007, 51(2):306-13). The inhibition of androgen receptor signaling with anti-androgens that antagonize the androgen receptor has been used or proposed for the treatment of prostate cancer. Exemplary compounds are disclosed in U.S. Pat. No. 11,149,007.

There is a need for methods of treatment using a combination therapy for androgen receptor mediated diseases with 3-((3-aminophenyl)amino)piperidine-2,6-dione compounds and a second agent.

### BRIEF SUMMARY

The compounds used in the methods herein are described in U.S. Pat. No. 11,149,007, the disclosure of which is incorporated herein by reference in its entirety.

In one embodiment, provided herein are methods of treating, preventing, managing, and/or ameliorating androgen receptor mediated diseases, by administering a compound of formula I as described herein in combination with one or more second agents selected from a PI3K inhibitor, an AKT inhibitor, a BET inhibitor, JAK inhibitor, and an EZH2 inhibitor. Thus, provided herein is a Piperidine Dione Compound for use in such methods, wherein the method comprises administering a Piperidine Dione Compound in combination with one or more second agents selected from a PI3K inhibitor, an AKT inhibitor, a BET inhibitor, JAK inhibitor, and an EZH2 inhibitor.

In certain embodiments, provided herein is a unit dosage form comprising a Piperidine Dione Compound and a second agent selected from a PI3K inhibitor, an AKT inhibitor, a BET inhibitor, JAK inhibitor, and an EZH2 inhibitor.

Further provided is a pharmaceutical pack or kit comprising a Piperidine Dione Compound and one or more second agents selected from a PI3K inhibitor, an AKT inhibitor, a BET inhibitor, JAK inhibitor, and an EZH2 inhibitor. Optionally associated with such pharmaceutical pack or kit can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like.

These and other aspects of the subject matter described herein will become evident upon reference to the following detailed description.

### DETAILED DESCRIPTION

### Definitions

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs. In general, the technical teaching of one embodiment can be combined with that disclosed in other embodiments provided herein.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 carbon atoms. In some embodiments, the alkyl group is a saturated alkyl group. Representative saturated alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. In some embodiments, the alkyl group is an unsaturated alkyl group, also termed an alkenyl or alkynyl group. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, heterocycloalkyloxy, cycloalkylalkyloxy, aralkyloxy, heterocyclylalkyloxy, heteroarylalkyloxy, heterocycloalkylalkyloxy; oxo (═O); amino, alkylamino, cycloalkylamino, arylamino, heterocyclylamino, heteroarylamino, heterocycloalkylamino, cycloalkylalkylamino, aralkylamino, heterocyclylalkylamino, heteroaralkylamino, heterocycloalkylalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)2. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

A “cycloalkyl” group is a saturated, or partially saturated cyclic alkyl group of from 3 to 10 carbon atoms having a single cyclic ring or multiple condensed or bridged rings which can be optionally substituted. In some embodiments, the cycloalkyl group has 3 to 8 ring members, whereas in other embodiments the number of ring carbon atoms ranges from 3 to 5, 3 to 6, or 3 to 7. In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. In other embodiments, the cycloalkyl groups are unsaturated cycloalkyl groups. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1(2H)-onyl), tetrahydroquinolinyl, quinoxalinyl, and quinazolinyl groups. A heteroaryl group can be substituted or unsubstituted.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocycloalkyl group can be substituted or unsubstituted. Heterocyclyl groups encompass unsaturated, partially saturated and saturated ring systems, such as, for example, imidazolyl, imidazolinyl and imidazolidinyl (e.g., imidazolidin-4-one or imidazolidin-2,4-dionyl) groups. The phrase heterocyclyl includes fused ring species, including those comprising fused aromatic and non-aromatic groups, such as, for example, 1- and 2-aminotetraline, benzotriazolyl (e.g., 1H-benzo[d][1,2,3]triazolyl), benzimidazolyl (e.g., 1H-benzo[d]imidazolyl), 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, azepanyl, oxetanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, benzisoxazolyl (e.g., benzo[d]isoxazolyl), thiazolyl, thiazolinyl, isothiazolyl, thiadiazolyl, oxadiazolyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, pyridyl, pyrimidyl, pyridazinyl, pyrazinyl, triazinyl, dihydropyridyl, dihydrodithionyl, dihydrodithionyl, 1,4-dioxaspiro[4.5]decanyl, homopiperazinyl, quinuclidyl, indolyl (e.g., indolyl-2-onyl or isoindolin-1-onyl), indolinyl, isoindolyl, isoindolinyl, azaindolyl (pyrrolopyridyl or 1H-pyrrolo[2,3-b]pyridyl), indazolyl, indolizinyl, benzotriazolyl (e.g. 1H-benzo[d][1,2,3]triazolyl), benzimidazolyl (e.g., 1H-benzo[d]imidazolyl or 1H-benzo[d]imidazol-2(3H)-onyl), benzofuranyl, benzothiophenyl, benzothiazolyl, benzoxadiazolyl, benzoxazinyl, benzodithiinyl, benzoxathiinyl, benzothiazinyl, benzoxazolyl (i.e., benzo[d]oxazolyl), benzothiazolyl, benzothiadiazolyl, benzo[1,3]dioxolyl, pyrazolopyridyl (for example, 1H-pyrazolo[3,4-b]pyridyl, 1H-pyrazolo[4,3-b]pyridyl), imidazopyridyl (e.g., azabenzimidazolyl or 1H-imidazo[4,5-b]pyridyl), triazolopyridyl, isoxazolopyridyl, purinyl, xanthinyl, adeninyl, guaninyl, quinolinyl, isoquinolinyl (e.g., 3,4-dihydroisoquinolin-1(2H)-onyl), quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, tetrahydropyrimidin-2(1H)-one and tetrahydroquinolinyl groups. Representative non-aromatic heterocyclyl groups do not include fused ring species that comprise a fused aromatic group. Examples of non-aromatic heterocyclyl groups include aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

As used herein and unless otherwise specified, a “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopropylmethyl, cyclobutylmethyl, cyclopentylmethyl, cyclohexylmethyl, cyclopropylethyl, cyclobutylethyl, cyclopentylethyl, cyclohexylethyl, cyclopentylpropyl, cyclohexylpropyl and the like.

As used herein and unless otherwise specified, an “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and aralkyl groups wherein the aryl group is fused to a cycloalkyl group such as indan-4-yl ethyl.

As used herein and unless otherwise specified, a “heterocyclylalkyl” group is a radical of the formula: -alkyl-heterocyclyl, wherein alkyl and heterocyclyl are defined above. A “heteroarylalkyl” group is a radical of the formula: -alkyl-heteroaryl, wherein alkyl and heteroaryl are defined above. A “heterocycloalkylalkyl” group is a radical of the formula: -alkyl-heterocycloalkyl, wherein alkyl and heterocycloalkyl are defined above. Substituted heterocyclylalkyl groups may be substituted at the alkyl, the heterocyclyl, or both the alkyl and the heterocyclyl portions of the group. Representative heterocylylalkyl groups include but are not limited to morpholin-4-yl ethyl, morpholin-4-yl propyl, furan-2-yl methyl, furan-3-yl methyl, pyridin-3-yl methyl, tetrahydrofuran-2-yl ethyl, and indol-2-yl propyl.

A “halogen” is fluorine, chlorine, bromine or iodine.

A “hydroxyalkyl” group is an alkyl group as described above substituted with one or more hydroxy groups.

An “alkoxy” group is —O-(alkyl), wherein alkyl is defined above.

An “alkoxyalkyl” group is -(alkyl)-O-(alkyl), wherein alkyl is defined above.

An “amino” group is a radical of the formula: —NH₂, —NH(R^{#}), or —N(R^{#})₂, wherein each R^{#} is independently an alkyl, cycloalkyl, cycloalkylalkyl, aryl, aralkyl, heterocyclyl (e.g., heteroaryl or heterocycloalkyl), or heterocyclylalkyl (e.g., heteroarylalkyl or heterocycloalkylalkyl) group defined above, each of which is independently substituted or unsubstituted.

In one embodiment, an “amino” group is an “alkylamino” group, which is a radical of the formula: —NH-alkyl or —N(alkyl)₂, wherein each alkyl is independently defined above. The term “cycloalkylamino”, “arylamino”, “heterocyclylamino”, “heteroarylamino”, “heterocycloalkylamino”, or the like, mirrors the above description for “alkylamino” where the term “alkyl” is replaced with “cycloalkyl”, “aryl”, “heterocyclyl”, “heteroaryl”, “heterocycloalkyl”, or the like, respectively.

A “carboxy” group is a radical of the formula: —C(O)OH.

As used herein and unless otherwise specified, an “acyl” group is a radical of the formula: —C(O)(R^{#}) or —C(O)H, wherein R⁴ is defined above. A “formyl” group is a radical of the formula: —C(O)H.

As used herein and unless otherwise specified, an “amido” group is a radical of the formula: —C(O)—NH₂, —C(O)—NH(R^{#}), —C(O)—N(R^{#})₂, —NH—C(O)H, —NH—C(O)—(R^{#}), —N(R^{#})—C(O)H, or —N(R^{#})—C(O)—(R^{#}), wherein each R^{#} is independently defined above.

In one embodiment, an “amido” group is an “aminocarbonyl” group, which is a radical of the formula: —C(O)—NH₂, —C(O)—NH(R^{#}), —C(O)—N(R⁴)₂, wherein each R^{#} is independently defined above.

In one embodiment, an “amido” group is an “acylamino” group, which is a radical of the formula: —NH—C(O)H, —NH—C(O)—(R^{#}), —N(R^{#})—C(O)H, or —N(R^{#})—C(O)—(R^{#}), wherein each R^{#} is independently defined above.

A “sulfonylamino” group is a radical of the formula: —NHSO₂(R^{#}) or —N(alkyl)SO₂(R^{#}), wherein each alkyl and R^{#} are defined above.

A “urea” group is a radical of the formula: —N(alkyl)C(O)N(R^{#})₂, —N(alkyl)C(O)NH(R^{#}), —N(alkyl)C(O)NH₂, —NHC(O)N(R^{#})₂, —NHC(O)NH(R^{#}), or —NH(CO)NH₂, wherein each alkyl and R^{#} are independently as defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

As used herein, the term “Piperidine Dione Compound” refers to compounds of formula (I) as well as to further embodiments provided herein. In one embodiment, an “Piperidine Dione Compound” is a compound set forth in Table 1. The term “Piperidine Dione Compound” includes pharmaceutically acceptable salts, tautomers, isotopologues, and stereoisomers of the compounds provided herein.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of formula (I) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride formic, and mesylate salts. Others are well known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereoisomerically pure” means one stereoisomer of a Piperidine Dione Compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The Piperidine Dione Compounds can have chiral centers and can occur as racemates, individual enantiomers or diastereomers, and mixtures thereof. All such isomeric forms are included within the embodiments disclosed herein, including mixtures thereof.

The use of stereoisomerically pure forms of such Piperidine Dione Compounds, as well as the use of mixtures of those forms, are encompassed by the embodiments disclosed herein. For example, mixtures comprising equal or unequal amounts of the enantiomers of a particular Piperidine Dione Compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuja, S., Chiral Separation Methods for Pharmaceutical and Biotechnological Products (John Wiley & Sons, 2011).

It should also be noted the Piperidine Dione Compounds can include E and Z isomers, or a mixture thereof, and cis and trans isomers or a mixture thereof. In certain embodiments, the Piperidine Dione Compounds are isolated as either the E or Z isomer. In other embodiments, the Piperidine Dione Compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

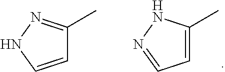

As readily understood by one skilled in the art, a wide variety of functional groups and other structures may exhibit tautomerism and all tautomers of compounds of formula (I) are within the scope of the present invention.

It should also be noted the Piperidine Dione Compounds can contain unnatural proportions of atomic isotopes at one or more of the atoms. For example, the compounds may be radiolabeled with radioactive isotopes, such as for example tritium (³H), iodine-125 (¹²⁵) sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of the Piperidine Dione Compounds as described herein, whether radioactive or not, are intended to be encompassed within the scope of the embodiments provided herein. In some embodiments, there are provided isotopologues of the Piperidine Dione Compounds, for example, the isotopologues are deuterium, carbon-13, and/or nitrogen-15 enriched Piperidine Dione Compounds. As used herein, “deuterated”, means a compound wherein at least one hydrogen (H) has been replaced by deuterium (indicated by D or ²H), that is, the compound is enriched in deuterium in at least one position.

It is understood that, independently of stereoisomerical or isotopic composition, each Piperidine Dione Compound referred to herein can be provided in the form of any of the pharmaceutically acceptable salts discussed herein. Equally, it is understood that the isotopic composition may vary independently from the stereoisomerical composition of each Piperidine Dione Compound referred to herein. Further, the isotopic composition, while being restricted to those elements present in the respective Piperidine Dione Compound or salt thereof, may otherwise vary independently from the selection of the pharmaceutically acceptable salt of the respective Piperidine Dione Compound.

It should be noted that if there is a discrepancy between a depicted structure and a name for that structure, the depicted structure is to be accorded more weight.

The term “subject” or “patient” refers to an animal, including, but not limited to, a mammal, including a primate (e.g., human), cow, sheep, goat, horse, dog, cat, rabbit, rat, or mouse. The terms “subject” and “patient” are used interchangeably herein in reference, for example, to a mammalian subject, such as a human subject.

The term “androgen receptor” or “AR” or “NR3C4” as used herein refers to a nuclear hormone receptor activated by binding of the androgenic hormones, including testosterone or dihydrotestosterone. The term “androgen receptor” may refer to the nucleotide sequence or protein sequence of human androgen receptor (e.g., Entrez 367, Uniprot P10275, RefSeq NM_000044, or RefSeq NP_000035).

The term “AR-full length” (AR-FL) as used herein refers to AR protein that contains all four functional domains, including the N-terminal transactivation domain (NTD, exon 1), the DNA-binding domain (DBD, exons 2-3), the hinge domain (exon 4), and the C-terminal ligand binding domain (LBD, exons 4-8).

The term “castration resistant prostate cancer” (CRPC) refers to advanced prostate cancer that is worsening or progressing while the patient remains on androgen deprivation therapy or other therapies to reduce testosterone, or prostate cancer which is considered hormone refractory, hormone naive, androgen independent or chemical or surgical castration resistant. Castration resistant prostate cancer (CRPC) is an advanced prostate cancer that developed despite ongoing ADT and/or surgical castration. Castration resistant prostate cancer is defined as prostate cancer that continues to progress or worsen or adversely affect the health of the patient despite prior surgical castration, continued treatment with gonadotropin releasing hormone agonists (e.g., leuprolide) or antagonists (e.g., degarelix or abarelix), antiandrogens (e.g., bicalutamide, flutamide, enzalutamide, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®), cabozantinib (Cometriq®, also known as XL184)) or other prostate cancer therapies (e.g., vaccines (sipuleucel-T (Provenge®), GVAX, etc.), herbal (PC-SPES) and lyase inhibitor (abiraterone)) as evidenced by increasing or higher serum levels of prostate specific antigen (PSA), metastasis, bone metastasis, pain, lymph node involvement, increasing size or serum markers for tumor growth, worsening diagnostic markers of prognosis, or patient condition.

As used herein, and unless otherwise specified, the expression “unit dose” refers to a physically discrete unit of a formulation appropriate for a subject to be treated (e.g., for a single dose); each unit containing a predetermined quantity of an active agent selected to produce a desired therapeutic effect (it being understood that multiple doses may be required to achieve a desired or optimum effect), optionally together with a pharmaceutically acceptable carrier, which may be provided in a predetermined amount. The unit dose may be, for example, a volume of liquid (e.g. an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may contain a variety of components in addition to the therapeutic agent(s). For example, acceptable carriers (e.g., pharmaceutically acceptable carriers), diluents, stabilizers, buffers, preservatives, etc., may be included as described infra. It will be understood, however, that the total daily usage of a formulation of the present disclosure will be decided by the attending physician within the scope of sound medical judgment. The specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

A “pharmaceutically acceptable excipient,” refers to a substance that aids the administration of an active agent to a subject by for example modifying the stability of an active agent or modifying the absorption by a subject upon administration. A pharmaceutically acceptable excipient typically has no significant adverse toxicological effect on the patient. Examples of pharmaceutically acceptable excipients include, for example, water, NaCl (including salt solutions), normal saline solutions, 1%2 normal saline, sucrose, glucose, bulking agents, buffers, binders, fillers, disintegrants, lubricants, coatings, sweeteners, flavors, alcohols, oils, gelatins, carbohydrates such as amylose or starch, fatty acid esters, hydroxymethycellulose, polyvinyl pyrrolidine, and colors, and the like. One of skill in the art will recognize that other pharmaceutical excipients known in the art are useful in the present invention and include those listed in for example the Handbook of Pharmaceutical Excipients, Rowe R. C., Shesky P. J., and Quinn M. E., 6^{th} Ed., The Pharmaceutical Press, RPS Publishing (2009). The terms “bulking agent”, and “buffer” are used in accordance with the plain and ordinary meaning within the art.

As used herein, and unless otherwise specified, the term “about,” when used in connection with doses, amounts, or weight percent of ingredients of a composition or a dosage form, means dose, amount, or weight percent that is recognized by those of ordinary skill in the art to provide a pharmacological effect equivalent to that obtained from the specified dose, amount, or weight percent is encompassed. Specifically, the term “about” contemplates a dose, amount, or weight percent within 30%, 25%, 20%, 15%, 10%, or 5% of the specified dose, amount, or weight percent is encompassed.

As used herein, “administer” or “administration” refers to the act of physically delivering a substance as it exists outside the body into a subject. Administration includes all forms known in the art for delivering therapeutic agents, including but not limited to topical, mucosal, injections, intradermal, intravenous, intramuscular delivery or other method of physical delivery described herein or known in the art (e.g., implantation of a slow-release device, such as a mini-osmotic pump to a subject; liposomal formulations; buccal; sublingual; palatal; gingival; nasal; vaginal; rectal; intra-arteriole; intraperitoneal; intraventricular; intracranial; or transdermal).

By “co-administer” it is meant that compounds, compositions or agents described herein are administered at the same time, just prior to, or just after the administration of one or more additional compounds, compositions or agents, including for example an anti-cancer agent. Co-administration is meant to include simultaneous or sequential administration of compounds, compositions or agents individually or in combination (more than one compound or agent). Co-administration includes administering two compounds, compositions or agents simultaneously, approximately simultaneously (e.g., within about 1, 5, 10, 15, 20, or 30 minutes of each other), or sequentially in any order. Thus, co-administration can include administering one active agent (e.g. a compound described herein) within 0.5, 1, 2, 4, 6, 8, 10, 12, 16, 20, or 24 hours of a second active agent. Co-administration can also be accomplished by co-formulation, e.g., preparing a single dosage form including both active agents. The active agents can be formulated separately. In such instances, the active agents are admixed and included together in the final form of the dosage unit. Alternatively, co-administration as described herein can include administering two separate unit dosage forms of at least two separate active agents (e.g., Piperidine Dione Compound and a second active agent described herein).

As used herein, the term “daily” is intended to mean that a therapeutic compound, such as a Piperidine Dione Compound, is administered once or more than once each day for a period of time. The term “continuous” is intended to mean that a therapeutic compound, such as a Piperidine Dione Compound, is administered daily for an uninterrupted period of at least 10 days to 52 weeks. The term “intermittent” or “intermittently” as used herein is intended to mean stopping and starting at either regular or irregular intervals. For example, intermittent administration of a Piperidine Dione Compound is administration for one to six days per week, administration in cycles (e.g., daily administration for one to ten consecutive days of a 28 day cycle, then a rest period with no administration for rest of the 28 day cycle or daily administration for two to eight consecutive weeks, then a rest period with no administration for up to one week), or administration on alternate days. The term “cycling” as used herein is intended to mean that a therapeutic compound, such as a Piperidine Dione Compound, is administered daily or continuously but with a rest period.

A “cycling therapy” refers to a regimen or therapy that includes an administration period as described herein and a rest period as described herein.

The term “administration period” as used herein refers to a period of time a subject is continuously or actively administered a compound or composition described herein.

The term “rest period” as used herein refers to a period of time, often following an administration period, where a subject is not administered a compound or composition described herein (e.g. discontinuation of treatment). In certain embodiments, a “rest period” refers to a period of time where a single agent is not administered to a subject or treatment using a particular compound is discontinued. In such embodiments, a second therapeutic agent (e.g., a different agent than the compound or composition administered in the previous administration period) can be administered to the subject.

An “effective amount” is an amount sufficient to achieve the effect for which it is administered (e.g., treat a disease or reduce one or more symptoms of a disease or condition). Thus, administration of an “amount” of a compound described herein to a subject refers to administration of “an amount effective,” to achieve the desired therapeutic result. A “therapeutically effective amount” of a compound described herein for purposes herein is thus determined by such considerations as are known in the art. The term “therapeutically effective amount” of a composition described herein refers to the amount of the composition that, when administered, is sufficient to treat one or more of the symptoms of a disease described herein. Administration of a compound described herein can be determined according to factors such as, for example, the disease state, age, sex, and weight of the individual. A therapeutically effective amount also refers to any toxic or detrimental effects of a Piperidine Dione Compound are outweighed by the therapeutically beneficial effects.

As used herein, “maintenance therapy” refers to the treatment given for a disease after remission or best response is achieved, in order to prevent or delay relapse. Maintenance therapy can include chemotherapy, hormone therapy or targeted therapy.

“Remission” as used herein, is a decrease in or disappearance of signs and symptoms of a cancer, for example, multiple myeloma. In partial remission, some, but not all, signs and symptoms of the cancer have disappeared. In complete remission, all signs and symptoms of the cancer have disappeared, although the cancer still may be in the body.

The term “relapsed” refers to a situation where patients who have had a remission of leukemia after therapy have a return of leukemia cells in the marrow and a decrease in normal blood cells.

The term “refractory or resistant” refers to a circumstance where patients, even after intensive treatment, have residual leukemia cells in their marrow.

The term “drug resistance” refers to the condition when a disease does not respond to the treatment of a certain drug or drugs. Drug resistance can be either intrinsic, which means the disease has never been responsive to the particular drug or drugs, or it can be acquired, which means the disease ceases responding to particular a drug or drugs that the disease had previously responded to. In certain embodiments, drug resistance is intrinsic. In certain embodiments, the drug resistance is acquired.

### Compounds

Provided herein are compounds having the following formula (I):

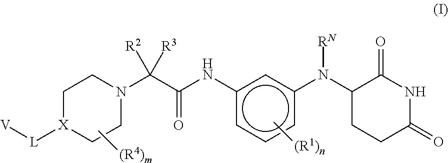

or a pharmaceutically acceptable salt, tautomer, isotopolog, or stereoisomer thereof,
wherein:
R^{N} is H;
each R¹ is independently selected from halogen, CN, and C₁₋₃ alkyl;
R² and R³ are each independently selected from H, and C₁-3 alkyl, or R² and R³ and the carbon to which they are attached form a substituted or unsubstituted C₃₋₆ cycloalkyl;
each R⁴ is independently substituted or unsubstituted C₁₋₃ alkyl, or two R⁴ groups, together with the same carbon atom or adjacent carbon atoms to which they are attached, form a substituted or unsubstituted C₃₋₆ cycloalkyl, or two R⁴ groups together with the non-adjacent carbon atoms to which they are attached form a substituted or unsubstituted 4-7-membered heterocyclyl;
X is N;
L is —O(C₁₋₆ alkyl)- or —(C₁₋₉ alkyl)-;
n is 0-4;
m is 0-8;
V is

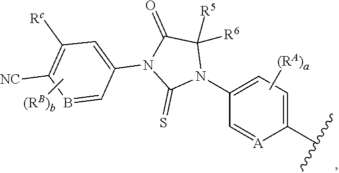

wherein
A is N, CH, or CR^{A};
B is N, CH, or CR^{B};
each R^{A} is independently selected from halogen, substituted or unsubstituted C₁₋₆ alkyl, and substituted or unsubstituted C₃₋₆ cycloalkyl;
each R^{B} is independently selected from halogen, and substituted or unsubstituted C₁₋₆ alkyl;
R^{C} is halogen or CF₃;
R⁵ and R⁶ are C₁₋₃ alkyl, or R⁵ and R⁶, together with the carbon atom to which they are attached, form a substituted or unsubstituted C₃₋₆ cycloalkyl or a 3-6 membered heterocyclyl;
a is 0-3; and
b is 0-2.

Provided herein are compounds having the following formula (I):

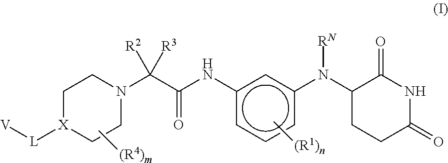

or a pharmaceutically acceptable salt, tautomer, isotopolog, or stereoisomer thereof,
wherein:
R^{N} is H;
each R¹ is independently selected from halogen, CN, and C₁₋₃ alkyl;
R² and R³ are each independently selected from H, and C₁-3 alkyl, or R² and R³ and the carbon to which they are attached form a substituted or unsubstituted C₃₋₆ cycloalkyl;
each R⁴ is independently substituted or unsubstituted C₁₋₃ alkyl, or two R⁴ groups, together with the same carbon atom or adjacent carbon atoms to which they are attached, form a substituted or unsubstituted C₃₋₆ cycloalkyl, or two R⁴ groups together with the non-adjacent carbon atoms to which they are attached form a substituted or unsubstituted 4-7-membered heterocyclyl;
X is N;
L is —O(CH₂)ₚ— or —(CH₂)ₚ—;
n is 0-4;
m is 0-8;
p is 1-3;
V is

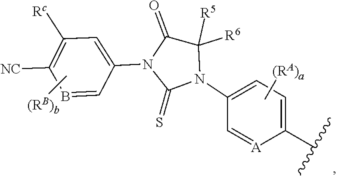

wherein
A is N, CH, or CR^{A};
B is N, CH, or CR^{B};
each R^{A} is independently selected from halogen, substituted or unsubstituted C₁₋₆ alkyl, and substituted or unsubstituted C₃₋₆ cycloalkyl;
each R^{B} is independently selected from halogen, and substituted or unsubstituted C₁₋₆ alkyl;
R^{C} is halogen or CF₃;
R⁵ and R⁶ are C₁₋₃ alkyl, or R⁵ and R⁶, together with the carbon atom to which they are attached, form a substituted or unsubstituted C₃₋₅ cycloalkyl or a 3-5 membered heterocyclyl;
a is 0-3; and
b is 0-2.

In one embodiment of a compound of formula (I), the compound is

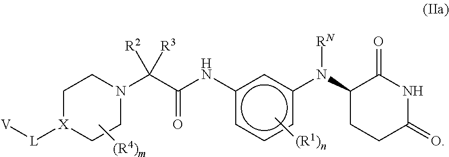

In another embodiment of a compound of formula (I), the compound is

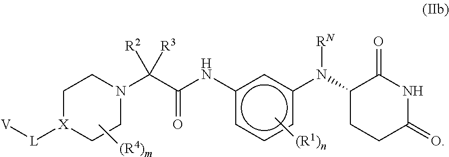

In some embodiments of compounds of formula (I), (IIa) and (IIb), each R¹ is independently selected from halogen, and C₁₋₃ alkyl. In some embodiments of compounds of formula (I), (IIa) and (IIb), each R¹ is independently selected from Cl, F, Br, CN, —CH₃, —CH₂CH₃, and isopropyl. In other embodiments, each R¹ is independently selected from Cl, F, CN, and —CH₃. In some other embodiments, each R¹ is independently selected from Cl, F, and CN.

In some embodiments of compounds of formula (I), n is 0. In other embodiments, n is 1 or 2.

In some embodiments of compounds of formula (I), the compound is

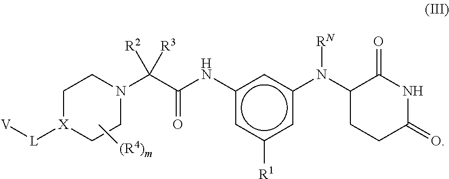

In other embodiments of compounds of formula (I), the compound is

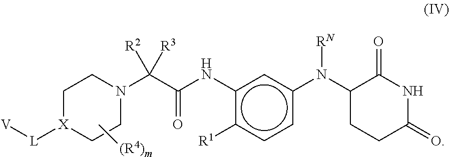

In still other embodiments of compounds of formula (I), the compound is

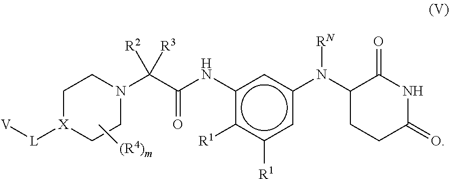

In some embodiments, the compound is a compound of formula (III), (IV) or (V), wherein each R¹ is independently selected from Cl, F, CN, and CH₃. In some such embodiments, the compound is a compound of formula (III), (IV) or (V), wherein R¹ is F or Cl. In some embodiments of compounds of formula (III), R¹ is F, Cl, or CN. In some embodiments of compounds of formula (IV), R¹ is F.

In some embodiments of compounds of formula (I), R² and R³ are each independently selected from H, substituted or unsubstituted methyl, and ethyl, or R² and R³ and the carbon to which they are attached form a substituted or unsubstituted cyclopropyl, cyclobutyl or cyclopentyl. In some such embodiments, R² and R³ are each independently selected from H and methyl, or R² and R³ and the carbon to which they are attached form an unsubstituted cyclopropyl. In some other embodiments, R² and R³ are both H or methyl, or R² and R³ and the carbon to which they are attached form an unsubstituted cyclopropyl. In some embodiments, R² and R³ are H.

In some embodiments of compounds of formula (I), each R⁴ is independently selected from substituted or unsubstituted methyl and ethyl, or two R⁴ groups, together with the same carbon atom to which they are attached, form a substituted or unsubstituted cyclopropyl or cyclobutyl. In some embodiments, each R⁴ is independently substituted or unsubstituted methyl, or two R⁴ groups, together with the same carbon atom to which they are attached, form an unsubstituted cyclopropyl. In yet other embodiments, each R⁴ is independently selected from methyl, CF₃, and CH₂OH, or two R⁴ groups, together with the same carbon atom to which they are attached, form an unsubstituted cyclopropyl. In still other embodiments, R⁴ is methyl. In some embodiments, R⁴ is ethyl.

In some embodiments of compounds of formula (I), m is 0, 1, 2, 3 or 4. In some embodiments, m is 0, 1, or 2.

In some embodiments of compounds of formula (I), two R⁴ groups together with the non-adjacent carbon atoms to which they are attached form an unsubstituted 4-7-membered heterocyclyl. In some such embodiments, the compound is

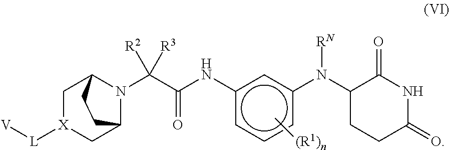

In some such embodiments, the compound is

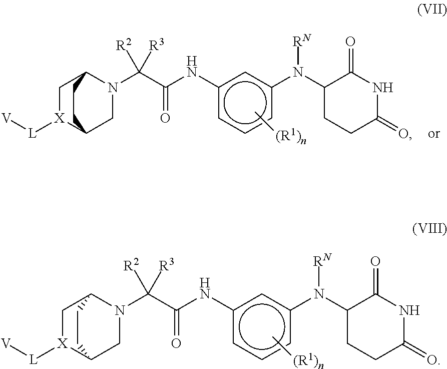

In some such embodiments, the compound is

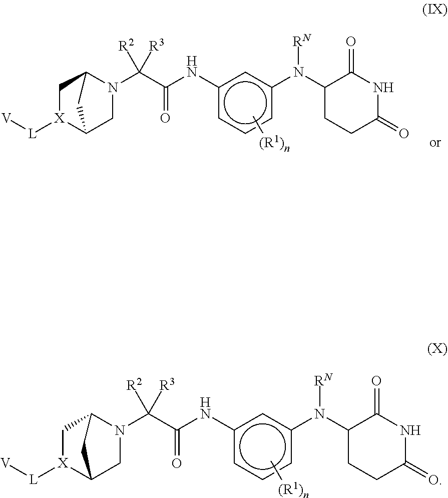

In some embodiments of compounds of formula (I), (IIa), (IIb), (III), (IV), (V), (VI), (VII), (VIII), (IX), and (X), exemplary L groups include, but are not limited to, —O(CH₂)(CH₂)—, —O(CH₂)(CH(CH₃))—, —O(CH₂)(C(CH₃)₂)—, —O(CH(CH₃))(CH₂)—, —O(C(CH₃)₂)(CH₂)—, —O(CH(CH₃))(CH(CH₃))—, —O(CH(CH₃))(C(CH₃)₂)—, —O(C(CH₃)₂)(CH(CH₃))—, —(CH₂)—, —(CH₂)(CH₂)—, —(CH₂)(CH₂)(CH₂)—, —(C(CH₃)₂)(C(CH₃)₂)—, —(CH(CH₃))—, —(C(CH₃)₂)—, —(CH(CH₃))(CH(CH₃))—, —(CH(CH₃))(C(CH₃)₂)—, —(C(CH₃)₂)(CH(CH₃))—, —(C(CH₃)₂)(C(CH₃)₂)—, —(CH₃)(CH₃)(CH(CH₃))—, —(CH₂)(CH(CH₃))(CH₂)—, —(CH(CH₃))(CH₂)(CH₂)—, —(CH₂)(CH₂)(C(CH₂)₂)—, —(CH₂)(C(CH₂)₂)(CH₂)—, —(C(CH₂)₂)(CH₂)(CH₂)—, —(CH₂)(CH(CH₃))(CH(CH₃))—, —(CH(CH₃))(CH(CH₃))(CH(CH₃))—, —(CH(CH₃))(CH(CH₃))(CH₂)—, —(CH(CH₃))(CH₂)(CH(CH₃))—, —(CH₂)(CH(CH₃))(C(CH₃)₂)—, —(CH(CH₃))(CH₂)(C(CH₂)₂)—, —(C(CH₃)₂)(CH₂)(C(CH₃)₂)—, —(CH₂)(C(CH₃)₂)(C(CH₃)₂)—, —(CH₂)(C(CH₃)₂)(CH(CH₃))—, —(CH(CH₃)(C(CH₃)₂)(CH₂)—, —(C(CH₃)₂)(CH₂)(CH(CH₃))—, —(C(CH₃)₂)(CH(CH₃))(CH₂)—, —(C(CH₃)₂)(C(CH₃)₂)(CH₂)—, —(CH(CH₃))(CH(CH₃))(C(CH₃)₂)—, —(CH(CH₃))(C(CH₃)₂)(C(CH₃)₂)—, —(C(CH₃)₂)(CH(CH₃))(C(CH₃)₂)—, —(CH(CH₃))(C(CH₃)₂)(CH(CH₃))—, —(C(CH₃)₂)(C(CH₃)₂)(CH(CH₃))—, —(C(CH₃)₂)(CH(CH₃))(CH(CH₃))—, and —(C(CH₃)₂)(C(CH₃)₂)(C(CH₃)₂)—.

In some embodiments of compounds of formula (I), L is —O(CH₂)(CH₂)—, —O(CH₂)(CH(CH₃))—, —O(CH₂)(C(CH₃)₂)—, —O(CH(CH₃))(CH₂)—, O(CH(CH₃))(CH(CH₃))—, —O(CH(CH₃)(C(CH₃)₂)—, —O(C(CH₃)₂)(CH₂)—, —(CH₂)—, —(CH₂)(CH₂)—, or —(CH₂)(CH₂)(CH₂)—. In some embodiments of compounds of formula (I), L is —O(CH₂)(CH₂)—, —(CH₂)—, —(CH₂)(CH₂)—, or —(CH₂)(CH₂)(CH₂)—. In other embodiments, L is —O(CH₂)(CH₂)—, or —(CH₂)(CH₂)(CH₂)—. In still other embodiments, L is —O(CH₂)(CH₂)—. In some embodiments of compounds of formula (I), L is —O(CH₂)(CH₂)—, —O(CH₂)(CH(CH₃))—, —O(CH(CH₃))(CH₂)—, or —(CH₂)(CH₂)(CH₂)—.

In some embodiments of compounds of formula (I), A is CH. In some other embodiments of compounds of formula (I), B is CH. In some other embodiments, B is N.

In some embodiments of compounds of formula (I), a is 0, 1 or 2.

In some embodiments of compounds of formula (I), each R^{A} is independently selected from Cl, Br, F, methyl, ethyl, n-propyl, isopropyl, n-butyl, isobutyl, sec-butyl, tert-butyl, n-pentyl, CH₂CH₂CH(CH₃)₂, CH(CH₃)CH(CH₃)₂, CF₃, CF(CH₃)₂, CF₂CH₃, CH₂CH₂F, CH₂CHF₂, CH₂CF₃, CH₂OH, CH(CH₃)OH, CH₂CH₂OH, CH(CH₃)CH₂OH, CH₂CH(CH₃)OH, cyclopropyl, cyclobutyl, and cyclopentyl. In some such embodiments, each R^{A} is independently selected from Cl, Br, F, methyl, ethyl, n-propyl, isopropyl, isobutyl, sec-butyl, CF₃, CF₂CH₃, CH₂CH₂F, CH₂CHF₂, CH₂OH, CH(CH₃)OH, cyclopropyl, cyclobutyl, and cyclopentyl. In some embodiments wherein B is CH, each R^{A} is independently selected from Cl, Br, F, methyl, ethyl, n-propyl, isopropyl, isobutyl, sec-butyl, CF₃, CF₂CH₃, CH₂CH₂F, CH₂CHF₂, cyclopropyl, cyclobutyl, and cyclopentyl. In other embodiments, wherein B is N, each R^{A} is independently selected from Cl, F, methyl, ethyl, n-propyl, isopropyl, sec-butyl, CF₂CH₃, CH₂CH₂F, CH₂CHF₂, CH₂OH, CH(CH₃)OH, cyclopropyl, and cyclobutyl. In some such embodiments, each R^{A} is independently selected from ethyl, isopropyl and cyclopropyl.

In some embodiments of compounds of formula (I), b is 0 or 1. In some embodiments of compounds of formula (I), R^{B} is methyl. In some embodiments of compounds of formula (I), R^{C} is CF₃ or Cl.

In some embodiments of compounds of formula (I), R⁵ and R⁶ are methyl, or R⁵ and R⁶, together with the carbon atom to which they are attached, form a cyclopropyl, cyclobutyl, tetrahydrofuranyl, or tetrahydropyranyl. In some embodiments, R⁵ and R⁶ are methyl, or R⁵ and R⁶, together with the carbon atom to which they are attached, form a cyclobutyl, or tetrahydrofuranyl. In some embodiments, R⁵ and R⁶ are methyl, or R⁵ and R⁶, together with the carbon atom to which they are attached, form a cyclobutyl, cyclopentyl, cyclohexyl, or tetrahydrofuranyl.

In some embodiments of compounds of formula (III), (IV) or (V), each R¹ is independently selected from Cl, F, CN, and CH₃, and R² and R³ are H. In some such embodiments, each R⁴ is independently selected from methyl, CF₃, and CH₂OH, or two R⁴ groups, together with the same carbon atom to which they are attached, form an unsubstituted cyclopropyl. In some such embodiments, R⁴ is methyl. In some such embodiments, R⁴ is ethyl. In some other such embodiments, L is —O(CH₂)(CH₂)—, or —(CH₂)(CH₂)(CH₂)—. In some other such embodiments, L is —O(CH₂)(CH(CH₃))—, —O(CH(CH₃))(CH₂)—. In still other such embodiments, A is CH. In yet other such embodiments, B is CH, and each R^{A} is independently selected from Cl, Br, F, methyl, ethyl, n-propyl, isopropyl, isobutyl, sec-butyl, CF₃, CF₂CH₃, CH₂CH₂F, CH₂CHF₂, cyclopropyl, cyclobutyl, and cyclopentyl. In other such embodiments, B is N, and each R^{A} is independently selected from Cl, F, methyl, ethyl, n-propyl, isopropyl, sec-butyl, CF₂CH₃, CH₂CH₂F, CH₂CHF₂, CH₂OH, CH(CH₃)OH, and cyclopropyl. In some other such embodiments, each R^{A} is ethyl, isopropyl or cyclopropyl. In still other such embodiments, R⁵ and R⁶ are methyl, or R⁵ and R⁶, together with the carbon atom to which they are attached, form a cyclobutyl, or tetrahydrofuranyl. In some embodiments, R⁵ and R⁶ are methyl, or R⁵ and R⁶, together with the carbon atom to which they are attached, form a cyclobutyl, cyclopentyl, cyclohexyl, or tetrahydrofuranyl.

In some embodiments of compounds of formula (I), (IIa), and (IIb),
R^{N} is H;
each R¹ is independently selected from Cl, F, Br, CN, —CH₃, —CH₂CH₃, and isopropyl;
R² and R³ are each independently selected from H, substituted or unsubstituted methyl and ethyl, or R² and R³ and the carbon to which they are attached form a substituted or unsubstituted cyclopropyl, cyclobutyl or cyclopentyl;
each R⁴ is independently selected from substituted or unsubstituted methyl or ethyl, or two R⁴ groups, together with the same carbon atom or adjacent carbon atoms to which they are attached, form a substituted or unsubstituted cyclopropyl or cyclobutyl, or two R⁴ groups together with the non-adjacent carbon atoms to which they are attached form a substituted or unsubstituted 4-7-membered heterocyclyl;
X is N;
L is —O(C₁-5 alkyl)- or -(C₁-5 alkyl)-;
n is 0-4;
m is 0-2;
V is

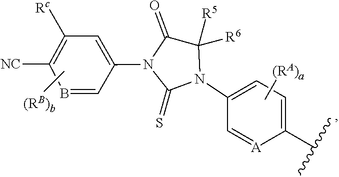

wherein
A is N, CH, or CR^{A};
B is N, CH, or CR^{B};
each R^{A} is independently selected from Cl, Br, F, methyl, ethyl, n-propyl, isopropyl, n-butyl, isobutyl, sec-butyl, tert-butyl, n-pentyl, CH₂CH₂CH(CH₃)₂, CH(CH₃)CH(CH₃)₂, CF₃, CF₂CH₃, CH₂CH₂F, CH₂CHF₂, CH₂CF₃, CH₂OH, CH(CH₃)OH, CH₂CH₂OH, CH(CH₃)CH₂OH, CH₂CH(CH₃)OH, cyclopropyl, cyclobutyl, and cyclopentyl;
each R^{B} is independently selected from halogen, and methyl;
R^{C} is halogen or CF₃;
R⁵ and R⁶ are methyl, or R⁵ and R⁶, together with the carbon atom to which they are attached, form a substituted or unsubstituted cyclobutyl, cyclopentyl, cyclohexyl, or tetrahydrofuranyl;
a is 0-3; and
b is 0-2.

In some embodiments of compounds of formula (I), (IIa), and (IIb),
R^{N} is H;
each R¹ is independently selected from Cl, F, Br, CN, —CH₃, —CH₂CH₃, and isopropyl;
R² and R³ are each independently selected from H, substituted or unsubstituted methyl and ethyl, or R² and R³ and the carbon to which they are attached form a substituted or unsubstituted cyclopropyl, cyclobutyl or cyclopentyl;
each R⁴ is independently selected from substituted or unsubstituted methyl or ethyl, or two R⁴ groups, together with the same carbon atom or adjacent carbon atoms to which they are attached, form a substituted or unsubstituted cyclopropyl or cyclobutyl, or two R⁴ groups together with the non-adjacent carbon atoms to which they are attached form a substituted or unsubstituted 4-7-membered heterocyclyl;
X is N;
L is —O(CH₂)ₚ— or —(CH₂)ₚ—;
n is 0-4;
m is 0-2;
p is 1-3;
V is

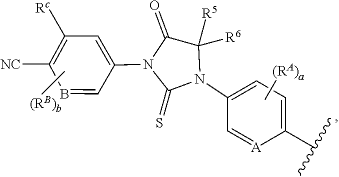

wherein
A is N, CH, or CR^{A};
B is N, CH, or CR^{B};
each R^{A} is independently selected from Cl, Br, F, methyl, ethyl, n-propyl, isopropyl, n-butyl, isobutyl, sec-butyl, tert-butyl, n-pentyl, CH₂CH₂CH(CH₃)₂, CH(CH₃)CH(CH₃)₂, CF₃, CF₂CH₃, CH₂CH₂F, CH₂CHF₂, CH₂CF₃, CH₂OH, CH(CH₃)OH, CH₂CH₂OH, CH(CH₃)CH₂OH, CH₂CH(CH₃)OH, cyclopropyl, cyclobutyl, and cyclopentyl;
each R^{B} is independently selected from halogen, and methyl;
R^{C} is halogen or CF₃;
R⁵ and R⁶ are methyl, or R⁵ and R⁶, together with the carbon atom to which they are attached, form a substituted or unsubstituted cyclobutyl, or tetrahydrofuranyl;
a is 0-3; and
b is 0-2.

In some embodiments of compounds of formula (III), (IV) and (V),
R^{N} is H;
each R¹ is independently selected from Cl, F, Br, CN, —CH₃, —CH₂CH₃, and isopropyl;
R² and R³ are each independently selected from H, substituted or unsubstituted methyl and ethyl, or R² and R³ and the carbon to which they are attached form a substituted or unsubstituted cyclopropyl, cyclobutyl or cyclopentyl;
each R⁴ is independently selected from substituted or unsubstituted methyl or ethyl, or two R⁴ groups, together with the same carbon atom or adjacent carbon atoms to which they are attached, form a substituted or unsubstituted cyclopropyl or cyclobutyl, or two R⁴ groups together with the non-adjacent carbon atoms to which they are attached form a substituted or unsubstituted 4-7-membered heterocyclyl;
X is N;
L is —O(C₁₋₅ alkyl)- or —(C₁₋₅ alkyl)-;
n is 0-4;
m is 0, 1 or 2;
V is

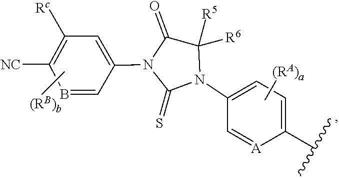

wherein
A is N, CH, or CR^{A};
B is N, CH, or CR^{B};
each R^{A} is independently selected from Cl, Br, F, methyl, ethyl, n-propyl, isopropyl, n-butyl, isobutyl, sec-butyl, tert-butyl, n-pentyl, CH₂CH₂CH(CH₃)₂, CH(CH₃)CH(CH₃)₂, CF₃, CF₂CH₃, CH₂CH₂F, CH₂CHF₂, CH₂CF₃, CH₂OH, CH(CH₃)OH, CH₂CH₂OH, CH(CH₃)CH₂OH, CH₂CH(CH₃)OH, cyclopropyl, cyclobutyl, and cyclopentyl;
each R^{B} is independently selected from halogen, and methyl;
R^{C} is halogen or CF₃;
R⁵ and R⁶ are methyl, or R⁵ and R⁶, together with the carbon atom to which they are attached, form a substituted or unsubstituted cyclobutyl, cyclopentyl, cyclohexyl, or tetrahydrofuranyl;
a is 0-3; and
b is 0-2.

In some embodiments of compounds of formula (III), (IV) and (V),
R^{N} is H;
each R¹ is independently selected from Cl, F, Br, CN, —CH₃, —CH₂CH₃, and isopropyl;
R² and R³ are each independently selected from H, substituted or unsubstituted methyl and ethyl, or R² and R³ and the carbon to which they are attached form a substituted or unsubstituted cyclopropyl, cyclobutyl or cyclopentyl;
each R⁴ is independently selected from substituted or unsubstituted methyl or ethyl, or two R⁴ groups, together with the same carbon atom or adjacent carbon atoms to which they are attached, form a substituted or unsubstituted cyclopropyl or cyclobutyl, or two R⁴ groups together with the non-adjacent carbon atoms to which they are attached form a substituted or unsubstituted 4-7-membered heterocycle;
X is N;
L is —O(CH₂)ₚ— or —(CH₂)ₚ—;
n is 0-4;
m is 0, 1 or 2;
p is 1-3;
V is

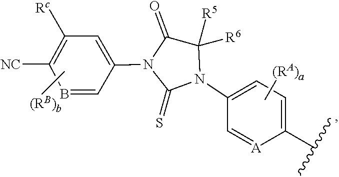

wherein
A is N, CH, or CR^{A};
B is N, CH, or CR^{B};
each R^{A} is independently selected from Cl, Br, F, methyl, ethyl, n-propyl, isopropyl, n-butyl, isobutyl, sec-butyl, tert-butyl, n-pentyl, CH₂CH₂CH(CH₃)₂, CH(CH₃)CH(CH₃)₂, CF₃, CF₂CH₃, CH₂CH₂F, CH₂CHF₂, CH₂CF₃, CH₂OH, CH(CH₃)OH, CH₂CH₂OH, CH(CH₃)CH₂OH, CH₂CH(CH₃)OH, cyclopropyl, cyclobutyl, and cyclopentyl;
each R^{B} is independently selected from halogen, and methyl;
R_{C} is halogen or CF₃;
R⁵ and R⁶ are methyl, or R⁵ and R⁶, together with the carbon atom to which they are attached, form a substituted or unsubstituted cyclobutyl, or tetrahydrofuranyl;
a is 0-3; and
b is 0-2.

Further embodiments provided herein include any combination of one or more of the particular embodiments set forth above.

In some embodiments of compounds of formula (I), the compound is a compound from Table 1.

Representative compounds of formula (I), (II), (III), (IV), (V), (VI), (VII), (VIII), (IX) or (X) are set forth in Table 1.

TABLE 1

| Cmpd | | |
|---|---|---|
| No. | Cmpd Structure | Cmpd Name |
| 1 | 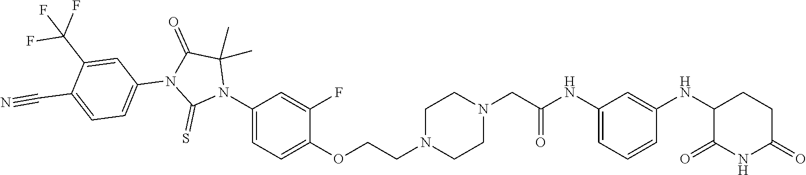 [SMILES: CC1(C)C(=O)N(c2ccc(C#N)c(C(F)(F)F)c2)C(=S)N1c1ccc(OCCN2CCN(CC(=O)Nc3cccc(NC4CCC(=O)NC4=O)c3)CC2)c(F)c1] | 2-(4-(2-(4-(3-(4-cyano-3-(trifluoromethyl)phenyl)- 5,5-dimethyl-4-oxo-2-thioxoimidazolidin-1-yl)-2- fluorophenoxy)ethyl)piperazin-1-yl)-N-(3-(2,6- dioxopiperidin-3-ylamino)phenyl)acetamide |
| 2 | 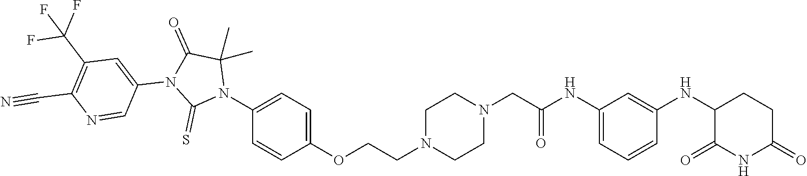 [SMILES: CC1(C)C(=O)N(c2cnc(C#N)c(C(F)(F)F)c2)C(=S)N1c1ccc(OCCN2CCN(CC(=O)Nc3cccc(NC4CCC(=O)NC4=O)c3)CC2)cc1] | 2-(4-(2-(4-(3-(6-cyano-5-(trifluoromethyl)pyridin- 3-yl)-5,5-dimethyl-4-oxo-2-thioxoimidazolidin-1- yl)phenoxy)ethyl)piperazin-1-yl)-N-(3-(2,6-dioxo- piperidin-3-ylamino)phenyl)acetamide |
| 3 | 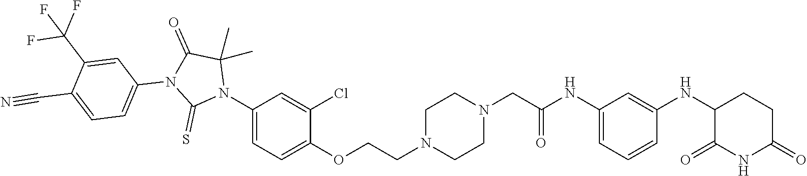 [SMILES: CC1(C)C(=O)N(c2ccc(C#N)c(C(F)(F)F)c2)C(=S)N1c1ccc(OCCN2CCN(CC(=O)Nc3cccc(NC4CCC(=O)NC4=O)c3)CC2)c(Cl)c1] | 2-(4-(2-(2-chloro-4-(3-(4-cyano-3-(trifluorometh- yl)phenyl)-5,5-dimethyl-4-oxo-2-thioxoimidazol- idin-1-yl)phenoxy)ethyl)piperazin-1-yl)-N-(3-(2,6- dioxopiperidin-3-ylamino)phenyl)acetamide |
| 4 | 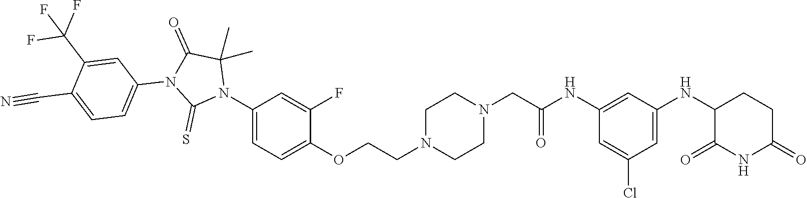 [SMILES: CC1(C)C(=O)N(c2ccc(C#N)c(C(F)(F)F)c2)C(=S)N1c1ccc(OCCN2CCN(CC(=O)Nc3cc(Cl)cc(NC4CCC(=O)NC4=O)c3)CC2)c(F)c1] | N-(3-chloro-5-(2,6-dioxopiperidin-3-ylamino)- phenyl)-2-(4-(2-(4-(3-(4-cyano-3-(trifluorometh- yl)phenyl)-5,5-dimethyl-4-oxo-2-thioxoimidazol- idin-1-yl)-2-fluorophenoxy)ethyl)piperazin-1-yl)- acetamide |
| 5 | 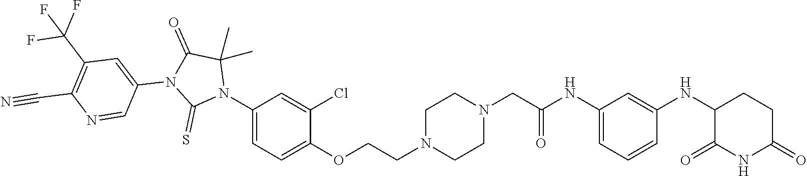 [SMILES: CC1(C)C(=O)N(c2cnc(C#N)c(C(F)(F)F)c2)C(=S)N1c1ccc(OCCN2CCN(CC(=O)Nc3cccc(NC4CCC(=O)NC4=O)c3)CC2)c(Cl)c1] | 2-(4-(2-(2-chloro-4-(3-(6-cyano-5-(trifluorometh- yl)pyridin-3-yl)-5,5-dimethyl-4-oxo-2-thioxoimid- azolidin-1-yl)phenoxy)ethyl)piperazin-1-yl)-N-(3- (2,6-dioxopiperidin-3-ylamino)phenyl)acetamide |
| 6 | 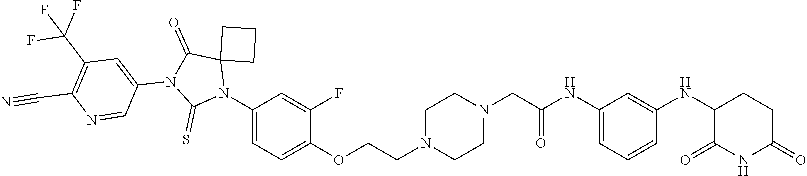 [SMILES: N#Cc1ncc(N2C(=O)C3(CCC3)N(c3ccc(OCCN4CCN(CC(=O)Nc5cccc(NC6CCC(=O)NC6=O)c5)CC4)c(F)c3)C2=S)cc1C(F)(F)F] | 2-(4-(2-(4-(7-(6-cyano-5-(trifluoromethyl)pyridin- 3-yl)-8-oxo-6-thioxo-5,7-diazaspiro[3.4]octan-5- yl)-2-fluorophenoxy)ethyl)piperazin-1-yl)-N-(3- (2,6-dioxopiperidin-3-ylamino)phenyl)acetamide |
| 7 | 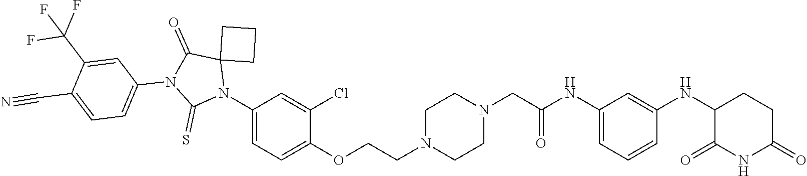 [SMILES: N#Cc1ccc(N2C(=O)C3(CCC3)N(c3ccc(OCCN4CCN(CC(=O)Nc5cccc(NC6CCC(=O)NC6=O)c5)CC4)c(Cl)c3)C2=S)cc1C(F)(F)F] | 2-(4-(2-(2-chloro-4-(7-(4-cyano-3-(trifluorometh- yl)phenyl)-8-oxo-6-thioxo-5,7-diazaspiro[3.4] octan-5-yl)phenoxy)ethyl)piperazin-1-yl)-N-(3- (2,6-dioxopiperidin-3-ylamino)phenyl)acetamide |
| 8 | 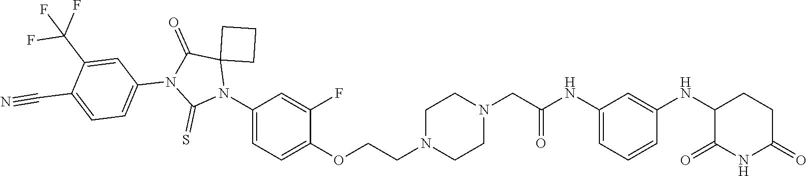 [SMILES: N#Cc1ccc(N2C(=O)C3(CCC3)N(c3ccc(OCCN4CCN(CC(=O)Nc5cccc(NC6CCC(=O)NC6=O)c5)CC4)c(F)c3)C2=S)cc1C(F)(F)F] | 2-(4-(2-(4-(7-(4-cyano-3-(trifluoromethyl)phenyl)- 8-oxo-6-thioxo-5,7-diazaspiro[3.4]octan-5-yl)-2- fluorophenoxy)ethyl)piperazin-1-yl)-N-(3-(2,6- dioxopiperidin-3-ylamino)phenyl)acetamide |
| 9 | 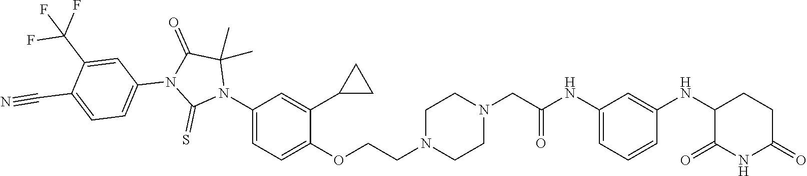 [SMILES: CC1(C)C(=O)N(c2ccc(C#N)c(C(F)(F)F)c2)C(=S)N1c1ccc(OCCN2CCN(CC(=O)Nc3cccc(NC4CCC(=O)NC4=O)c3)CC2)c(C2CC2)c1] | 2-(4-(2-(4-(3-(4-cyano-3-(trifluoromethyl)phenyl)- 5,5-dimethyl-4-oxo-2-thioxoimidazolidin-1-yl)-2- cyclopropylphenoxy)ethyl)piperazin-1-yl)-N-(3- (2,6-dioxopiperidin-3-ylamino)phenyl)acetamide |
| 10 | 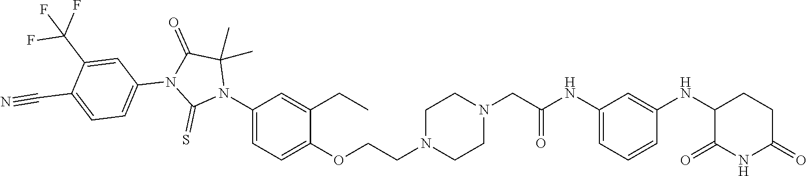 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)CC1] | 2-(4-(2-(4-(3-(4-cyano-3-(trifluoromethyl)phenyl)- 5,5-dimethyl-4-oxo-2-thioxoimidazolidin-1-yl)-2- ethylphenoxy)ethyl)piperazin-1-yl)-N-(3-(2,6- dioxopiperidin-3-ylamino)phenyl)acetamide |
| 11 | 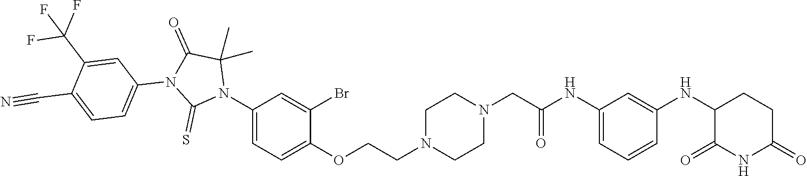 [SMILES: CC1(C)C(=O)N(c2ccc(C#N)c(C(F)(F)F)c2)C(=S)N1c1ccc(OCCN2CCN(CC(=O)Nc3cccc(NC4CCC(=O)NC4=O)c3)CC2)c(Br)c1] | 2-(4-(2-(2-bromo-4-(3-(4-cyano-3-(trifluorometh- yl)phenyl)-5,5-dimethyl-4-oxo-2-thioxoimidazol- idin-1-yl)phenoxy)ethyl)piperazin-1-yl)-N-(3-(2,6- dioxopiperidin-3-ylamino)phenyl)acetamide |
| 12 | 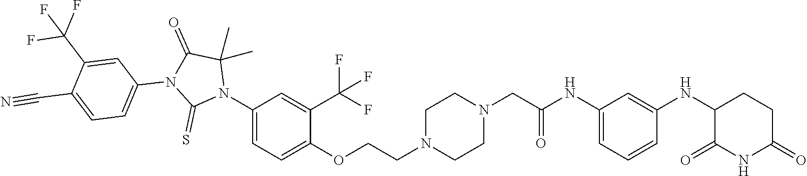 [SMILES: CC1(C)C(=O)N(c2ccc(C#N)c(C(F)(F)F)c2)C(=S)N1c1ccc(OCCN2CCN(CC(=O)Nc3cccc(NC4CCC(=O)NC4=O)c3)CC2)c(C(F)(F)F)c1] | 2-(4-(2-(4-(3-(4-cyano-3-(trifluoromethyl)phenyl)- 5,5-dimethyl-4-oxo-2-thioxoimidazolidin-1-yl)-2- (trifluoromethyl)phenoxy)ethyl)piperazin-1-yl)- N-(3-(2,6-dioxopiperidin-3-ylamino)phenyl)- acetamide |
| 13 | 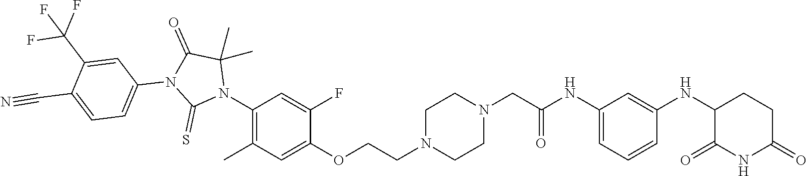 [SMILES: Cc1cc(OCCN2CCN(CC(=O)Nc3cccc(NC4CCC(=O)NC4=O)c3)CC2)c(F)cc1N1C(=S)N(c2ccc(C#N)c(C(F)(F)F)c2)C(=O)C1(C)C] | 2-(4-(2-(4-(3-(4-cyano-3-(trifluoromethyl)phenyl)- 5,5-dimethyl-4-oxo-2-thioxoimidazolidin-1-yl)-2- fluoro-5-methylphenoxy)ethyl)piperazin-1-yl)- N-(3-(2,6-dioxopiperidin-3-ylamino)phenyl)- acetamide |
| 14 | 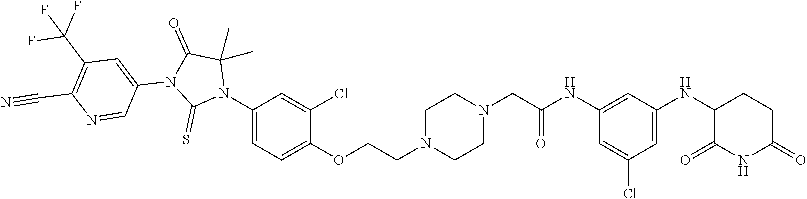 [SMILES: CC1(C)C(=O)N(c2cnc(C#N)c(C(F)(F)F)c2)C(=S)N1c1ccc(OCCN2CCN(CC(=O)Nc3cc(Cl)cc(NC4CCC(=O)NC4=O)c3)CC2)c(Cl)c1] | 2-(4-(2-(2-chloro-4-(3-(6-cyano-5-(trifluorometh- yl)pyridin-3-yl)-5,5-dimethyl-4-oxo-2-thioxoimid- azolidin-1-yl)phenoxy)ethyl)piperazin-1-yl)-N-(3- chloro-5-(2,6-dioxopiperidin-3-ylamino)phenyl)- acetamide |
| 15 | 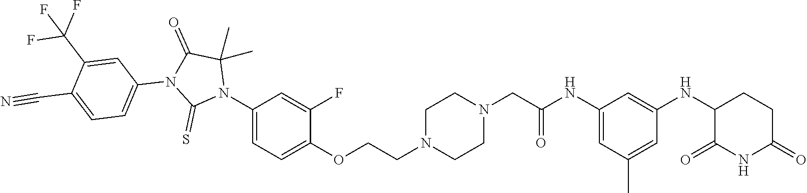 [SMILES: Cc1cc(NC(=O)CN2CCN(CCOc3ccc(N4C(=S)N(c5ccc(C#N)c(C(F)(F)F)c5)C(=O)C4(C)C)cc3F)CC2)cc(NC2CCC(=O)NC2=O)c1] | 2-(4-(2-(4-(3-(4-cyano-3-(trifluoromethyl)phenyl)- 5,5-dimethyl-4-oxo-2-thioxoimidazolidin-1-yl)- 2-fluorophenoxy)ethyl)piperazin-1-yl)-N-(3- (2,6-dioxopiperidin-3-ylamino)-5-methylphenyl)- acetamide |
| 16 | 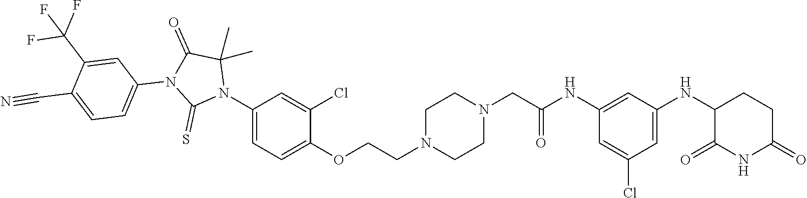 [SMILES: CC1(C)C(=O)N(c2ccc(C#N)c(C(F)(F)F)c2)C(=S)N1c1ccc(OCCN2CCN(CC(=O)Nc3cc(Cl)cc(NC4CCC(=O)NC4=O)c3)CC2)c(Cl)c1] | 2-(4-(2-(2-chloro-4-(3-(4-cyano-3-(trifluorometh- yl)phenyl)-5,5-dimethyl-4-oxo-2-thioxoimid- azolidin-1-yl)phenoxy)ethyl)piperazin-1-yl)-N- (3-chloro-5-(2,6-dioxopiperidin-3-ylamino)phen- yl)acetamide |
| 17 | 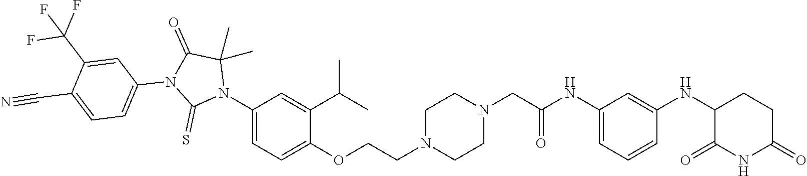 [SMILES: CC(C)c1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)CC1] | 2-(4-(2-(4-(3-(4-cyano-3-(trifluoromethyl)phenyl)- 5,5-dimethyl-4-oxo-2-thioxoimidazolidin-1-yl)-2- isopropylphenoxy)ethyl)piperazin-1-yl)-N-(3- (2,6-dioxopiperidin-3-ylamino)phenyl)acetamide |
| 18 | 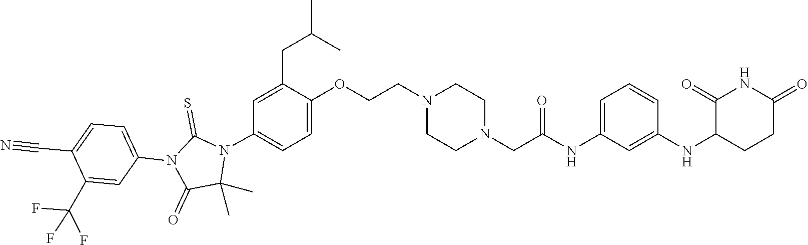 [SMILES: CC(C)Cc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)CC1] | 2-(4-(2-(4-(3-(4-cyano-3-(trifluoromethyl)phenyl)- 5,5-dimethyl-4-oxo-2-thioxoimidazolidin-1-yl)-2- isobutylphenoxy)ethyl)piperazin-1-yl)-N-(3-(2,6- dioxopiperidin-3-ylamino)phenyl)acetamide |
| 19 | 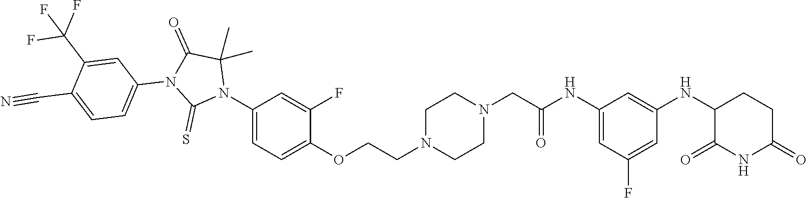 [SMILES: CC1(C)C(=O)N(c2ccc(C#N)c(C(F)(F)F)c2)C(=S)N1c1ccc(OCCN2CCN(CC(=O)Nc3cc(F)cc(NC4CCC(=O)NC4=O)c3)CC2)c(F)c1] | 2-(4-(2-(4-(3-(4-cyano-3-(trifluoromethyl)phenyl)- 5,5-dimethyl-4-oxo-2-thioxoimidazolidin-1-yl)- 2-fluorophenoxy)ethyl)piperazin-1-yl)-N-(3- (2,6-dioxopiperidin-3-ylamino)-5-fluorophenyl)- acetamide |
| 20 | 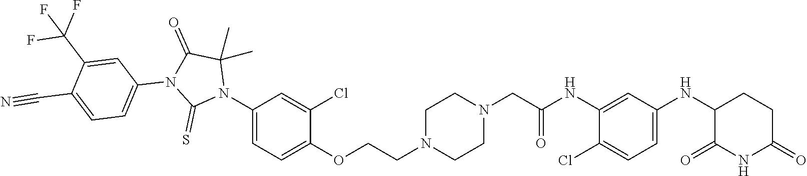 [SMILES: CC1(C)C(=O)N(c2ccc(C#N)c(C(F)(F)F)c2)C(=S)N1c1ccc(OCCN2CCN(CC(=O)Nc3cc(NC4CCC(=O)NC4=O)ccc3Cl)CC2)c(Cl)c1] | 2-(4-(2-(2-chloro-4-(3-(4-cyano-3-(trifluorometh- yl)phenyl)-5,5-dimethyl-4-oxo-2-thioxoimidazol- idin-1-yl)phenoxy)ethyl)piperazin-1-yl)-N-(2- chloro-5-(2,6-dioxopiperidin-3-ylamino)phenyl)- acetamide |
| 21 | 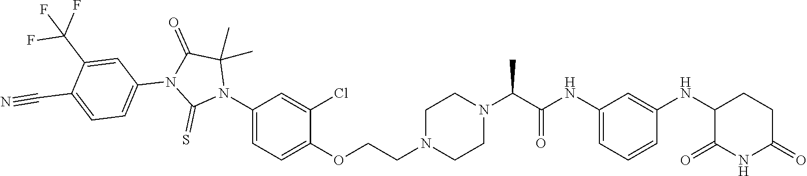 [SMILES: C[C@@H](C(=O)Nc1cccc(NC2CCC(=O)NC2=O)c1)N1CCN(CCOc2ccc(N3C(=S)N(c4ccc(C#N)c(C(F)(F)F)c4)C(=O)C3(C)C)cc2Cl)CC1] | (2S)-2-(4-(2-(2-chloro-4-(3-(4-cyano-3-(trifluoro- methyl)phenyl)-5,5-dimethyl-4-oxo-2-thioxoimid- azolidin-1-yl)phenoxy)ethyl)piperazin-1-yl)-N- (3-(2,6-dioxopiperidin-3-ylamino)phenyl)propan- amide |
| 22 | 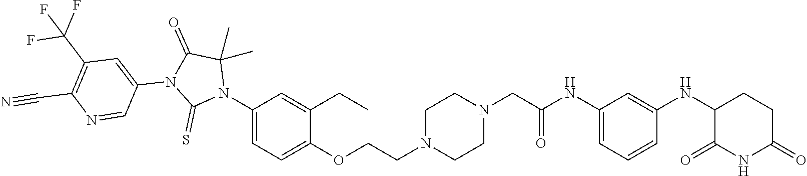 [SMILES: CCc1cc(N2C(=S)N(c3cnc(C#N)c(C(F)(F)F)c3)C(=O)C2(C)C)ccc1OCCN1CCN(CC(=O)Nc2cccc(NC3CCC(=O)NC3=O)c2)CC1] | 2-(4-(2-(4-(3-(6-cyano-5-(trifluoromethyl)pyridin- 3-yl)-5,5-dimethyl-4-oxo-2-thioxoimidazolidin-1- yl)-2-ethylphenoxy)ethyl)piperazin-1-yl)-N-(3- (2,6-dioxopiperidin-3-ylamino)phenyl)acetamide |
| 23 | 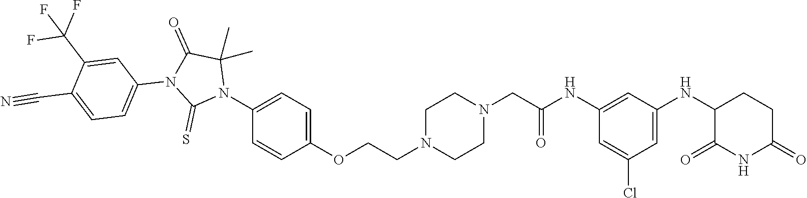 [SMILES: CC1(C)C(=O)N(c2ccc(C#N)c(C(F)(F)F)c2)C(=S)N1c1ccc(OCCN2CCN(CC(=O)Nc3cc(Cl)cc(NC4CCC(=O)NC4=O)c3)CC2)cc1] | N-(3-chloro-5-(2,6-dioxopiperidin-3-ylamino)- phenyl)-2-(4-(2-(4-(3-(4-cyano-3-(trifluorometh- yl)phenyl)-5,5-dimethyl-4-oxo-2-thioxoimidazol- idin-1-yl)phenoxy)ethyl)piperazin-1-yl)acetamide |
| 24 | 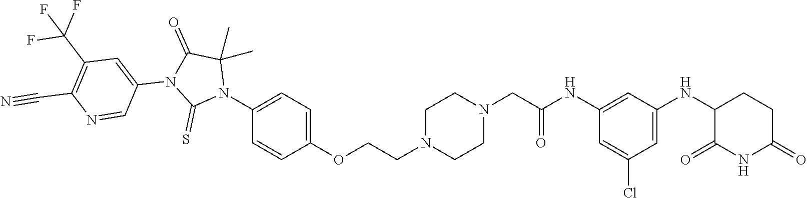 [SMILES: CC1(C)C(=O)N(c2cnc(C#N)c(C(F)(F)F)c2)C(=S)N1c1ccc(OCCN2CCN(CC(=O)Nc3cc(Cl)cc(NC4CCC(=O)NC4=O)c3)CC2)cc1] | N-(3-chloro-5-(2,6-dioxopiperidin-3-ylamino)- phenyl)-2-(4-(2-(4-(3-(6-cyano-5-(trifluorometh- yl)pyridin-3-yl)-5,5-dimethyl-4-oxo-2-thioxoimid- azolidin-1-yl)phenoxy)ethyl)piperazin-1-yl)- acetamide |
| 25 | 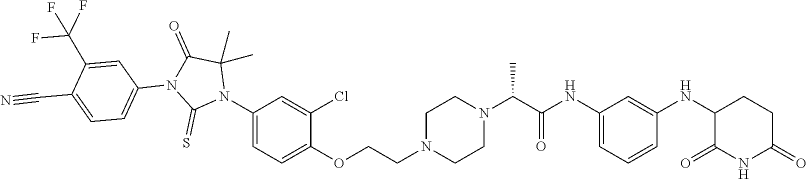 [SMILES: C[C@H](C(=O)Nc1cccc(NC2CCC(=O)NC2=O)c1)N1CCN(CCOc2ccc(N3C(=S)N(c4ccc(C#N)c(C(F)(F)F)c4)C(=O)C3(C)C)cc2Cl)CC1] | (2R)-2-(4-(2-(2-chloro-4-(3-(4-cyano-3-(trifluoro- methyl)phenyl)-5,5-dimethyl-4-oxo-2-thioxoimid- azolidin-1-yl)phenoxy)ethyl)piperazin-1-yl)-N- (3-(2,6-dioxopiperidin-3-ylamino)phenyl)propan- amide |
| 26 | 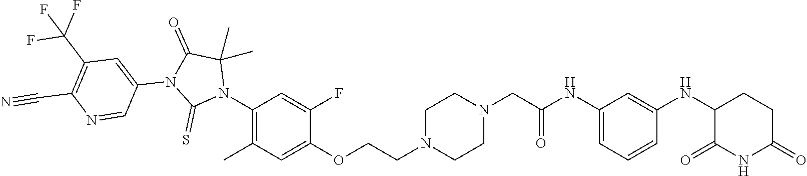 [SMILES: Cc1cc(OCCN2CCN(CC(=O)Nc3cccc(NC4CCC(=O)NC4=O)c3)CC2)c(F)cc1N1C(=S)N(c2cnc(C#N)c(C(F)(F)F)c2)C(=O)C1(C)C] | 2-(4-(2-(4-(3-(6-cyano-5-(trifluoromethyl)pyridin- 3-yl)-5,5-dimethyl-4-oxo-2-thioxoimidazolidin-1- yl)-2-fluoro-5-methylphenoxy)ethyl)piperazin-1- yl)-N-(3-(2,6-dioxopiperidin-3-ylamino)phenyl)- acetamide |
| 27 | 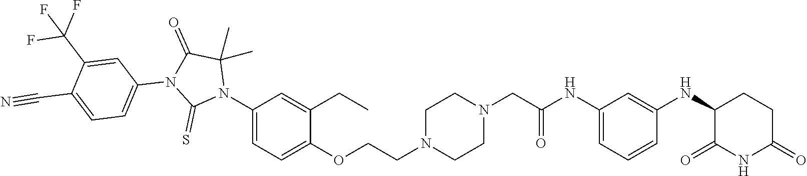 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@H]3CCC(=O)NC3=O)c2)CC1] | (S)-2-(4-(2-(4-(3-(4-cyano-3-(trifluoromethyl)- phenyl)-5,5-dimethyl-4-oxo-2-thioxoimidazolidin- 1-yl)-2-ethylphenoxy)ethyl)piperazin-1-yl)-N-(3- (2,6-dioxopiperidin-3-ylamino)phenyl)acetamide |
| 28 | 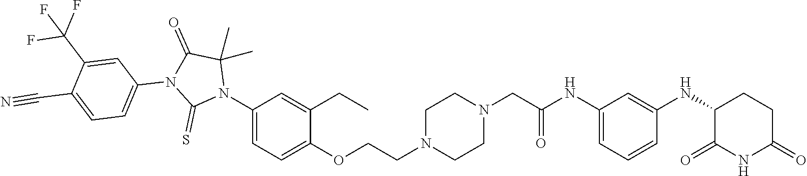 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)CC1] | (R)-2-(4-(2-(4-(3-(4-cyano-3-(trifluoromethyl)- phenyl)-5,5-dimethyl-4-oxo-2-thioxoimidazolidin- 1-yl)-2-ethylphenoxy)ethyl)piperazin-1-yl)-N-(3- (2,6-dioxopiperidin-3-ylamino)phenyl)acetamide |
| 29 | 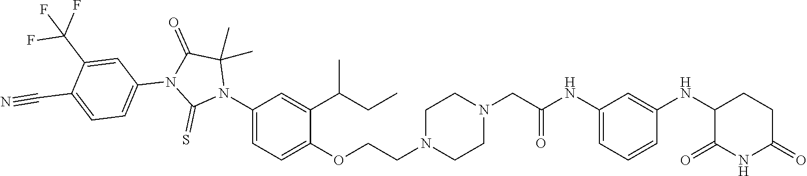 [SMILES: CCC(C)c1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)CC1] | 2-(4-(2-(2-sec-butyl-4-(3-(4-cyano-3-(trifluoro- methyl)phenyl)-5,5-dimethyl-4-oxo-2-thioxoimid- azolidin-1-yl)phenoxy)ethyl)piperazin-1-yl)-N- (3-(2,6-dioxopiperidin-3-ylamino)phenyl)- acetamide |
| 30 | 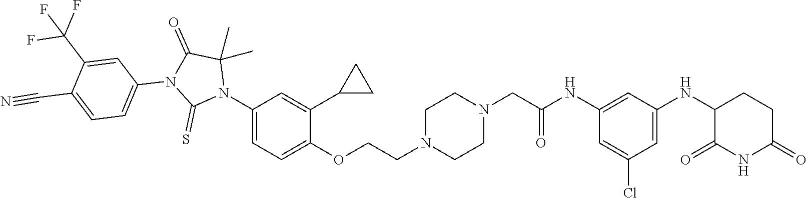 [SMILES: CC1(C)C(=O)N(c2ccc(C#N)c(C(F)(F)F)c2)C(=S)N1c1ccc(OCCN2CCN(CC(=O)Nc3cc(Cl)cc(NC4CCC(=O)NC4=O)c3)CC2)c(C2CC2)c1] | N-(3-chloro-5-(2,6-dioxopiperidin-3-ylamino)- phenyl)-2-(4-(2-(4-(3-(4-cyano-3-(trifluorometh- yl)phenyl)-5,5-dimethyl-4-oxo-2-thioxoimidazol- idin-1-yl)-2-cyclopropylphenoxy)ethyl)piperazin- 1-yl)acetamide |
| 31 | 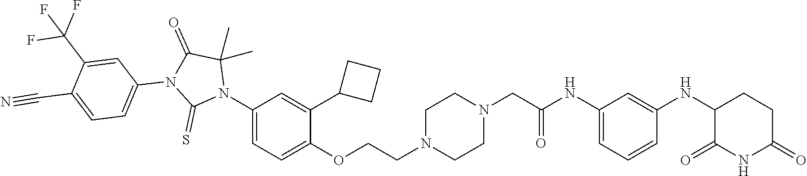 [SMILES: CC1(C)C(=O)N(c2ccc(C#N)c(C(F)(F)F)c2)C(=S)N1c1ccc(OCCN2CCN(CC(=O)Nc3cccc(NC4CCC(=O)NC4=O)c3)CC2)c(C2CCC2)c1] | 2-(4-(2-(4-(3-(4-cyano-3-(trifluoromethyl)phenyl)- 5,5-dimethyl-4-oxo-2-thioxoimidazolidin-1-yl)-2- cyclobutylphenoxy)ethyl)piperazin-1-yl)-N-(3- (2,6-dioxopiperidin-3-ylamino)phenyl)acetamide |
| 32 | 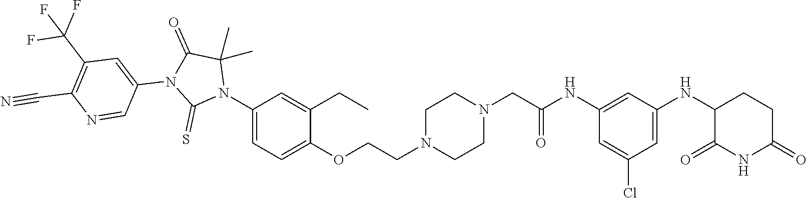 [SMILES: CCc1cc(N2C(=S)N(c3cnc(C#N)c(C(F)(F)F)c3)C(=O)C2(C)C)ccc1OCCN1CCN(CC(=O)Nc2cc(Cl)cc(NC3CCC(=O)NC3=O)c2)CC1] | N-(3-chloro-5-(2,6-dioxopiperidin-3-ylamino)- phenyl)-2-(4-(2-(4-(3-(6-cyano-5-(trifluorometh- yl)pyridin-3-yl)-5,5-dimethyl-4-oxo-2-thioxo- imidazolidin-1-yl)-2-ethylphenoxy)ethyl)pipera- zin-1-yl)acetamide |
| 33 | 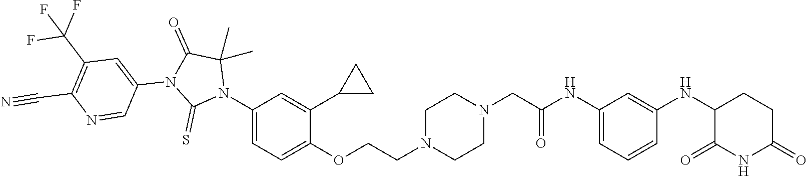 [SMILES: CC1(C)C(=O)N(c2cnc(C#N)c(C(F)(F)F)c2)C(=S)N1c1ccc(OCCN2CCN(CC(=O)Nc3cccc(NC4CCC(=O)NC4=O)c3)CC2)c(C2CC2)c1] | 2-(4-(2-(4-(3-(6-cyano-5-(trifluoromethyl)pyridin- 3-yl)-5,5-dimethyl-4-oxo-2-thioxoimidazolidin-1- yl)-2-cyclopropylphenoxy)ethyl)piperazin-1-yl)- N-(3-(2,6-dioxopiperidin-3-ylamino)phenyl)- acetamide |
| 34 | 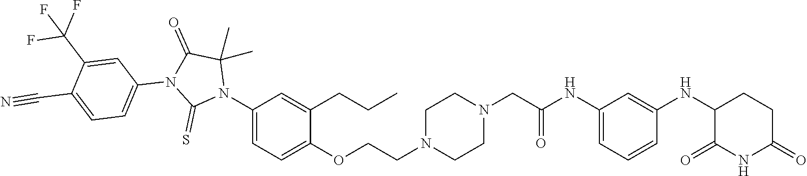 [SMILES: CCCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)CC1] | 2-(4-(2-(4-(3-(4-cyano-3-(trifluoromethyl)phenyl)- 5,5-dimethyl-4-oxo-2-thioxoimidazolidin-1-yl)-2- propylphenoxy)ethyl)piperazin-1-yl)-N-(3-(2,6- dioxopiperidin-3-ylamino)phenyl)acetamide |
| 35 | 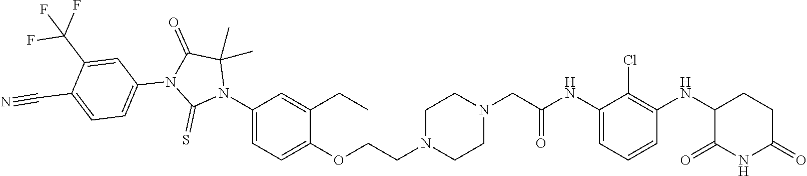 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2Cl)CC1] | N-(2-chloro-3-(2,6-dioxopiperidin-3-ylamino)- phenyl)-2-(4-(2-(4-(3-(4-cyano-3-(trifluorometh- yl)phenyl)-5,5-dimethyl-4-oxo-2-thioxoimidazol- idin-1-yl)-2-ethylphenoxy)ethyl)piperazin-1-yl)- acetamide |
| 36 | 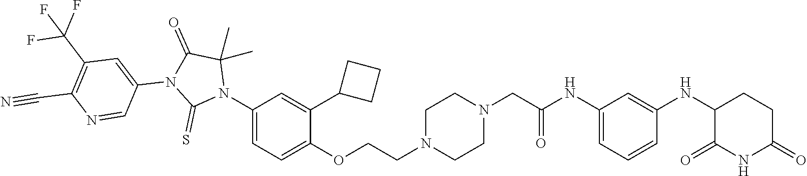 [SMILES: CC1(C)C(=O)N(c2cnc(C#N)c(C(F)(F)F)c2)C(=S)N1c1ccc(OCCN2CCN(CC(=O)Nc3cccc(NC4CCC(=O)NC4=O)c3)CC2)c(C2CCC2)c1] | 2-(4-(2-(4-(3-(6-cyano-5-(trifluoromethyl)pyridin- 3-yl)-5,5-dimethyl-4-oxo-2-thioxoimidazolidin-1- yl)-2-cyclobutylphenoxy)ethyl)piperazin-1-yl)- N-(3-(2,6-dioxopiperidin-3-ylamino)phenyl)- acetamide |
| 37 | 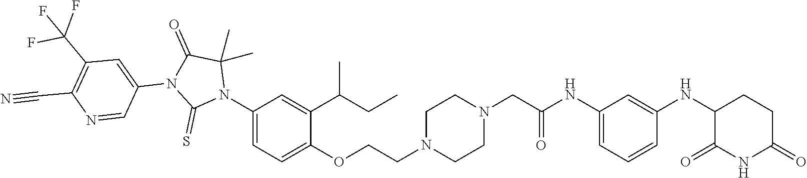 [SMILES: CCC(C)c1cc(N2C(=S)N(c3cnc(C#N)c(C(F)(F)F)c3)C(=O)C2(C)C)ccc1OCCN1CCN(CC(=O)Nc2cccc(NC3CCC(=O)NC3=O)c2)CC1] | 2-(4-(2-(2-sec-butyl-4-(3-(6-cyano-5-(trifluoro- methyl)pyridin-3-yl)-5,5-dimethyl-4-oxo-2- thioxoimidazolidin-1-yl)phenoxy)ethyl)piperazin- 1-yl)-N-(3-(2,6-dioxopiperidin-3-ylamino)phen- yl)acetamide |
| 38 | 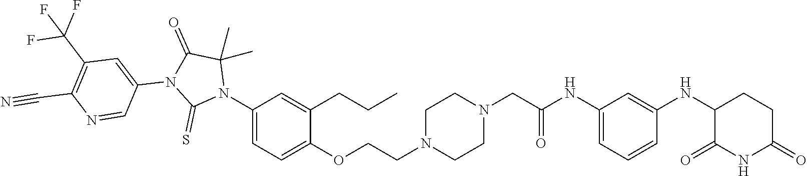 [SMILES: CCCc1cc(N2C(=S)N(c3cnc(C#N)c(C(F)(F)F)c3)C(=O)C2(C)C)ccc1OCCN1CCN(CC(=O)Nc2cccc(NC3CCC(=O)NC3=O)c2)CC1] | 2-(4-(2-(4-(3-(6-cyano-5-(trifluoromethyl)pyridin- 3-yl)-5,5-dimethyl-4-oxo-2-thioxoimidazolidin-1- yl)-2-propylphenoxy)ethyl)piperazin-1-yl)-N-(3- (2,6-dioxopiperidin-3-ylamino)phenyl)acetamide |
| 39 | 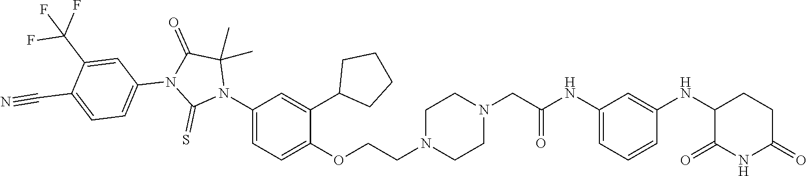 [SMILES: CC1(C)C(=O)N(c2ccc(C#N)c(C(F)(F)F)c2)C(=S)N1c1ccc(OCCN2CCN(CC(=O)Nc3cccc(NC4CCC(=O)NC4=O)c3)CC2)c(C2CCCC2)c1] | 2-(4-(2-(4-(3-(4-cyano-3-(trifluoromethyl)phenyl)- 5,5-dimethyl-4-oxo-2-thioxoimidazolidin-1-yl)-2- cyclopentylphenoxy)ethyl)piperazin-1-yl)-N-(3- (2,6-dioxopiperidin-3-ylamino)phenyl)acetamide |
| 40 | 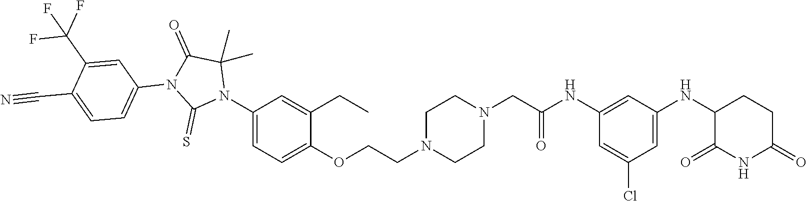 [SMILES: CCc1cc(N2C(=S)N(c3ccc(C#N)c(C(F)(F)F)c3)C(=O)C2(C)C)ccc1OCCN1CCN(CC(=O)Nc2cc(Cl)cc(NC3CCC(=O)NC3=O)c2)CC1] | N-(3-chloro-5-(2,6-dioxopiperidin-3-ylamino)- phenyl)-2-(4-(2-(4-(3-(4-cyano-3-(trifluorometh- yl)phenyl)-5,5-dimethyl-4-oxo-2-thioxoimidazol- idin-1-yl)-2-ethylphenoxy)ethyl)piperazin-1-yl)- acetamide |
| 41 | 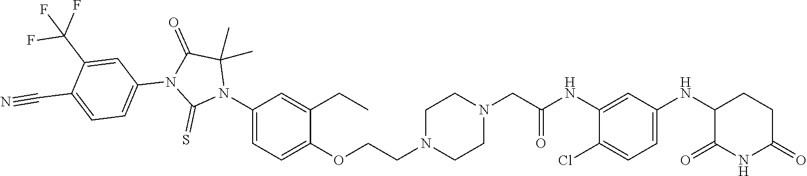 [SMILES: CCc1cc(N2C(=S)N(c3ccc(C#N)c(C(F)(F)F)c3)C(=O)C2(C)C)ccc1OCCN1CCN(CC(=O)Nc2cc(NC3CCC(=O)NC3=O)ccc2Cl)CC1] | N-(2-chloro-5-(2,6-dioxopiperidin-3-ylamino)- phenyl)-2-(4-(2-(4-(3-(4-cyano-3-(trifluorometh- yl)phenyl)-5,5-dimethyl-4-oxo-2-thioxoimidazol- idin-1-yl)-2-ethylphenoxy)ethyl)piperazin-1-yl)- acetamide |
| 42 | 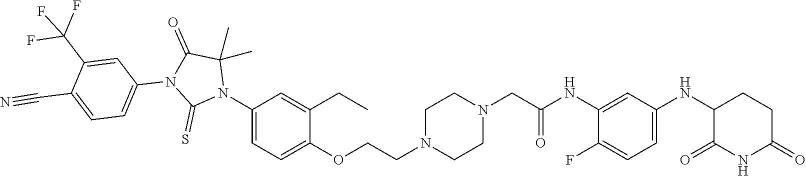 [SMILES: CCc1cc(N2C(=S)N(c3ccc(C#N)c(C(F)(F)F)c3)C(=O)C2(C)C)ccc1OCCN1CCN(CC(=O)Nc2cc(NC3CCC(=O)NC3=O)ccc2F)CC1] | 2-(4-(2-(4-(3-(4-cyano-3-(trifluoromethyl)phenyl)- 5,5-dimethyl-4-oxo-2-thioxoimidazolidin-1-yl)- 2-ethylphenoxy)ethyl)piperazin-1-yl)-N-(5-(2,6- dioxopiperidin-3-ylamino)-2-fluorophenyl)- acetamide |
| 43 | 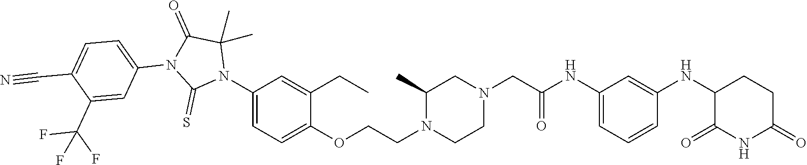 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)C[C@@H]1C] | 2-((S)-4-(2-(4-(3-(4-cyano-3-(trifluoromethyl)- phenyl)-5,5-dimethyl-4-oxo-2-thioxoimidazolidin- 1-yl)-2-ethylphenoxy)ethyl)-3-methylpiperazin-1- yl)-N-(3-(2,6-dioxopiperidin-3-ylamino)phenyl)- acetamide |
| 44 | 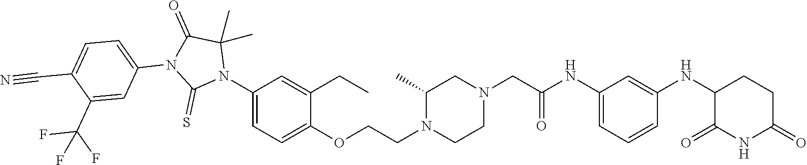 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)C[C@H]1C] | 2-((R)-4-(2-(4-(3-(4-cyano-3-(trifluoromethyl)- phenyl)-5,5-dimethyl-4-oxo-2-thioxoimidazolidin- 1-yl)-2-ethylphenoxy)ethyl)-3-methylpiperazin-1- yl)-N-(3-(2,6-dioxopiperidin-3-ylamino)phenyl)- acetamide |
| 45 | 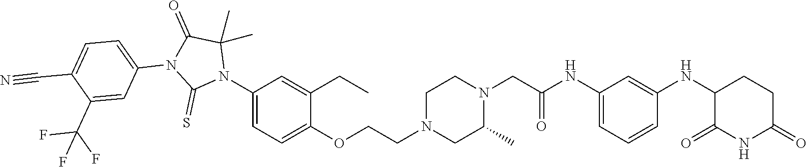 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)[C@H](C)C1] | 2-((R)-4-(2-(4-(3-(4-cyano-3-(trifluoromethyl)- phenyl)-5,5-dimethyl-4-oxo-2-thioxoimidazolidin- 1-yl)-2-ethylphenoxy)ethyl)-2-methylpiperazin-1- yl)-N-(3-(2,6-dioxopiperidin-3-ylamino)phenyl)- acetamide |
| 46 | 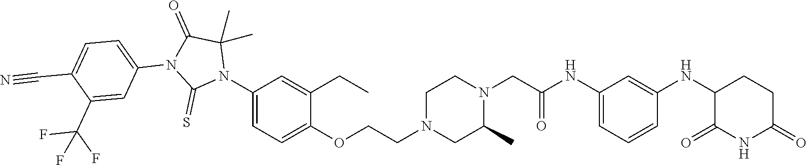 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)[C@@H](C)C1] | 2-((S)-4-(2-(4-(3-(4-cyano-3-(trifluoromethyl)- phenyl)-5,5-dimethyl-4-oxo-2-thioxoimidazolidin- 1-yl)-2-ethylphenoxy)ethyl)-2-methylpiperazin-1- yl)-N-(3-(2,6-dioxopiperidin-3-ylamino)phenyl)- acetamide |
| 47 | 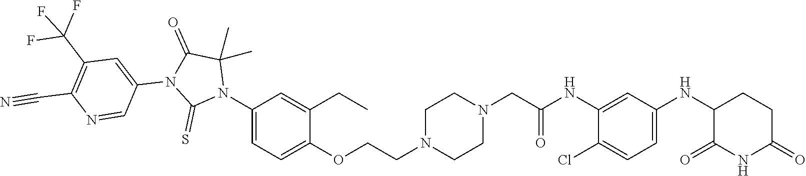 [SMILES: CCc1cc(N2C(=S)N(c3cnc(C#N)c(C(F)(F)F)c3)C(=O)C2(C)C)ccc1OCCN1CCN(CC(=O)Nc2cc(NC3CCC(=O)NC3=O)ccc2Cl)CC1] | N-(2-chloro-5-(2,6-dioxopiperidin-3-ylamino)- phenyl)-2-(4-(2-(4-(3-(6-cyano-5-(trifluorometh- yl)pyridin-3-yl)-5,5-dimethyl-4-oxo-2-thioxoimid- azolidin-1-yl)-2-ethylphenoxy)ethyl)piperazin-1- yl)acetamide |
| 48 | 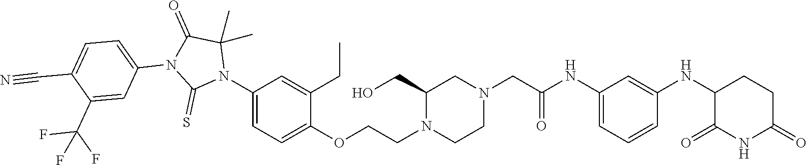 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)C[C@@H]1CO] | 2-((R)-4-(2-(4-(3-(4-cyano-3-(trifluoromethyl)- phenyl)-5,5-dimethyl-4-oxo-2-thioxoimidazolidin- 1-yl)-2-ethylphenoxy)ethyl)-3-(hydroxymethyl)- piperazin-1-yl)-N-(3-(2,6-dioxopiperidin-3-yl- amino)phenyl)acetamide |
| 49 | 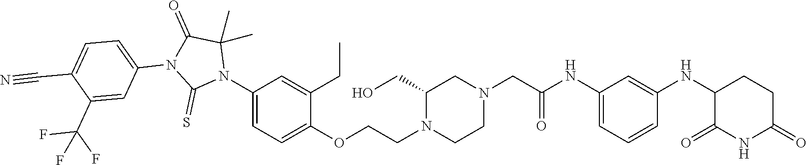 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)C[C@H]1CO] | 2-((S)-4-(2-(4-(3-(4-cyano-3-(trifluoromethyl)- phenyl)-5,5-dimethyl-4-oxo-2-thioxoimidazolidin- 1-yl)-2-ethylphenoxy)ethyl)-3-(hydroxymethyl)- piperazin-1-yl)-N-(3-(2,6-dioxopiperidin-3-yl- amino)phenyl)acetamide |
| 50 | 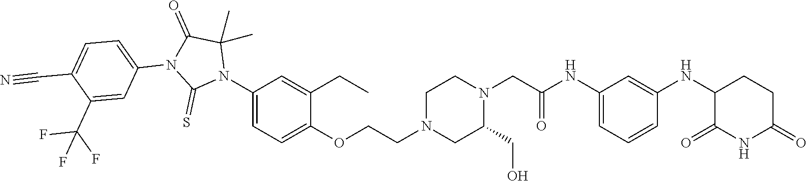 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)[C@H](CO)C1] | 2-((S)-4-(2-(4-(3-(4-cyano-3-(trifluoromethyl)- phenyl)-5,5-dimethyl-4-oxo-2-thioxoimidazolidin- 1-yl)-2-ethylphenoxy)ethyl)-2-(hydroxymethyl)- piperazin-1-yl)-N-(3-(2,6-dioxopiperidin-3-yl- amino)phenyl)acetamide |
| 51 | 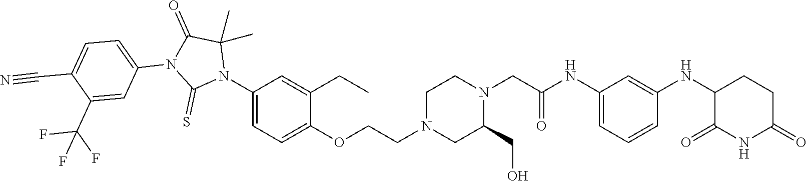 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)[C@@H](CO)C1] | 2-((R)-4-(2-(4-(3-(4-cyano-3-(trifluoromethyl)- phenyl)-5,5-dimethyl-4-oxo-2-thioxoimidazolidin- 1-yl)-2-ethylphenoxy)ethyl)-2-(hydroxymethyl)- piperazin-1-yl)-N-(3-(2,6-dioxopiperidin-3-yl- amino)phenyl)acetamide |
| 52 | 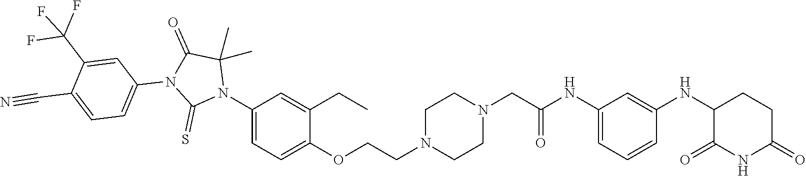 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)CC1] | 2-(4-(2-(4-(3-(4-cyano-3-(trifluoromethyl)phenyl)- 5,5-dimethyl-2,4-dioxoimidazolidin-1-yl)-2-ethyl- phenoxy)ethyl)piperazin-1-yl)-N-(3-(2,6-dioxo- piperidin-3-ylamino)phenyl)acetamide |
| 53 | 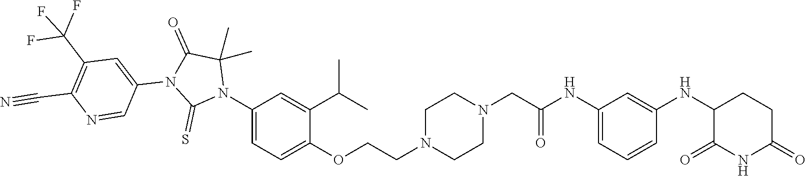 [SMILES: CC(C)c1cc(N2C(=S)N(c3cnc(C#N)c(C(F)(F)F)c3)C(=O)C2(C)C)ccc1OCCN1CCN(CC(=O)Nc2cccc(NC3CCC(=O)NC3=O)c2)CC1] | 2-(4-(2-(4-(3-(6-cyano-5-(trifluoromethyl)pyridin- 3-yl)-5,5-dimethyl-4-oxo-2-thioxoimidazolidin- 1-yl)-2-isopropylphenoxy)ethyl)piperazin-1-yl)- N-(3-(2,6-dioxopiperidin-3-ylamino)phenyl)- acetamide |
| 54 | 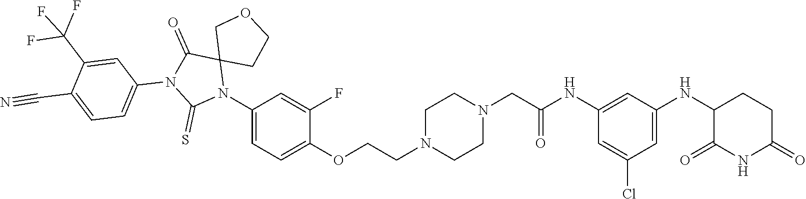 [SMILES: N#Cc1ccc(N2C(=O)C3(CCOC3)N(c3ccc(OCCN4CCN(CC(=O)Nc5cc(Cl)cc(NC6CCC(=O)NC6=O)c5)CC4)c(F)c3)C2=S)cc1C(F)(F)F] | N-(3-chloro-5-(2,6-dioxopiperidin-3-ylamino)- phenyl)-2-(4-(2-(4-(3-(4-cyano-3-(trifluorometh- yl)phenyl)-4-oxo-2-thioxo-7-oxa-1,3-diazaspiro [4.4]nonan-1-yl)-2-fluorophenoxy)ethyl)piper- azin-1-yl)acetamide (THP-enantiomer 1) |
| 55 | 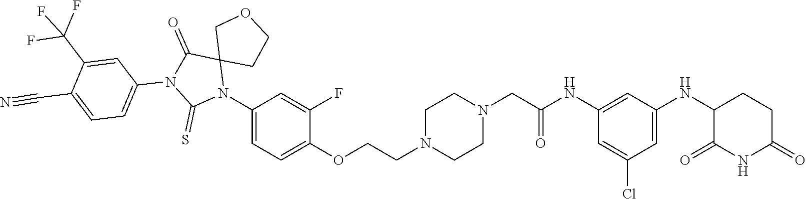 [SMILES: N#Cc1ccc(N2C(=O)C3(CCOC3)N(c3ccc(OCCN4CCN(CC(=O)Nc5cc(Cl)cc(NC6CCC(=O)NC6=O)c5)CC4)c(F)c3)C2=S)cc1C(F)(F)F] | N-(3-chloro-5-(2,6-dioxopiperidin-3-ylamino)- phenyl)-2-(4-(2-(4-(3-(4-cyano-3-(trifluorometh- yl)phenyl)-4-oxo-2-thioxo-7-(oxa-1,3-diazaspiro [4.4]nonan-1-yl)-2-fluorophenoxy)ethyl)piper- azin-1-yl)acetamide (THP-enantiomer 2) |
| 56 | 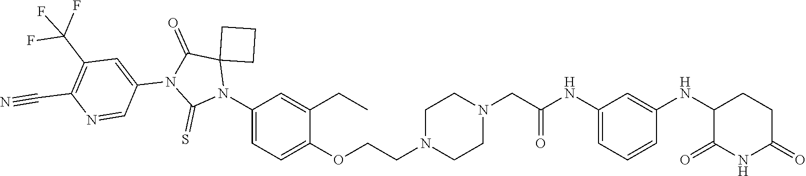 [SMILES: CCc1cc(N2C(=S)N(c3cnc(C#N)c(C(F)(F)F)c3)C(=O)C23CCC3)ccc1OCCN1CCN(CC(=O)Nc2cccc(NC3CCC(=O)NC3=O)c2)CC1] | 2-(4-(2-(4-(7-(6-cyano-5-(trifluoromethyl)pyridin- 3-yl)-8-oxo-6-thioxo-5,7-diazaspiro[3.4]octan-5- yl)-2-ethylphenoxy)ethyl)piperazin-1-yl)-N-(3- (2,6-dioxopiperidin-3-ylamino)phenyl)acetamide |
| 57 | 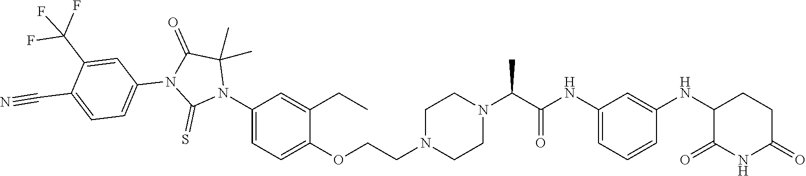 [SMILES: CCc1cc(N2C(=S)N(c3ccc(C#N)c(C(F)(F)F)c3)C(=O)C2(C)C)ccc1OCCN1CCN([C@@H](C)C(=O)Nc2cccc(NC3CCC(=O)NC3=O)c2)CC1] | (2S)-2-(4-(2-(4-(3-(4-cyano-3-(trifluoromethyl)- phenyl)-5,5-dimethyl-4-oxo-2-thioxoimidazolidin- 1-yl)-2-ethylphenoxy)ethyl)piperazin-1-yl)-N-(3- (2,6-dioxopiperidin-3-ylamino)phenyl)propan- amide |
| 58 | 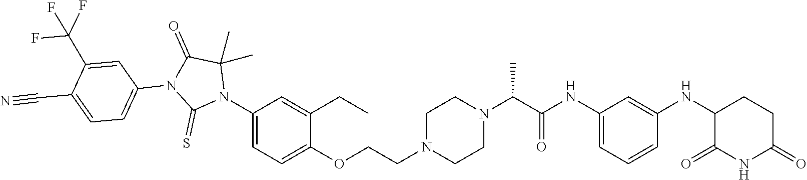 [SMILES: CCc1cc(N2C(=S)N(c3ccc(C#N)c(C(F)(F)F)c3)C(=O)C2(C)C)ccc1OCCN1CCN([C@H](C)C(=O)Nc2cccc(NC3CCC(=O)NC3=O)c2)CC1] | (2R)-2-(4-(2-(4-(3-(4-cyano-3-(trifluoromethyl)- phenyl)-5,5-dimethyl-4-oxo-2-thioxoimidazolidin- 1-yl)-2-ethylphenoxy)ethyl)piperazin-1-yl)-N-(3- (2,6-dioxopiperidin-3-ylamino)phenyl)propan- amide |
| 59 | 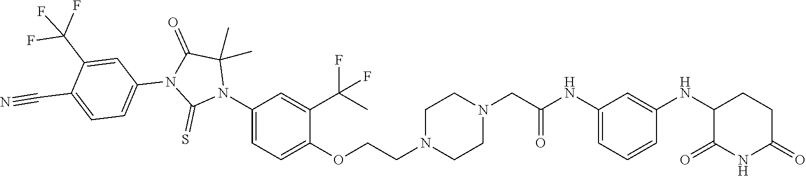 [SMILES: CC(F)(F)c1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)CC1] | 2-(4-(2-(4-(3-(4-cyano-3-(trifluoromethyl)phenyl)- 5,5-dimethyl-4-oxo-2-thioxoimidazolidin-1-yl)-2- (1,1-difluoroethyl)phenoxy)ethyl)piperazin-1-yl)- N-(3-(2,6-dioxopiperidin-3-ylamino)phenyl)acet- amide |
| 60 | 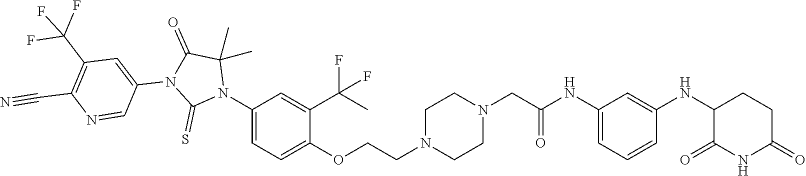 [SMILES: CC(F)(F)c1cc(N2C(=S)N(c3cnc(C#N)c(C(F)(F)F)c3)C(=O)C2(C)C)ccc1OCCN1CCN(CC(=O)Nc2cccc(NC3CCC(=O)NC3=O)c2)CC1] | 2-(4-(2-(4-(3-(6-cyano-5-(trifluoromethyl)pyridin- 3-yl)-5,5-dimethyl-4-oxo-2-thioxoimidazolidin-1- yl)-2-(1,1-difluoroethyl)phenoxy)ethyl)piperazin- 1-yl)-N-(3-(2,6-dioxopiperidin-3-ylamino)phenyl)- acetamide |
| 61 | 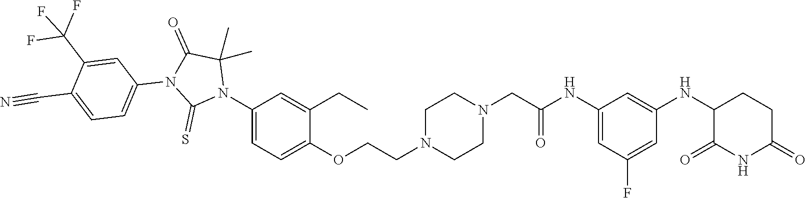 [SMILES: CCc1cc(N2C(=S)N(c3ccc(C#N)c(C(F)(F)F)c3)C(=O)C2(C)C)ccc1OCCN1CCN(CC(=O)Nc2cc(F)cc(NC3CCC(=O)NC3=O)c2)CC1] | 2-(4-(2-(4-(3-(4-cyano-3-(trifluoromethyl)phenyl)- 5,5-dimethyl-4-oxo-2-thioxoimidazolidin-1-yl)-2- ethylphenoxy)ethyl)piperazin-1-yl)-N-(3-(2,6- dioxopiperidin-3-ylamino)-5-fluorophenyl)acet- amide |
| 62 | 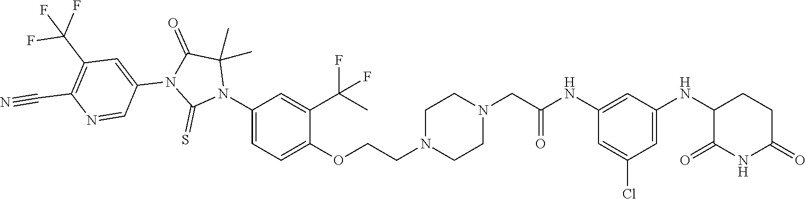 [SMILES: CC(F)(F)c1cc(N2C(=S)N(c3cnc(C#N)c(C(F)(F)F)c3)C(=O)C2(C)C)ccc1OCCN1CCN(CC(=O)Nc2cc(Cl)cc(NC3CCC(=O)NC3=O)c2)CC1] | N-(3-chloro-5-(2,6-dioxopiperidin-3-ylamino)- phenyl)-2-(4-(2-(4-(3-(6-cyano-5-(trifluorometh- yl)pyridin-3-yl)-5,5-dimethyl-4-oxo-2-thioxo- imidazolidin-1-yl)-2-(1,1-difluoroethyl)phenoxy)- ethyl)piperazin-1-yl)acetamide |
| 63 | 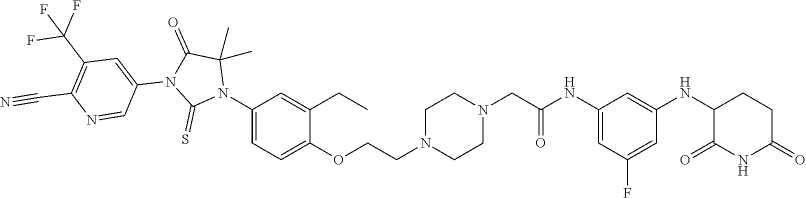 [SMILES: CCc1cc(N2C(=S)N(c3cnc(C#N)c(C(F)(F)F)c3)C(=O)C2(C)C)ccc1OCCN1CCN(CC(=O)Nc2cc(F)cc(NC3CCC(=O)NC3=O)c2)CC1] | 2-(4-(2-(4-(3-(6-cyano-5-(trifluoromethyl)pyridin- 3-yl)-5,5-dimethyl-4-oxo-2-thioxoimidazolidin-1- yl)-2-ethylphenoxy)ethyl)piperazin-1-yl)-N-(3- (2,6-dioxopiperidin-3-ylamino)-5-fluorophenyl)- acetamide |
| 64 | 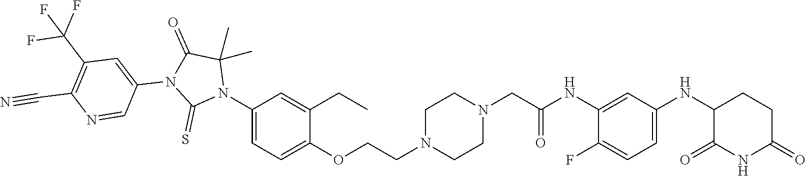 [SMILES: CCc1cc(N2C(=S)N(c3cnc(C#N)c(C(F)(F)F)c3)C(=O)C2(C)C)ccc1OCCN1CCN(CC(=O)Nc2cc(NC3CCC(=O)NC3=O)ccc2F)CC1] | 2-(4-(2-(4-(3-(6-cyano-5-(trifluoromethyl)pyridin- 3-yl)-5,5-dimethyl-4-oxo-2-thioxoimidazolidin-1- yl)-2-ethylphenoxy)ethyl)piperazin-1-yl)-N-(5- (2,6-dioxopiperidin-3-ylamino)-2-fluorophenyl)- acetamide |
| 65 | 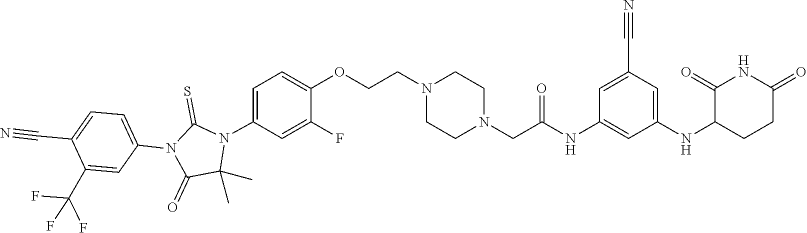 [SMILES: CC1(C)C(=O)N(c2ccc(C#N)c(C(F)(F)F)c2)C(=S)N1c1ccc(OCCN2CCN(CC(=O)Nc3cc(C#N)cc(NC4CCC(=O)NC4=O)c3)CC2)c(F)c1] | 2-(4-(2-(4-(3-(4-cyano-3-(trifluoromethyl)phenyl)- 5,5-dimethyl-4-oxo-2-thioxoimidazolidin-1-yl)-2- fluorophenoxy)ethyl)piperazin-1-yl)-N-(3-cyano- 5-(2,6-dioxopiperidin-3-ylamino)phenyl)acetamide |
| 66 | 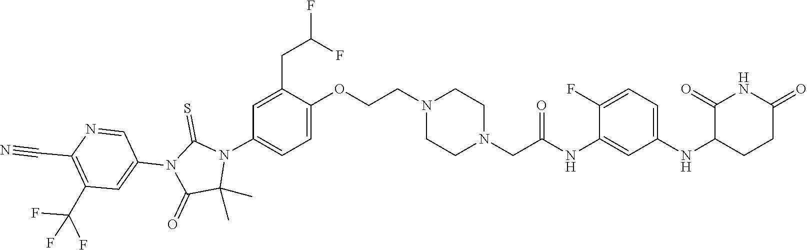 [SMILES: CC1(C)C(=O)N(c2cnc(C#N)c(C(F)(F)F)c2)C(=S)N1c1ccc(OCCN2CCN(CC(=O)Nc3cc(NC4CCC(=O)NC4=O)ccc3F)CC2)c(CC(F)F)c1] | 2-(4-(2-(4-(3-(6-cyano-5-(trifluoromethyl)pyridin- 3-yl)-5,5-dimethyl-4-oxo-2-thioxoimidazolidin-1- yl)-2-(2,2-difluoroethyl)phenoxy)ethyl)piperazin- 1-yl)-N-(5-(2,6-dioxopiperidin-3-ylamino)-2- fluorophenyl)acetamide |
| 67 | 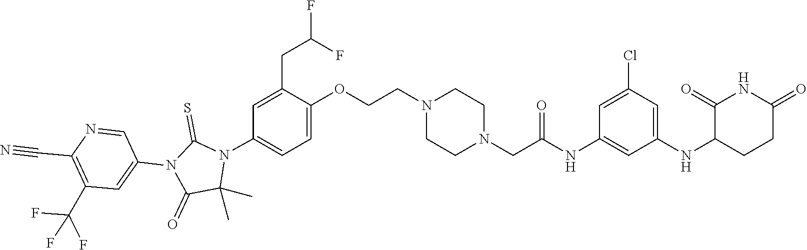 [SMILES: CC1(C)C(=O)N(c2cnc(C#N)c(C(F)(F)F)c2)C(=S)N1c1ccc(OCCN2CCN(CC(=O)Nc3cc(Cl)cc(NC4CCC(=O)NC4=O)c3)CC2)c(CC(F)F)c1] | N-(3-chloro-5-(2,6-dioxopiperidin-3-ylamino)- phenyl)-2-(4-(2-(4-(3-(6-cyano-5-(trifluorometh- yl)pyridin-3-yl)-5,5-dimethyl-4-oxo-2-thioxo- imidazolidin-1-yl)-2-(2,2-difluoroethyl)phenoxy)- ethyl)piperazin-1-yl)acetamide |
| 68 | 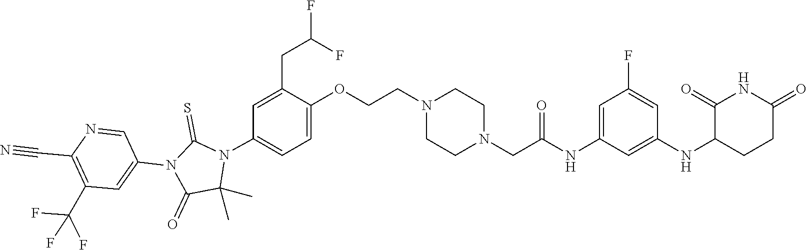 [SMILES: CC1(C)C(=O)N(c2cnc(C#N)c(C(F)(F)F)c2)C(=S)N1c1ccc(OCCN2CCN(CC(=O)Nc3cc(F)cc(NC4CCC(=O)NC4=O)c3)CC2)c(CC(F)F)c1] | 2-(4-(2-(4-(3-(6-cyano-5-(trifluoromethyl)pyridin- 3-yl)-5,5-dimethyl-4-oxo-2-thioxoimidazolidin-1- yl)-2-(2,2-difluoroethyl)phenoxy)ethyl)piperazin- 1-yl)-N-(3-(2,6-dioxopiperidin-3-ylamino)-5- fluorophenyl)acetamide |
| 69 | 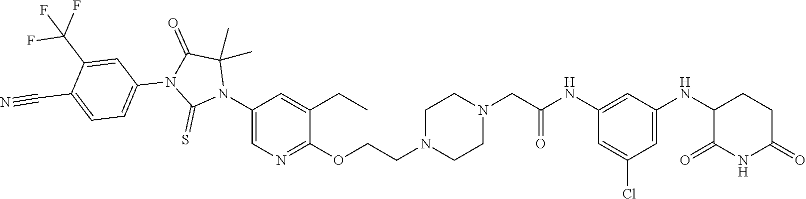 [SMILES: CCc1cc(N2C(=S)N(c3ccc(C#N)c(C(F)(F)F)c3)C(=O)C2(C)C)cnc1OCCN1CCN(CC(=O)Nc2cc(Cl)cc(NC3CCC(=O)NC3=O)c2)CC1] | N-(3-chloro-5-(2,6-dioxopiperidin-3-ylamino)- phenyl)-2-(4-(2-(5-(3-(4-cyano-3-trifluoromethyl)- phenyl)-5,5-dimethyl-4-oxo-2-thioxoimidazolidin- 1-yl)-3-ethylpyridin-2-yloxy)ethyl)piperazin-1- yl)acetamide |
| 70 | 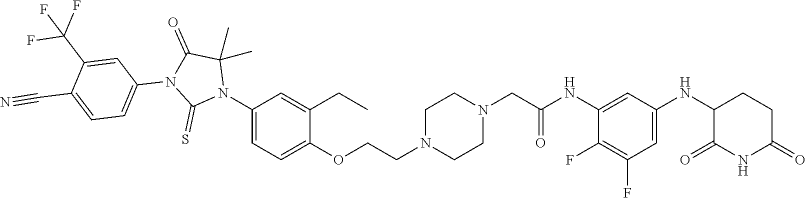 [SMILES: CCc1cc(N2C(=S)N(c3ccc(C#N)c(C(F)(F)F)c3)C(=O)C2(C)C)ccc1OCCN1CCN(CC(=O)Nc2cc(NC3CCC(=O)NC3=O)cc(F)c2F)CC1] | 2-(4-(2-(4-(3-(4-cyano-3-(trifluoromethyl)phenyl)- 5,5-dimethyl-4-oxo-2-thioxoimidazolidin-1-yl)-2- ethylphenoxy)ethyl)piperazin-1-yl)-N-(5-(2,6- dioxopiperidin-3-ylamino)-2,3-difluorophenyl)- acetamide |
| 71 | 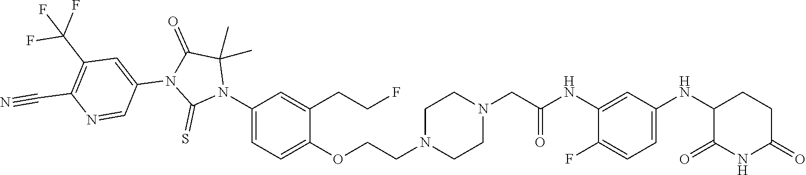 [SMILES: CC1(C)C(=O)N(c2cnc(C#N)c(C(F)(F)F)c2)C(=S)N1c1ccc(OCCN2CCN(CC(=O)Nc3cc(NC4CCC(=O)NC4=O)ccc3F)CC2)c(CCF)c1] | 2-(4-(2-(4-(3-(6-cyano-5-(trifluoromethyl)pyridin- 3-yl)-5,5-dimethyl-4-oxo-2-thioxoimidazolidin-1- yl)-2-(2-fluoroethyl)phenoxy)ethyl)piperazin-1- yl)-N-(5-(2,6-dioxopiperidin-3-ylamino)-2-fluoro- phenyl)acetamide |
| 72 | 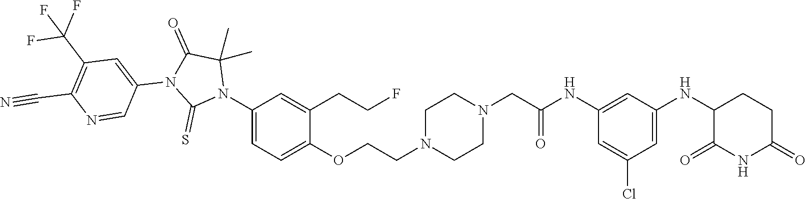 [SMILES: CC1(C)C(=O)N(c2cnc(C#N)c(C(F)(F)F)c2)C(=S)N1c1ccc(OCCN2CCN(CC(=O)Nc3cc(Cl)cc(NC4CCC(=O)NC4=O)c3)CC2)c(CCF)c1] | N-(3-chloro-5-(2,6-dioxopiperidin-3-ylamino)- phenyl)-2-(4-(2-(4-(3-(6-cyano-5-(trifluorometh- yl)pyridin-3-yl)-5,5-dimethyl-4-oxo-2-thioxoimid- azolidin-1-yl)-2-(2-fluoroethyl)phenoxy)ethyl)- piperazin-1-yl)acetamide |
| 73 | 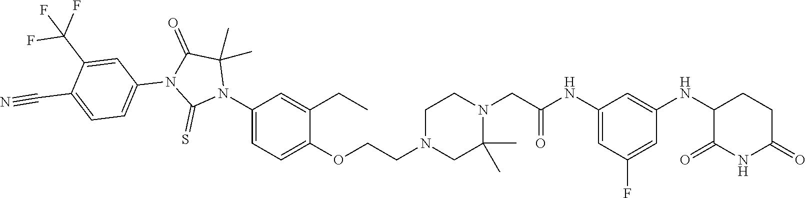 [SMILES: CCc1cc(N2C(=S)N(c3ccc(C#N)c(C(F)(F)F)c3)C(=O)C2(C)C)ccc1OCCN1CCN(CC(=O)Nc2cc(F)cc(NC3CCC(=O)NC3=O)c2)C(C)(C)C1] | 2-(4-(2-(4-(3-(4-cyano-3-(trifluoromethyl)phenyl)- 5,5-dimethyl-4-oxo-2-thioxoimidazolidin-1-yl)-2- ethylphenoxy)ethyl)-2,2-dimethylpiperazin-1-yl)- N-(3-(2,6-dioxopiperidin-3-ylamino)-5-fluoro- phenyl)acetamide |
| 74 | 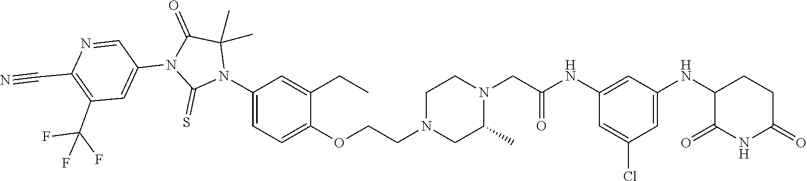 [SMILES: CCc1cc(N2C(=S)N(c3cnc(C#N)c(C(F)(F)F)c3)C(=O)C2(C)C)ccc1OCCN1CCN(CC(=O)Nc2cc(Cl)cc(NC3CCC(=O)NC3=O)c2)[C@H](C)C1] | N-(3-chloro-5-(2,6-dioxopiperidin-3-ylamino)- phenyl)-2-((R)-4-(2-(4-(3-(6-cyano-5-(trifluoro- methyl)pyridin-3-yl)-5,5-dimethyl-4-oxo-2- thioxoimidazolidin-1-yl)-2-ethylphenoxy)ethyl)- 2-methylpiperazin-1-yl)acetamide |
| 75 | 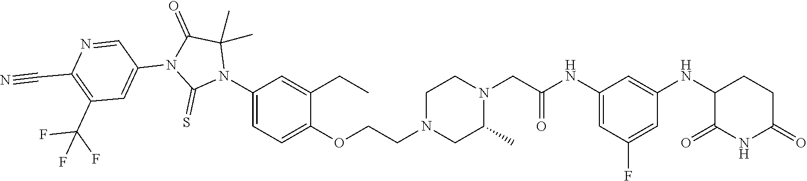 [SMILES: CCc1cc(N2C(=S)N(c3cnc(C#N)c(C(F)(F)F)c3)C(=O)C2(C)C)ccc1OCCN1CCN(CC(=O)Nc2cc(F)cc(NC3CCC(=O)NC3=O)c2)[C@H](C)C1] | 2-((R)-4-(2-(4-(3-(6-cyano-5-(trifluoromethyl)- pyridin-3-yl)-5,5-dimethyl-4-oxo-2-thioxoimid- azolidin-1-yl)-2-ethylphenoxy)ethyl)-2-methyl- piperazin-1-yl)-N-(3-(2,6-dioxopiperidin-3-yl- amino)-5-fluorophenyl)acetamide |
| 76 | 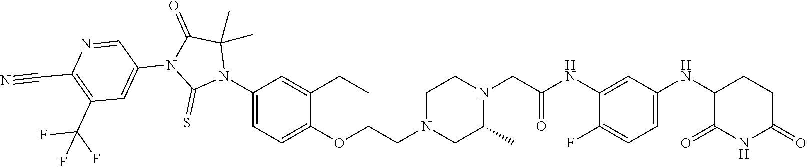 [SMILES: CCc1cc(N2C(=S)N(c3cnc(C#N)c(C(F)(F)F)c3)C(=O)C2(C)C)ccc1OCCN1CCN(CC(=O)Nc2cc(NC3CCC(=O)NC3=O)ccc2F)[C@H](C)C1] | 2-((R)-4-(2-(4-(3-(6-cyano-5-(trifluoromethyl)- pyridin-3-yl)-5,5-dimethyl-4-oxo-2-thioxoimid- azolidin-1-yl)-2-ethylphenoxy)ethyl)-2-methyl- piperazin-1-yl)-N-(5-(2,6-dioxopiperidin-3-yl- amino)-2-fluorophenyl)acetamide |
| 77 | 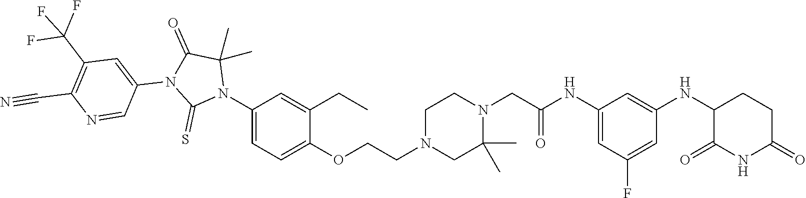 [SMILES: CCc1cc(N2C(=S)N(c3cnc(C#N)c(C(F)(F)F)c3)C(=O)C2(C)C)ccc1OCCN1CCN(CC(=O)Nc2cc(F)cc(NC3CCC(=O)NC3=O)c2)C(C)(C)C1] | 2-(4-(2-(4-(3-(6-cyano-5-(trifluoromethyl)pyridin- 3-yl)-5,5-dimethyl-4-oxo-2-thioxoimidazolidin-1- yl)-2-ethylphenoxy)ethyl)-2,2-dimethylpiperazin- 1-yl)-N-(3-(2,6-dioxopiperidin-3-ylamino)-5- fluorophenyl)acetamide |
| 78 | 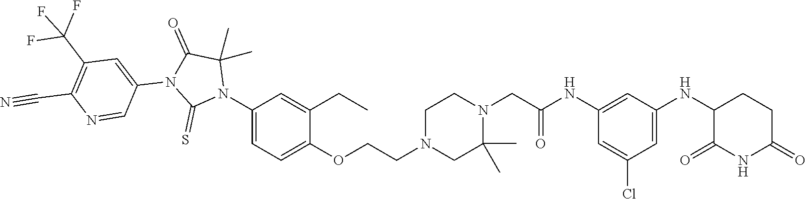 [SMILES: CCc1cc(N2C(=S)N(c3cnc(C#N)c(C(F)(F)F)c3)C(=O)C2(C)C)ccc1OCCN1CCN(CC(=O)Nc2cc(Cl)cc(NC3CCC(=O)NC3=O)c2)C(C)(C)C1] | N-(3-chloro-5-(2,6-dioxopiperidin-3-ylamino)- phenyl)-2-(4-(2-(4-(3-(6-cyano-5-(trifluorometh- yl)pyridin-3-yl)-5,5-dimethyl-4-oxo-2-thioxoimid- azolidin-1-yl)-2-ethylphenoxy)ethyl)-2,2-dimeth- ylpiperazin-1-yl)acetamide |
| 79 | 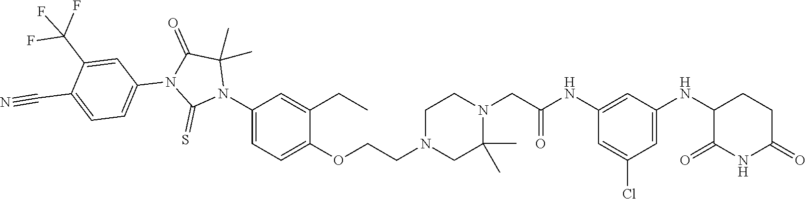 [SMILES: CCc1cc(N2C(=S)N(c3ccc(C#N)c(C(F)(F)F)c3)C(=O)C2(C)C)ccc1OCCN1CCN(CC(=O)Nc2cc(Cl)cc(NC3CCC(=O)NC3=O)c2)C(C)(C)C1] | N-(3-chloro-5-(2,6-dioxopiperidin-3-ylamino)- phenyl)-2-(4-(2-(4-(3-(4-cyano-3-(trifluorometh- yl)phenyl)-5,5-dimethyl-4-oxo-2-thioxoimidazol- idin-1-yl)-2-ethylphenoxy)ethyl)-2,2-dimethyl- piperazin-1-yl)acetamide |
| 80 | 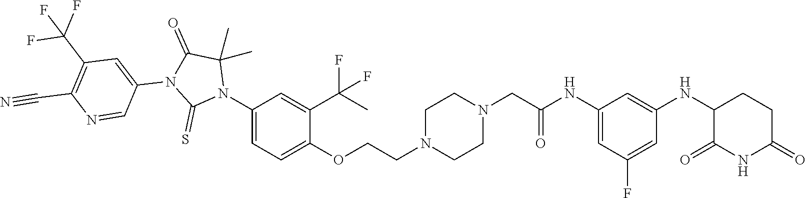 [SMILES: CC(F)(F)c1cc(N2C(=S)N(c3cnc(C#N)c(C(F)(F)F)c3)C(=O)C2(C)C)ccc1OCCN1CCN(CC(=O)Nc2cc(F)cc(NC3CCC(=O)NC3=O)c2)CC1] | 2-(4-(2-(4-(3-(6-cyano-5-(trifluoromethyl)pyridin- 3-yl)-5,5-dimethyl-4-oxo-2-thioxoimidazolidin-1- yl)-2-(1,1-difluoroethyl)phenoxy)ethyl)piperazin- 1-yl)-N-(3-(2,6-dioxopiperidin-3-ylamino)-5- fluorophenyl)acetamide |
| 81 | 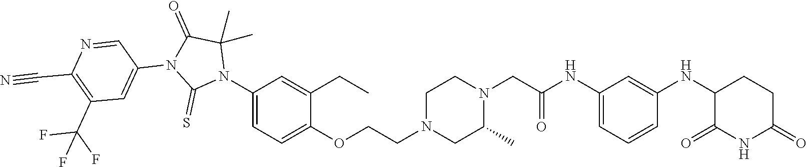 [SMILES: CCc1cc(N2C(=S)N(c3cnc(C#N)c(C(F)(F)F)c3)C(=O)C2(C)C)ccc1OCCN1CCN(CC(=O)Nc2cccc(NC3CCC(=O)NC3=O)c2)[C@H](C)C1] | 2-((R)-4-(2-(4-(3-(6-cyano-5-(trifluoromethyl)- pyridin-3-yl)-5,5-dimethyl-4-oxo-2-thioxoimid- azolidin-1-yl)-2-ethylphenoxy)ethyl)-2-methyl- piperazin-1-yl)-N-(3-(2,6-dioxopiperidin-3-yl- amino)phenyl)acetamide |
| 82 | 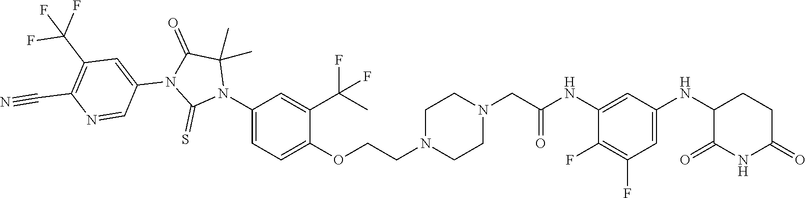 [SMILES: CC(F)(F)c1cc(N2C(=S)N(c3cnc(C#N)c(C(F)(F)F)c3)C(=O)C2(C)C)ccc1OCCN1CCN(CC(=O)Nc2cc(NC3CCC(=O)NC3=O)cc(F)c2F)CC1] | 2-(4-(2-(4-(3-(6-cyano-5-(trifluoromethyl)pyridin- 3-yl)-5,5-dimethyl-4-oxo-2-thioxoimidazolidin-1- yl)-2-(1,1-difluoroethyl)phenoxy)ethyl)piperazin- 1-yl)-N-(5-(2,6-dioxopiperidin-3-ylamino)-2,3- difluorophenyl)acetamide |
| 83 | 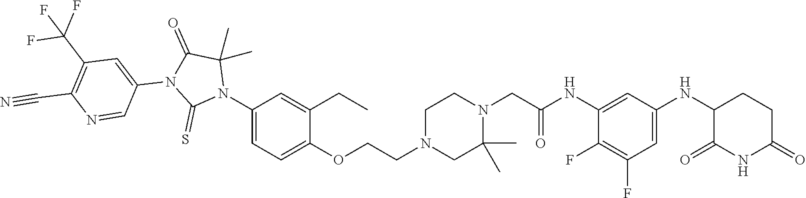 [SMILES: CCc1cc(N2C(=S)N(c3cnc(C#N)c(C(F)(F)F)c3)C(=O)C2(C)C)ccc1OCCN1CCN(CC(=O)Nc2cc(NC3CCC(=O)NC3=O)cc(F)c2F)C(C)(C)C1] | 2-(4-(2-(4-(3-(6-cyano-5-(trifluoromethyl)pyridin- 3-yl)-5,5-dimethyl-4-oxo-2-thioxoimidazolidin-1- yl)-2-ethylphenoxy)ethyl)-2,2-dimethylpiperazin- 1-yl)-N-(5-(2,6-dioxopiperidin-3-ylamino)-2,3- difluorophenyl)acetamide |
| 84 | 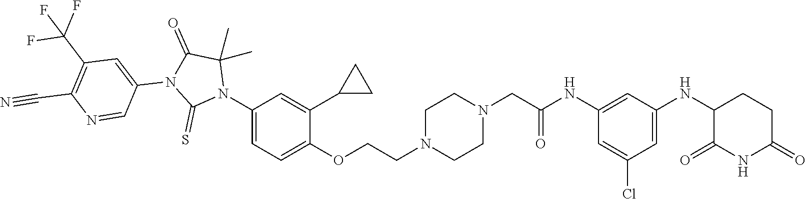 [SMILES: CC1(C)C(=O)N(c2cnc(C#N)c(C(F)(F)F)c2)C(=S)N1c1ccc(OCCN2CCN(CC(=O)Nc3cc(Cl)cc(NC4CCC(=O)NC4=O)c3)CC2)c(C2CC2)c1] | N-(3-chloro-5-(2,6-dioxopiperidin-3-ylamino)- phenyl)-2-(4-(2-(4-(3-(6-cyano-5-(trifluorometh- yl)pyridin-3-yl)-5,5-dimethyl-4-oxo-2-thioxo- imidazolidin-1-yl)-2-cyclopropylphenoxy)ethyl)- piperazin-1-yl)acetamide |
| 85 | 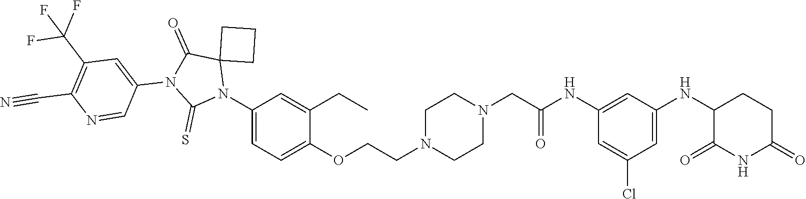 [SMILES: CCc1cc(N2C(=S)N(c3cnc(C#N)c(C(F)(F)F)c3)C(=O)C23CCC3)ccc1OCCN1CCN(CC(=O)Nc2cc(Cl)cc(NC3CCC(=O)NC3=O)c2)CC1] | N-(3-chloro-5-(2,6-dioxopiperidin-3-ylamino)- phenyl)-2-(4-(2-(4-(7-(6-cyano-5-(trifluorometh- yl)pyridin-3-yl)-8-oxo-6-thioxo-5,7-diazaspiro [3.4]octan-5-yl)-2-ethylphenoxy)ethyl)piperazin- 1-yl)acetamide |
| 86 | 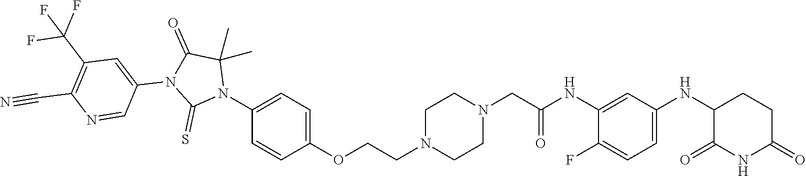 [SMILES: CC1(C)C(=O)N(c2cnc(C#N)c(C(F)(F)F)c2)C(=S)N1c1ccc(OCCN2CCN(CC(=O)Nc3cc(NC4CCC(=O)NC4=O)ccc3F)CC2)cc1] | 2-(4-(2-(4-(3-(6-cyano-5-(trifluoromethyl)pyridin- 3-yl)-5,5-dimethyl-4-oxo-2-thioxoimidazolidin-1- yl)phenoxy)ethyl)piperazin-1-yl)-N-(5-(2,6-dioxo- piperidin-3-ylamino)-2-fluorophenyl)acetamide |
| 87 | 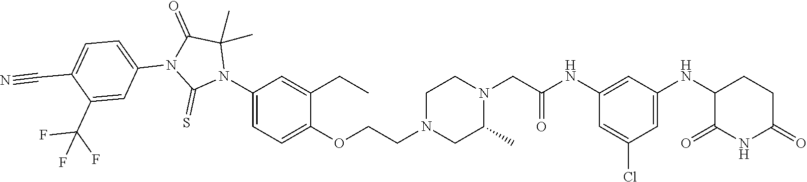 [SMILES: CCc1cc(N2C(=S)N(c3ccc(C#N)c(C(F)(F)F)c3)C(=O)C2(C)C)ccc1OCCN1CCN(CC(=O)Nc2cc(Cl)cc(NC3CCC(=O)NC3=O)c2)[C@H](C)C1] | N-(3-chloro-5-(2,6-dioxopiperidin-3-ylamino)- phenyl)-2-((R)-4-(2-(4-(3-(4-cyano-3-(trifluoro- methyl)phenyl)-5,5-dimethyl-4-oxo-2-thioxoimid- azolidin-1-yl)-2-ethylphenoxy)ethyl)-2-methyl- piperazin-1-yl)acetamide |
| 88 | 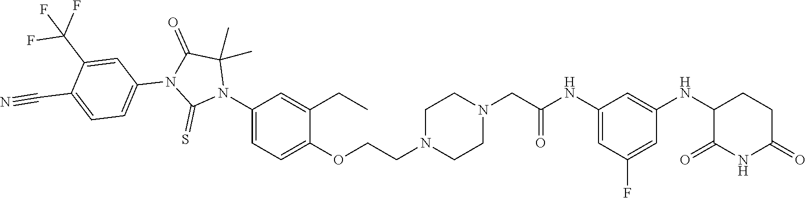 [SMILES: CCc1cc(N2C(=S)N(c3ccc(C#N)c(C(F)(F)F)c3)C(=O)C2(C)C)ccc1OCCN1CCN(CC(=O)Nc2cc(F)cc(NC3CCC(=O)NC3=O)c2)CC1] | 2-(4-(3-(4-(3-(4-cyano-3-(trifluoromethyl)phenyl)- 5,5-dimethyl-4-oxo-2-thioxoimidazolidin-1-yl)-2- ethylphenyl)propyl)piperazin-1-yl)-N-(3-(2,6- dioxopiperidin-3-ylamino)-5-fluorophenyl)acet- amide |
| 89 | 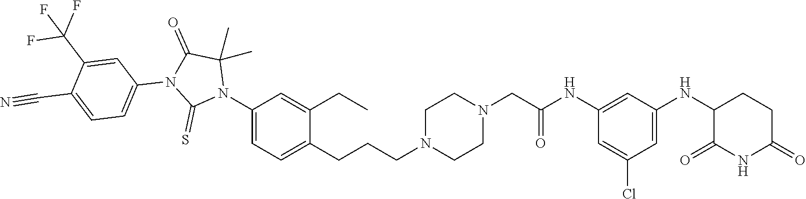 [SMILES: CCc1cc(N2C(=S)N(c3ccc(C#N)c(C(F)(F)F)c3)C(=O)C2(C)C)ccc1CCCN1CCN(CC(=O)Nc2cc(Cl)cc(NC3CCC(=O)NC3=O)c2)CC1] | N-(3-chloro-5-(2,6-dioxopiperidin-3-ylamino)- phenyl)-2-(4-(3-(4-(3-(4-cyano-3-(trifluorometh- yl)phenyl)-5,5-dimethyl-4-oxo-2-thioxoimidazol- idin-1-yl)-2-ethylphenyl)propyl)piperazin-1-yl)- acetamide |
| 90 | 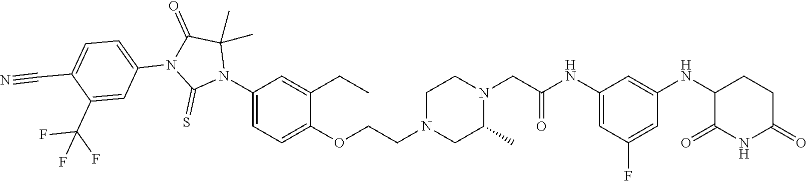 [SMILES: CCc1cc(N2C(=S)N(c3ccc(C#N)c(C(F)(F)F)c3)C(=O)C2(C)C)ccc1OCCN1CCN(CC(=O)Nc2cc(F)cc(NC3CCC(=O)NC3=O)c2)[C@H](C)C1] | 2-((R)-4-(2-(4-(3-(4-cyano-3-(trifluoromethyl)- phenyl)-5,5-dimethyl-4-oxo-2-thioxoimidazolidin- 1-yl)-2-ethylphenoxy)ethyl)-2-methylpiperazin- 1-yl)-N-(3-(2,6-dioxopiperidin-3-ylamino)-5- fluorophenyl)acetamide |
| 91 | 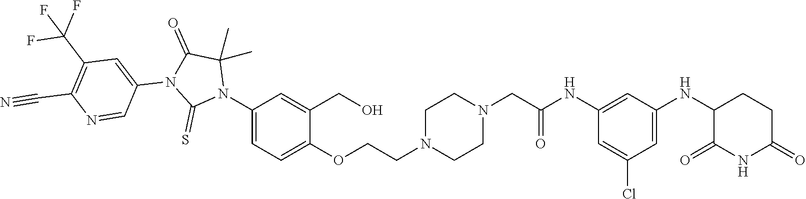 [SMILES: CC1(C)C(=O)N(c2cnc(C#N)c(C(F)(F)F)c2)C(=S)N1c1ccc(OCCN2CCN(CC(=O)Nc3cc(Cl)cc(NC4CCC(=O)NC4=O)c3)CC2)c(CO)c1] | N-(3-chloro-5-(2,6-dioxopiperidin-3-ylamino)- phenyl)-2-(4-(2-(4-(3-(6-cyano-5-(trifluorometh- yl)pyridin-3-yl)-5,5-dimethyl-4-oxo-2-thioxoimid- azolidin-1-yl)-2-(hydroxymethyl)phenoxy)ethyl)- piperazin-1-yl)acetamide |
| 92 | 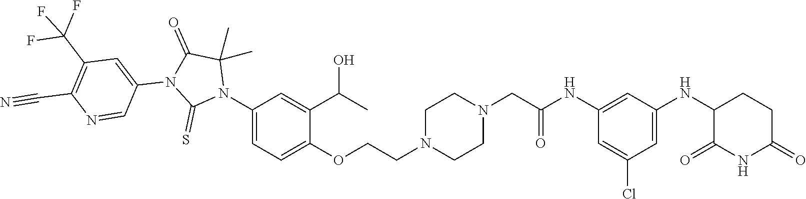 [SMILES: CC(O)c1cc(N2C(=S)N(c3cnc(C#N)c(C(F)(F)F)c3)C(=O)C2(C)C)ccc1OCCN1CCN(CC(=O)Nc2cc(Cl)cc(NC3CCC(=O)NC3=O)c2)CC1] | N-(3-chloro-5-(2,6-dioxopiperidin-3-ylamino)- phenyl)-2-(4-(2-(4-(3-(6-cyano-5-(trifluorometh- yl)pyridin-3-yl)-5,5-dimethyl-4-oxo-2-thioxoimid- azolidin-1-yl)-2-(1-hydroxyethyl)phenoxy)ethyl)- piperazin-1-yl)acetamide |
| 93 | 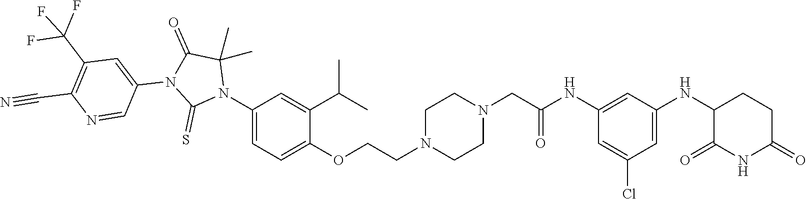 [SMILES: CC(C)c1cc(N2C(=S)N(c3cnc(C#N)c(C(F)(F)F)c3)C(=O)C2(C)C)ccc1OCCN1CCN(CC(=O)Nc2cc(Cl)cc(NC3CCC(=O)NC3=O)c2)CC1] | N-(3-chloro-5-(2,6-dioxopiperidin-3-ylamino)- phenyl)-2-(4-(2-(4-(3-(6-cyano-5-(trifluorometh- yl)pyridin-3-yl)-5,5-dimethyl-4-oxo-2-thioxo- imidazolidin-1-yl)-2-isopropylphenoxy)ethyl)- piperazin-1-yl)acetamide |
| 94 | 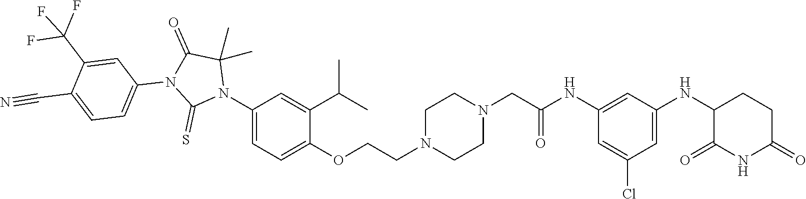 [SMILES: CC(C)c1cc(N2C(=S)N(c3ccc(C#N)c(C(F)(F)F)c3)C(=O)C2(C)C)ccc1OCCN1CCN(CC(=O)Nc2cc(Cl)cc(NC3CCC(=O)NC3=O)c2)CC1] | N-(3-chloro-5-(2,6-dioxopiperidin-3-ylamino)- phenyl)-2-(4-(2-(4-(3-(4-cyano-3-(trifluorometh- yl)phenyl)-5,5-dimethyl-4-oxo-2-thioxoimidazol- idin-1-yl)-2-isopropylphenoxy)ethyl)piperazin- 1-yl)acetamide |
| 95 | 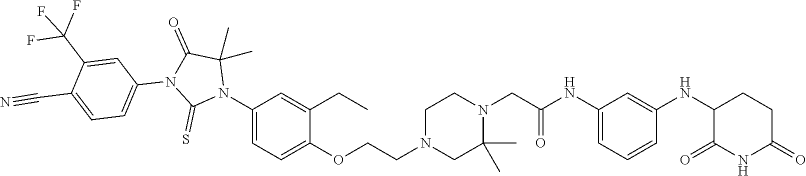 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)C(C)(C)C1] | 2-(4-(2-(4-(3-(4-cyano-3-(trifluoromethyl)phenyl)- 5,5-dimethyl-4-oxo-2-thioxoimidazolidin-1-yl)-2- ethylphenoxy)ethyl)-2,2-dimethylpiperazin-1-yl)- N-(3-(2,6-dioxopiperidin-3-ylamino)phenyl)acet- amide |
| 96 | 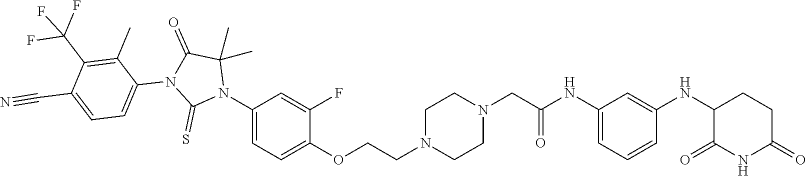 [SMILES: Cc1c(N2C(=O)C(C)(C)N(c3ccc(OCCN4CCN(CC(=O)Nc5cccc(NC6CCC(=O)NC6=O)c5)CC4)c(F)c3)C2=S)ccc(C#N)c1C(F)(F)F] | 2-(4-(2-(4-(3-(4-cyano-2-methyl-3-(trifluorometh- yl)phenyl)-5,5-dimethyl-4-oxo-2-thioxoimidazol- idin-1-yl)-2-fluorophenoxy)ethyl)piperazin-1-yl)- N-(3-(2,6-dioxopiperidin-3-ylamino)phenyl)acet- amide |
| 97 | 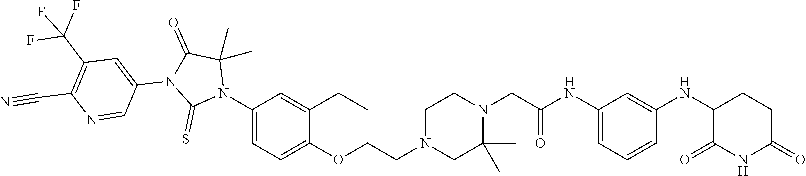 [SMILES: CCc1cc(N2C(=S)N(c3cnc(C#N)c(C(F)(F)F)c3)C(=O)C2(C)C)ccc1OCCN1CCN(CC(=O)Nc2cccc(NC3CCC(=O)NC3=O)c2)C(C)(C)C1] | 2-(4-(2-(4-(3-(6-cyano-5-(trifluoromethyl)pyridin- 3-yl)-5,5-dimethyl-4-oxo-2-thioxoimidazolidin-1- yl)-2-ethylphenoxy)ethyl)-2,2-dimethylpiperazin- 1-yl)-N-(3-(2,6-dioxopiperidin-3-ylamino)phen- yl)acetamide |
| 98 | 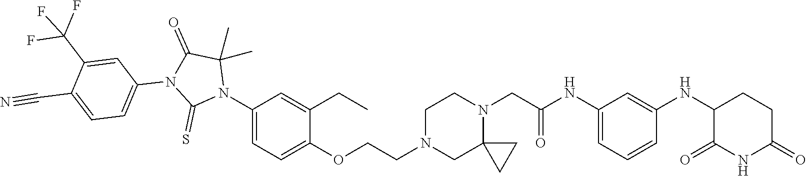 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)C2(CC2)C1] | 2-(7-(2-(4-(3-(4-cyano-3-(trifluoromethyl)phenyl)- 5,5-dimethyl-4-oxo-2-thioxoimidazolidin-1-yl)-2- ethylphenoxy)ethyl)-4,7-diazaspiro[2.5]octan-4- yl)-N-(3-(2,6-dioxopiperidin-3-ylamino)phenyl)- acetamide |
| 99 | 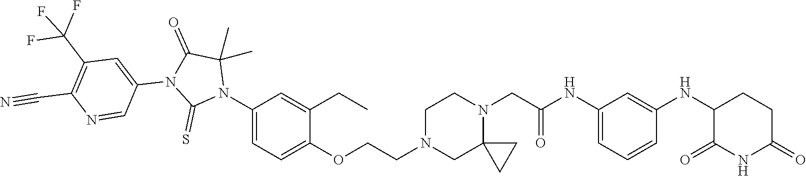 [SMILES: CCc1cc(N2C(=S)N(c3cnc(C#N)c(C(F)(F)F)c3)C(=O)C2(C)C)ccc1OCCN1CCN(CC(=O)Nc2cccc(NC3CCC(=O)NC3=O)c2)C2(CC2)C1] | 2-(7-(2-(4-(3-(6-cyano-5-(trifluoromethyl)pyridin- 3-yl)-5,5-dimethyl-4-oxo-2-thioxoimidazolidin- 1-yl)-2-ethylphenoxy)ethyl)-4,7-diazaspiro[2.5] octan-4-yl)-N-(3-(2,6-dioxopiperidin-3-ylamino)- phenyl)acetamide |
| 100 | 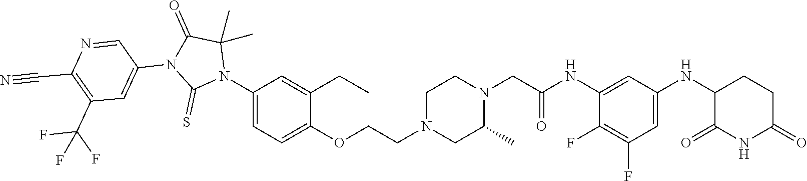 [SMILES: CCc1cc(N2C(=S)N(c3cnc(C#N)c(C(F)(F)F)c3)C(=O)C2(C)C)ccc1OCCN1CCN(CC(=O)Nc2cc(NC3CCC(=O)NC3=O)cc(F)c2F)[C@H](C)C1] | 2-((R)-4-(2-(4-(3-(6-cyano-5-(trifluoromethyl)- pyridin-3-yl)-5,5-dimethyl-4-oxo-2-thioxoimid- azolidin-1-yl)-2-ethylphenoxy)ethyl)-2-ethyl- piperazin-1-yl)-N-(5-(2,6-dioxopiperidin-3-yl- amino)-2,3-difluorophenyl)acetamide |
| 101 | 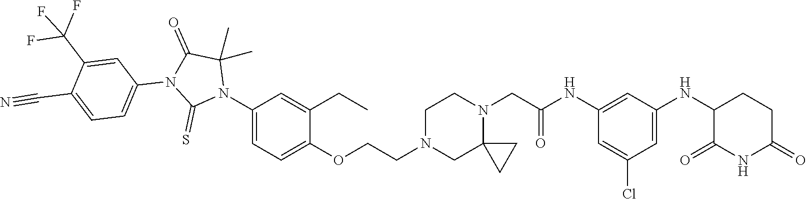 [SMILES: CCc1cc(N2C(=S)N(c3ccc(C#N)c(C(F)(F)F)c3)C(=O)C2(C)C)ccc1OCCN1CCN(CC(=O)Nc2cc(Cl)cc(NC3CCC(=O)NC3=O)c2)C2(CC2)C1] | N-(3-chloro-5-(2,6-dioxopiperidin-3-ylamino)- phenyl)-2-(7-(2-(4-(3-(4-cyano-3-(trifluorometh- yl)phenyl)-5,5-dimethyl-4-oxo-2-thioxoimidazol- idin-1-yl)-2-ethylphenoxy)ethyl)-4,7-diazaspiro [2.5]octan-4-yl)acetamide |
| 102 | 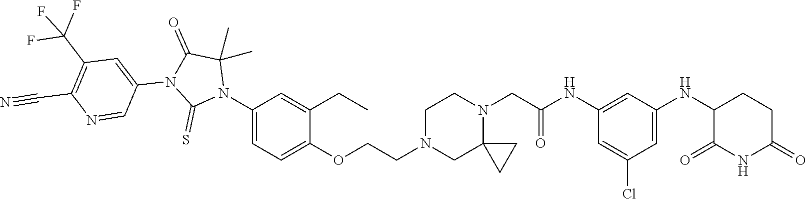 [SMILES: CCc1cc(N2C(=S)N(c3cnc(C#N)c(C(F)(F)F)c3)C(=O)C2(C)C)ccc1OCCN1CCN(CC(=O)Nc2cc(Cl)cc(NC3CCC(=O)NC3=O)c2)C2(CC2)C1] | N-(3-chloro-5-(2,6-dioxopiperidin-3-ylamino)- phenyl)-2-(7-(2-(4-(3-(6-cyano-5-(trifluorometh- yl)pyridin-3-yl)-5,5-dimethyl-4-oxo-2-thioxo- imidazolidin-1-yl)-2-ethylphenoxy)ethyl)-4,7- diazaspiro[2.5]octan-4-yl)acetamide |
| 103 | 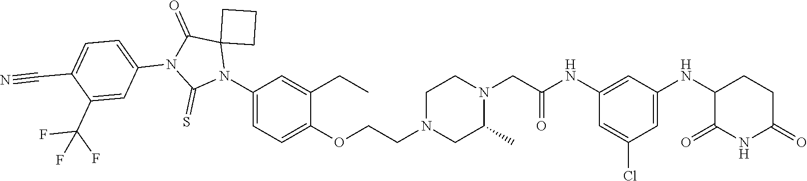 [SMILES: CCc1cc(N2C(=S)N(c3ccc(C#N)c(C(F)(F)F)c3)C(=O)C23CCC3)ccc1OCCN1CCN(CC(=O)Nc2cc(Cl)cc(NC3CCC(=O)NC3=O)c2)[C@H](C)C1] | N-(3-chloro-5-(2,6-dioxopiperidin-3-ylamino)- phenyl)-2-((R)-4-(2-(4-(7-(4-cyano-3-(trifluoro- methyl)phenyl)-8-oxo-6-thioxo-5,7-diazaspiro [3.4]octan-5-yl)-2-ethylphenoxy)ethyl)-2-meth- ylpiperazin-1-yl)acetamide |
| 104 | 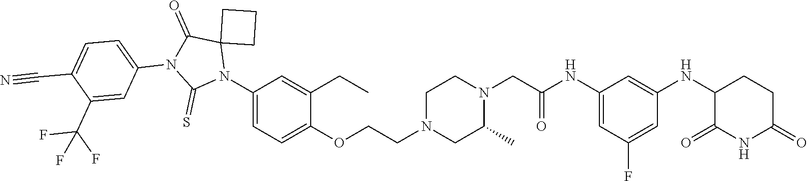 [SMILES: CCc1cc(N2C(=S)N(c3ccc(C#N)c(C(F)(F)F)c3)C(=O)C23CCC3)ccc1OCCN1CCN(CC(=O)Nc2cc(F)cc(NC3CCC(=O)NC3=O)c2)[C@H](C)C1] | 2-((R)-4-(2-(4-(7-(4-cyano-3-(trifluoromethyl)- phenyl)-8-oxo-6-thioxo-5,7-diazaspiro[3.4]octan- 5-yl)-2-ethylphenoxy)ethyl)-2-methylpiperazin- 1-yl)-N-(3-(2,6-dioxopiperidin-3-ylamino)-5- fluorophenyl)acetamide |
| 105 | 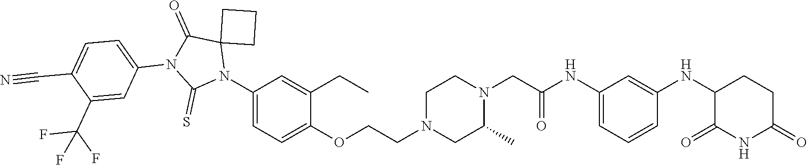 [SMILES: CCc1cc(N2C(=S)N(c3ccc(C#N)c(C(F)(F)F)c3)C(=O)C23CCC3)ccc1OCCN1CCN(CC(=O)Nc2cccc(NC3CCC(=O)NC3=O)c2)[C@H](C)C1] | 2-((R)-4-(2-(4-(7-(4-cyano-3-(trifluoromethyl)- phenyl)-8-oxo-6-thioxo-5,7-diazaspiro[3.4]octan- 5-yl)-2-ethylphenoxy)ethyl)-2-methylpiperazin- 1-yl)-N-(3-(2,6-dioxopiperidin-3-ylamino)phen- yl)acetamide |
| 106 | 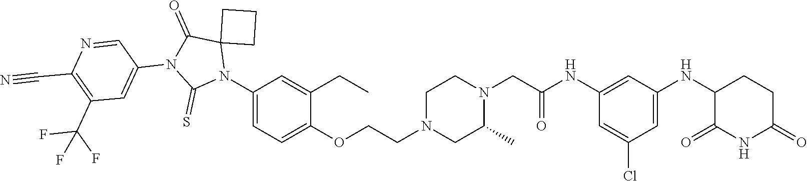 [SMILES: CCc1cc(N2C(=S)N(c3cnc(C#N)c(C(F)(F)F)c3)C(=O)C23CCC3)ccc1OCCN1CCN(CC(=O)Nc2cc(Cl)cc(NC3CCC(=O)NC3=O)c2)[C@H](C)C1] | N-(3-chloro-5-(2,6-dioxopiperidin-3-ylamino)- phenyl)-2-((R)-4-(2-(4-(7-(6-cyano-5-(trifluoro- methyl)pyridin-3-yl)-8-oxo-6-thioxo-5,7-diaza- spiro[3.4]octan-5-yl)-2-ethylphenoxy)ethyl)-2- methylpiperazin-1-yl)acetamide |
| 107 | 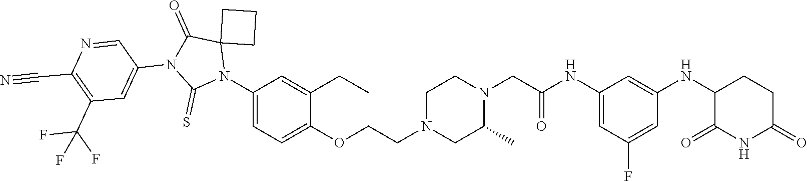 [SMILES: CCc1cc(N2C(=S)N(c3cnc(C#N)c(C(F)(F)F)c3)C(=O)C23CCC3)ccc1OCCN1CCN(CC(=O)Nc2cc(F)cc(NC3CCC(=O)NC3=O)c2)[C@H](C)C1] | 2-((R)-4-(2-(4-(7-(6-cyano-5-(trifluoromethyl)- pyridin-3-yl)-8-oxo-6-thioxo-5,7-diazaspiro[3.4] octan-5-yl)-2-ethylphenoxy)ethyl)-2-methylpiper- azin-1-yl)-N-(3-(2,6-dioxopiperidin-3-ylamino)- 5-fluorophenyl)acetamide |
| 108 | 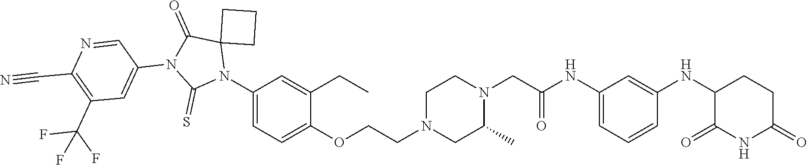 [SMILES: CCc1cc(N2C(=S)N(c3cnc(C#N)c(C(F)(F)F)c3)C(=O)C23CCC3)ccc1OCCN1CCN(CC(=O)Nc2cccc(NC3CCC(=O)NC3=O)c2)[C@H](C)C1] | 2-((R)-4-(2-(4-(7-(6-cyano-5-(trifluoromethyl)- pyridin-3-yl)-8-oxo-6-thioxo-5,7-diazaspiro[3.4] octan-5-yl)-2-ethylphenoxy)ethyl)-2-methylpiper- azin-1-yl)-N-(3-(2,6-dioxopiperidin-3-ylamino)- phenyl)acetamide |
| 109 | 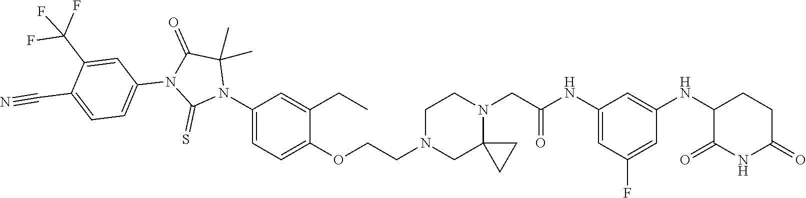 [SMILES: CCc1cc(N2C(=S)N(c3ccc(C#N)c(C(F)(F)F)c3)C(=O)C2(C)C)ccc1OCCN1CCN(CC(=O)Nc2cc(F)cc(NC3CCC(=O)NC3=O)c2)C2(CC2)C1] | 2-(7-(2-(4-(3-(4-cyano-3-(trifluoromethyl)phen- yl)-5,5-dimethyl-4-oxo-2-thioxoimidazolidin-1- yl)-2-ethylphenoxy)ethyl)-4,7-diazaspiro[2.5] octan-4-yl)-N-(3-(2,6-dioxopiperidin-3-ylamino)- 5-fluorophenyl)acetamide |
| 110 | 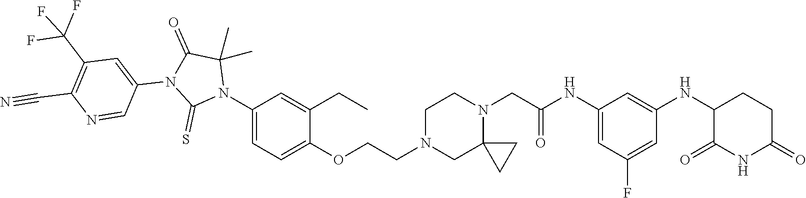 [SMILES: CCc1cc(N2C(=S)N(c3cnc(C#N)c(C(F)(F)F)c3)C(=O)C2(C)C)ccc1OCCN1CCN(CC(=O)Nc2cc(F)cc(NC3CCC(=O)NC3=O)c2)C2(CC2)C1] | 2-(7-(2-(4-(3-(6-cyano-5-(trifluoromethyl)pyridin- 3-yl)-5,5-dimethyl-4-oxo-2-thioxoimidazolidin-1- yl)-2-ethylphenoxy)ethyl)-4,7-diazaspiro[2.5] octan-4-yl)-N-(3-(2,6-dioxopiperidin-3-ylamino)- 5-fluorophenyl)acetamide |
| 111 | 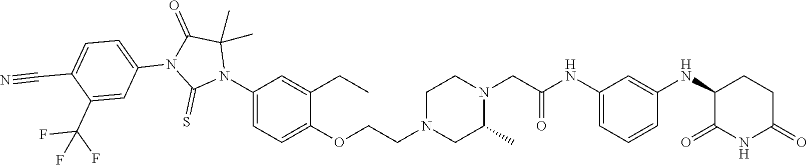 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@H]3CCC(=O)NC3=O)c2)[C@H](C)C1] | 2-((R)-4-(2-(4-(3-(4-cyano-3-(trifluoromethyl)- phenyl)-5,5-dimethyl-4-oxo-2-thioxoimidazol- idin-1-yl)-2-ethylphenoxy)ethyl)-2-methylpiper- azin-1-yl)-N-(3-((S)-2,6-dioxopiperidin-3-yl- amino)phenyl)acetamide |
| 112 | 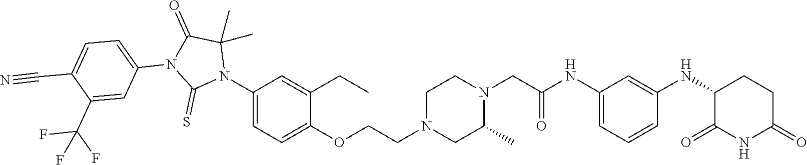 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1] | 2-((R)-4-(2-(4-(3-(4-cyano-3-(trifluoromethyl)- phenyl)-5,5-dimethyl-4-oxo-2-thioxoimidazol- idin-1-yl)-2-ethylphenoxy)ethyl)-2-methylpiper- azin-1-yl)-N-(3-((R)-2,6-dioxopiperidin-3-yl- amino)phenyl)acetamide |
| 113 (peak 1) 114 (peak 2) | 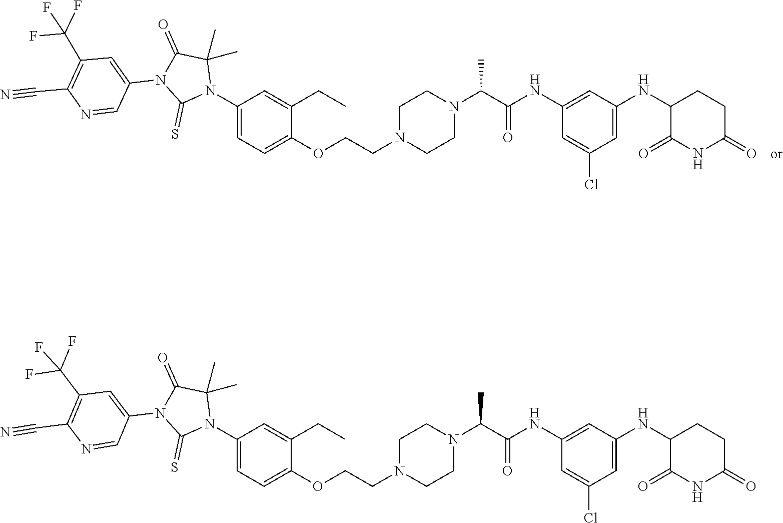 [SMILES: CCc1cc(N2C(=S)N(c3cnc(C#N)c(C(F)(F)F)c3)C(=O)C2(C)C)ccc1OCCN1CCN([C@H](C)C(=O)Nc2cc(Cl)cc(NC3CCC(=O)NC3=O)c2)CC1 ; CCc1cc(N2C(=S)N(c3cnc(C#N)c(C(F)(F)F)c3)C(=O)C2(C)C)ccc1OCCN1CCN([C@@H](C)C(=O)Nc2cc(Cl)cc(NC3CCC(=O)NC3=O)c2)CC1] | (2R)-N-(3-chloro-5-(2,6-dioxopiperidin-3-yl- amino)phenyl)-2-(4-(2-(4-(3-(6-cyano-5-(tri- fluoromethyl)pyridin-3-yl)-5,5-dimethyl-4-oxo- 2-thioxoimidazolidin-1-yl)-2-ethylphenoxy)- ethyl)piperazin-1-yl)propanamide or (2S)-N-(3-chloro-5-(2,6-dioxopiperidin-3-yl- amino)phenyl)-2-(4-(2-(4-(3-(6-cyano-5-(tri- fluoromethyl)pyridin-3-yl)-5,5-dimethyl-4-(oxo- 2-thioxoimidazolidin-1-yl)-2-ethylphenoxy)eth- yl)piperazin-1-yl)propanamide |
| 115 | 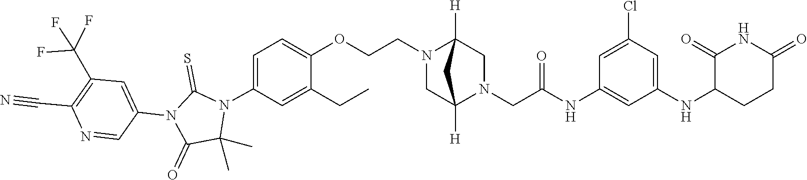 [SMILES: CCc1cc(N2C(=S)N(c3cnc(C#N)c(C(F)(F)F)c3)C(=O)C2(C)C)ccc1OCCN1C[C@@H]2C[C@H]1CN2CC(=O)Nc1cc(Cl)cc(NC2CCC(=O)NC2=O)c1] | N-(3-chloro-5-(2,6-dioxopiperidin-3-ylamino)- phenyl)-2-((1S,4S)-5-(2-(4-(3-(6-cyano-5-(tri- fluoromethyl)pyridin-3-yl)-5,5-dimethyl-4-oxo- 2-thioxoimidazolidin-1-yl)-2-ethylphenoxy)eth- yl)-2,5-diazabicyclo[2.2.1]heptan-2-yl)acetamide |
| 116 | 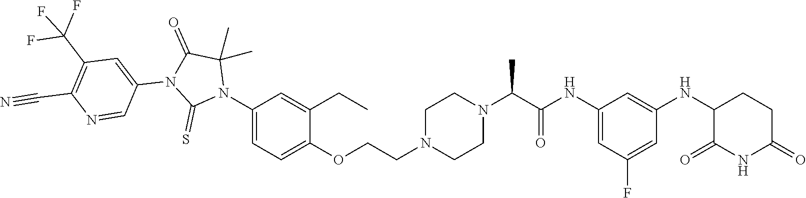 [SMILES: CCc1cc(N2C(=S)N(c3cnc(C#N)c(C(F)(F)F)c3)C(=O)C2(C)C)ccc1OCCN1CCN([C@@H](C)C(=O)Nc2cc(F)cc(NC3CCC(=O)NC3=O)c2)CC1] | (2S)-2-(4-(2-(4-(3-(6-cyano-5-(trifluoromethyl)- pyridin-3-yl)-5,5-dimethyl-4-oxo-2-thioxoimid- azolidin-1-yl)-2-ethylphenoxy)ethyl)piperazin-1- yl)-N-(3-(2,6-dioxopiperidin-3-ylamino)-5-fluoro- phenyl)propanamide |
| 117 | 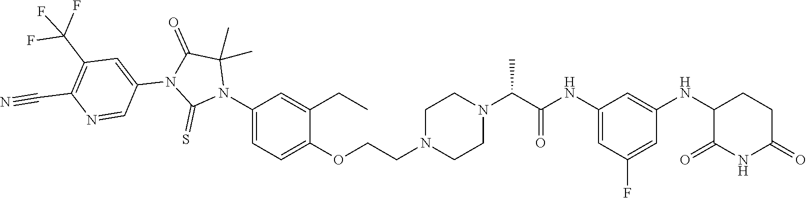 [SMILES: CCc1cc(N2C(=S)N(c3cnc(C#N)c(C(F)(F)F)c3)C(=O)C2(C)C)ccc1OCCN1CCN([C@H](C)C(=O)Nc2cc(F)cc(NC3CCC(=O)NC3=O)c2)CC1] | (2R)-2-(4-(2-(4-(3-(6-cyano-5-(trifluoromethyl)- pyridin-3-yl)-5,5-dimethyl-4-oxo-2-thioxoimid- azolidin-1-yl)-2-ethylphenoxy)ethyl)piperazin-1- yl)-N-(3-(2,6-dioxopiperidin-3-ylamino)-5-fluoro- phenyl)propanamide |
| 118 | 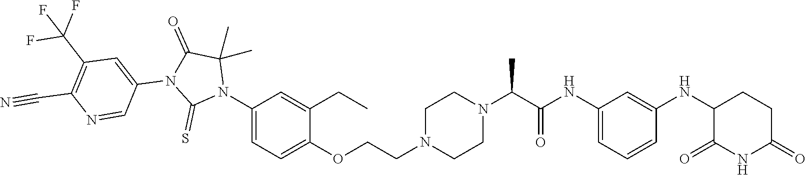 [SMILES: CCc1cc(N2C(=S)N(c3cnc(C#N)c(C(F)(F)F)c3)C(=O)C2(C)C)ccc1OCCN1CCN([C@@H](C)C(=O)Nc2cccc(NC3CCC(=O)NC3=O)c2)CC1] | (2S)-2-(4-(2-(4-(3-(6-cyano-5-(trifluoromethyl)- pyridin-3-yl)-5,5-dimethyl-4-oxo-2-thioxoimid- azolidin-1-yl)-2-ethylphenoxy)ethyl)piperazin-1- yl)-N-(3-(2,6-dioxopiperidin-3-ylamino)phenyl)- propanamide |
| 119 | 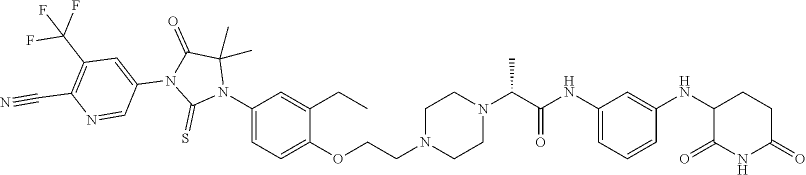 [SMILES: CCc1cc(N2C(=S)N(c3cnc(C#N)c(C(F)(F)F)c3)C(=O)C2(C)C)ccc1OCCN1CCN([C@H](C)C(=O)Nc2cccc(NC3CCC(=O)NC3=O)c2)CC1] | (2R)-2-(4-(2-(4-(3-(6-cyano-5-(trifluoromethyl)- pyridin-3-yl)-5,5-dimethyl-4-oxo-2-thioxoimid- azolidin-1-yl)-2-ethylphenoxy)ethyl)piperazin-1- yl)-N-(3-(2,6-dioxopiperidin-3-ylamino)phenyl)- propanamide |
| 120 | 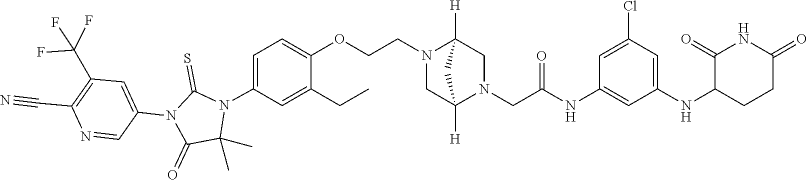 [SMILES: CCc1cc(N2C(=S)N(c3cnc(C#N)c(C(F)(F)F)c3)C(=O)C2(C)C)ccc1OCCN1C[C@H]2C[C@@H]1CN2CC(=O)Nc1cc(Cl)cc(NC2CCC(=O)NC2=O)c1] | N-(3-chloro-5-(2,6-dioxopiperidin-3-ylamino)- phenyl)-2-((1R,4R)-5-(2-(4-(3-(6-cyano-5- (trifluoromethyl)pyridin-3-yl)-5,5-dimethyl-4- oxo-2-thioxoimidazolidin-1-yl)-2-ethylphenoxy)- ethyl)-2,5-diazabicyclo[2.2.1]heptan-2-yl)acet- amide |
| 121 | 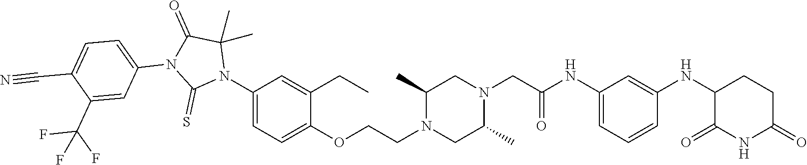 [SMILES: CCc1cc(N2C(=S)N(c3ccc(C#N)c(C(F)(F)F)c3)C(=O)C2(C)C)ccc1OCCN1C[C@@H](C)N(CC(=O)Nc2cccc(NC3CCC(=O)NC3=O)c2)C[C@@H]1C] | 2-((2R,5S)-4-(2-(4-(3-(4-cyano-3-(trifluorometh- yl)phenyl)-5,5-dimethyl-4-oxo-2-thioxoimidazol- idin-1-yl)-2-ethylphenoxy)ethyl)-2,5-dimethyl- piperazin-1-yl)-N-(3-(2,6-dioxopiperidin-3-yl- amino)phenyl)acetamide |
| 122 | 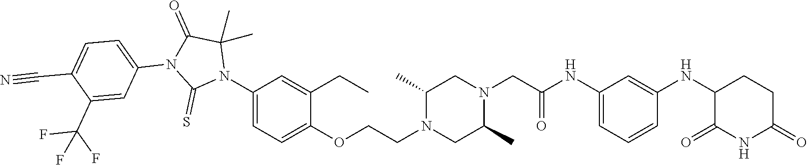 [SMILES: CCc1cc(N2C(=S)N(c3ccc(C#N)c(C(F)(F)F)c3)C(=O)C2(C)C)ccc1OCCN1C[C@H](C)N(CC(=O)Nc2cccc(NC3CCC(=O)NC3=O)c2)C[C@H]1C] | 2-((2S,5R)-4-(2-(4-(3-(4-cyano-3-(trifluorometh- yl)phenyl)-5,5-dimethyl-4-oxo-2-thioxoimidazol- idin-1-yl)-2-ethylphenoxy)ethyl)-2,5-dimethyl- piperazin-1-yl)-N-(3-(2,6-dioxopiperidin-3-yl- amino)phenyl)acetamide |
| 123 | 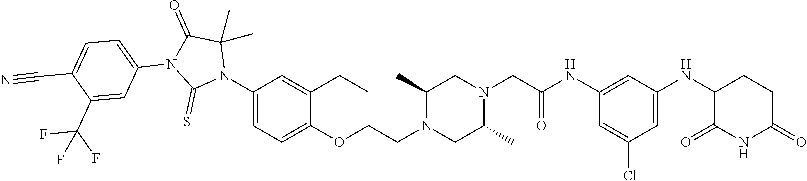 [SMILES: CCc1cc(N2C(=S)N(c3ccc(C#N)c(C(F)(F)F)c3)C(=O)C2(C)C)ccc1OCCN1C[C@@H](C)N(CC(=O)Nc2cc(Cl)cc(NC3CCC(=O)NC3=O)c2)C[C@@H]1C] | N-(3-chloro-5-(2,6-dioxopiperidin-3-ylamino)- phenyl)-2-((2R,5S)-4-(2-(4-(3-(4-cyano-3-(tri- fluoromethyl)phenyl)-5,5-dimethyl-4-oxo-2- thioxoimidazolidin-1-yl)-2-ethylphenoxy)ethyl)- 2,5-dimethylpiperazin-1-yl)acetamide |
| 124 | 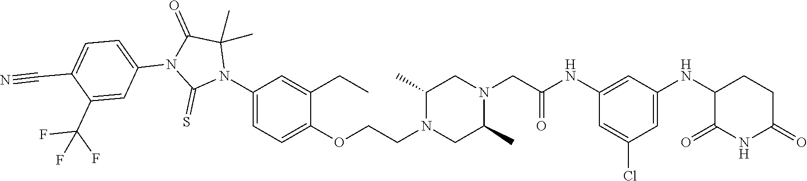 [SMILES: CCc1cc(N2C(=S)N(c3ccc(C#N)c(C(F)(F)F)c3)C(=O)C2(C)C)ccc1OCCN1C[C@H](C)N(CC(=O)Nc2cc(Cl)cc(NC3CCC(=O)NC3=O)c2)C[C@H]1C] | N-(3-chloro-5-(2,6-dioxopiperidin-3-ylamino)- phenyl)-2-((2S,5R)-4-(2-(4-(3-(4-cyano-3-(tri- fluoromethyl)phenyl)-5,5-dimethyl-4-oxo-2- thioxoimidazolidin-1-yl)-2-ethylphenoxy)ethyl)- 2,5-dimethylpiperazin-1-yl)acetamide |
| 125 | 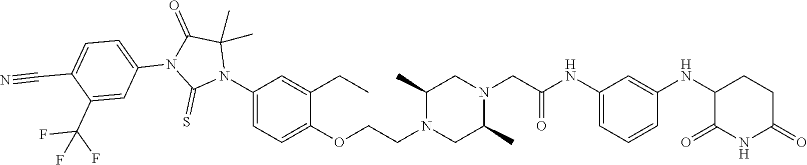 [SMILES: CCc1cc(N2C(=S)N(c3ccc(C#N)c(C(F)(F)F)c3)C(=O)C2(C)C)ccc1OCCN1C[C@H](C)N(CC(=O)Nc2cccc(NC3CCC(=O)NC3=O)c2)C[C@@H]1C] | 2-((2S,5S)-4-(2-(4-(3-(4-cyano-3-(trifluorometh- yl)phenyl)-5,5-dimethyl-4-oxo-2-thioxoimidazol- idin-1-yl)-2-ethylphenoxy)ethyl)-2,5-dimethyl- piperazin-1-yl)-N-(3-(2,6-dioxopiperidin-3-yl- amino)phenyl)acetamide |
| 126 | 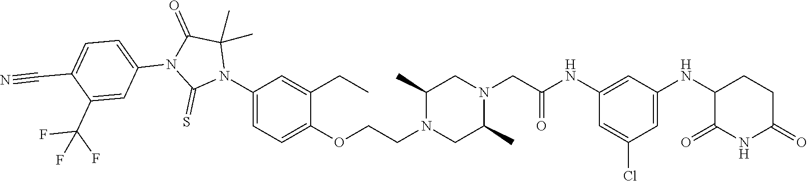 [SMILES: CCc1cc(N2C(=S)N(c3ccc(C#N)c(C(F)(F)F)c3)C(=O)C2(C)C)ccc1OCCN1C[C@H](C)N(CC(=O)Nc2cc(Cl)cc(NC3CCC(=O)NC3=O)c2)C[C@@H]1C] | N-(3-chloro-5-(2,6-dioxopiperidin-3-ylamino)- phenyl)-2-((2S,5S)-4-(2-(4-(3-(4-cyano-3-(tri- fluoromethyl)phenyl)-5,5-dimethyl-4-oxo-2- thioxoimidazolidin-1-yl)-2-ethylphenoxy)ethyl)- 2,5-dimethylpiperazin-1-yl)acetamide |
| 127 | 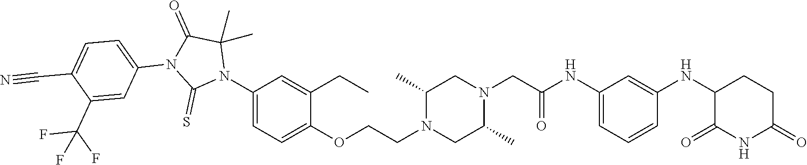 [SMILES: CCc1cc(N2C(=S)N(c3ccc(C#N)c(C(F)(F)F)c3)C(=O)C2(C)C)ccc1OCCN1C[C@@H](C)N(CC(=O)Nc2cccc(NC3CCC(=O)NC3=O)c2)C[C@H]1C] | 2-((2R,5R)-4-(2-(4-(3-(4-cyano-3-(trifluorometh- yl)phenyl)-5,5-dimethyl-4-oxo-2-thioxoimidazol- idin-1-yl)-2-ethylphenoxy)ethyl)-2,5-dimethyl- piperazin-1-yl)-N-(3-(2,6-dioxopiperidin-3-yl- amino)phenyl)acetamide |
| 128 | 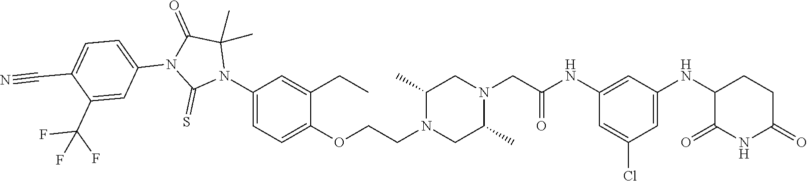 [SMILES: CCc1cc(N2C(=S)N(c3ccc(C#N)c(C(F)(F)F)c3)C(=O)C2(C)C)ccc1OCCN1C[C@@H](C)N(CC(=O)Nc2cc(Cl)cc(NC3CCC(=O)NC3=O)c2)C[C@H]1C] | N-(3-chloro-5-(2,6-dioxopiperidin-3-ylamino)- phenyl)-2-((2R,5R)-4-(2-(4-(3-(4-cyano-3- (trifluoromethyl)phenyl)-5,5-dimethyl-4-oxo-2- thioxoimidazolidin-1-yl)-2-ethylphenoxy)ethyl)- 2,5-dimethylpiperazin-1-yl)acetamide |
| 129 | 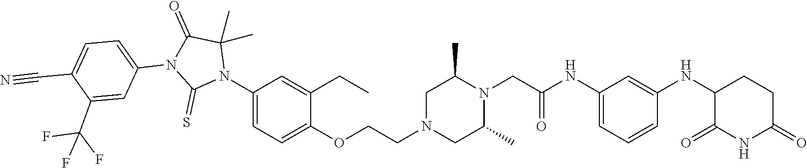 [SMILES: CCc1cc(N2C(=S)N(c3ccc(C#N)c(C(F)(F)F)c3)C(=O)C2(C)C)ccc1OCCN1C[C@@H](C)N(CC(=O)Nc2cccc(NC3CCC(=O)NC3=O)c2)[C@H](C)C1] | 2-((2R,6R)-4-(2-(4-(3-(4-cyano-3-(trifluorometh- yl)phenyl)-5,5-dimethyl-4-oxo-2-thioxoimidazol- idin-1-yl)-2-ethylphenoxy)ethyl)-2,6-dimethyl- piperazin-1-yl)-N-(3-(2,6-dioxopiperidin-3-yl- amino)phenyl)acetamide |
| 130 | 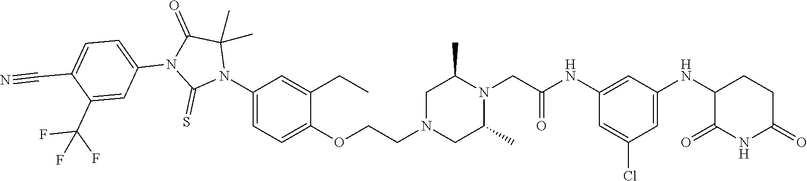 [SMILES: CCc1cc(N2C(=S)N(c3ccc(C#N)c(C(F)(F)F)c3)C(=O)C2(C)C)ccc1OCCN1C[C@@H](C)N(CC(=O)Nc2cc(Cl)cc(NC3CCC(=O)NC3=O)c2)[C@H](C)C1] | N-(3-chloro-5-(2,6-dioxopiperidin-3-ylamino)- phenyl)-2-((2R,6R)-4-(2-(4-(3-(4-cyano-3- (trifluoromethyl)phenyl)-5,5-dimethyl-4-oxo-2- thioxoimidazolidin-1-yl)-2-ethylphenoxy)ethyl)- 2,6-dimethylpiperazin-1-yl)acetamide |
| 131 | 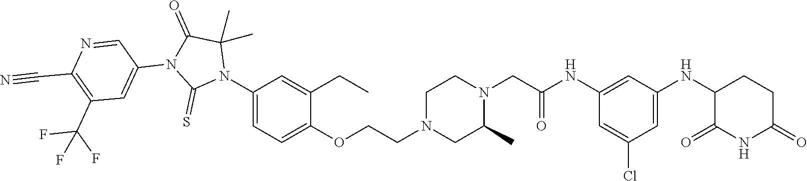 [SMILES: CCc1cc(N2C(=S)N(c3cnc(C#N)c(C(F)(F)F)c3)C(=O)C2(C)C)ccc1OCCN1CCN(CC(=O)Nc2cc(Cl)cc(NC3CCC(=O)NC3=O)c2)[C@@H](C)C1] | N-(3-chloro-5-(2,6-dioxopiperidin-3-ylamino)- phenyl)-2-((S)-4-(2-(4-(3-(6-cyano-5-(trifluoro- methyl)pyridin-3-yl)-5,5-dimethyl-4-oxo-2- thioxoimidazolidin-1-yl)-2-ethylphenoxy)ethyl)- 2-methylpiperazin-1-yl)acetamide |
| 132 | 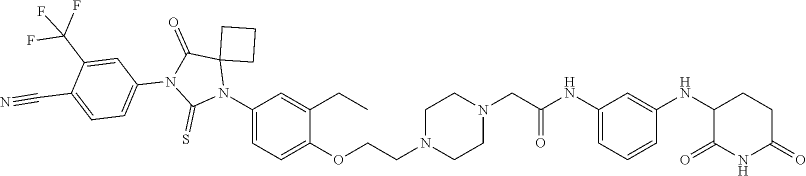 [SMILES: CCc1cc(N2C(=S)N(c3ccc(C#N)c(C(F)(F)F)c3)C(=O)C23CCC3)ccc1OCCN1CCN(CC(=O)Nc2cccc(NC3CCC(=O)NC3=O)c2)CC1] | 2-(4-(2-(4-(7-(4-cyano-3-(trifluoromethyl)phen- yl)-8-oxo-6-thioxo-5,7-diazaspiro[3.4]octan-5-yl)- 2-ethylphenoxy)ethyl)piperazin-1-yl)-N-(3-(2,6- dioxopiperidin-3-ylamino)phenyl)acetamide |
| 133 | 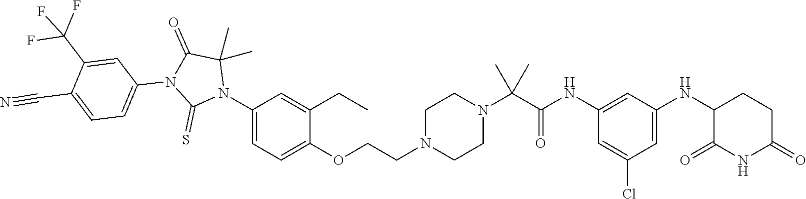 [SMILES: CCc1cc(N2C(=S)N(c3ccc(C#N)c(C(F)(F)F)c3)C(=O)C2(C)C)ccc1OCCN1CCN(C(C)(C)C(=O)Nc2cc(Cl)cc(NC3CCC(=O)NC3=O)c2)CC1] | N-(3-chloro-5-(2,6-dioxopiperidin-3-ylamino)- phenyl)-2-(4-(2-(4-(3-(4-cyano-3-(trifluorometh- yl)phenyl)-5,5-dimethyl-4-oxo-2-thioxoimidazol- idin-1-yl)-2-ethylphenoxy)ethyl)piperazin-1-yl)- 2-methylpropanamide |
| 134 | 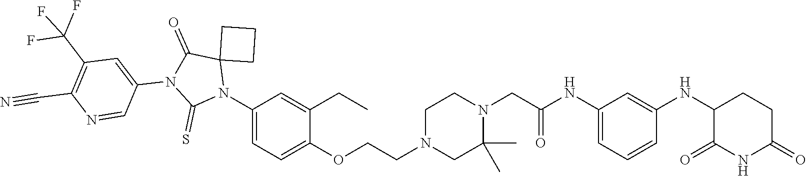 [SMILES: CCc1cc(N2C(=S)N(c3cnc(C#N)c(C(F)(F)F)c3)C(=O)C23CCC3)ccc1OCCN1CCN(CC(=O)Nc2cccc(NC3CCC(=O)NC3=O)c2)C(C)(C)C1] | 2-(4-(2-(4-(7-(6-cyano-5-(trifluoromethyl)pyridin- 3-yl)-8-oxo-6-thioxo-5,7-diazaspiro[3.4]octan-5- yl)-2-ethylphenoxy)ethyl)-2,2-dimethylpiperazin- 1-yl)-N-(3-(2,6-dioxopiperidin-3-ylamino)phen- yl)acetamide |
| 135 | 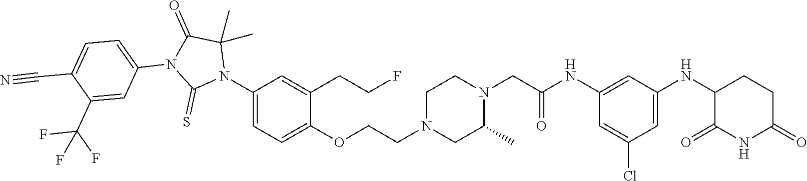 [SMILES: C[C@@H]1CN(CCOc2ccc(N3C(=S)N(c4ccc(C#N)c(C(F)(F)F)c4)C(=O)C3(C)C)cc2CCF)CCN1CC(=O)Nc1cc(Cl)cc(NC2CCC(=O)NC2=O)c1] | N-(3-chloro-5-(2,6-dioxopiperidin-3-ylamino)- phenyl)-2-((R)-4-(2-(4-(3-(4-cyano-3-(trifluoro- methyl)phenyl)-5,5-dimethyl-4-oxo-2-thioxo- imidazolidin-1-yl)-2-(2-fluoroethyl)phenoxy)- ethyl)-2-methylpiperazin-1-yl)acetamide |
| 136 | 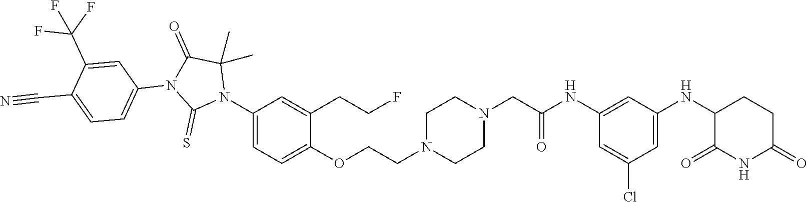 [SMILES: CC1(C)C(=O)N(c2ccc(C#N)c(C(F)(F)F)c2)C(=S)N1c1ccc(OCCN2CCN(CC(=O)Nc3cc(Cl)cc(NC4CCC(=O)NC4=O)c3)CC2)c(CCF)c1] | N-(3-chloro-5-(2,6-dioxopiperidin-3-ylamino)- phenyl)-2-(4-(2-(4-(3-(4-cyano-3-(trifluorometh- yl)phenyl)-5,5-dimethyl-4-oxo-2-thioxoimidazol- idin-1-yl)-2-(2-fluoroethyl)phenoxy)ethyl)piper- azin-1-yl)acetamide |
| 137 | 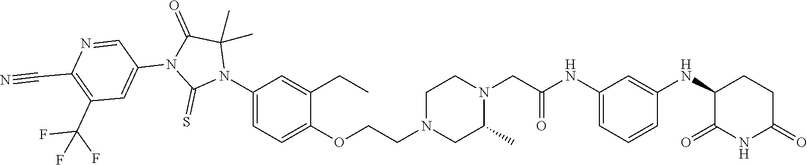 [SMILES: CCc1cc(N2C(=S)N(c3cnc(C#N)c(C(F)(F)F)c3)C(=O)C2(C)C)ccc1OCCN1CCN(CC(=O)Nc2cccc(N[C@H]3CCC(=O)NC3=O)c2)[C@H](C)C1] | 2-((R)-4-(2-(4-(3-(6-cyano-5-(trifluoromethyl)- pyridin-3-yl)-5,5-dimethyl-4-oxo-2-thioxoimid- azolidin-1-yl)-2-ethylphenoxy)ethyl)-2-methyl- piperazin-1-yl)-N-(3-((S)-2,6-dioxopiperidin-3- ylamino)phenyl)acetamide |
| 138 | 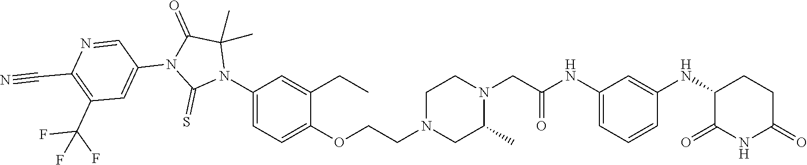 [SMILES: CCc1cc(N2C(=S)N(c3cnc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1] | 2-((R)-4-(2-(4-(3-(6-cyano-5-(trifluoromethyl)- pyridin-3-yl)-5,5-dimethyl-4-oxo-2-thioxoimid- azolidin-1-yl)-2-ethylphenoxy)ethyl)-2-methyl- piperazin-1-yl)-N-(3-((R)-2,6-dioxopiperidin-3- ylamino)phenyl)acetamide |
| 139 | 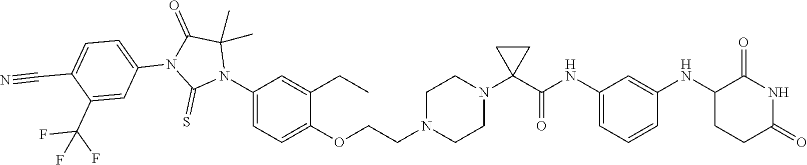 [SMILES: CCc1cc(N2C(=S)N(c3ccc(C#N)c(C(F)(F)F)c3)C(=O)C2(C)C)ccc1OCCN1CCN(C2(C(=O)Nc3cccc(NC4CCC(=O)NC4=O)c3)CC2)CC1] | 1-(4-(2-(4-(3-(4-cyano-3-(trifluoromethyl)phen- yl)-5,5-dimethyl-4-oxo-2-thioxoimidazolidin-1- yl)-2-ethylphenoxy)ethyl)piperazin-1-yl)-N-(3- (2,6-dioxopiperidin-3-ylamino)phenyl)cyclopro- panecarboxamide |
| 140 | 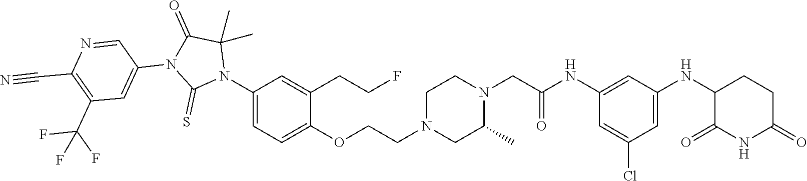 [SMILES: C[C@@H]1CN(CCOc2ccc(N3C(=S)N(c4cnc(C#N)c(C(F)(F)F)c4)C(=O)C3(C)C)cc2CCF)CCN1CC(=O)Nc1cc(Cl)cc(NC2CCC(=O)NC2=O)c1] | N-(3-chloro-5-(2,6-dioxopiperidin-3-ylamino)- phenyl)-2-((R)-4-(2-(4-(3-(6-cyano-5-(trifluoro- methyl)pyridin-3-yl)-5,5-dimethyl-4-oxo-2- thioxoimidazolidin-1-yl)-2-(2-fluoroethyl)phen- oxy)ethyl)-2-methylpiperazin-1-yl)acetamide |
| 141 | 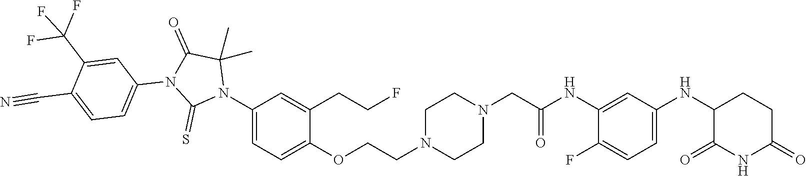 [SMILES: CC1(C)C(=O)N(c2ccc(C#N)c(C(F)(F)F)c2)C(=S)N1c1ccc(OCCN2CCN(CC(=O)Nc3cc(NC4CCC(=O)NC4=O)ccc3F)CC2)c(CCF)c1] | 2-(4-(2-(4-(3-(4-cyano-3-(trifluoromethyl)phenyl)- 5,5-dimethyl-4-oxo-2-thioxoimidazolidin-1-yl)-2- (2-fluoroethyl)phenoxy)ethyl)piperazin-1-yl)-N- (5-(2,6-dioxopiperidin-3-ylamino)-2-fluorophen- yl)acetamide |
| 142 | 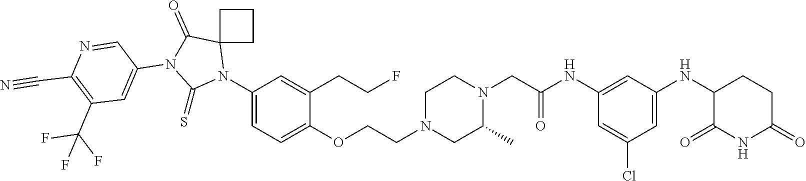 [SMILES: C[C@@H]1CN(CCOc2ccc(N3C(=S)N(c4cnc(C#N)c(C(F)(F)F)c4)C(=O)C34CCC4)cc2CCF)CCN1CC(=O)Nc1cc(Cl)cc(NC2CCC(=O)NC2=O)c1] | N-(3-chloro-5-(2,6-dioxopiperidin-3-ylamino)- phenyl)-2-((R)-4-(2-(4-(7-(6-cyano-5-(trifluoro- methyl)pyridin-3-yl)-8-oxo-6-thioxo-5,7-diaza- spiro[3.4]octan-5-yl)-2-(2-fluoroethyl)phenoxy)- ethyl)-2-methylpiperazin-1-yl)acetamide |
| 143 | 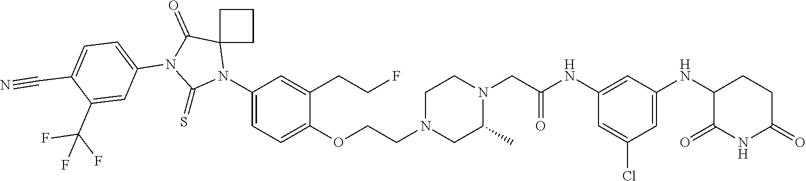 [SMILES: C[C@@H]1CN(CCOc2ccc(N3C(=S)N(c4ccc(C#N)c(C(F)(F)F)c4)C(=O)C34CCC4)cc2CCF)CCN1CC(=O)Nc1cc(Cl)cc(NC2CCC(=O)NC2=O)c1] | N-(3-chloro-5-(2,6-dioxopiperidin-3-ylamino)- phenyl)-2-((R)-4-(2-(4-(7-(4-cyano-3-(trifluoro- methyl)phenyl)-8-oxo-6-thioxo-5,7-diazaspiro [3.4]octan-5-yl)-2-(2-fluoroethyl)phenoxy)ethyl)- 2-methylpiperazin-1-yl)acetamide |
| 144 | 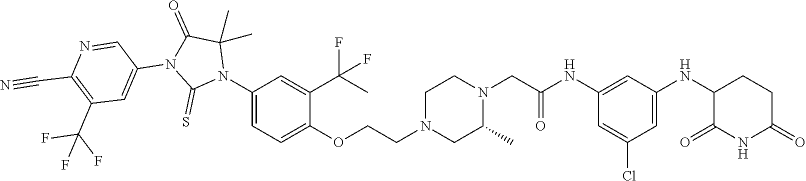 [SMILES: C[C@@H]1CN(CCOc2ccc(N3C(=S)N(c4cnc(C#N)c(C(F)(F)F)c4)C(=O)C3(C)C)cc2C(C)(F)F)CCN1CC(=O)Nc1cc(Cl)cc(NC2CCC(=O)NC2=O)c1] | N-(3-chloro-5-(2,6-dioxopiperidin-3-ylamino)- phenyl)-2-((R)-4-(2-(4-(3-(6-cyano-5-(trifluoro- methyl)pyridin-3-yl)-5,5-dimethyl-4-oxo-2- thioxoimidazolidin-1-yl)-2-ethylphenoxy)ethyl)- 2-methylpiperazin-1-yl)acetamide |
| 145 | 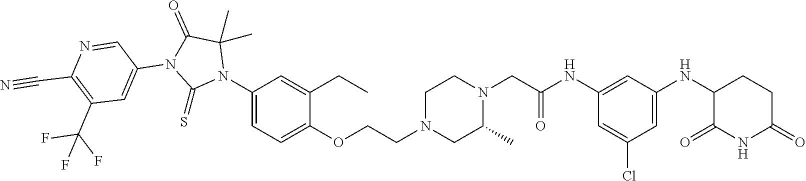 [SMILES: CCc1cc(N2C(=S)N(c3cnc(C#N)c(C(F)(F)F)c3)C(=O)C2(C)C)ccc1OCCN1CCN(CC(=O)Nc2cc(Cl)cc(NC3CCC(=O)NC3=O)c2)[C@H](C)C1] | N-(3-chloro-5-(2,6-dioxopiperidin-3-ylamino)- phenyl)-2-((R)-4-(2-(4-(3-(6-cyano-5-(trifluoro- methyl)pyridin-3-yl)-5,5-dimethyl-4-oxo-2- thioxoimidazolidin-1-yl)-2-ethylphenoxy)ethyl)- 2-methylpiperazin-1-yl)acetamide |
| 146 | 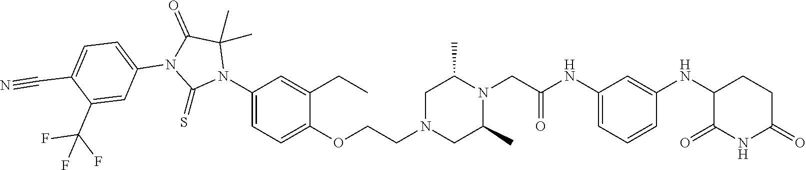 [SMILES: CCc1cc(N2C(=S)N(c3ccc(C#N)c(C(F)(F)F)c3)C(=O)C2(C)C)ccc1OCCN1C[C@H](C)N(CC(=O)Nc2cccc(NC3CCC(=O)NC3=O)c2)[C@@H](C)C1] | 2-((2S,6S)-4-(2-(4-(3-(4-cyano-3-(trifluorometh- yl)phenyl)-5,5-dimethyl-4-oxo-2-thioxoimidazol- idin-1-yl)-2-ethylphenoxy)ethyl)-2,6-dimethyl- piperazin-1-yl)-N-(3-(2,6-dioxopiperidin-3-yl- amino)phenyl)acetamide |
| 147 | 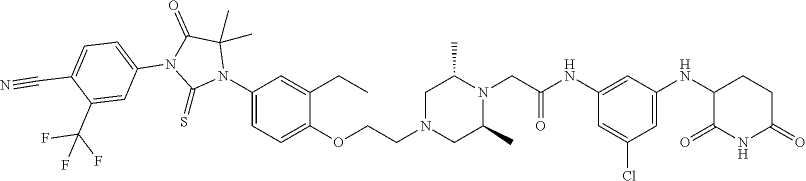 [SMILES: CCc1cc(N2C(=S)N(c3ccc(C#N)c(C(F)(F)F)c3)C(=O)C2(C)C)ccc1OCCN1C[C@H](C)N(CC(=O)Nc2cc(Cl)cc(NC3CCC(=O)NC3=O)c2)[C@@H](C)C1] | N-(3-chloro-5-(2,6-dioxopiperidin-3-ylamino)- phenyl)-2-((2S,6S)-4-(2-(4-(3-(4-cyano-3-(tri- fluoromethyl)phenyl)-5,5-dimethyl-4-oxo-2- thioxoimidazolidin-1-yl)-2-ethylphenoxy)ethyl)- 2,6-dimethylpiperazin-1-yl)acetamide |
| 148 | 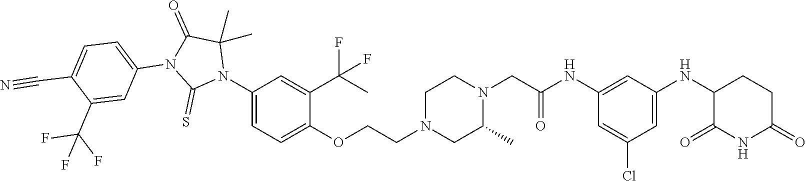 [SMILES: C[C@@H]1CN(CCOc2ccc(N3C(=S)N(c4ccc(C#N)c(C(F)(F)F)c4)C(=O)C3(C)C)cc2C(C)(F)F)CCN1CC(=O)Nc1cc(Cl)cc(NC2CCC(=O)NC2=O)c1] | N-(3-chloro-5-(2,6-dioxopiperidin-3-ylamino)- phenyl)-2-((R)-4-(2-(4-(3-(4-cyano-3-(trifluoro- methyl)phenyl)-5,5-dimethyl-4-oxo-2-thioxo- imidazolidin-1-yl)-2-(1,1-difluoroethyl)phen- oxy)ethyl)-2-methylpiperazin-1-yl)acetamide |
| 149 | 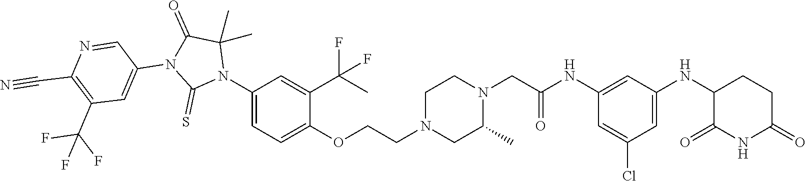 [SMILES: C[C@@H]1CN(CCOc2ccc(N3C(=S)N(c4cnc(C#N)c(C(F)(F)F)c4)C(=O)C3(C)C)cc2C(C)(F)F)CCN1CC(=O)Nc1cc(Cl)cc(NC2CCC(=O)NC2=O)c1] | N-(3-chloro-5-(2,6-dioxopiperidin-3-ylamino)- phenyl)-2-((R)-4-(2-(4-(3-(6-cyano-5-(trifluoro- methyl)pyridin-3-yl)-5,5-dimethyl-4-oxo-2- thioxoimidazolidin-1-yl)-2-(1,1-difluoroethyl)- phenoxy)ethyl)-2-methylpiperazin-1-yl)acetamide |
| 150 | 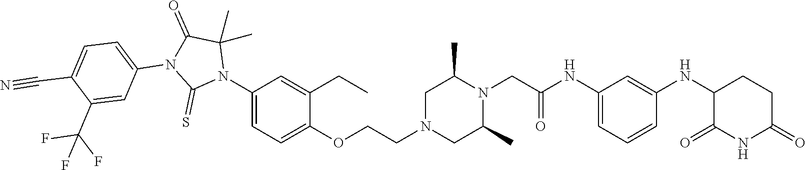 [SMILES: CCc1cc(N2C(=S)N(c3ccc(C#N)c(C(F)(F)F)c3)C(=O)C2(C)C)ccc1OCCN1C[C@@H](C)N(CC(=O)Nc2cccc(NC3CCC(=O)NC3=O)c2)[C@@H](C)C1] | 2-((2S,6R)-4-(2-(4-(3-(4-cyano-3-(trifluorometh- yl)phenyl)-5,5-dimethyl-4-oxo-2-thioxoimidazol- idin-1-yl)-2-ethylphenoxy)ethyl)-2,6-dimethyl- piperazin-1-yl)-N-(3-(2,6-dioxopiperidin-3-yl- amino)phenyl)acetamide |
| 151 | 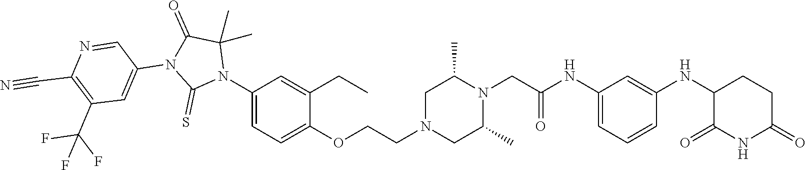 [SMILES: CCc1cc(N2C(=S)N(c3cnc(C#N)c(C(F)(F)F)c3)C(=O)C2(C)C)ccc1OCCN1C[C@@H](C)N(CC(=O)Nc2cccc(NC3CCC(=O)NC3=O)c2)[C@@H](C)C1] | 2-((2R,6S)-4-(2-(4-(3-(6-cyano-5-(trifluorometh- yl)pyridin-3-yl)-5,5-dimethyl-4-oxo-2-thioxo- imidazolidin-1-yl)-2-ethylphenoxy)ethyl)-2,6- dimethylpiperazin-1-yl)-N-(3-(2,6-dioxopiper- idin-3-ylamino)phenyl)acetamide |
| 152 | 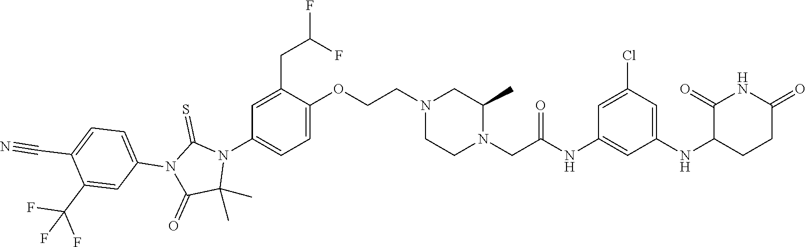 [SMILES: C[C@@H]1CN(CCOc2ccc(N3C(=S)N(c4ccc(C#N)c(C(F)(F)F)c4)C(=O)C3(C)C)cc2CC(F)F)CCN1CC(=O)Nc1cc(Cl)cc(NC2CCC(=O)NC2=O)c1] | N-(3-chloro-5-(2,6-dioxopiperidin-3-ylamino)- phenyl)-2-((R)-4-(2-(4-(3-(4-cyano-3-(trifluoro- methyl)phenyl)-5,5-dimethyl-4-oxo-2-thioxo- imidazolidin-1-yl)-2-(2,2-difluoroethyl)phen- oxy)ethyl)-2-methylpiperazin-1-yl)acetamide |
| 153 | 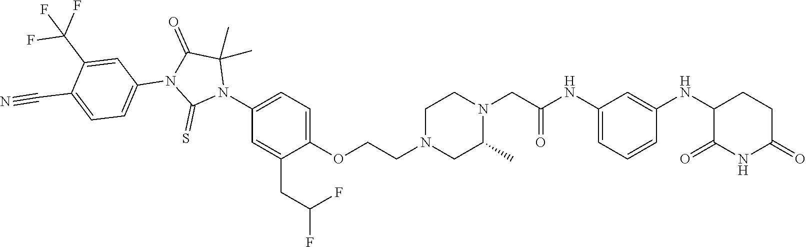 [SMILES: C[C@@H]1CN(CCOc2ccc(N3C(=S)N(c4ccc(C#N)c(C(F)(F)F)c4)C(=O)C3(C)C)cc2CC(F)F)CCN1CC(=O)Nc1cccc(NC2CCC(=O)NC2=O)c1] | 2-((R)-4-(2-(4-(3-(4-cyano-3-(trifluoromethyl)- phenyl)-5,5-dimethyl-4-oxo-2-thioxoimidazol- idin-1-yl)-2-(2,2-difluoroethyl)phenoxy)ethyl)- 2-methylpiperazin-1-yl)-N-(3-(2,6-dioxopiper- idin-3-ylamino)phenyl)acetamide |
| 154 | 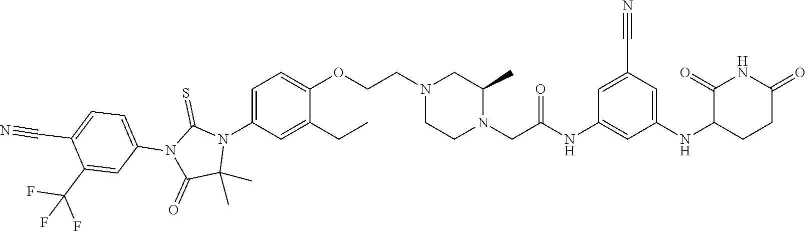 [SMILES: CCc1cc(N2C(=S)N(c3ccc(C#N)c(C(F)(F)F)c3)C(=O)C2(C)C)ccc1OCCN1CCN(CC(=O)Nc2cc(C#N)cc(NC3CCC(=O)NC3=O)c2)[C@H](C)C1] | 2-((R)-4-(2-(4-(3-(4-cyano-3-(trifluoromethyl)- phenyl)-5,5-dimethyl-4-oxo-2-thioxoimidazol- idin-1-yl)-2-ethylphenoxy)ethyl)-2-methylpipe- razin-1-yl)-N-(3-cyano-5-(2,6-dioxopiperidin-3- ylamino)phenyl)acetamide |
| 155 | 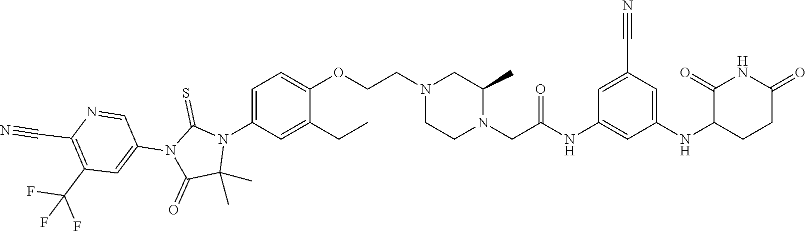 [SMILES: CCc1cc(N2C(=S)N(c3cnc(C#N)c(C(F)(F)F)c3)C(=O)C2(C)C)ccc1OCCN1CCN(CC(=O)Nc2cc(C#N)cc(NC3CCC(=O)NC3=O)c2)[C@H](C)C1] | N-(3-cyano-5-(2,6-dioxopiperidin-3-ylamino)- phenyl)-2-((R)-4-(2-(4-(3-(6-cyano-5-(trifluoro- methyl)pyridin-3-yl)-5,5-dimethyl-4-oxo-2- thioxoimidazolidin-1-yl)-2-ethylphenoxy)ethyl)- 2-methylpiperazin-1-yl)acetamide |
| 156 | 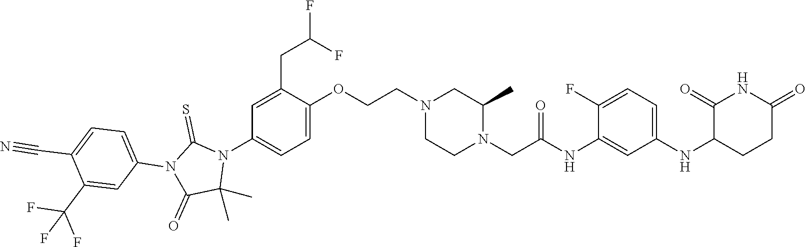 [SMILES: C[C@@H]1CN(CCOc2ccc(N3C(=S)N(c4ccc(C#N)c(C(F)(F)F)c4)C(=O)C3(C)C)cc2CC(F)F)CCN1CC(=O)Nc1cc(NC2CCC(=O)NC2=O)ccc1F] | 2-((R)-4-(2-(4-(3-(4-cyano-3-(trifluoromethyl)- phenyl)-5,5-dimethyl-4-oxo-2-thioxoimidazol- idin-1-yl)-2-(2,2-difluoroethyl)phenoxy)ethyl)- 2-methylpiperazin-1-yl)-N-(5-(2,6-dioxopiper- idin-3-ylamino)-2-fluorophenyl)acetamide |
| 157 | 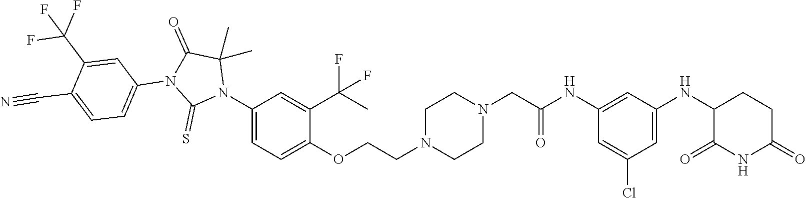 [SMILES: CC(F)(F)c1cc(N2C(=S)N(c3ccc(C#N)c(C(F)(F)F)c3)C(=O)C2(C)C)ccc1OCCN1CCN(CC(=O)Nc2cc(Cl)cc(NC3CCC(=O)NC3=O)c2)CC1] | N-(3-chloro-5-(2,6-dioxopiperidin-3-ylamino)- phenyl)-2-(4-(2-(4-(3-(4-cyano-3-(trifluorometh- yl)phenyl)-5,5-dimethyl-4-oxo-2-thioxoimidazol- idin-1-yl)-2-(1,1-difluoroethyl)phenoxy)ethyl)- piperazin-1-yl)acetamide |
| 158 | 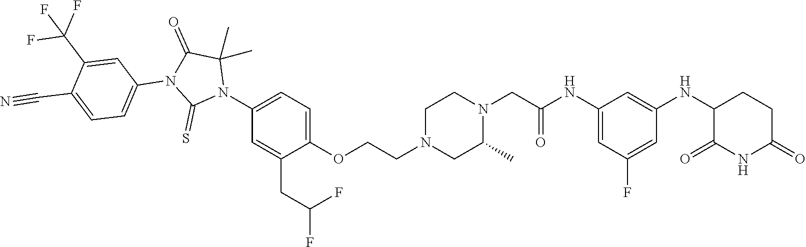 [SMILES: C[C@@H]1CN(CCOc2ccc(N3C(=S)N(c4ccc(C#N)c(C(F)(F)F)c4)C(=O)C3(C)C)cc2CC(F)F)CCN1CC(=O)Nc1cc(F)cc(NC2CCC(=O)NC2=O)c1] | 2-((R)-4-(2-(4-(3-(4-cyano-3-(trifluoromethyl)- phenyl)-5,5-dimethyl-4-oxo-2-thioxoimidazol- idin-1-yl)-2-(2,2-difluoroethyl)phenoxy)ethyl)- 2-methylpiperazin-1-yl)-N-(3-(2,6-dioxopiper- idin-3-ylamino)-5-fluorophenyl)acetamide |
| 159 | 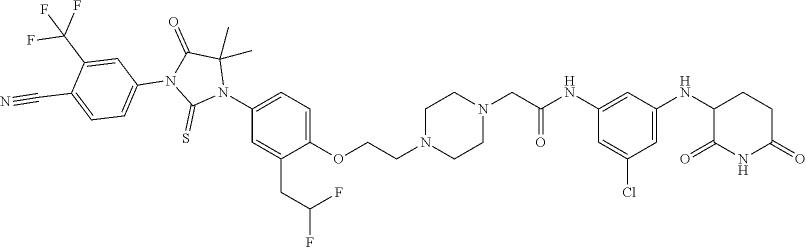 [SMILES: CC1(C)C(=O)N(c2ccc(C#N)c(C(F)(F)F)c2)C(=S)N1c1ccc(OCCN2CCN(CC(=O)Nc3cc(Cl)cc(NC4CCC(=O)NC4=O)c3)CC2)c(CC(F)F)c1] | N-(3-chloro-5-(2,6-dioxopiperidin-3-ylamino)- phenyl)-2-(4-(2-(4-(3-(4-cyano-3-(trifluorometh- yl)phenyl)-5,5-dimethyl-4-oxo-2-thioxoimidazol- idin-1-yl)-2-(2,2-difluoroethyl)phenoxy)ethyl)- piperazin-1-yl)acetamide |
| 160 | 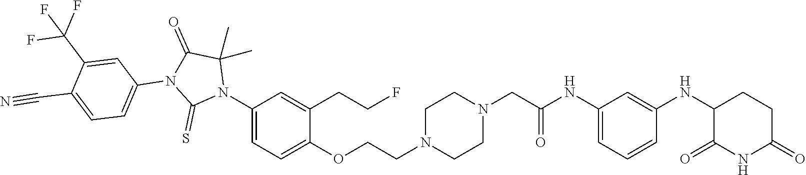 [SMILES: CC1(C)C(=O)N(c2ccc(C#N)c(C(F)(F)F)c2)C(=S)N1c1ccc(OCCN2CCN(CC(=O)Nc3cccc(NC4CCC(=O)NC4=O)c3)CC2)c(CCF)c1] | 2-(4-(2-(4-(3-(4-cyano-3-(trifluoromethyl)phen- yl)-5,5-dimethyl-4-oxo-2-thioxoimidazolidin-1- yl)-2-(2-fluoroethyl)phenoxy)ethyl)piperazin-1- yl)-N-(3-(2,6-dioxopiperidin-3-ylamino)phen- yl)acetamide |
| 161 | 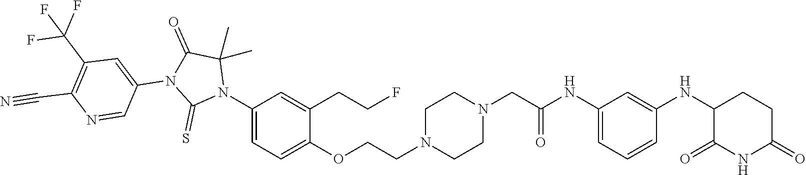 [SMILES: CC1(C)C(=O)N(c2cnc(C#N)c(C(F)(F)F)c2)C(=S)N1c1ccc(OCCN2CCN(CC(=O)Nc3cccc(NC4CCC(=O)NC4=O)c3)CC2)c(CCF)c1] | 2-(4-(2-(4-(3-(6-cyano-5-(trifluoromethyl)pyridin- 3-yl)-5,5-dimethyl-4-oxo-2-thioxoimidazolidin- 1-yl)-2-(2-fluoroethyl)phenoxy)ethyl)piperazin- 1-yl)-N-(3-(2,6-dioxopiperidin-3-ylamino)phen- yl)acetamide |
| 162 | 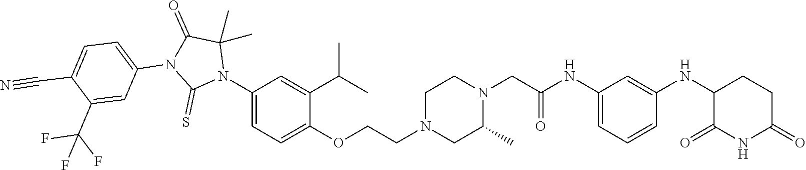 [SMILES: CC(C)c1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)[C@H](C)C1] | 2-((R)-4-(2-(4-(3-(4-cyano-3-(trifluoromethyl)- phenyl)-5,5-dimethyl-4-oxo-2-thioxoimidazol- idin-1-yl)-2-isopropylphenoxy)ethyl)-2-meth- ylpiperazin-1-yl)-N-(3-(2,6-dioxopiperidin-3- ylamino)phenyl)acetamide |
| 163 | 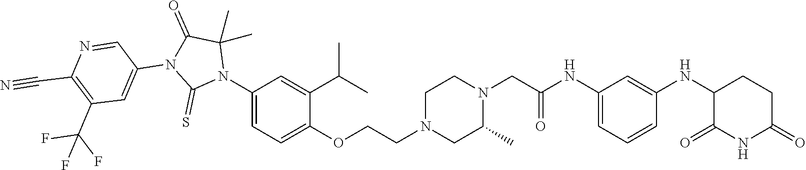 [SMILES: CC(C)c1cc(N2C(=S)N(c3cnc(C#N)c(C(F)(F)F)c3)C(=O)C2(C)C)ccc1OCCN1CCN(CC(=O)Nc2cccc(NC3CCC(=O)NC3=O)c2)[C@H](C)C1] | 2-((R)-4-(2-(4-(3-(6-cyano-5-(trifluoromethyl)- pyridin-3-yl)-5,5-dimethyl-4-oxo-2-thioxoimid- azol-idin-1-yl)-2-isopropylphenoxy)ethyl)-2- methylpiperazin-1-yl)-N-(3-(2,6-dioxopiperidin- 3-ylamino)phenyl)acetamide |
| 164 | 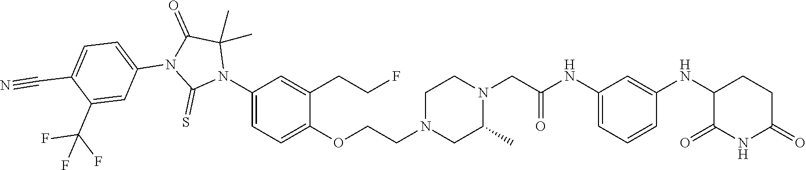 [SMILES: C[C@@H]1CN(CCOc2ccc(N3C(=S)N(c4ccc(C#N)c(C(F)(F)F)c4)C(=O)C3(C)C)cc2CCF)CCN1CC(=O)Nc1cccc(NC2CCC(=O)NC2=O)c1] | 2-((R)-4-(2-(4-(3-(4-cyano-3-(trifluoromethyl)- phenyl)-5,5-dimethyl-4-oxo-2-thioxoimidazol- idin-1-yl)-2-(2-fluoroethyl)phenoxy)ethyl)-2- methylpiperazin-1-yl)-N-(3-(2,6-dioxopiperidin- 3-ylamino)phenyl)acetamide |
| 165 | 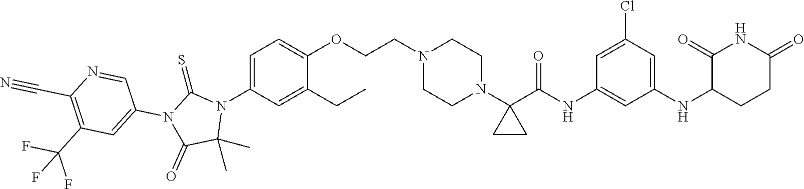 [SMILES: CCc1cc(N2C(=S)N(c3cnc(C#N)c(C(F)(F)F)c3)C(=O)C2(C)C)ccc1OCCN1CCN(C2(C(=O)Nc3cc(Cl)cc(NC4CCC(=O)NC4=O)c3)CC2)CC1] | N-(3-chloro-5-(2,6-dioxopiperidin-3-ylamino)- phenyl)-1-(4-(2-(4-(3-(6-cyano-5-(trifluorometh- yl)pyridin-3-yl)-5,5-dimethyl-4-oxo-2-thioxo- imidazolidin-1-yl)-2-ethylphenoxy)ethyl)piper- azin-1-yl)cyclopropanecarboxamide |
| 166 | 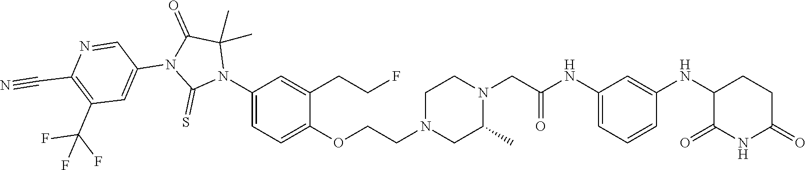 [SMILES: C[C@@H]1CN(CCOc2ccc(N3C(=S)N(c4cnc(C#N)c(C(F)(F)F)c4)C(=O)C3(C)C)cc2CCF)CCN1CC(=O)Nc1cccc(NC2CCC(=O)NC2=O)c1] | 2-((R)-4-(2-(4-(3-(6-cyano-5-(trifluoromethyl)- pyridin-3-yl)-5,5-dimethyl-4-oxo-2-thioxo- imidazolidin-1-yl)-2-(2-fluoroethyl)phenoxy)- ethyl)-2-methylpiperazin-1-yl)-N-(3-(2,6-dioxo- piperidin-3-ylamino)phenyl)acetamide |
| 167 | 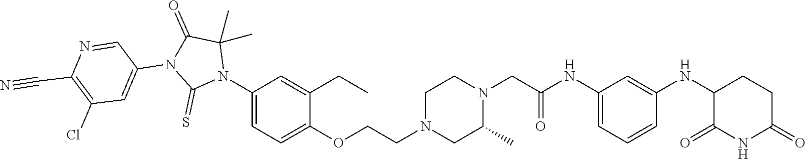 [SMILES: CCc1cc(N2C(=S)N(c3cnc(C#N)c(Cl)c3)C(=O)C2(C)C)ccc1OCCN1CCN(CC(=O)Nc2cccc(NC3CCC(=O)NC3=O)c2)[C@H](C)C1] | 2-((R)-4-(2-(4-(3-(5-chloro-6-cyanopyridin-3-yl)- 5,5-dimethyl-4-oxo-2-thioxoimidazolidin-1-yl)- 2-ethylphenoxy)ethyl)-2-methylpiperazin-1-yl)- N-(3-(2,6-dioxopiperidin-3-ylamino)phenyl)- acetamide |
| 168 | 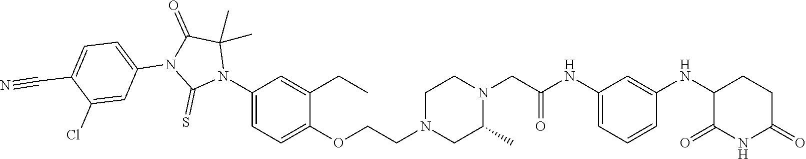 [SMILES: CCc1cc(N2C(=S)N(c3ccc(C#N)c(Cl)c3)C(=O)C2(C)C)ccc1OCCN1CCN(CC(=O)Nc2cccc(NC3CCC(=O)NC3=O)c2)[C@H](C)C1] | 2-((R)-4-(2-(4-(3-(3-chloro-4-cyanophenyl)-5,5- dimethyl-4-oxo-2-thioxoimidazolidin-1-yl)-2- ethylphenoxy)ethyl)-2-methylpiperazin-1-yl)-N- (3-(2,6-dioxopiperidin-3-ylamino)phenyl)acet- amide |
| 169 | 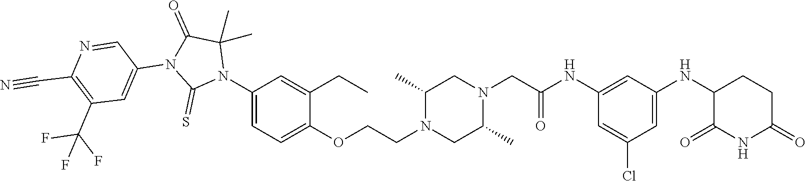 [SMILES: CCc1cc(N2C(=S)N(c3cnc(C#N)c(C(F)(F)F)c3)C(=O)C2(C)C)ccc1OCCN1C[C@@H](C)N(CC(=O)Nc2cc(Cl)cc(NC3CCC(=O)NC3=O)c2)C[C@H]1C] | N-(3-chloro-5-(2,6-dioxopiperidin-3-ylamino)- phenyl)-2-((2R,5R)-4-(2-(4-(3-(6-cyano-5- (trifluoromethyl)pyridin-3-yl)-5,5-dimethyl-4- oxo-2-thioxoimidazolidin-1-yl)-2-ethylphenoxy)- ethyl)-2,5-dimethylpiperazin-1-yl)acetamide |
| 170 | 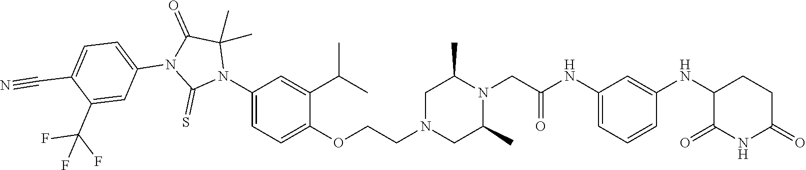 [SMILES: CC(C)c1cc(N2C(=S)N(c3ccc(C#N)c(C(F)(F)F)c3)C(=O)C2(C)C)ccc1OCCN1C[C@@H](C)N(CC(=O)Nc2cccc(NC3CCC(=O)NC3=O)c2)[C@@H](C)C1] | 2-((2S,6R)-4-(2-(4-(3-(4-cyano-3-(trifluorometh- yl)phenyl)-5,5-dimethyl-4-oxo-2-thioxoimidazol- idin-1-yl)-2-isopropylphenoxy)ethyl)-2,6-dimeth- ylpiperazin-1-yl)-N-(3-(2,6-dioxopiperidin-3- ylamino)phenyl)acetamide |
| 171 | 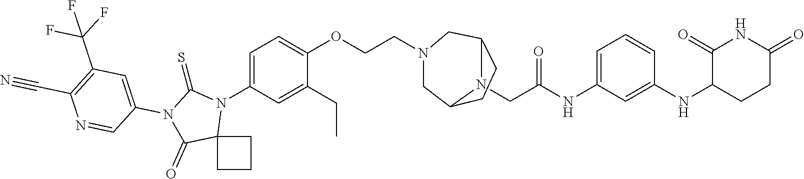 [SMILES: CCc1cc(N2C(=S)N(c3cnc(C#N)c(C(F)(F)F)c3)C(=O)C23CCC3)ccc1OCCN1CC2CCC(C1)N2CC(=O)Nc1cccc(NC2CCC(=O)NC2=O)c1] | 2-(3-(2-(4-(7-(6-cyano-5-(trifluoromethyl)pyridin- 3-yl)-8-oxo-6-thioxo-5,7-diazaspiro[3.4]octan-5- yl)-2-ethylphenoxy)ethyl)-3,8-diazabicyclo[3.2.1] octan-8-yl)-N-(3-(2,6-dioxopiperidin-3-ylamino)- phenyl)acetamide |
| 172 | 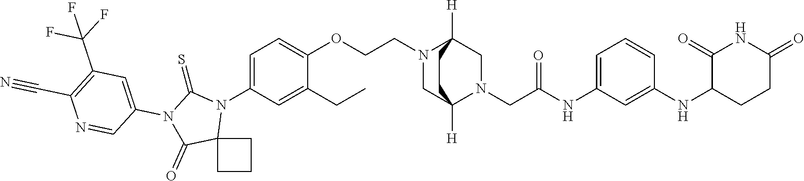 [SMILES: CCc1cc(N2C(=S)N(c3cnc(C#N)c(C(F)(F)F)c3)C(=O)C23CCC3)ccc1OCCN1C[C@@H]2CC[C@H]1CN2CC(=O)Nc1cccc(NC2CCC(=O)NC2=O)c1] | 2-((1S,4S)-5-(2-(4-(7-(6-cyano-5-(trifluorometh- yl)pyridin-3-yl)-8-oxo-6-thioxo-5,7-diazaspiro [3.4]octan-5-yl)-2-ethylphenoxy)ethyl)-2,5- diazabicyclo[2.2.2]octan-2-yl)-N-(3-(2,6-dioxo- piperidin-3-ylamino)phenyl)acetamide |
| 173 | 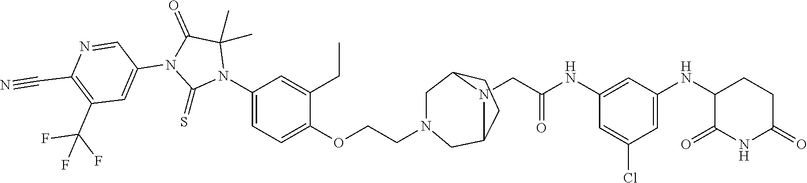 [SMILES: CCc1cc(N2C(=S)N(c3cnc(C#N)c(C(F)(F)F)c3)C(=O)C2(C)C)ccc1OCCN1CC2CCC(C1)N2CC(=O)Nc1cc(Cl)cc(NC2CCC(=O)NC2=O)c1] | N-(3-chloro-5-(2,6-dioxopiperidin-3-ylamino)- phenyl)-2-(3-(2-(4-(3-(6-cyano-5-(trifluorometh- yl)pyridin-3-yl)-5,5-dimethyl-4-oxo-2-thioxo- imidazolidin-1-yl)-2-ethylphenoxy)ethyl)-3,8- diazabicyclo[3.2.1]octan-8-yl)acetamide |
| 174 | 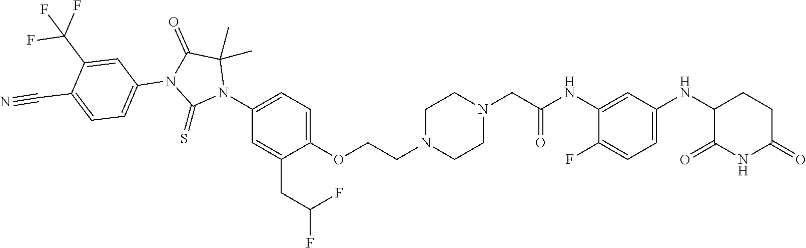 [SMILES: CC1(C)C(=O)N(c2ccc(C#N)c(C(F)(F)F)c2)C(=S)N1c1ccc(OCCN2CCN(CC(=O)Nc3cc(NC4CCC(=O)NC4=O)ccc3F)CC2)c(CC(F)F)c1] | 2-(4-(2-(4-(3-(4-cyano-3-(trifluoromethyl)phen- yl)-5,5-dimethyl-4-oxo-2-thioxoimidazolidin-1- yl)-2-(2,2-difluoroethyl)phenoxy)ethyl)piperazin- 1-yl)-N-(5-(2,6-dioxopiperidin-3-ylamino)-2- fluorophenyl)acetamide |
| 175 | 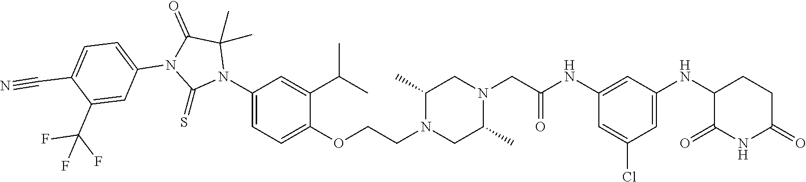 [SMILES: CC(C)c1cc(N2C(=S)N(c3ccc(C#N)c(C(F)(F)F)c3)C(=O)C2(C)C)ccc1OCCN1C[C@@H](C)N(CC(=O)Nc2cc(Cl)cc(NC3CCC(=O)NC3=O)c2)C[C@H]1C] | N-(3-chloro-5-(2,6-dioxopiperidin-3-ylamino)- phenyl)-2-((2R,5R)-4-(2-(4-(3-(4-cyano-3- (trifluoromethyl)phenyl)-5,5-dimethyl-4-oxo-2- thioxoimidazolidin-1-yl)-2-isopropylphenoxy)- ethyl)-2,5-dimethylpiperazin-1-yl)acetamide |
| 176 | 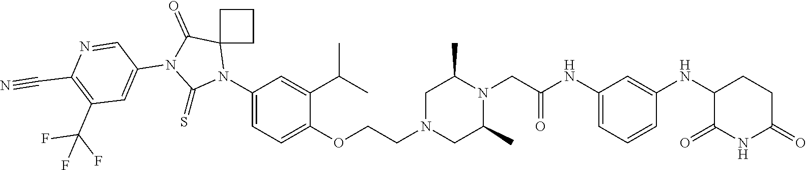 [SMILES: CC(C)c1cc(N2C(=S)N(c3cnc(C#N)c(C(F)(F)F)c3)C(=O)C23CCC3)ccc1OCCN1C[C@@H](C)N(CC(=O)Nc2cccc(NC3CCC(=O)NC3=O)c2)[C@@H](C)C1] | 2-((2S,6R)-4-(2-(4-(7-(6-cyano-5-(trifluorometh- yl)pyridin-3-yl)-8-oxo-6-thioxo-5,7-diazaspiro [3.4]octan-5-yl)-2-isopropylphenoxy)ethyl)-2,6- dimethylpiperazin-1-yl)-N-(3-(2,6-dioxopiper- idin-3-ylamino)phenyl)acetamide |
| 177 | 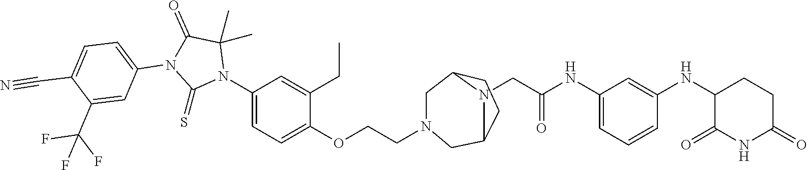 [SMILES: CCc1cc(N2C(=S)N(c3ccc(C#N)c(C(F)(F)F)c3)C(=O)C2(C)C)ccc1OCCN1CC2CCC(C1)N2CC(=O)Nc1cccc(NC2CCC(=O)NC2=O)c1] | 2-(3-(2-(4-(3-(4-cyano-3-(trifluoromethyl)phen- yl)-5,5-dimethyl-4-oxo-2-thioxoimidazolidin-1- yl)-2-ethylphenoxy)ethyl)-3,8-diazabicyclo[3.2.1] octan-8-yl)-N-(3-(2,6-dioxopiperidin-3-ylamino)- phenyl)acetamide |
| 178 | 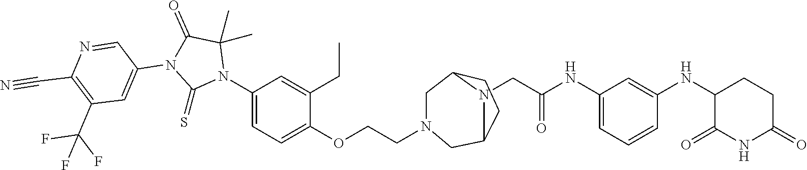 [SMILES: CCc1cc(N2C(=S)N(c3cnc(C#N)c(C(F)(F)F)c3)C(=O)C2(C)C)ccc1OCCN1CC2CCC(C1)N2CC(=O)Nc1cccc(NC2CCC(=O)NC2=O)c1] | 2-(3-(2-(4-(3-(6-cyano-5-(trifluoromethyl)pyri- din-3-yl)-5,5-dimethyl-4-oxo-2-thioxoimidazol- idin-1-yl)-2-ethylphenoxy)ethyl)-3,8-diazabicyclo [3.2.1]octan-8-yl)-N-(3-(2,6-dioxopiperidin-3- ylamino)phenyl)acetamide |
| 179 | 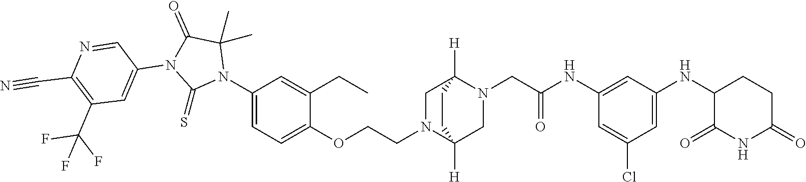 [SMILES: CCc1cc(N2C(=S)N(c3cnc(C#N)c(C(F)(F)F)c3)C(=O)C2(C)C)ccc1OCCN1C[C@@H]2CC[C@H]1CN2CC(=O)Nc1cc(Cl)cc(NC2CCC(=O)NC2=O)c1] | N-(3-chloro-5-(2,6-dioxopiperidin-3-ylamino)- phenyl)-2-((1S,4S)-5-(2-(4-(3-(6-cyano-5- (trifluoromethyl)pyridin-3-yl)-5,5-dimethyl-4- oxo-2-thioxoimidazolidin-1-yl)-2-ethylphenoxy)- ethyl)-2,5-diazabicyclo[2.2.2]octan-2-yl)acet- amide |
| 180 | 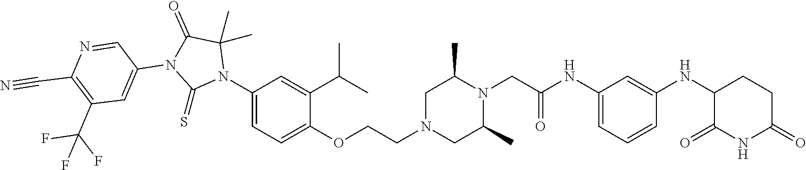 [SMILES: CC(C)c1cc(N2C(=S)N(c3cnc(C#N)c(C(F)(F)F)c3)C(=O)C2(C)C)ccc1OCCN1C[C@@H](C)N(CC(=O)Nc2cccc(NC3CCC(=O)NC3=O)c2)[C@@H](C)C1] | 2-((2S,6R)-4-(2-(4-(3-(6-cyano-5-(trifluorometh- yl)pyridin-3-yl)-5,5-dimethyl-4-oxo-2-thioxoimid- azolidin-1-yl)-2-isopropylphenoxy)ethyl)-2,6- dimethylpiperazin-1-yl)-N-(3-(2,6-dioxopiperi- din-3-ylamino)phenyl)acetamide |
| 181 | 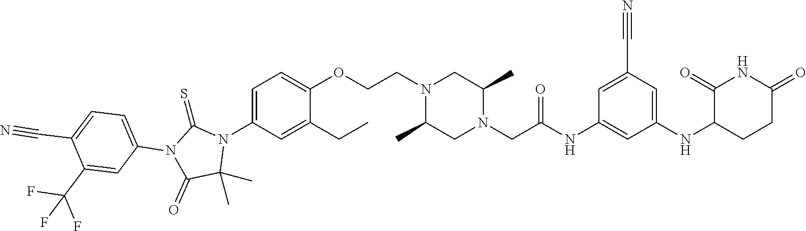 [SMILES: CCc1cc(N2C(=S)N(c3ccc(C#N)c(C(F)(F)F)c3)C(=O)C2(C)C)ccc1OCCN1C[C@@H](C)N(CC(=O)Nc2cc(C#N)cc(NC3CCC(=O)NC3=O)c2)C[C@H]1C] | 2-((2R,5R)-4-(2-(4-(3-(4-cyano-3-(trifluorometh- yl)phenyl)-5,5-dimethyl-4-oxo-2-thioxoimidazol- idin-1-yl)-2-ethylphenoxy)ethyl)-2,5-dimethyl- piperazin-1-yl)-N-(3-cyano-5-(2,6-dioxopiperidin- 3-ylamino)phenyl)acetamide |
| 182 | 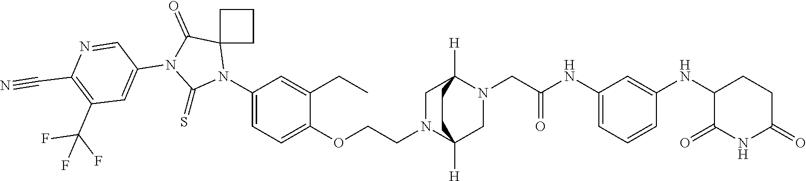 [SMILES: CCc1cc(N2C(=S)N(c3cnc(C#N)c(C(F)(F)F)c3)C(=O)C23CCC3)ccc1OCCN1C[C@H]2CC[C@@H]1CN2CC(=O)Nc1cccc(NC2CCC(=O)NC2=O)c1] | 2-((1R,4R)-5-(2-(4-(7-(6-cyano-5-(trifluorometh- yl)pyridin-3-yl)-8-oxo-6-thioxo-5,7-diazaspiro [3.4]octan-5-yl)-2-ethylphenoxy)ethyl)-2,5- diazabicyclo[2.2.2]octan-2-yl)-N-(3-(2,6-dioxo- piperidin-3-ylamino)phenyl)acetamide |
| 183 | 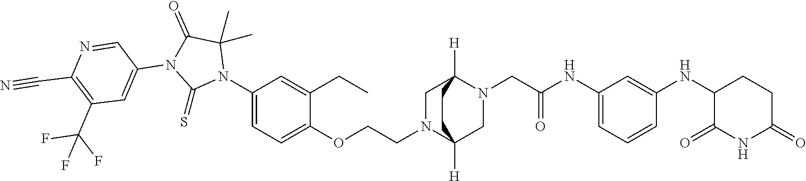 [SMILES: CCc1cc(N2C(=S)N(c3cnc(C#N)c(C(F)(F)F)c3)C(=O)C2(C)C)ccc1OCCN1C[C@H]2CC[C@@H]1CN2CC(=O)Nc1cccc(NC2CCC(=O)NC2=O)c1] | 2-((1R,4R)-5-(2-(4-(3-(6-cyano-5-(trifluorometh- yl)pyridin-3-yl)-5,5-dimethyl-4-oxo-2-thioxo- imidazolidin-1-yl)-2-ethylphenoxy)ethyl)-2,5- diazabicyclo[2.2.2]octan-2-yl)-N-(3-(2,6-dioxo- piperidin-3-ylamino)phenyl)acetamide |
| 184 | 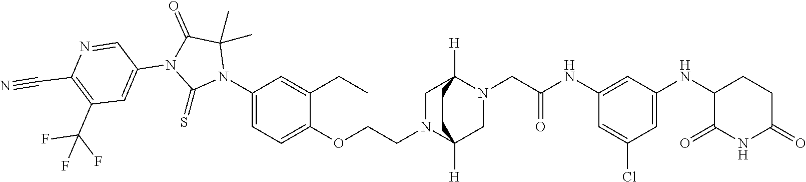 [SMILES: CCc1cc(N2C(=S)N(c3cnc(C#N)c(C(F)(F)F)c3)C(=O)C2(C)C)ccc1OCCN1C[C@H]2CC[C@@H]1CN2CC(=O)Nc1cc(Cl)cc(NC2CCC(=O)NC2=O)c1] | N-(3-chloro-5-(2,6-dioxopiperidin-3-ylamino)- phenyl)-2-((1R,4R)-5-(2-(4-(3-(6-cyano-5- (trifluoromethyl)pyridin-3-yl)-5,5-dimethyl-4- oxo-2-thioxoimidazolidin-1-yl)-2-ethylphen- oxy)ethyl)-2,5-diazabicyclo[2.2.2]octan-2-yl)- acetamide |
| 185 | 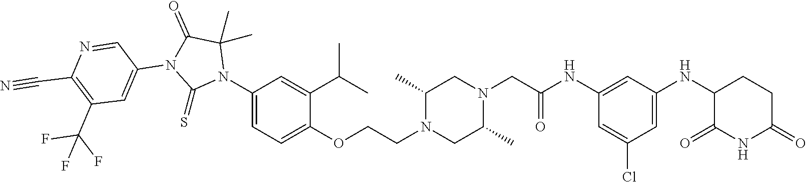 [SMILES: CC(C)c1cc(N2C(=S)N(c3cnc(C#N)c(C(F)(F)F)c3)C(=O)C2(C)C)ccc1OCCN1C[C@@H](C)N(CC(=O)Nc2cc(Cl)cc(NC3CCC(=O)NC3=O)c2)C[C@H]1C] | N-(3-chloro-5-(2,6-dioxopiperidin-3-ylamino)- phenyl)-2-((2R,5R)-4-(2-(4-(3-(6-cyano-5- (trifluoromethyl)pyridin-3-yl)-5,5-dimethyl-4- oxo-2-thioxoimidazolidin-1-yl)-2-isopropyl- phenoxy)ethyl)-2,5-dimethylpiperazin-1-yl)- acetamide |
| 186 | 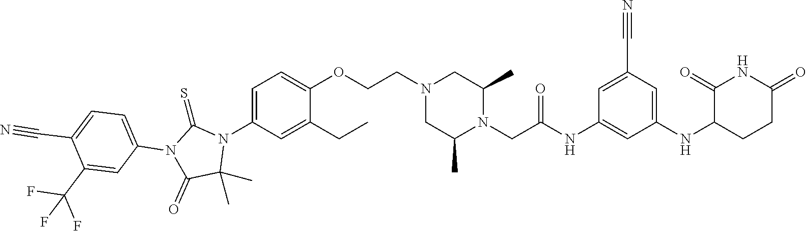 [SMILES: CCc1cc(N2C(=S)N(c3ccc(C#N)c(C(F)(F)F)c3)C(=O)C2(C)C)ccc1OCCN1C[C@@H](C)N(CC(=O)Nc2cc(C#N)cc(NC3CCC(=O)NC3=O)c2)[C@@H](C)C1] | 2-((2S,6R)-4-(2-(4-(3-(4-cyano-3-(trifluorometh- yl)phenyl)-5,5-dimethyl-4-oxo-2-thioxoimidazol- idin-1-yl)-2-ethylphenoxy)ethyl)-2,6-dimethyl- piperazin-1-yl)-N-(3-cyano-5-(2,6-dioxopiperidin- 3-ylamino)phenyl)acetamide |
| 187 | 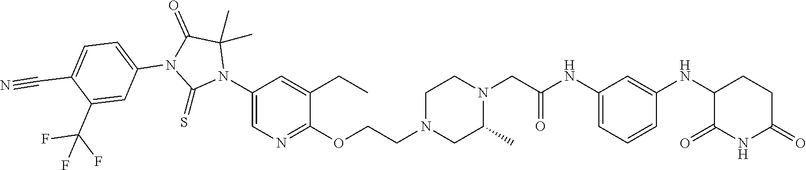 [SMILES: CCc1cc(N2C(=S)N(c3ccc(C#N)c(C(F)(F)F)c3)C(=O)C2(C)C)cnc1OCCN1CCN(CC(=O)Nc2cccc(NC3CCC(=O)NC3=O)c2)[C@H](C)C1] | 2-((R)-4-(2-(5-(3-(4-cyano-3-(trifluoromethyl)- phenyl)-5,5-dimethyl-4-oxo-2-thioxoimidazolidin- 1-yl)-3-ethylpyridin-2-yloxy)ethyl)-2-methyl- piperazin-1-yl)-N-(3-(2,6-dioxopiperidin-3-yl- amino)phenyl)acetamide |
| 188 | 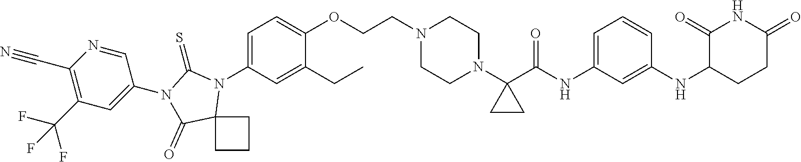 [SMILES: CCc1cc(N2C(=S)N(c3cnc(C#N)c(C(F)(F)F)c3)C(=O)C23CCC3)ccc1OCCN1CCN(C2(C(=O)Nc3cccc(NC4CCC(=O)NC4=O)c3)CC2)CC1] | 1-(4-(2-(4-(7-(6-cyano-5-(trifluoromethyl)pyridin- 3-yl)-8-oxo-6-thioxo-5,7-diazaspiro[3.4]octan-5- yl)-2-ethylphenoxy)ethyl)piperazin-1-yl)-N-(3- (2,6-dioxopiperidin-3-ylamino)phenyl)cyclopro- panecarboxamide |
| 189 | 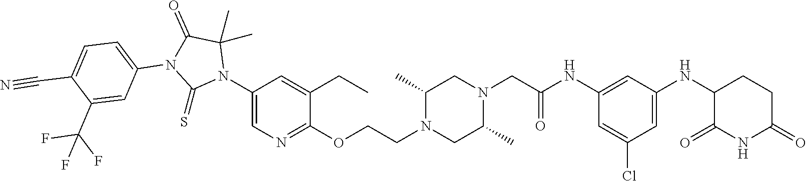 [SMILES: CCc1cc(N2C(=S)N(c3ccc(C#N)c(C(F)(F)F)c3)C(=O)C2(C)C)cnc1OCCN1C[C@@H](C)N(CC(=O)Nc2cc(Cl)cc(NC3CCC(=O)NC3=O)c2)C[C@H]1C] | N-(3-chloro-5-(2,6-dioxopiperidin-3-ylamino)- phenyl)-2-((2R,5R)-4-(2-(5-(3-(4-cyano-3- (trifluoromethyl)phenyl)-5,5-dimethyl-4-oxo-2- thioxoimidazolidin-1-yl)-3-ethylpyridin-2-yl- oxy)ethyl)-2,5-dimethylpiperazin-1-yl)acetamide |
| 190 | 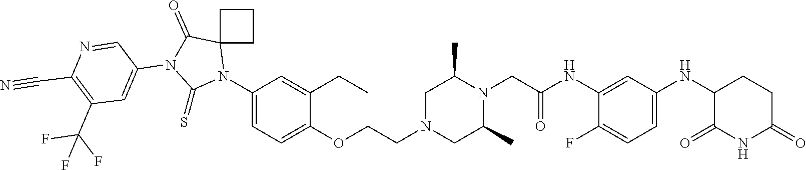 [SMILES: CCc1cc(N2C(=S)N(c3cnc(C#N)c(C(F)(F)F)c3)C(=O)C23CCC3)ccc1OCCN1C[C@@H](C)N(CC(=O)Nc2cc(NC3CCC(=O)NC3=O)ccc2F)[C@@H](C)C1] | 2-((2S,6R)-4-(2-(4-(7-(6-cyano-5-(trifluorometh- yl)pyridin-3-yl)-8-oxo-6-thioxo-5,7-diazaspiro [3.4]octan-5-yl)-2-ethylphenoxy)ethyl)-2,6- dimethylpiperazin-1-yl)-N-(5-(2,6-dioxopiper- idin-3-ylamino)-2-fluorophenyl)acetamide |
| 191 | 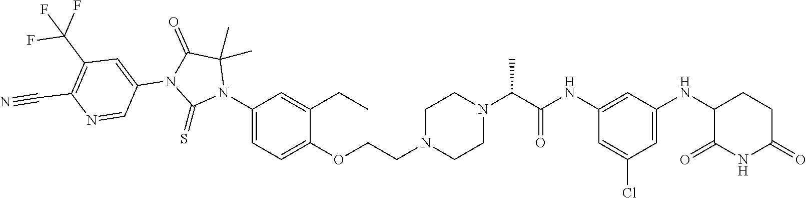 [SMILES: CCc1cc(N2C(=S)N(c3cnc(C#N)c(C(F)(F)F)c3)C(=O)C2(C)C)ccc1OCCN1CCN([C@H](C)C(=O)Nc2cc(Cl)cc(NC3CCC(=O)NC3=O)c2)CC1] | (2R)-N-(3-chloro-5-(2,6-dioxopiperidin-3-yl- amino)phenyl)-2-(4-(2-(4-(3-(6-cyano-5- (trifluoromethyl)pyridin-3-yl)-5,5-dimethyl-4- oxo-2-thioxoimidazolidin-1-yl)-2-ethylphen- oxy)ethyl)piperazin-1-yl)propanamide |
| 192 | 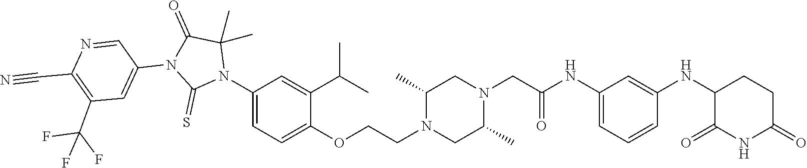 [SMILES: CC(C)c1cc(N2C(=S)N(c3cnc(C#N)c(C(F)(F)F)c3)C(=O)C2(C)C)ccc1OCCN1C[C@@H](C)N(CC(=O)Nc2cccc(NC3CCC(=O)NC3=O)c2)C[C@H]1C] | 2-((2R,5R)-4-(2-(4-(3-(6-cyano-5-(trifluorometh- yl)pyridin-3-yl)-5,5-dimethyl-4-oxo-2-thioxoimid- azolidin-1-yl)-2-isopropylphenoxy)ethyl)-2,5- dimethylpiperazin-1-yl)-N-(3-(2,6-dioxopiper- idin-3-ylamino)phenyl)acetamide |
| 193 | 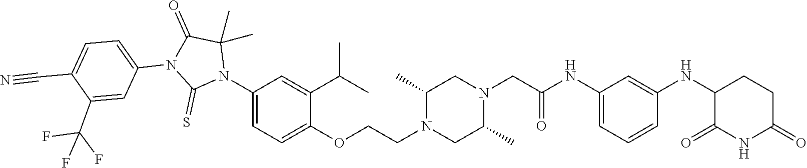 [SMILES: CC(C)c1cc(N2C(=S)N(c3ccc(C#N)c(C(F)(F)F)c3)C(=O)C2(C)C)ccc1OCCN1C[C@@H](C)N(CC(=O)Nc2cccc(NC3CCC(=O)NC3=O)c2)C[C@H]1C] | 2-((2R,5R)-4-(2-(4-(3-(4-cyano-3-(trifluorometh- yl)phenyl)-5,5-dimethyl-4-oxo-2-thioxoimidazol- idin-1-yl)-2-isopropylphenoxy)ethyl)-2,5-dimeth- ylpiperazin-1-yl)-N-(3-(2,6-dioxopiperidin-3-yl- amino)phenyl)acetamide |
| 194 | 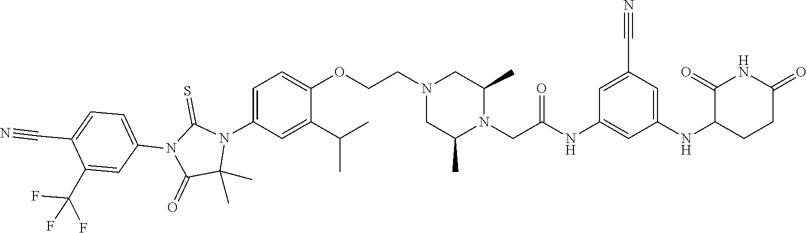 [SMILES: CC(C)c1cc(N2C(=S)N(c3ccc(C#N)c(C(F)(F)F)c3)C(=O)C2(C)C)ccc1OCCN1C[C@@H](C)N(CC(=O)Nc2cc(C#N)cc(NC3CCC(=O)NC3=O)c2)[C@@H](C)C1] | 2-((2S,6R)-4-(2-(4-(3-(4-cyano-3-(trifluorometh- yl)phenyl)-5,5-dimethyl-4-oxo-2-thioxoimidazol- idin-1-yl)-2-isopropylphenoxy)ethyl)-2,6-dimeth- ylpiperazin-1-yl)-N-(3-cyano-5-(2,6-dioxopiper- idin-3-ylamino)phenyl)acetamide |
| 195 | 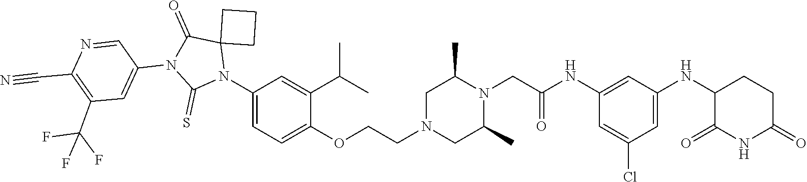 [SMILES: CC(C)c1cc(N2C(=S)N(c3cnc(C#N)c(C(F)(F)F)c3)C(=O)C23CCC3)ccc1OCCN1C[C@@H](C)N(CC(=O)Nc2cc(Cl)cc(NC3CCC(=O)NC3=O)c2)[C@@H](C)C1] | N-(3-chloro-5-(2,6-dioxopiperidin-3-ylamino)- phenyl)-2-((2S,6R)-4-(2-(4-(7-(6-cyano-5- (trifluoromethyl)pyridin-3-yl)-8-oxo-6-thioxo- 5,7-diazaspiro[3.4]octan-5-yl)-2-isopropylphen- oxy)ethyl)-2,6-dimethylpiperazin-1-yl)acetamide |
| 196 | 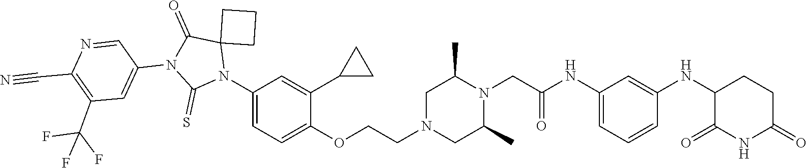 [SMILES: C[C@@H]1CN(CCOc2ccc(N3C(=S)N(c4cnc(C#N)c(C(F)(F)F)c4)C(=O)C34CCC4)cc2C2CC2)C[C@H](C)N1CC(=O)Nc1cccc(NC2CCC(=O)NC2=O)c1] | 2-((2S,6R)-4-(2-(4-(7-(6-cyano-5-(trifluorometh- yl)pyridin-3-yl)-8-oxo-6-thioxo-5,7-diazaspiro [3.4]octan-5-yl)-2-cyclopropylphenoxy)ethyl)- 2,6-dimethylpiperazin-1-yl)-N-(3-(2,6-dioxo- piperidin-3-ylamino)phenyl)acetamide |
| 197 | 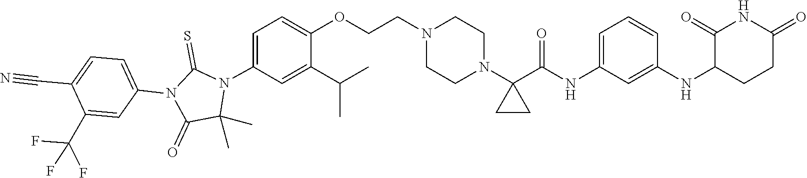 [SMILES: CC(C)c1cc(N2C(=S)N(c3ccc(C#N)c(C(F)(F)F)c3)C(=O)C2(C)C)ccc1OCCN1CCN(C2(C(=O)Nc3cccc(NC4CCC(=O)NC4=O)c3)CC2)CC1] | 1-(4-(2-(4-(3-(4-cyano-3-(trifluoromethyl)phen- yl)-5,5-dimethyl-4-oxo-2-thioxoimidazolidin-1- yl)-2-isopropylphenoxy)ethyl)piperazin-1-yl)-N- (3-(2,6-dioxopiperidin-3-ylamino)phenyl)cyclo- propanecarboxamide |
| 198 | 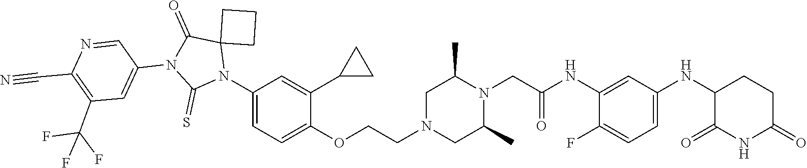 [SMILES: C[C@@H]1CN(CCOc2ccc(N3C(=S)N(c4cnc(C#N)c(C(F)(F)F)c4)C(=O)C34CCC4)cc2C2CC2)C[C@H](C)N1CC(=O)Nc1cc(NC2CCC(=O)NC2=O)ccc1F] | 2-((2S,6R)-4-(2-(4-(7-(6-cyano-5-(trifluorometh- yl)pyridin-3-yl)-8-oxo-6-thioxo-5,7-diazaspiro [3.4]octan-5-yl)-2-cyclopropylphenoxy)ethyl)- 2,6-dimethylpiperazin-1-yl)-N-(5-(2,6-dioxo- piperidin-3-ylamino)-2-fluorophenyl)acetamide |
| 199 | 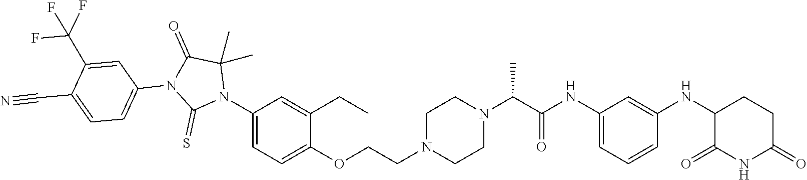 [SMILES: CCc1cc(N2C(=S)N(c3ccc(C#N)c(C(F)(F)F)c3)C(=O)C2(C)C)ccc1OCCN1CCN([C@H](C)C(=O)Nc2cccc(NC3CCC(=O)NC3=O)c2)CC1] | (2R)-2-(4-(2-(4-(3-(4-cyano-3-(trifluorometh- yl)phenyl)-5,5-dimethyl-4-oxo-2-thioxoimidazol- idin-1-yl)-2-ethylphenoxy)ethyl)piperazin-1-yl)- N-(3-(2,6-dioxopiperidin-3-ylamino)phenyl)- propanamide |
| 200 | 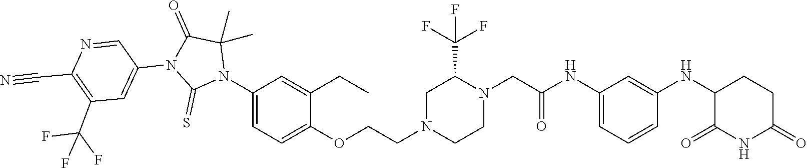 [SMILES: CCc1cc(N2C(=S)N(c3cnc(C#N)c(C(F)(F)F)c3)C(=O)C2(C)C)ccc1OCCN1CCN(CC(=O)Nc2cccc(NC3CCC(=O)NC3=O)c2)[C@@H](C(F)(F)F)C1] | 2-((R)-4-(2-(4-(3-(6-cyano-5-(trifluoromethyl)- pyridin-3-yl)-5,5-dimethyl-4-oxo-2-thioxoimid- azolidin-1-yl)-2-ethylphenoxy)ethyl)-2-(trifluoro- methyl)piperazin-1-yl)-N-(3-(2,6-dioxopiperidin- 3-ylamino)phenyl)acetamide |
| 201 | 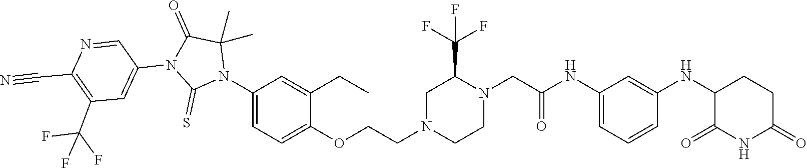 [SMILES: CCc1cc(N2C(=S)N(c3cnc(C#N)c(C(F)(F)F)c3)C(=O)C2(C)C)ccc1OCCN1CCN(CC(=O)Nc2cccc(NC3CCC(=O)NC3=O)c2)[C@H](C(F)(F)F)C1] | 2-((S)-4-(2-(4-(3-(6-cyano-5-(trifluoromethyl)- pyridin-3-yl)-5,5-dimethyl-4-oxo-2-thioxoimid- azolidin-1-yl)-2-ethylphenoxy)ethyl)-2-(trifluoro- methyl)piperazin-1-yl)-N-(3-(2,6-dioxopiperidin- 3-ylamino)phenyl)acetamide |
| 202 | 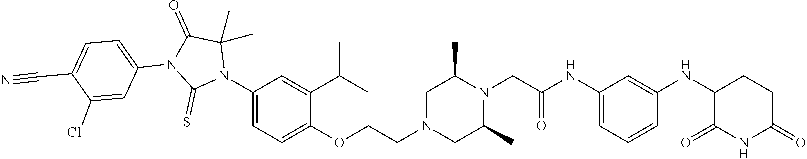 [SMILES: CC(C)c1cc(N2C(=S)N(c3ccc(C#N)c(Cl)c3)C(=O)C2(C)C)ccc1OCCN1C[C@@H](C)N(CC(=O)Nc2cccc(NC3CCC(=O)NC3=O)c2)[C@@H](C)C1] | 2-((2S,6R)-4-(2-(4-(3-(3-chloro-4-cyanophenyl)- 5,5-dimethyl-4-oxo-2-thioxoimidazolidin-1-yl)- 2-isopropylphenoxy)ethyl)-2,6-dimethylpiperazin- 1-yl)-N-(3-(2,6-dioxopiperidin-3-ylamino)phen- yl)acetamide |
| 203 | 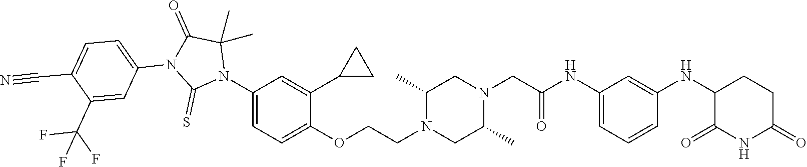 [SMILES: C[C@@H]1CN(CC(=O)Nc2cccc(NC3CCC(=O)NC3=O)c2)[C@H](C)CN1CCOc1ccc(N2C(=S)N(c3ccc(C#N)c(C(F)(F)F)c3)C(=O)C2(C)C)cc1C1CC1] | 2-((2R,5R)-4-(2-(4-(3-(4-cyano-3-(trifluorometh- yl)phenyl)-5,5-dimethyl-4-oxo-2-thioxoimidazol- idin-1-yl)-2-cyclopropylphenoxy)ethyl)-2,5- dimethylpiperazin-1-yl)-N-(3-(2,6-dioxopiper- idin-3-ylamino)phenyl)acetamide |
| 204 | 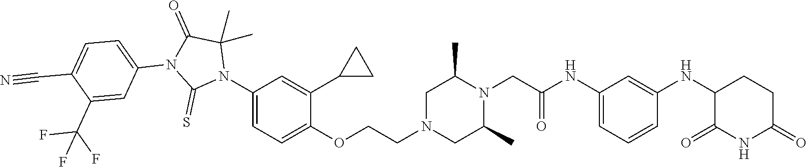 [SMILES: C[C@@H]1CN(CCOc2ccc(N3C(=S)N(c4ccc(C#N)c(C(F)(F)F)c4)C(=O)C3(C)C)cc2C2CC2)C[C@H](C)N1CC(=O)Nc1cccc(NC2CCC(=O)NC2=O)c1] | 2-((2S,6R)-4-(2-(4-(3-(4-cyano-3-(trifluorometh- yl)phenyl)-5,5-dimethyl-4-oxo-2-thioxoimidazol- idin-1-yl)-2-cyclopropylphenoxy)ethyl)-2,6- dimethylpiperazin-1-yl)-N-(3-(2,6-dioxopiperidin- 3-ylamino)phenyl)acetamide |
| 205 | 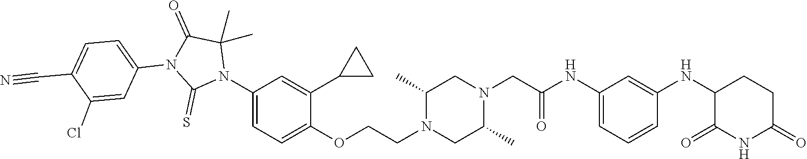 [SMILES: C[C@@H]1CN(CC(=O)Nc2cccc(NC3CCC(=O)NC3=O)c2)[C@H](C)CN1CCOc1ccc(N2C(=S)N(c3ccc(C#N)c(Cl)c3)C(=O)C2(C)C)cc1C1CC1] | 2-((2R,5R)-4-(2-(4-(3-(3-chloro-4-cyanophenyl)- 5,5-dimethyl-4-oxo-2-thioxoimidazolidin-1-yl)- 2-cyclopropylphenoxy)ethyl)-2,5-dimethylpiper- azin-1-yl)-N-(3-(2,6-dioxopiperidin-3-ylamino)- phenyl)acetamide |
| 206 | 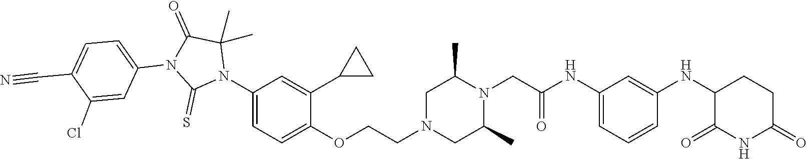 [SMILES: C[C@@H]1CN(CCOc2ccc(N3C(=S)N(c4ccc(C#N)c(Cl)c4)C(=O)C3(C)C)cc2C2CC2)C[C@H](C)N1CC(=O)Nc1cccc(NC2CCC(=O)NC2=O)c1] | 2-((2S,6R)-4-(2-(4-(3-(3-chloro-4-cyanophenyl)- 5,5-dimethyl-4-oxo-2-thioxoimidazolidin-1-yl)- 2-cyclopropylphenoxy)ethyl)-2,6-dimethylpiper- azin-1-yl)-N-(3-(2,6-dioxopiperidin-3-ylamino)- phenyl)acetamide |
| 207 | 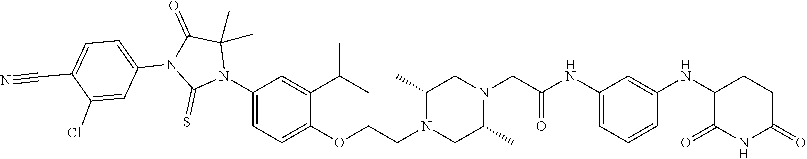 [SMILES: CC(C)c1cc(N2C(=S)N(c3ccc(C#N)c(Cl)c3)C(=O)C2(C)C)ccc1OCCN1C[C@@H](C)N(CC(=O)Nc2cccc(NC3CCC(=O)NC3=O)c2)C[C@H]1C] | 2-((2R,5R)-4-(2-(4-(3-(3-chloro-4-cyanophenyl)- 5,5-dimethyl-4-oxo-2-thioxoimidazolidin-1-yl)- 2-isopropylphenoxy)ethyl)-2,5-dimethylpiperazin- 1-yl)-N-(3-(2,6-dioxopiperidin-3-ylamino)phen- yl)acetamide |
| 208 | 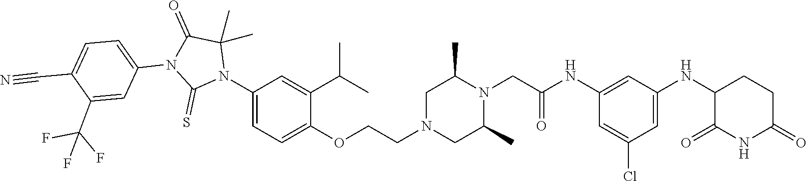 [SMILES: CC(C)c1cc(N2C(=S)N(c3ccc(C#N)c(C(F)(F)F)c3)C(=O)C2(C)C)ccc1OCCN1C[C@@H](C)N(CC(=O)Nc2cc(Cl)cc(NC3CCC(=O)NC3=O)c2)[C@@H](C)C1] | N-(3-chloro-5-(2,6-dioxopiperidin-3-ylamino)- phenyl)-2-((2S,6R)-4-(2-(4-(3-(4-cyano-3- (trifluoromethyl)phenyl)-5,5-dimethyl-4-oxo-2- thioxoimidazolidin-1-yl)-2-isopropylphenoxy)- ethyl)-2,6-dimethylpiperazin-1-yl)acetamide |
| 209 | 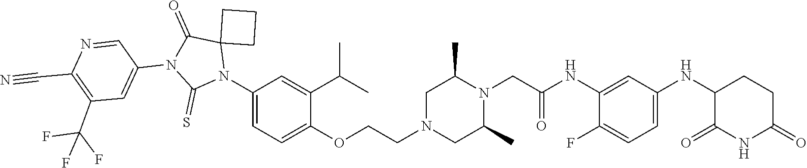 [SMILES: CC(C)c1cc(N2C(=S)N(c3cnc(C#N)c(C(F)(F)F)c3)C(=O)C23CCC3)ccc1OCCN1C[C@@H](C)N(CC(=O)Nc2cc(NC3CCC(=O)NC3=O)ccc2F)[C@@H](C)C1] | 2-((2S,6R)-4-(2-(4-(7-(6-cyano-5-(trifluorometh- yl)pyridin-3-yl)-8-oxo-6-thioxo-5,7-diazaspiro [3.4]octan-5-yl)-2-isopropylphenoxy)ethyl)-2,6- dimethylpiperazin-1-yl)-N-(5-(2,6-dioxopiper- idin-3-ylamino)-2-fluorophenyl)acetamide |
| 210 | 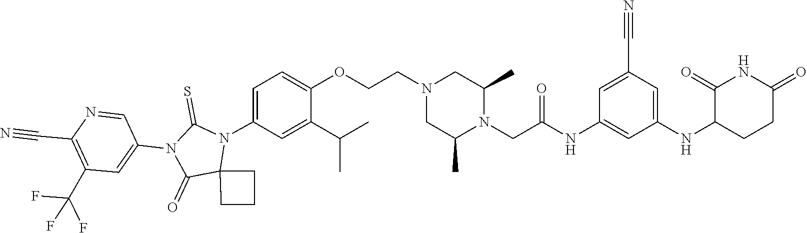 [SMILES: CC(C)c1cc(N2C(=S)N(c3cnc(C#N)c(C(F)(F)F)c3)C(=O)C23CCC3)ccc1OCCN1C[C@@H](C)N(CC(=O)Nc2cc(C#N)cc(NC3CCC(=O)NC3=O)c2)[C@@H](C)C1] | N-(3-cyano-5-(2,6-dioxopiperidin-3-ylamino)- phenyl)-2-((2S,6R)-4-(2-(4-(7-(6-cyano-5- (trifluoromethyl)pyridin-3-yl)-8-oxo-6-thioxo- 5,7-diazaspiro[3.4]octan-5-yl)-2-isopropylphen- oxy)ethyl)-2,6-dimethylpiperazin-1-yl)acetamide |
| 211 | 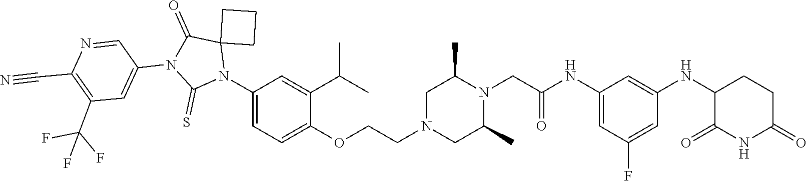 [SMILES: CC(C)c1cc(N2C(=S)N(c3cnc(C#N)c(C(F)(F)F)c3)C(=O)C23CCC3)ccc1OCCN1C[C@@H](C)N(CC(=O)Nc2cc(F)cc(NC3CCC(=O)NC3=O)c2)[C@@H](C)C1] | 2-((2S,6R)-4-(2-(4-(7-(6-cyano-5-(trifluorometh- yl)pyridin-3-yl)-8-oxo-6-thioxo-5,7-diazaspiro [3.4]octan-5-yl)-2-isopropylphenoxy)ethyl)-2,6- dimethylpiperazin-1-yl)-N-(3-(2,6-dioxopiper- idin-3-ylamino)-5-fluorophenyl)acetamide |
| 212 | 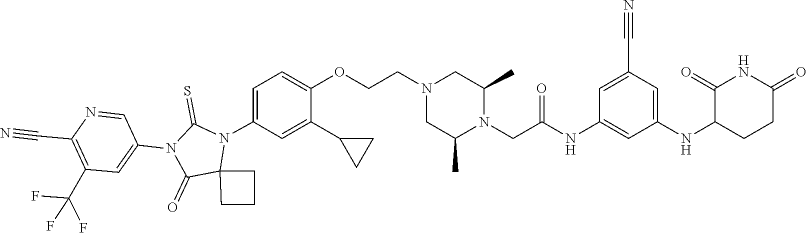 [SMILES: C[C@@H]1CN(CCOc2ccc(N3C(=S)N(c4cnc(C#N)c(C(F)(F)F)c4)C(=O)C34CCC4)cc2C2CC2)C[C@H](C)N1CC(=O)Nc1cc(C#N)cc(NC2CCC(=O)NC2=O)c1] | N-(3-cyano-5-(2,6-dioxopiperidin-3-ylamino)- phenyl)-2-((2S,6R)-4-(2-(4-(7-(6-cyano-5- (trifluoromethyl)pyridin-3-yl)-8-oxo-6-thioxo- 5,7-diazaspiro[3.4]octan-5-yl)-2-cyclopropyl- phenoxy)ethyl)-2,6-dimethylpiperazin-1-yl)- acetamide |
| 213 | 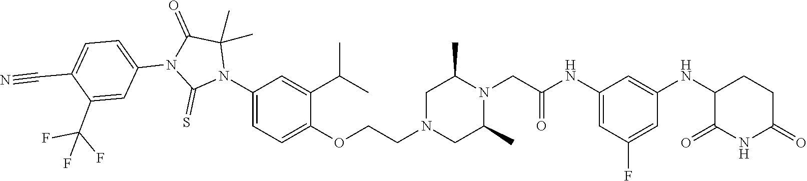 [SMILES: CC(C)c1cc(N2C(=S)N(c3ccc(C#N)c(C(F)(F)F)c3)C(=O)C2(C)C)ccc1OCCN1C[C@@H](C)N(CC(=O)Nc2cc(F)cc(NC3CCC(=O)NC3=O)c2)[C@@H](C)C1] | 2-((2S,6R)-4-(2-(4-(3-(4-cyano-3-(trifluorometh- yl)phenyl)-5,5-dimethyl-4-oxo-2-thioxoimidazol- idin-1-yl)-2-isopropylphenoxy)ethyl)-2,6-dimeth- ylpiperazin-1-yl)-N-(3-(2,6-dioxopiperidin-3- ylamino)-5-fluorophenyl)acetamide |
| 214 | 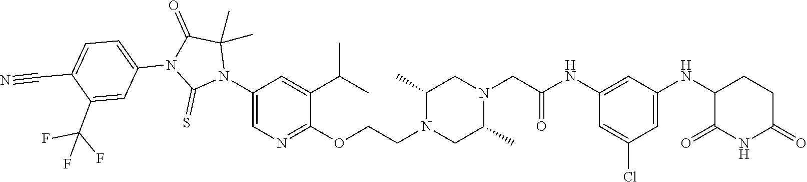 [SMILES: CC(C)c1cc(N2C(=S)N(c3ccc(C#N)c(C(F)(F)F)c3)C(=O)C2(C)C)cnc1OCCN1C[C@@H](C)N(CC(=O)Nc2cc(Cl)cc(NC3CCC(=O)NC3=O)c2)C[C@H]1C] | N-(3-chloro-5-(2,6-dioxopiperidin-3-ylamino)- phenyl)-2-((2R,5R)-4-(2-(5-(3-(4-cyano-3- (trifluoromethyl)phenyl)-5,5-dimethyl-4-oxo-2- thioxoimidazolidin-1-yl)-3-isopropylpyridin-2- yloxy)ethyl)-2,5-dimethylpiperazin-1-yl)acet- amide |
| 215 | 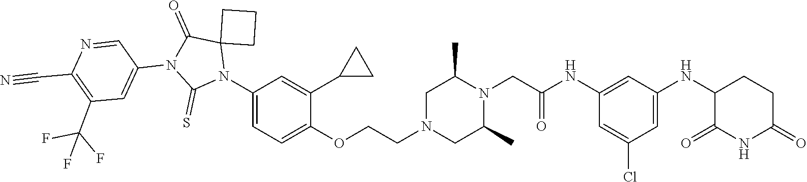 [SMILES: C[C@@H]1CN(CCOc2ccc(N3C(=S)N(c4cnc(C#N)c(C(F)(F)F)c4)C(=O)C34CCC4)cc2C2CC2)C[C@H](C)N1CC(=O)Nc1cc(Cl)cc(NC2CCC(=O)NC2=O)c1] | N-(3-chloro-5-(2,6-dioxopiperidin-3-ylamino)- phenyl)-2-((2S,6R)-4-(2-(4-(7-(6-cyano-5- (trifluoromethyl)pyridin-3-yl)-8-oxo-6-thioxo- 5,7-diazaspiro[3.4]octan-5-yl)-2-cyclopropyl- phenoxy)ethyl)-2,6-dimethylpiperazin-1-yl)- acetamide |
| 216 | 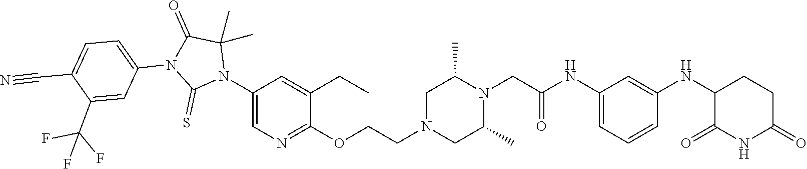 [SMILES: CCc1cc(N2C(=S)N(c3ccc(C#N)c(C(F)(F)F)c3)C(=O)C2(C)C)cnc1OCCN1C[C@@H](C)N(CC(=O)Nc2cccc(NC3CCC(=O)NC3=O)c2)[C@@H](C)C1] | 2-((2R,6S)-4-(2-(5-(3-(4-cyano-3-(trifluorometh- yl)phenyl)-5,5-dimethyl-4-oxo-2-thioxoimidazol- idin-1-yl)-3-ethylpyridin-2-yloxy)ethyl)-2,6- dimethylpiperazin-1-yl)-N-(3-(2,6-dioxopiper- idin-3-ylamino)phenyl)acetamide |
| 217 | 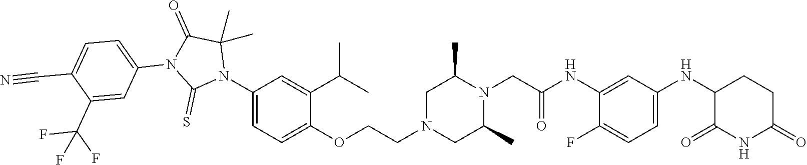 [SMILES: CC(C)c1cc(N2C(=S)N(c3ccc(C#N)c(C(F)(F)F)c3)C(=O)C2(C)C)ccc1OCCN1C[C@@H](C)N(CC(=O)Nc2cc(NC3CCC(=O)NC3=O)ccc2F)[C@@H](C)C1] | 2-((2S,6R)-4-(2-(4-(3-(4-cyano-3-(trifluorometh- yl)phenyl)-5,5-dimethyl-4-oxo-2-thioxoimidazol- idin-1-yl)-2-isopropylphenoxy)ethyl)-2,6-(dimeth- ylpiperazin-1-yl)-N-(5-(2,6-dioxopiperidin-3-yl- amino)-2-fluorophenyl)acetamide |
| 218 | 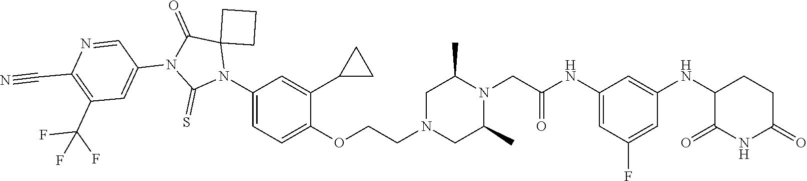 [SMILES: C[C@@H]1CN(CCOc2ccc(N3C(=S)N(c4cnc(C#N)c(C(F)(F)F)c4)C(=O)C34CCC4)cc2C2CC2)C[C@H](C)N1CC(=O)Nc1cc(F)cc(NC2CCC(=O)NC2=O)c1] | 2-((2S,6R)-4-(2-(4-(7-(6-cyano-5-(trifluorometh- yl)pyridin-3-yl)-8-oxo-6-thioxo-5,7-diazaspiro [3.4]octan-5-yl)-2-cyclopropylphenoxy)ethyl)- 2,6-dimethylpiperazin-1-yl)-N-(3-(2,6-dioxo- piperidin-3-ylamino)-5-fluorophenyl)acetamide |
| 219 | 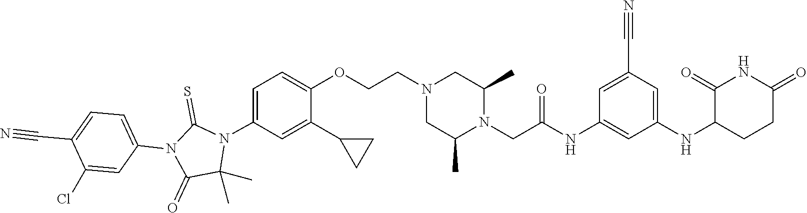 [SMILES: C[C@@H]1CN(CCOc2ccc(N3C(=S)N(c4ccc(C#N)c(Cl)c4)C(=O)C3(C)C)cc2C2CC2)C[C@H](C)N1CC(=O)Nc1cc(C#N)cc(NC2CCC(=O)NC2=O)c1] | 2-((2S,6R)-4-(2-(4-(3-(3-chloro-4-cyanophenyl)- 5,5-dimethyl-4-oxo-2-thioxoimidazolidin-1-yl)- 2-cyclopropylphenoxy)ethyl)-2,6-dimethylpiper- azin-1-yl)-N-(3-cyano-5-(2,6-dioxopiperidin-3- ylamino)phenyl)acetamide |
| 220 | 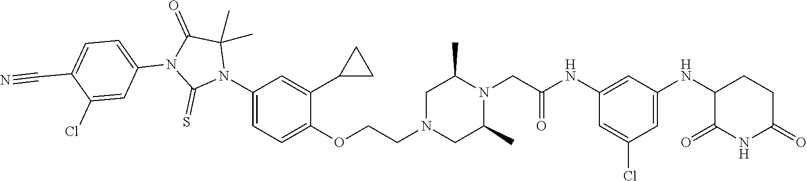 [SMILES: C[C@@H]1CN(CCOc2ccc(N3C(=S)N(c4ccc(C#N)c(Cl)c4)C(=O)C3(C)C)cc2C2CC2)C[C@H](C)N1CC(=O)Nc1cc(Cl)cc(NC2CCC(=O)NC2=O)c1] | 2-((2S,6R)-4-(2-(4-(3-(3-chloro-4-cyanophenyl)- 5,5-dimethyl-4-oxo-2-thioxoimidazolidin-1-yl)- 2-cyclopropylphenoxy)ethyl)-2,6-dimethylpiper- azin-1-yl)-N-(3-chloro-5-(2,6-dioxopiperidin-3- ylamino)phenyl)acetamide |
| 221 | 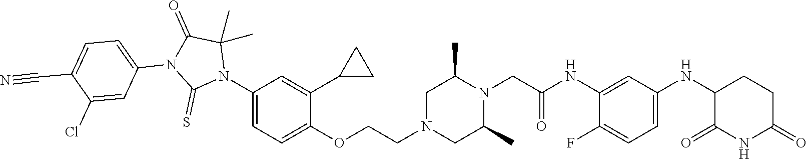 [SMILES: C[C@@H]1CN(CCOc2ccc(N3C(=S)N(c4ccc(C#N)c(Cl)c4)C(=O)C3(C)C)cc2C2CC2)C[C@H](C)N1CC(=O)Nc1cc(NC2CCC(=O)NC2=O)ccc1F] | 2-((2S,6R)-4-(2-(4-(3-(3-chloro-4-cyanophenyl)- 5,5-dimethyl-4-oxo-2-thioxoimidazolidin-1-yl)- 2-cyclopropylphenoxy)ethyl)-2,6-dimethylpiper- azin-1-yl)-N-(5-(2,6-dioxopiperidin-3-ylamino)- 2-fluorophenyl)acetamide |
| 222 | 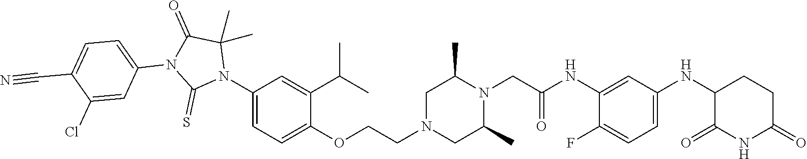 [SMILES: CC(C)c1cc(N2C(=S)N(c3ccc(C#N)c(Cl)c3)C(=O)C2(C)C)ccc1OCCN1C[C@@H](C)N(CC(=O)Nc2cc(NC3CCC(=O)NC3=O)ccc2F)[C@@H](C)C1] | 2-((2S,6R)-4-(2-(4-(3-(3-chloro-4-cyanophenyl)- 5,5-dimethyl-4-oxo-2-thioxoimidazolidin-1-yl)- 2-isopropylphenoxy)ethyl)-2,6-dimethylpiper- azin-1-yl)-N-(5-(2,6-dioxopiperidin-3-ylamino)- 2-fluorophenyl)acetamide |
| 223 | 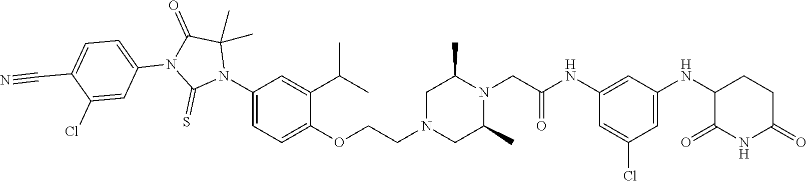 [SMILES: CC(C)c1cc(N2C(=S)N(c3ccc(C#N)c(Cl)c3)C(=O)C2(C)C)ccc1OCCN1C[C@@H](C)N(CC(=O)Nc2cc(Cl)cc(NC3CCC(=O)NC3=O)c2)[C@@H](C)C1] | 2-((2S,6R)-4-(2-(4-(3-(3-chloro-4-cyanophenyl)- 5,5-dimethyl-4-oxo-2-thioxoimidazolidin-1-yl)- 2-isopropylphenoxy)ethyl)-2,6-dimethylpiper- azin-1-yl)-N-(3-chloro-5-(2,6-dioxopiperidin-3- ylamino)phenyl)acetamide |
| 224 | 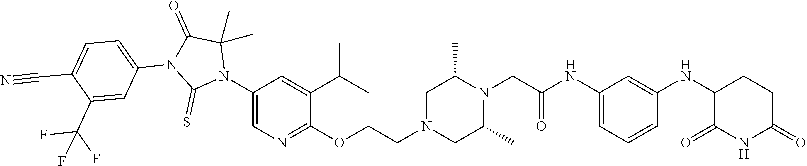 [SMILES: CC(C)c1cc(N2C(=S)N(c3ccc(C#N)c(C(F)(F)F)c3)C(=O)C2(C)C)cnc1OCCN1C[C@@H](C)N(CC(=O)Nc2cccc(NC3CCC(=O)NC3=O)c2)[C@@H](C)C1] | 2-((2R,6S)-4-(2-(5-(3-(4-cyano-3-(trifluorometh- yl)phenyl)-5,5-dimethyl-4-oxo-2-thioxoimidazol- idin-1-yl)-3-isopropylpyridin-2-yloxy)ethyl)-2,6- dimethylpiperazin-1-yl)-N-(3-(2,6-dioxopiper- idin-3-ylamino)phenyl)acetamide |
| 225 | 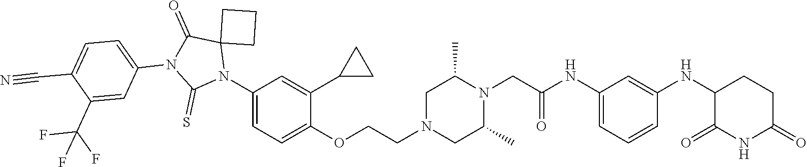 [SMILES: C[C@@H]1CN(CCOc2ccc(N3C(=S)N(c4ccc(C#N)c(C(F)(F)F)c4)C(=O)C34CCC4)cc2C2CC2)C[C@H](C)N1CC(=O)Nc1cccc(NC2CCC(=O)NC2=O)c1] | 2-((2R,6S)-4-(2-(4-(7-(4-cyano-3-(trifluorometh- yl)phenyl)-8-oxo-6-thioxo-5,7-diazaspiro[3.4] octan-5-yl)-2-cyclopropylphenoxy)ethyl)-2,6- dimethylpiperazin-1-yl)-N-(3-(2,6-dioxopiper- idin-3-ylamino)phenyl)acetamide |
| 226 | 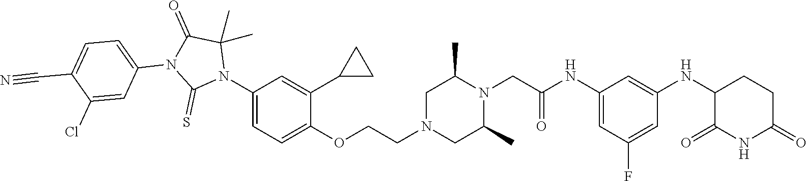 [SMILES: C[C@@H]1CN(CCOc2ccc(N3C(=S)N(c4ccc(C#N)c(Cl)c4)C(=O)C3(C)C)cc2C2CC2)C[C@H](C)N1CC(=O)Nc1cc(F)cc(NC2CCC(=O)NC2=O)c1] | 2-((2S,6R)-4-(2-(4-(3-(3-chloro-4-cyanophenyl)- 5,5-dimethyl-4-oxo-2-thioxoimidazolidin-1-yl)- 2-cyclopropylphenoxy)ethyl)-2,6-dimethylpiper- azin-1-yl)-N-(3-(2,6-dioxopiperidin-3-ylamino)- 5-fluorophenyl)acetamide |
| 227 | 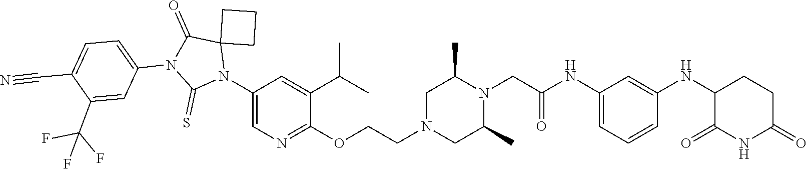 [SMILES: CC(C)c1cc(N2C(=S)N(c3ccc(C#N)c(C(F)(F)F)c3)C(=O)C23CCC3)cnc1OCCN1C[C@@H](C)N(CC(=O)Nc2cccc(NC3CCC(=O)NC3=O)c2)[C@@H](C)C1] | 2-((2S,6R)-4-(2-(5-(7-(4-cyano-3-(trifluorometh- yl)phenyl)-8-oxo-6-thioxo-5,7-diazaspiro[3.4] octan-5-yl)-3-isopropylpyridin-2-yloxy)ethyl)- 2,6-dimethylpiperazin-1-yl)-N-(3-(2,6-dioxo- piperidin-3-ylamino)phenyl)acetamide |
| 228 | 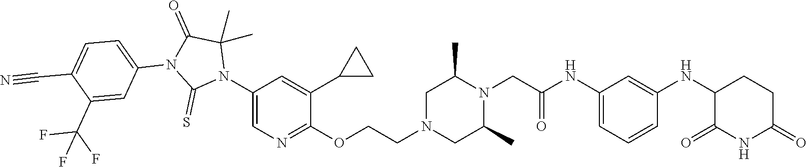 [SMILES: C[C@@H]1CN(CCOc2ncc(N3C(=S)N(c4ccc(C#N)c(C(F)(F)F)c4)C(=O)C3(C)C)cc2C2CC2)C[C@H](C)N1CC(=O)Nc1cccc(NC2CCC(=O)NC2=O)c1] | 2-((2S,6R)-4-(2-(5-(3-(4-cyano-3-(trifluorometh- yl)phenyl)-5,5-dimethyl-4-oxo-2-thioxoimidazol- idin-1-yl)-3-cyclopropylpyridin-2-yloxy)ethyl)- 2,6-dimethylpiperazin-1-yl)-N-(3-(2,6-dioxo- piperidin-3-ylamino)phenyl)acetamide |
| 229 | 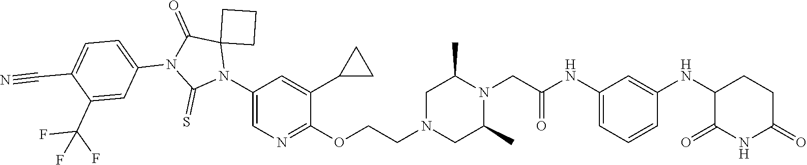 [SMILES: C[C@@H]1CN(CCOc2ncc(N3C(=S)N(c4ccc(C#N)c(C(F)(F)F)c4)C(=O)C34CCC4)cc2C2CC2)C[C@H](C)N1CC(=O)Nc1cccc(NC2CCC(=O)NC2=O)c1] | 2-((2S,6R)-4-(2-(5-(7-(4-cyano-3-(trifluorometh- yl)phenyl)-8-oxo-6-thioxo-5,7-diazaspiro[3.4] octan-5-yl)-3-cyclopropylpyridin-2-yloxy)ethyl)- 2,6-dimethylpiperazin-1-yl)-N-(3-(2,6-dioxo- piperidin-3-ylamino)phenyl)acetamide |
| 230 | 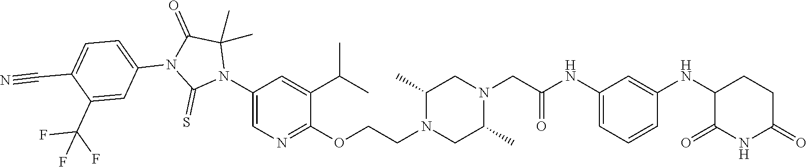 [SMILES: CC(C)c1cc(N2C(=S)N(c3ccc(C#N)c(C(F)(F)F)c3)C(=O)C2(C)C)cnc1OCCN1C[C@@H](C)N(CC(=O)Nc2cccc(NC3CCC(=O)NC3=O)c2)C[C@H]1C] | 2-((2R,5R)-4-(2-(5-(3-(4-cyano-3-(trifluorometh- yl)phenyl)-5,5-dimethyl-4-oxo-2-thioxoimidazol- idin-1-yl)-3-isopropylpyridin-2-yloxy)ethyl)-2,5- dimethylpiperazin-1-yl)-N-(3-(2,6-dioxopiper- idin-3-ylamino)phenyl)acetamide |
| 231 | 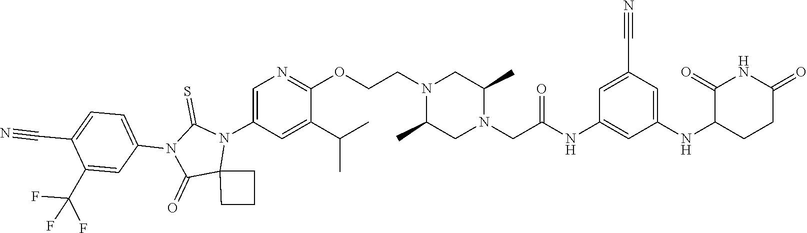 [SMILES: CC(C)c1cc(N2C(=S)N(c3ccc(C#N)c(C(F)(F)F)c3)C(=O)C23CCC3)cnc1OCCN1C[C@@H](C)N(CC(=O)Nc2cc(C#N)cc(NC3CCC(=O)NC3=O)c2)C[C@H]1C] | 2-((2R,5R)-4-(2-(5-(7-(4-cyano-3-(trifluorometh- yl)phenyl)-8-oxo-6-thioxo-5,7-diazaspiro[3.4] octan-5-yl)-3-isopropylpyridin-2-yloxy)ethyl)- 2,5-dimethylpiperazin-1-yl)-N-(3-cyano-5-(2,6- dioxopiperidin-3-ylamino)phenyl)acetamide |
| 232 | 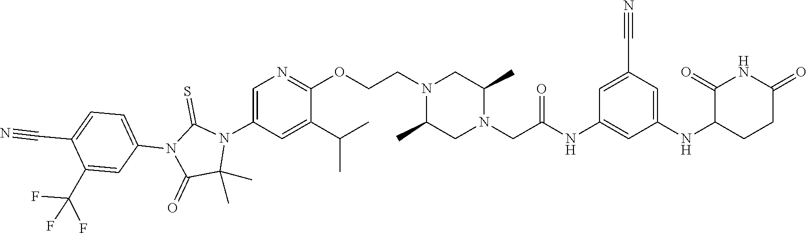 [SMILES: CC(C)c1cc(N2C(=S)N(c3ccc(C#N)c(C(F)(F)F)c3)C(=O)C2(C)C)cnc1OCCN1C[C@@H](C)N(CC(=O)Nc2cc(C#N)cc(NC3CCC(=O)NC3=O)c2)C[C@H]1C] | 2-((2R,5R)-4-(2-(5-(3-(4-cyano-3-(trifluorometh- yl)phenyl)-5,5-dimethyl-4-oxo-2-thioxoimidazol- idin-1-yl)-3-isopropylpyridin-2-yloxy)ethyl)-2,5- dimethylpiperazin-1-yl)-N-(3-cyano-5-(2,6-dioxo- piperidin-3-ylamino)phenyl)acetamide |
| 233 | 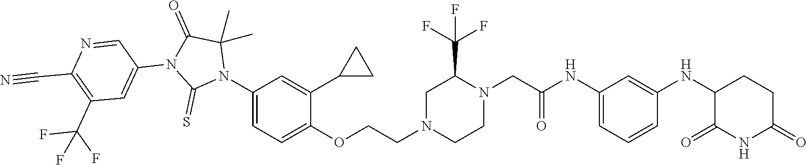 [SMILES: CC1(C)C(=O)N(c2cnc(C#N)c(C(F)(F)F)c2)C(=S)N1c1ccc(OCCN2CCN(CC(=O)Nc3cccc(NC4CCC(=O)NC4=O)c3)[C@H](C(F)(F)F)C2)c(C2CC2)c1] | 2-((S)-4-(2-(4-(3-(6-cyano-5-(trifluoromethyl)- pyridin-3-yl)-5,5-dimethyl-4-oxo-2-thioxoimid- azolidin-1-yl)-2-cyclopropylphenoxy)ethyl)-2- (trifluoromethyl)piperazin-1-yl)-N-(3-(2,6- dioxopiperidin-3-ylamino)phenyl)acetamide |
| 234 | 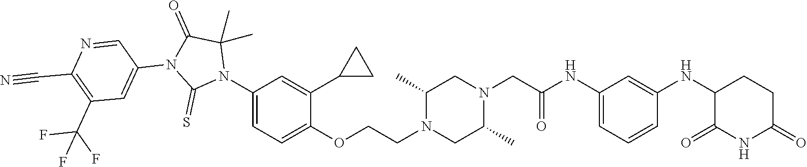 [SMILES: C[C@@H]1CN(CC(=O)Nc2cccc(NC3CCC(=O)NC3=O)c2)[C@H](C)CN1CCOc1ccc(N2C(=S)N(c3cnc(C#N)c(C(F)(F)F)c3)C(=O)C2(C)C)cc1C1CC1] | 2-((2R,5R)-4-(2-(4-(3-(6-cyano-5-(trifluorometh- yl)pyridin-3-yl)-5,5-dimethyl-4-oxo-2-thioxo- imidazolidin-1-yl)-2-cyclopropylphenoxy)ethyl)- 2,5-dimethylpiperazin-1-yl)-N-(3-(2,6-dioxo- piperidin-3-ylamino)phenyl)acetamide |
| 235 | 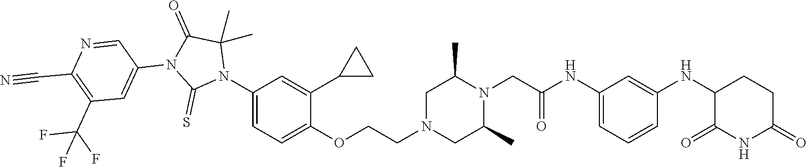 [SMILES: C[C@@H]1CN(CCOc2ccc(N3C(=S)N(c4cnc(C#N)c(C(F)(F)F)c4)C(=O)C3(C)C)cc2C2CC2)C[C@H](C)N1CC(=O)Nc1cccc(NC2CCC(=O)NC2=O)c1] | 2-((2S,6R)-4-(2-(4-(3-(6-cyano-5-(trifluorometh- yl)pyridin-3-yl)-5,5-dimethyl-4-oxo-2-thioxo- imidazolidin-1-yl)-2-cyclopropylphenoxy)ethyl)- 2,6-dimethylpiperazin-1-yl)-N-(3-(2,6-dioxo- piperidin-3-ylamino)phenyl)acetamide |
| 236 (peak 1) 237 (peak 2) | 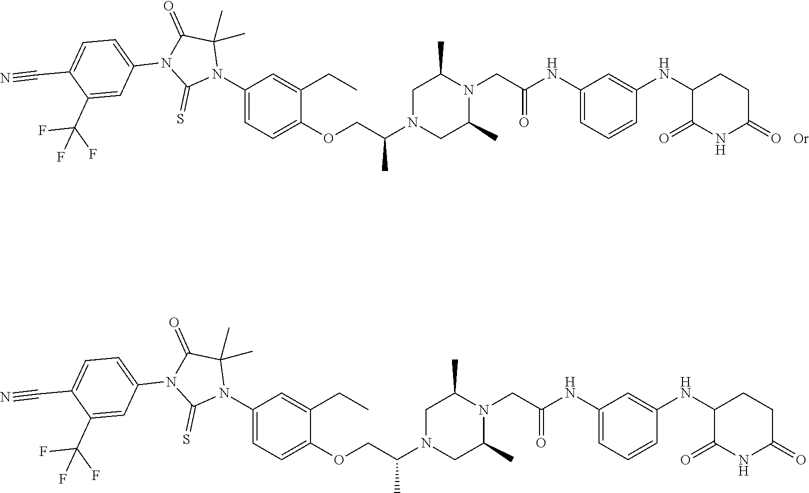 [SMILES: CCc1cc(N2C(=S)N(c3ccc(C#N)c(C(F)(F)F)c3)C(=O)C2(C)C)ccc1OC[C@H](C)N1C[C@@H](C)N(CC(=O)Nc2cccc(NC3CCC(=O)NC3=O)c2)[C@@H](C)C1 ; CCc1cc(N2C(=S)N(c3ccc(C#N)c(C(F)(F)F)c3)C(=O)C2(C)C)ccc1OC[C@@H](C)N1C[C@@H](C)N(CC(=O)Nc2cccc(NC3CCC(=O)NC3=O)c2)[C@@H](C)C1] | 2-((2S,6R)-4-((S)-1-(4-(3-(4-cyano-3-(trifluoro- methyl)phenyl)-5,5-dimethyl-4-oxo-2-thioxo- imidazolidin-1-yl)-2-ethylphenoxy)propan-2-yl)- 2,6-dimethylpiperazin-1-yl)-N-(3-(2,6-dioxo- piperidin-3-ylamino)phenyl)acetamide or 2-((2S,6R)-4-((R)-1-(4-(3-(4-cyano-3-(trifluoro- methyl)phenyl)-5,5-dimethyl-4-oxo-2-thioxo- imidazolidin-1-yl)-2-ethylphenoxy)propan-2-yl)- 2,6-dimethylpiperazin-1-yl)-N-(3-(2,6-dioxo- piperidin-3-ylamino)phenyl)acetamide |
| 238 | 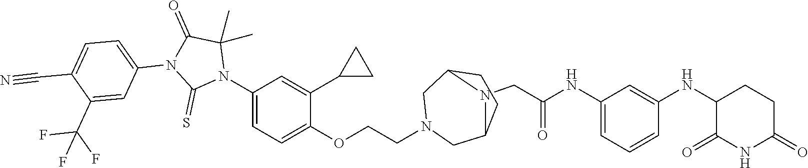 [SMILES: CC1(C)C(=O)N(c2ccc(C#N)c(C(F)(F)F)c2)C(=S)N1c1ccc(OCCN2CC3CCC(C2)N3CC(=O)Nc2cccc(NC3CCC(=O)NC3=O)c2)c(C2CC2)c1] | 2-(3-(2-(4-(3-(4-cyano-3-(trifluoromethyl)phen- yl)-5,5-dimethyl-4-oxo-2-thioxoimidazolidin-1- yl)-2-cyclopropylphenoxy)ethyl)-3,8-diazabicyclo [3.2.1]octan-8-yl)-N-(3-(2,6-dioxopiperidin-3- ylamino)phenyl)acetamide |
| 239 (Peak 1) 240 (Peak 2) | 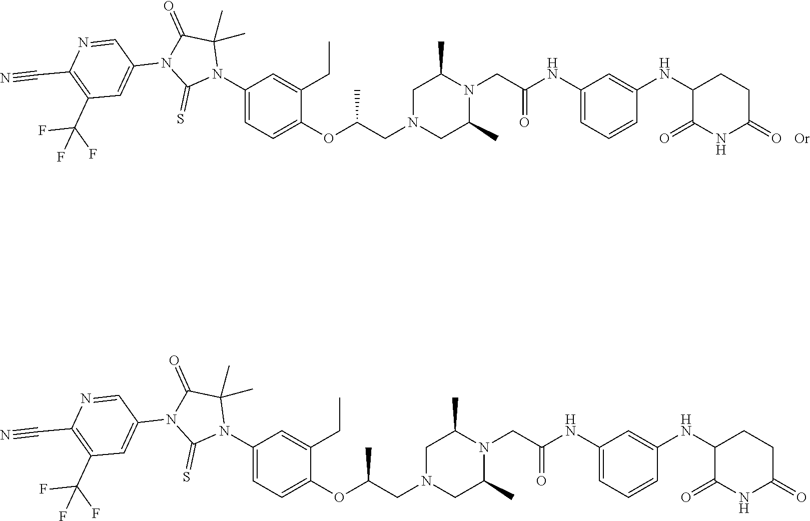 [SMILES: CCc1cc(N2C(=S)N(c3cnc(C#N)c(C(F)(F)F)c3)C(=O)C2(C)C)ccc1O[C@H](C)CN1C[C@@H](C)N(CC(=O)Nc2cccc(NC3CCC(=O)NC3=O)c2)[C@@H](C)C1 ; CCc1cc(N2C(=S)N(c3cnc(C#N)c(C(F)(F)F)c3)C(=O)C2(C)C)ccc1O[C@@H](C)CN1C[C@@H](C)N(CC(=O)Nc2cccc(NC3CCC(=O)NC3=O)c2)[C@@H](C)C1] | 2-((2S,6R)-4-((R)-2-(4-(3-(6-cyano-5-(trifluoro- methyl)pyridin-3-yl)-5,5-dimethyl-4-oxo-2- thioxoimidazolidin-1-yl)-2-ethylphenoxy)prop- yl)-2,6-dimethylpiperazin-1-yl)-N-(3-(2,6-dioxo- piperidin-3-ylamino)phenyl)acetamide or 2-((2S,6R)-4-((S)-2-(4-(3-(6-cyano-5-(trifluoro- methyl)pyridin-3-yl)-5,5-dimethyl-4-oxo-2- thioxoimidazolidin-1-yl)-2-ethylphenoxy)propyl)- 2,6-dimethylpiperazin-1-yl)-N-(3-(2,6-dioxo- piperidin-3-ylamino)phenyl)acetamide |
| 241 (Peak 1) 242 (Peak 2) | 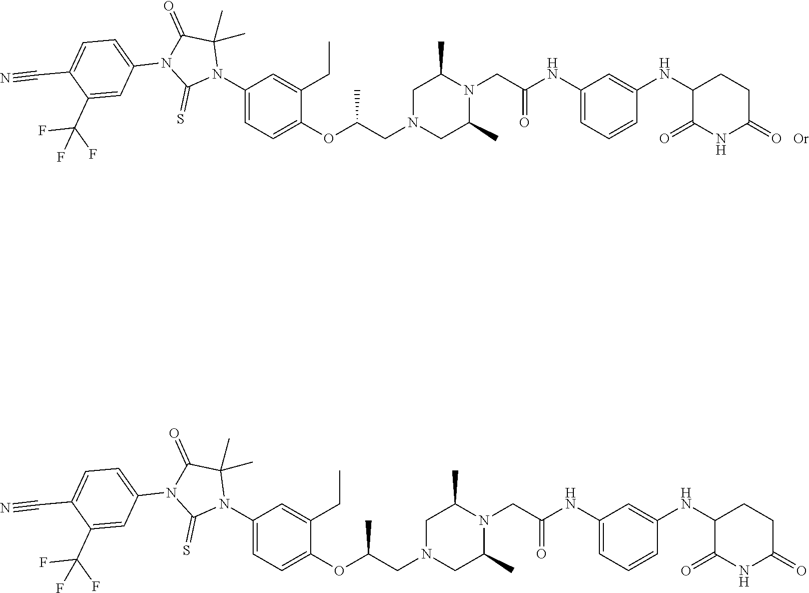 [SMILES: CCc1cc(N2C(=S)N(c3ccc(C#N)c(C(F)(F)F)c3)C(=O)C2(C)C)ccc1O[C@H](C)CN1C[C@@H](C)N(CC(=O)Nc2cccc(NC3CCC(=O)NC3=O)c2)[C@@H](C)C1 ; CCc1cc(N2C(=S)N(c3ccc(C#N)c(C(F)(F)F)c3)C(=O)C2(C)C)ccc1O[C@@H](C)CN1C[C@@H](C)N(CC(=O)Nc2cccc(NC3CCC(=O)NC3=O)c2)[C@@H](C)C1] | 2-((2S,6R)-4-((R)-2-(4-(3-(4-cyano-3-(trifluoro- methyl)phenyl)-5,5-dimethyl-4-oxo-2-thioxo- imidazolidin-1-yl)-2-ethylphenoxy)propyl)-2,6- dimethylpiperazin-1-yl)-N-(3-(2,6-dioxopiper- idin-3-ylamino)phenyl)acetamide or 2-((2S,6R)-4-((S)-2-(4-(3-(4-cyano-3-(trifluoro- methyl)phenyl)-5,5-dimethyl-4-oxo-2-thioxo- imidazolidin-1-yl)-2-ethylphenoxy)propyl)-2,6- dimethylpiperazin-1-yl)-N-(3-(2,6-dioxopiper- idin-3-ylamino)phenyl)acetamide |
| 243 | 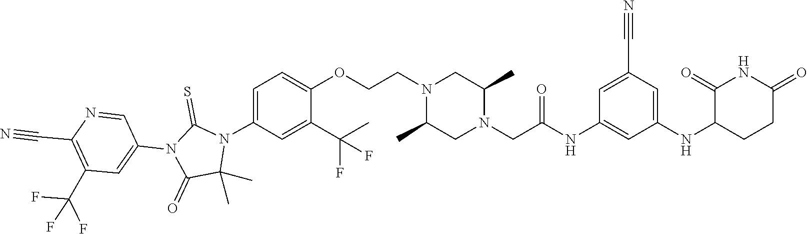 [SMILES: C[C@@H]1CN(CC(=O)Nc2cc(C#N)cc(NC3CCC(=O)NC3=O)c2)[C@H](C)CN1CCOc1ccc(N2C(=S)N(c3cnc(C#N)c(C(F)(F)F)c3)C(=O)C2(C)C)cc1C(C)(F)F] | N-(3-cyano-5-(2,6-dioxopiperidin-3-ylamino)- phenyl)-2-((2R,5R)-4-(2-(4-(3-(6-cyano-5- (trifluoromethyl)pyridin-3-yl)-5,5-dimethyl-4- oxo-2-thioxoimidazolidin-1-yl)-2-(1,1-difluoro- ethyl)phenoxy)ethyl)-2,5-dimethylpiperazin-1- yl)acetamide |
| 244 | 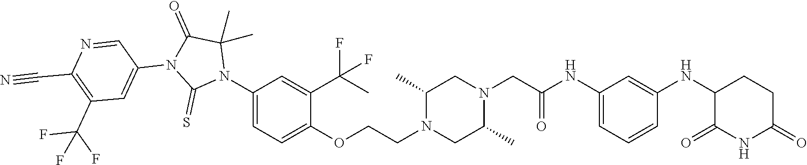 [SMILES: C[C@@H]1CN(CC(=O)Nc2cccc(NC3CCC(=O)NC3=O)c2)[C@H](C)CN1CCOc1ccc(N2C(=S)N(c3cnc(C#N)c(C(F)(F)F)c3)C(=O)C2(C)C)cc1C(C)(F)F] | 2-((2R,5R)-4-(2-(4-(3-(6-cyano-5-(trifluorometh- yl)pyridin-3-yl)-5,5-dimethyl-4-oxo-2-thioxo- imidazolidin-1-yl)-2-(1,1-difluoroethyl)phenoxy)- ethyl)-2,5-dimethylpiperazin-1-yl)-N-(3-(2,6- dioxopiperidin-3-ylamino)phenyl)acetamide |
| 245 | 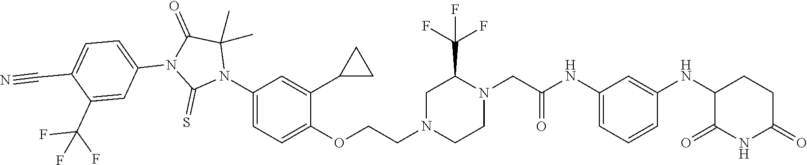 [SMILES: CC1(C)C(=O)N(c2ccc(C#N)c(C(F)(F)F)c2)C(=S)N1c1ccc(OCCN2CCN(CC(=O)Nc3cccc(NC4CCC(=O)NC4=O)c3)[C@H](C(F)(F)F)C2)c(C2CC2)c1] | 2-((S)-4-(2-(4-(3-(4-cyano-3-(trifluoromethyl)- phenyl)-5,5-dimethyl-4-oxo-2-thioxoimidazol- idin-1-yl)-2-cyclopropylphenoxy)ethyl)-2-(tri- fluoromethyl)piperazin-1-yl)-N-(3-(2,6-dioxo- piperidin-3-ylamino)phenyl)acetamide |
| 246 | 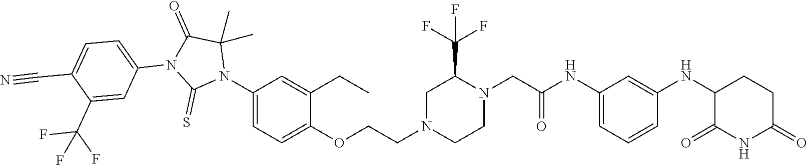 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)[C@H](C(F)(F)F)C1] | 2-((S)-4-(2-(4-(3-(4-cyano-3-(trifluoromethyl)- phenyl)-5,5-dimethyl-4-oxo-2-thioxoimidazol- idin-1-yl)-2-ethylphenoxy)ethyl)-2-(trifluoro- methyl)piperazin-1-yl)-N-(3-(2,6-dioxopiperidin- 3-ylamino)phenyl)acetamide |
| 247 | 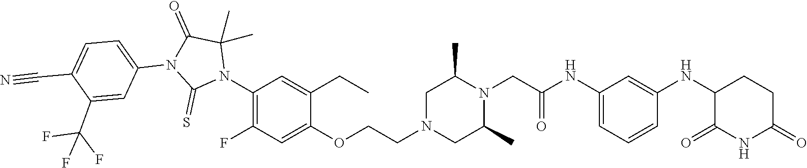 [SMILES: CCc1cc(N2C(=S)N(c3ccc(C#N)c(C(F)(F)F)c3)C(=O)C2(C)C)c(F)cc1OCCN1C[C@@H](C)N(CC(=O)Nc2cccc(NC3CCC(=O)NC3=O)c2)[C@@H](C)C1] | 2-((2S,6R)-4-(2-(4-(3-(4-cyano-3-(trifluorometh- yl)phenyl)-5,5-dimethyl-4-oxo-2-thioxoimidazol- idin-1-yl)-2-ethyl-5-fluorophenoxy)ethyl)-2,6- dimethylpiperazin-1-yl)-N-(3-(2,6-dioxopiper- idin-3-ylamino)phenyl)acetamide |
| 248 | 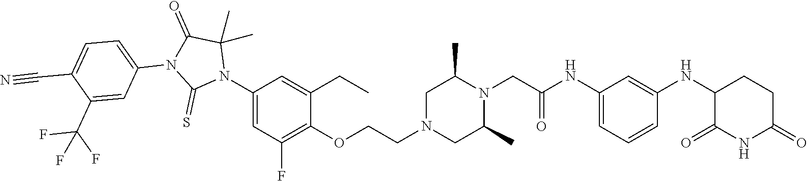 [SMILES: CCc1cc(N2C(=S)N(c3ccc(C#N)c(C(F)(F)F)c3)C(=O)C2(C)C)cc(F)c1OCCN1C[C@@H](C)N(CC(=O)Nc2cccc(NC3CCC(=O)NC3=O)c2)[C@@H](C)C1] | 2-((2S,6R)-4-(2-(4-(3-(4-cyano-3-(trifluorometh- yl)phenyl)-5,5-dimethyl-4-oxo-2-thioxoimidazol- idin-1-yl)-2-ethyl-6-fluorophenoxy)ethyl)-2,6- dimethylpiperazin-1-yl)-N-(3-(2,6-dioxopiper- idin-3-ylamino)phenyl)acetamide |
| 249 | 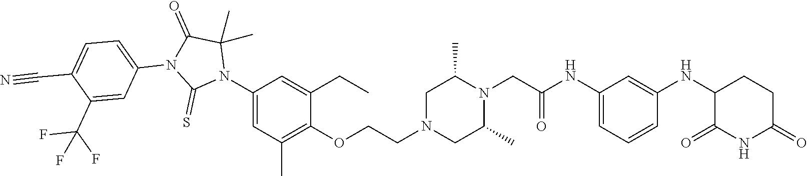 [SMILES: CCc1cc(N2C(=S)N(c3ccc(C#N)c(C(F)(F)F)c3)C(=O)C2(C)C)cc(C)c1OCCN1C[C@@H](C)N(CC(=O)Nc2cccc(NC3CCC(=O)NC3=O)c2)[C@@H](C)C1] | 2-((2R,6S)-4-(2-(4-(3-(4-cyano-3-(trifluorometh- yl)phenyl)-5,5-dimethyl-4-oxo-2-thioxoimidazol- idin-1-yl)-2-ethyl-6-methylphenoxy)ethyl)-2,6- dimethylpiperazin-1-yl)-N-(3-(2,6-dioxopiper- idin-3-ylamino)phenyl)acetamide |
| 250 | 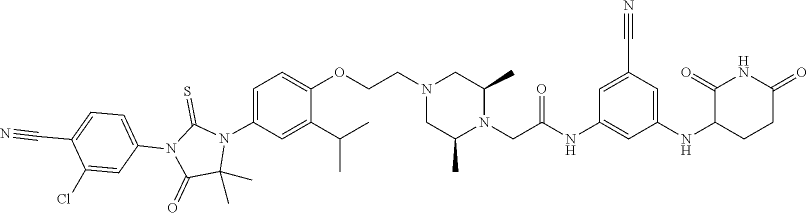 [SMILES: CC(C)c1cc(N2C(=S)N(c3ccc(C#N)c(Cl)c3)C(=O)C2(C)C)ccc1OCCN1C[C@@H](C)N(CC(=O)Nc2cc(C#N)cc(NC3CCC(=O)NC3=O)c2)[C@@H](C)C1] | 2-((2S,6R)-4-(2-(4-(3-(3-chloro-4-cyanophenyl)- 5,5-dimethyl-4-oxo-2-thioxoimidazolidin-1-yl)-2- isopropylphenoxy)ethyl)-2,6-dimethylpiperazin- 1-yl)-N-(3-cyano-5-(2,6-dioxopiperidin-3-yl- amino)phenyl)acetamide |
| 251 | 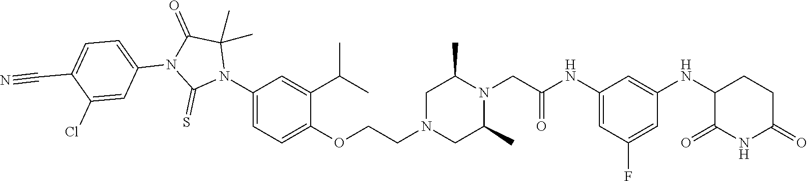 [SMILES: CC(C)c1cc(N2C(=S)N(c3ccc(C#N)c(Cl)c3)C(=O)C2(C)C)ccc1OCCN1C[C@@H](C)N(CC(=O)Nc2cc(F)cc(NC3CCC(=O)NC3=O)c2)[C@@H](C)C1] | 2-((2S,6R)-4-(2-(4-(3-(3-chloro-4-cyanophenyl)- 5,5-dimethyl-4-oxo-2-thioxoimidazolidin-1-yl)-2- isopropylphenoxy)ethyl)-2,6-dimethylpiperazin- 1-yl)-N-(3-(2,6-dioxopiperidin-3-ylamino)-5- fluorophenyl)acetamide |
| 252 | 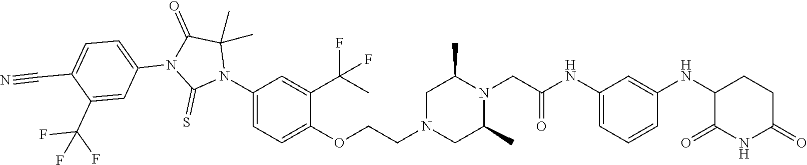 [SMILES: C[C@@H]1CN(CCOc2ccc(N3C(=S)N(c4ccc(C#N)c(C(F)(F)F)c4)C(=O)C3(C)C)cc2C(C)(F)F)C[C@H](C)N1CC(=O)Nc1cccc(NC2CCC(=O)NC2=O)c1] | 2-((2S,6R)-4-(2-(4-(3-(4-cyano-3-(trifluorometh- yl)phenyl)-5,5-dimethyl-4-oxo-2-thioxoimidazol- idin-1-yl)-2-(1,1-difluoroethyl)phenoxy)ethyl)- 2,6-dimethylpiperazin-1-yl)-N-(3-(2,6-dioxo- piperidin-3-ylamino)phenyl)acetamide |
| 253 | 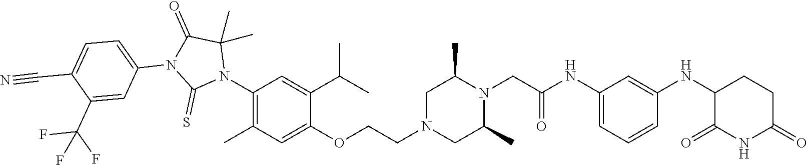 [SMILES: Cc1cc(OCCN2C[C@@H](C)N(CC(=O)Nc3cccc(NC4CCC(=O)NC4=O)c3)[C@@H](C)C2)c(C(C)C)cc1N1C(=S)N(c2ccc(C#N)c(C(F)(F)F)c2)C(=O)C1(C)C] | 2-((2S,6R)-4-(2-(4-(3-(4-cyano-3-(trifluorometh- yl)phenyl)-5,5-dimethyl-4-oxo-2-thioxoimidazol- idin-1-yl)-2-isopropyl-5-methylphenoxy)ethyl)- 2,6-dimethylpiperazin-1-yl)-N-(3-(2,6-dioxopiper- idin-3-ylamino)phenyl)acetamide |
| 254 | 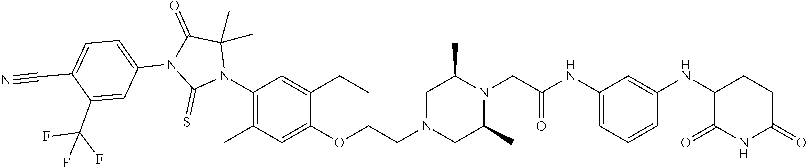 [SMILES: CCc1cc(N2C(=S)N(c3ccc(C#N)c(C(F)(F)F)c3)C(=O)C2(C)C)c(C)cc1OCCN1C[C@@H](C)N(CC(=O)Nc2cccc(NC3CCC(=O)NC3=O)c2)[C@@H](C)C1] | 2-((2S,6R)-4-(2-(4-(3-(4-cyano-3-(trifluorometh- yl)phenyl)-5,5-dimethyl-4-oxo-2-thioxoimidazol- idin-1-yl)-2-ethyl-5-methylphenoxy)ethyl)-2,6- dimethylpiperazin-1-yl)-N-(3-(2,6-dioxopiperidin- 3-ylamino)phenyl)acetamide |
| 255 | 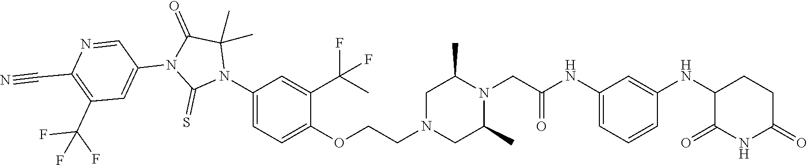 [SMILES: C[C@@H]1CN(CCOc2ccc(N3C(=S)N(c4cnc(C#N)c(C(F)(F)F)c4)C(=O)C3(C)C)cc2C(C)(F)F)C[C@H](C)N1CC(=O)Nc1cccc(NC2CCC(=O)NC2=O)c1] | 2-((2S,6R)-4-(2-(4-(3-(6-cyano-5-(trifluorometh- yl)pyridin-3-yl)-5,5-dimethyl-4-oxo-2-thioxoimid- azolidin-1-yl)-2-(1,1-difluoroethyl)phenoxy)- ethyl)-2,6-dimethylpiperazin-1-yl)-N-(3-(2,6- dioxopiperidin-3-ylamino)phenyl)acetamide |
| 256 | 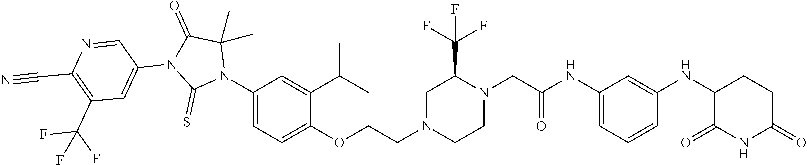 [SMILES: CC(C)c1cc(N2C(=S)N(c3cnc(C#N)c(C(F)(F)F)c3)C(=O)C2(C)C)ccc1OCCN1CCN(CC(=O)Nc2cccc(NC3CCC(=O)NC3=O)c2)[C@H](C(F)(F)F)C1] | 2-((S)-4-(2-(4-(3-(6-cyano-5-(trifluoromethyl)- pyridin-3-yl)-5,5-dimethyl-4-(oxo-2-thioxoimid- azolidin-1-yl)-2-isopropylphenoxy)ethyl)-2- (trifluoromethyl)piperazin-1-yl)-N-(3-(2,6- dioxopiperidin-3-ylamino)phenyl)acetamide |
| 257 | 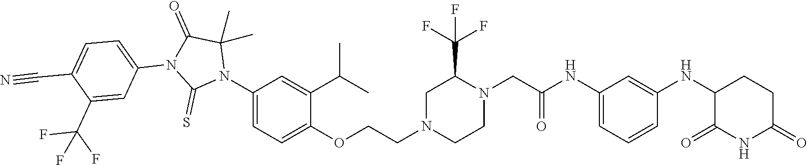 [SMILES: CC(C)c1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)[C@H](C(F)(F)F)C1] | 2-((S)-4-(2-(4-(3-(4-cyano-3-(trifluoromethyl)- phenyl)-5,5-dimethyl-4-oxo-2-thioxoimidazol- idin-1-yl)-2-isopropylphenoxy)ethyl)-2-(trifluoro- methyl)piperazin-1-yl)-N-(3-(2,6-dioxopiperidin- 3-ylamino)phenyl)acetamide |
| 258 | 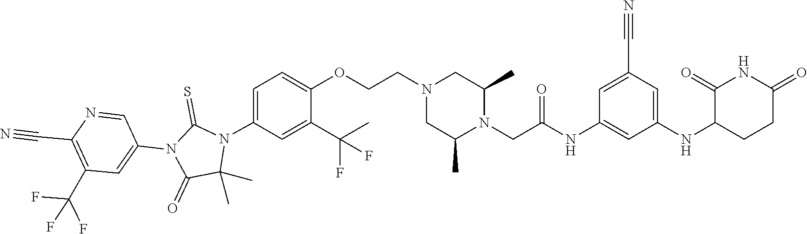 [SMILES: C[C@@H]1CN(CCOc2ccc(N3C(=S)N(c4cnc(C#N)c(C(F)(F)F)c4)C(=O)C3(C)C)cc2C(C)(F)F)C[C@H](C)N1CC(=O)Nc1cc(C#N)cc(NC2CCC(=O)NC2=O)c1] | N-(3-cyano-5-(2,6-dioxopiperidin-3-ylamino) phenyl)-2-((2S,6R)-4-(2-(4-(3-(6-cyano-5-(tri- fluoromethyl)pyridin-3-yl)-5,5-dimethyl-4-oxo- 2-thioxoimidazolidin-1-yl)-2-(1,1-difluoroethyl)- phenoxy)ethyl)-2,6-dimethylpiperazin-1-yl)acet- amide |
| 259 | 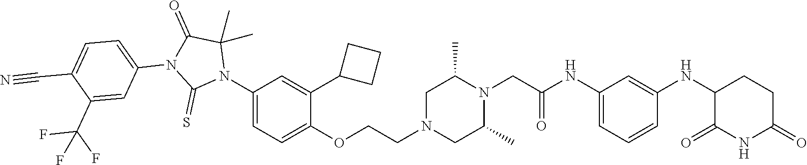 [SMILES: C[C@@H]1CN(CCOc2ccc(N3C(=S)N(c4ccc(C#N)c(C(F)(F)F)c4)C(=O)C3(C)C)cc2C2CCC2)C[C@H](C)N1CC(=O)Nc1cccc(NC2CCC(=O)NC2=O)c1] | 2-((2R,6S)-4-(2-(4-(3-(4-cyano-3-(trifluorometh- yl)phenyl)-5,5-dimethyl-4-oxo-2-thioxoimidazol- idin-1-yl)-2-cyclobutylphenoxy)ethyl)-2,6- dimethylpiperazin-1-yl)-N-(3-(2,6-dioxopiper- idin-3-ylamino)phenyl)acetamide |
| 260 | 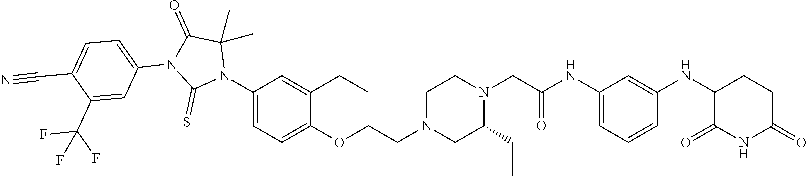 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)[C@H](CC)C1] | 2-((R)-4-(2-(4-(3-(4-cyano-3-(trifluoromethyl)- phenyl)-5,5-dimethyl-4-oxo-2-thioxoimidazol- idin-1-yl)-2-ethylphenoxy)ethyl)-2-ethylpiper- azin-1-yl)-N-(3-(2,6-dioxopiperidin-3-ylamino)- phenyl)acetamide |
| 261 | 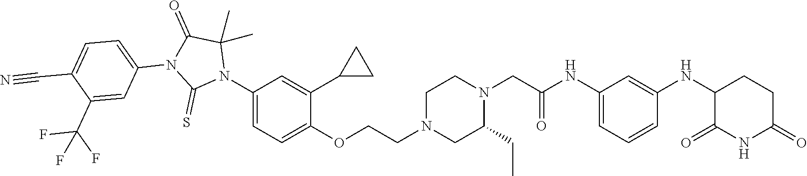 [SMILES: CC[C@@H]1CN(CCOc2ccc(N3C(=S)N(c4ccc(C#N)c(C(F)(F)F)c4)C(=O)C3(C)C)cc2C2CC2)CCN1CC(=O)Nc1cccc(NC2CCC(=O)NC2=O)c1] | 2-((R)-4-(2-(4-(3-(4-cyano-3-(trifluoromethyl)- phenyl)-5,5-dimethyl-4-oxo-2-thioxoimidazol- idin-1-yl)-2-cyclopropylphenoxy)ethyl)-2-ethyl- piperazin-1-yl)-N-(3-(2,6-dioxopiperidin-3-yl- amino)phenyl)acetamide |
| 262 | 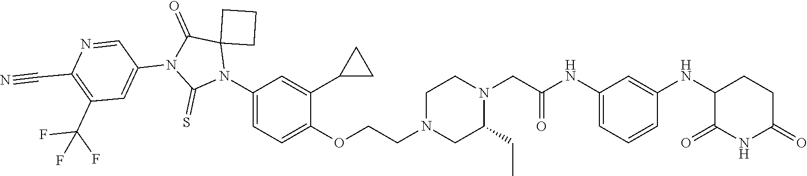 [SMILES: CC[C@@H]1CN(CCOc2ccc(N3C(=S)N(c4cnc(C#N)c(C(F)(F)F)c4)C(=O)C34CCC4)cc2C2CC2)CCN1CC(=O)Nc1cccc(NC2CCC(=O)NC2=O)c1] | 2-((R)-4-(2-(4-(7-(6-cyano-5-(trifluoromethyl)- pyridin-3-yl)-8-oxo-6-thioxo-5,7-diazaspiro[3.4] octan-5-yl)-2-cyclopropylphenoxy)ethyl)-2-ethyl- piperazin-1-yl)-N-(3-(2,6-dioxopiperidin-3-yl- amino)phenyl)acetamide |
| 263 | 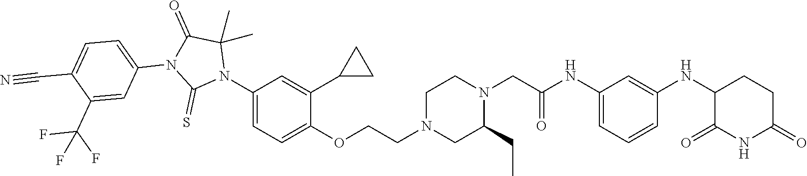 [SMILES: CC[C@H]1CN(CCOc2ccc(N3C(=S)N(c4ccc(C#N)c(C(F)(F)F)c4)C(=O)C3(C)C)cc2C2CC2)CCN1CC(=O)Nc1cccc(NC2CCC(=O)NC2=O)c1] | 2-((S)-4-(2-(4-(3-(4-cyano-3-(trifluoromethyl)- phenyl)-5,5-dimethyl-4-oxo-2-thioxoimidazol- idin-1-yl)-2-cyclopropylphenoxy)ethyl)-2-ethyl- piperazin-1-yl)-N-(3-(2,6-dioxopiperidin-3-yl- amino)phenyl)acetamide |
| 264 | 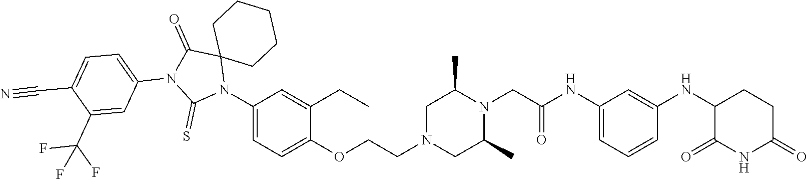 [SMILES: CCc1cc(N2C(=S)N(c3ccc(C#N)c(C(F)(F)F)c3)C(=O)C23CCCCC3)ccc1OCCN1C[C@@H](C)N(CC(=O)Nc2cccc(NC3CCC(=O)NC3=O)c2)[C@@H](C)C1] | 2-((2S,6R)-4-(2-(4-(3-(4-cyano-3-(trifluorometh- yl)-phenyl)-4-oxo-2-thioxo-1,3-diazaspiro[4.5] decan-1-yl)-2-ethylphenoxy)ethyl)-2,6-dimethyl- piperazin-1-yl)-N-(3-(2,6-dioxopiperidin-3-yl- amino)phenyl)acetamide |
| 265 | 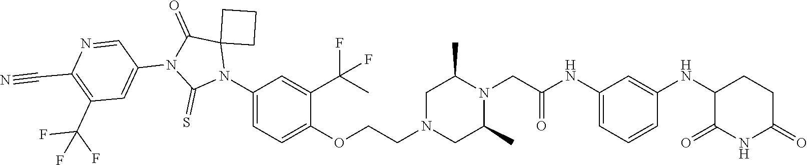 [SMILES: C[C@@H]1CN(CCOc2ccc(N3C(=S)N(c4cnc(C#N)c(C(F)(F)F)c4)C(=O)C34CCC4)cc2C(C)(F)F)C[C@H](C)N1CC(=O)Nc1cccc(NC2CCC(=O)NC2=O)c1] | 2-((2S,6R)-4-(2-(4-(7-(6-cyano-5-(trifluorometh- yl)pyridin-3-yl)-8-oxo-6-thioxo-5,7-diazaspiro [3.4]octan-5-yl)-2-(1,1-difluoroethyl)phenoxy)- ethyl)-2,6-dimethylpiperazin-1-yl)-N-(3-(2,6- dioxopiperidin-3-ylamino)phenyl)acetamide |
| 266 | 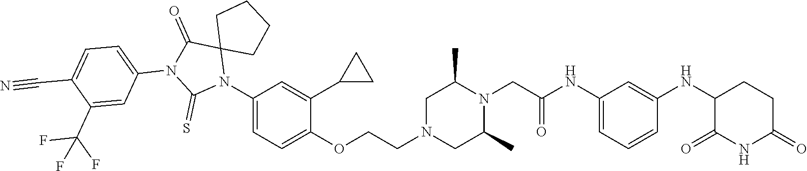 [SMILES: C[C@@H]1CN(CCOc2ccc(N3C(=S)N(c4ccc(C#N)c(C(F)(F)F)c4)C(=O)C34CCCC4)cc2C2CC2)C[C@H](C)N1CC(=O)Nc1cccc(NC2CCC(=O)NC2=O)c1] | 2-((2S,6R)-4-(2-(4-(3-(4-cyano-3-(trifluorometh- yl)phenyl)-4-oxo-2-thioxo-1,3-diazaspiro[4.4] nonan-1-yl)-2-cyclopropylphenoxy)ethyl)-2,6- dimethylpiperazin-1-yl)-N-(3-(2,6-dioxopiper- idin-3-ylamino)phenyl)acetamide |
| 267 | 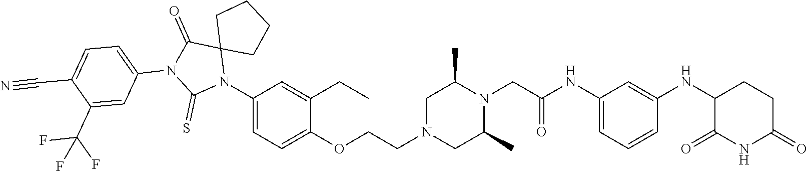 [SMILES: CCc1cc(N2C(=S)N(c3ccc(C#N)c(C(F)(F)F)c3)C(=O)C23CCCC3)ccc1OCCN1C[C@@H](C)N(CC(=O)Nc2cccc(NC3CCC(=O)NC3=O)c2)[C@@H](C)C1] | 2-((2S,6R)-4-(2-(4-(3-(4-cyano-3-(trifluorometh- yl)phenyl)-4-oxo-2-thioxo-1,3-diazaspiro[4.4] nonan-1-yl)-2-ethylphenoxy)ethyl)-2,6-dimethyl- piperazin-1-yl)-N-(3-(2,6-dioxopiperidin-3-yl- amino)phenyl)acetamide |
| 268 | 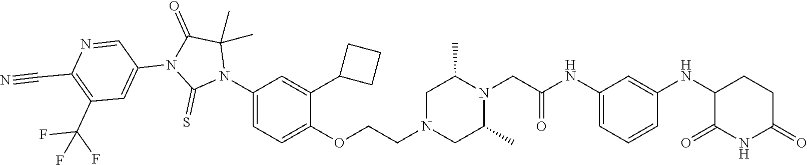 [SMILES: C[C@@H]1CN(CCOc2ccc(N3C(=S)N(c4cnc(C#N)c(C(F)(F)F)c4)C(=O)C3(C)C)cc2C2CCC2)C[C@H](C)N1CC(=O)Nc1cccc(NC2CCC(=O)NC2=O)c1] | 2-((2R,6S)-4-(2-(4-(3-(6-cyano-5-(trifluorometh- yl)pyridin-3-yl)-5,5-dimethyl-4-oxo-2-thioxoimid- azolidin-1-yl)-2-cyclobutylphenoxy)ethyl)-2,6- dimethylpiperazin-1-yl)-N-(3-(2,6-dioxopiperidin- 3-ylamino)phenyl)acetamide |
| 269 | 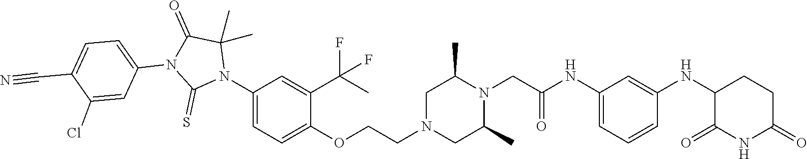 [SMILES: C[C@@H]1CN(CCOc2ccc(N3C(=S)N(c4ccc(C#N)c(Cl)c4)C(=O)C3(C)C)cc2C(C)(F)F)C[C@H](C)N1CC(=O)Nc1cccc(NC2CCC(=O)NC2=O)c1] | 2-((2S,6R)-4-(2-(4-(3-(3-chloro-4-cyanophenyl)- 5,5-dimethyl-4-oxo-2-thioxoimidazolidin-1-yl)-2- (1,1-difluoroethyl)phenoxy)ethyl)-2,6-dimethyl- piperazin-1-yl)-N-(3-(2,6-dioxopiperidin-3-yl- amino)phenyl)acetamide |
| 270 | 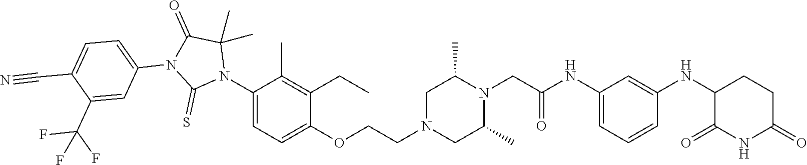 [SMILES: CCc1c(OCCN2C[C@@H](C)N(CC(=O)Nc3cccc(NC4CCC(=O)NC4=O)c3)[C@@H](C)C2)ccc(N2C(=S)N(c3ccc(C#N)c(C(F)(F)F)c3)C(=O)C2(C)C)c1C] | 2-((2R,6S)-4-(2-(4-(3-(4-cyano-3-(trifluorometh- yl)phenyl)-5,5-dimethyl-4-oxo-2-thioxoimidazol- idin-1-yl)-2-ethyl-3-methylphenoxy)ethyl)-2,6- dimethylpiperazin-1-yl)-N-(3-(2,6-dioxopiperidin- 3-ylamino)phenyl)acetamide |
| 271 | 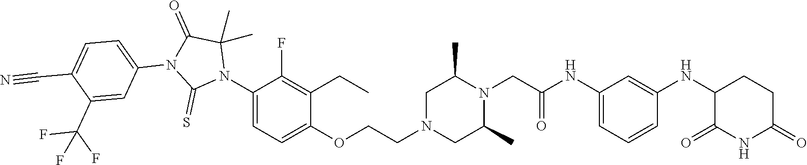 [SMILES: CCc1c(OCCN2C[C@@H](C)N(CC(=O)Nc3cccc(NC4CCC(=O)NC4=O)c3)[C@@H](C)C2)ccc(N2C(=S)N(c3ccc(C#N)c(C(F)(F)F)c3)C(=O)C2(C)C)c1F] | 2-((2S,6R)-4-(2-(4-(3-(4-cyano-3-(trifluorometh- yl)phenyl)-5,5-dimethyl-4-oxo-2-thioxoimidazol- idin-1-yl)-2-ethyl-3-fluorophenoxy)ethyl)-2,6- dimethylpiperazin-1-yl)-N-(3-(2,6-dioxopiperidin- 3-ylamino)phenyl)acetamide |
| 272 | 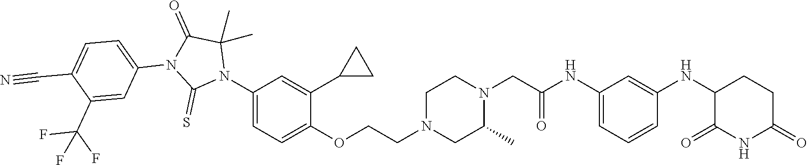 [SMILES: C[C@@H]1CN(CCOc2ccc(N3C(=S)N(c4ccc(C#N)c(C(F)(F)F)c4)C(=O)C3(C)C)cc2C2CC2)CCN1CC(=O)Nc1cccc(NC2CCC(=O)NC2=O)c1] | 2-((R)-4-(2-(4-(3-(4-cyano-3-(trifluoromethyl)- phenyl)-5,5-dimethyl-4-oxo-2-thioxoimidazolidin- 1-yl)-2-cyclopropylphenoxy)ethyl)-2-methyl- piperazin-1-yl)-N-(3-(2,6-dioxopiperidin-3-yl- amino)phenyl)acetamide |
| 273 | 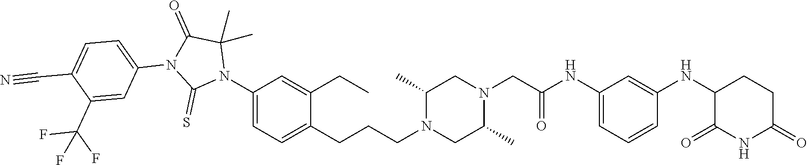 [SMILES: CCc1cc(N2C(=S)N(c3ccc(C#N)c(C(F)(F)F)c3)C(=O)C2(C)C)ccc1CCCN1C[C@@H](C)N(CC(=O)Nc2cccc(NC3CCC(=O)NC3=O)c2)C[C@H]1C] | 2-((2R,5R)-4-(3-(4-(3-(4-cyano-3-(trifluorometh- yl)phenyl)-5,5-dimethyl-4-oxo-2-thioxoimidazol- idin-1-yl)-2-ethylphenyl)propyl)-2,5-dimethyl- piperazin-1-yl)-N-(3-(2,6-dioxopiperidin-3-yl- amino)phenyl)acetamide |
| 274 | 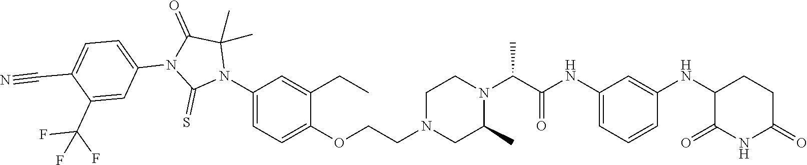 [SMILES: CCc1cc(N2C(=S)N(c3ccc(C#N)c(C(F)(F)F)c3)C(=O)C2(C)C)ccc1OCCN1CCN([C@H](C)C(=O)Nc2cccc(NC3CCC(=O)NC3=O)c2)[C@@H](C)C1] | (2R)-2-((S)-4-(2-(4-(3-(4-cyano-3-(trifluorometh- yl)phenyl)-5,5-dimethyl-4-oxo-2-thioxoimidazol- idin-1-yl)-2-ethylphenoxy)ethyl)-2-methylpiper- azin-1-yl)-N-(3-(2,6-dioxopiperidin-3-ylamino)- phenyl)propanamide |
| 275 | 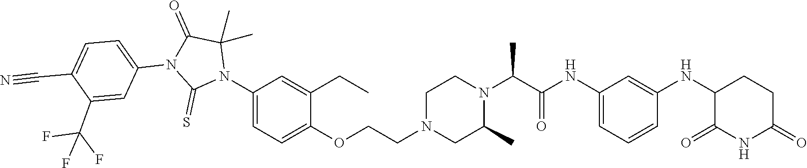 [SMILES: CCc1cc(N2C(=S)N(c3ccc(C#N)c(C(F)(F)F)c3)C(=O)C2(C)C)ccc1OCCN1CCN([C@@H](C)C(=O)Nc2cccc(NC3CCC(=O)NC3=O)c2)[C@@H](C)C1] | (2S)-2-((S)-4-(2-(4-(3-(4-cyano-3-(trifluorometh- yl)phenyl)-5,5-dimethyl-4-oxo-2-thioxoimidazol- idin-1-yl)-2-ethylphenoxy)ethyl)-2-methylpiper- azin-1-yl)-N-(3-(2,6-dioxopiperidin-3-ylamino)- phenyl)propanamide |
| 276 | 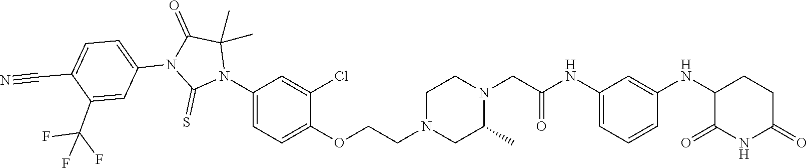 [SMILES: C[C@@H]1CN(CCOc2ccc(N3C(=S)N(c4ccc(C#N)c(C(F)(F)F)c4)C(=O)C3(C)C)cc2Cl)CCN1CC(=O)Nc1cccc(NC2CCC(=O)NC2=O)c1] | 2-((R)-4-(2-(2-chloro-4-(3-(4-cyano-3-trifluoro- methyl)phenyl)-5,5-dimethyl-4-oxo-2-thioxoimid- azolidin-1-yl)phenoxy)ethyl)-2-methylpiperazin- 1-yl)-N-(3-(2,6-dioxopiperidin-3-ylamino)phenyl)- acetamide |
| 277 | 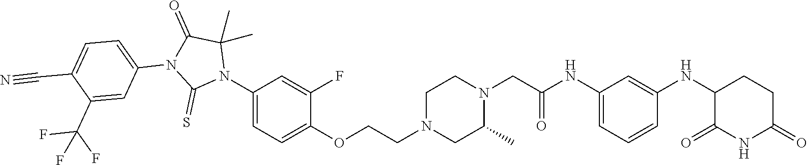 [SMILES: C[C@@H]1CN(CCOc2ccc(N3C(=S)N(c4ccc(C#N)c(C(F)(F)F)c4)C(=O)C3(C)C)cc2F)CCN1CC(=O)Nc1cccc(NC2CCC(=O)NC2=O)c1] | 2-((R)-4-(2-(4-(3-(4-cyano-3-(trifluoromethyl)- phenyl)-5,5-dimethyl-4-oxo-2-thioxoimidazolidin- 1-yl)-2-fluorophenoxy)ethyl)-2-methylpiperazin- 1-yl)-N-(3-(2,6-dioxopiperidin-3-ylamino)phen- yl)acetamide |
| 278 | 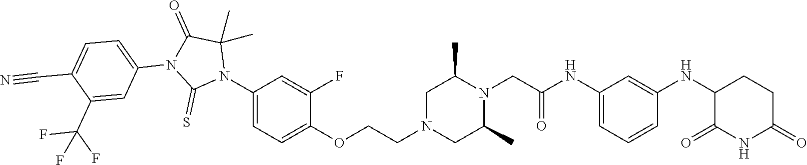 [SMILES: C[C@@H]1CN(CCOc2ccc(N3C(=S)N(c4ccc(C#N)c(C(F)(F)F)c4)C(=O)C3(C)C)cc2F)C[C@H](C)N1CC(=O)Nc1cccc(NC2CCC(=O)NC2=O)c1] | 2-((2S,6R)-4-(2-(4-(3-(4-cyano-3-(trifluorometh- yl)phenyl)-5,5-dimethyl-4-oxo-2-thioxoimidazol- idin-1-yl)-2-fluorophenoxy)ethyl)-2,6-dimethyl- piperazin-1-yl)-N-(3-(2,6-dioxopiperidin-3-yl- amino)phenyl)acetamide |
| 279 | 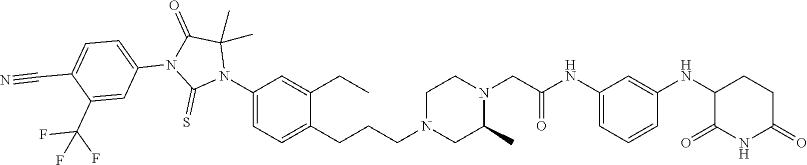 [SMILES: CCc1cc(N2C(=S)N(c3ccc(C#N)c(C(F)(F)F)c3)C(=O)C2(C)C)ccc1CCCN1CCN(CC(=O)Nc2cccc(NC3CCC(=O)NC3=O)c2)[C@@H](C)C1] | 2-((S)-4-(3-(4-(3-(4-cyano-3-(trifluoromethyl)- phenyl)-5,5-dimethyl-4-oxo-2-thioxoimidazol- idin-1-yl)-2-ethylphenyl)propyl)-2-methylpiper- azin-1-yl)-N-(3-(2,6-dioxopiperidin-3-ylamino)- phenyl)acetamide |
| 280 | 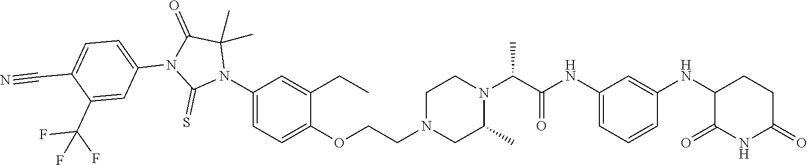 [SMILES: CCc1cc(N2C(=S)N(c3ccc(C#N)c(C(F)(F)F)c3)C(=O)C2(C)C)ccc1OCCN1CCN([C@H](C)C(=O)Nc2cccc(NC3CCC(=O)NC3=O)c2)[C@H](C)C1] | (2R)-2-((R)-4-(2-(4-(3-(4-cyano-3-(trifluorometh- yl)phenyl)-5,5-dimethyl-4-oxo-2-thioxoimidazol- idin-1-yl)-2-ethylphenoxy)ethyl)-2-methylpiper- azin-1-yl)-N-(3-(2,6-dioxopiperidin-3-ylamino)- phenyl)propanamide |
| 281 | 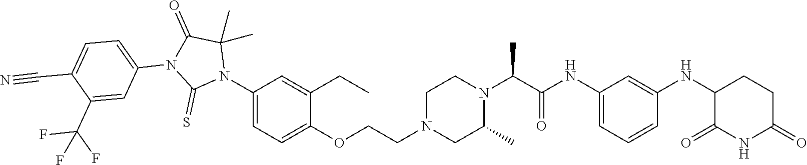 [SMILES: CCc1cc(N2C(=S)N(c3ccc(C#N)c(C(F)(F)F)c3)C(=O)C2(C)C)ccc1OCCN1CCN([C@@H](C)C(=O)Nc2cccc(NC3CCC(=O)NC3=O)c2)[C@H](C)C1] | (2S)-2-((R)-4-(2-(4-(3-(4-cyano-3-(trifluorometh- yl)phenyl)-5,5-dimethyl-4-oxo-2-thioxoimidazol- idin-1-yl)-2-ethylphenoxy)ethyl)-2-methylpiper- azin-1-yl)-N-(3-(2,6-dioxopiperidin-3-ylamino)- phenyl)propanamide |
| 282 | 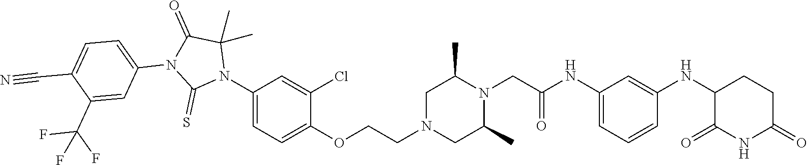 [SMILES: C[C@@H]1CN(CCOc2ccc(N3C(=S)N(c4ccc(C#N)c(C(F)(F)F)c4)C(=O)C3(C)C)cc2Cl)C[C@H](C)N1CC(=O)Nc1cccc(NC2CCC(=O)NC2=O)c1] | 2-((2S,6R)-4-(2-(2-chloro-4-(3-(4-cyano-3-(tri- fluoromethyl)phenyl)-5,5-dimethyl-4-oxo-2- thioxoimidazolidin-1-yl)phenoxy)ethyl)-2,6- dimethylpiperazin-1-yl)-N-(3-(2,6-dioxopiper- idin-3-ylamino)phenyl)acetamide |
| 283 | 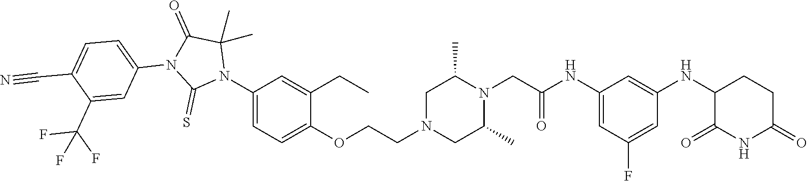 [SMILES: CCc1cc(N2C(=S)N(c3ccc(C#N)c(C(F)(F)F)c3)C(=O)C2(C)C)ccc1OCCN1C[C@@H](C)N(CC(=O)Nc2cc(F)cc(NC3CCC(=O)NC3=O)c2)[C@@H](C)C1] | 2-((2R,6S)-4-(2-(4-(3-(4-cyano-3-(trifluorometh- yl)phenyl)-5,5-dimethyl-4-oxo-2-thioxoimidazol- idin-1-yl)-2-ethylphenoxy)ethyl)-2,6-dimethyl- piperazin-1-yl)-N-(3-(2,6-dioxopiperidin-3-yl- amino)-5-fluorophenyl)acetamide |
| 284 | 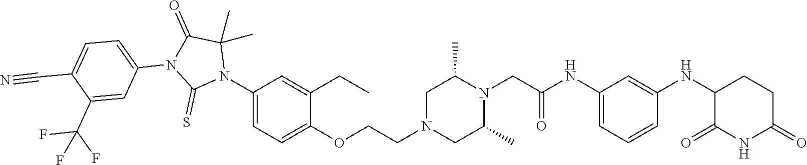 [SMILES: CCc1cc(N2C(=S)N(c3ccc(C#N)c(C(F)(F)F)c3)C(=O)C2(C)C)ccc1OCCN1C[C@@H](C)N(CC(=O)Nc2cccc(NC3CCC(=O)NC3=O)c2)[C@@H](C)C1] | 2-((2R,6S)-4-(3-(4-(3-(4-cyano-3-(trifluorometh- yl)phenyl)-5,5-dimethyl-4-oxo-2-thioxoimidazol- idin-1-yl)-2-ethylphenyl)propyl)-2,6-dimethyl- piperazin-1-yl)-N-(3-(2,6-dioxopiperidin-3-yl- amino)phenyl)acetamide |
| 285 | 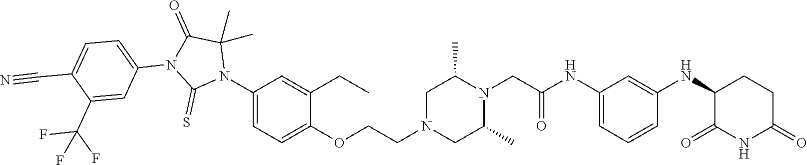 [SMILES: CCc1cc(N2C(=S)N(c3ccc(C#N)c(C(F)(F)F)c3)C(=O)C2(C)C)ccc1OCCN1C[C@@H](C)N(CC(=O)Nc2cccc(N[C@H]3CCC(=O)NC3=O)c2)[C@@H](C)C1] | 2-((2R,6S)-4-(2-(4-(3-(4-cyano-3-(trifluorometh- yl)phenyl)-5,5-dimethyl-4-oxo-2-thioxoimidazol- idin-1-yl)-2-ethylphenoxy)ethyl)-2,6-dimethyl- piperazin-1-yl)-N-(3-((S)-2,6-dioxopiperidin-3- ylamino)phenyl)acetamide |
| 286 | 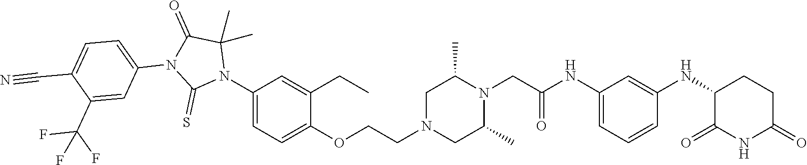 [SMILES: CCc1cc(N2C(=S)N(c3ccc(C#N)c(C(F)(F)F)c3)C(=O)C2(C)C)ccc1OCCN1C[C@@H](C)N(CC(=O)Nc2cccc(N[C@@H]3CCC(=O)NC3=O)c2)[C@@H](C)C1] | 2-((2R,6S)-4-(2-(4-(3-(4-cyano-3-(trifluorometh- yl)phenyl)-5,5-dimethyl-4-oxo-2-thioxoimidazol- idin-1-yl)-2-ethylphenoxy)ethyl)-2,6-dimethyl- piperazin-1-yl)-N-(3-((R)-2,6-dioxopiperidin-3- ylamino)phenyl)acetamide |
| 287 | 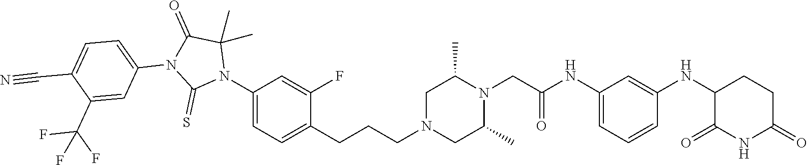 [SMILES: C[C@@H]1CN(CCCc2ccc(N3C(=S)N(c4ccc(C#N)c(C(F)(F)F)c4)C(=O)C3(C)C)cc2F)C[C@H](C)N1CC(=O)Nc1cccc(NC2CCC(=O)NC2=O)c1] | 2-((2R,6S)-4-(3-(4-(3-(4-cyano-3-(trifluorometh- yl)phenyl)-5,5-dimethyl-4-oxo-2-thioxoimidazol- idin-1-yl)-2-fluorophenyl)propyl)-2,6-dimethyl- piperazin-1-yl)-N-(3-(2,6-dioxopiperidin-3-yl- amino)phenyl)acetamide |
| 288 | 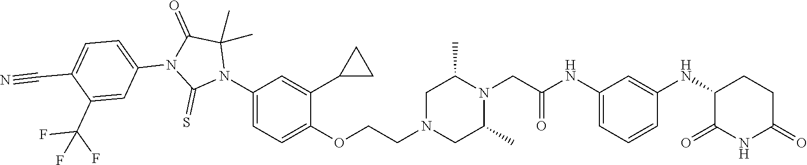 [SMILES: C[C@@H]1CN(CCOc2ccc(N3C(=S)N(c4ccc(C#N)c(C(F)(F)F)c4)C(=O)C3(C)C)cc2C2CC2)C[C@H](C)N1CC(=O)Nc1cccc(N[C@@H]2CCC(=O)NC2=O)c1] | 2-((2R,6S)-4-(2-(4-(3-(4-cyano-3-(trifluorometh- yl)phenyl)-5,5-dimethyl-4-oxo-2-thioxoimidazol- idin-1-yl)-2-cyclopropylphenoxy)ethyl)-2,6- dimethylpiperazin-1-yl)-N-(3-((R)-2,6-dioxo- piperidin-3-ylamino)phenyl)acetamide |
| 289 | 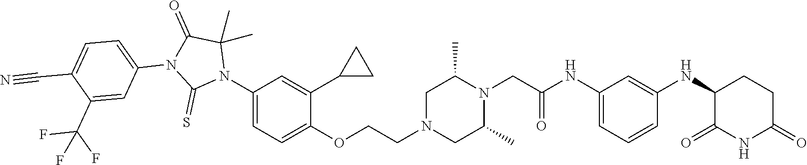 [SMILES: C[C@@H]1CN(CCOc2ccc(N3C(=S)N(c4ccc(C#N)c(C(F)(F)F)c4)C(=O)C3(C)C)cc2C2CC2)C[C@H](C)N1CC(=O)Nc1cccc(N[C@H]2CCC(=O)NC2=O)c1] | 2-((2R,6S)-4-(2-(4-(3-(4-cyano-3-(trifluorometh- yl)phenyl)-5,5-dimethyl-4-oxo-2-thioxoimidazol- idin-1-yl)-2-cyclopropylphenoxy)ethyl)-2,6- dimethylpiperazin-1-yl)-N-(3-((S)-2,6-dioxo- piperidin-3-ylamino)phenyl)acetamide |
| 290 | 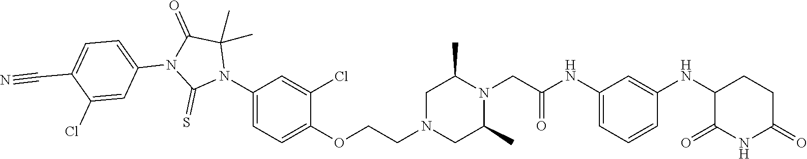 [SMILES: C[C@@H]1CN(CCOc2ccc(N3C(=S)N(c4ccc(C#N)c(Cl)c4)C(=O)C3(C)C)cc2Cl)C[C@H](C)N1CC(=O)Nc1cccc(NC2CCC(=O)NC2=O)c1] | 2-((2S,6R)-4-(2-(2-chloro-4-(3-(3-chloro-4-cyano- phenyl)-5,5-dimethyl-4-oxo-2-thioxoimidazolidin- 1-yl)phenoxy)ethyl)-2,6-dimethylpiperazin-1-yl)- N-(3-((2,6-dioxopiperidin-3-yl)amino)phenyl)- acetamide |
| 291 | 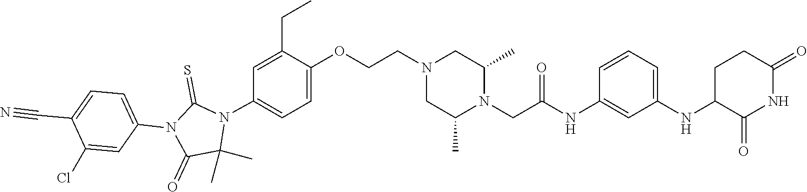 [SMILES: CCc1cc(N2C(=S)N(c3ccc(C#N)c(Cl)c3)C(=O)C2(C)C)ccc1OCCN1C[C@@H](C)N(CC(=O)Nc2cccc(NC3CCC(=O)NC3=O)c2)[C@@H](C)C1] | 2-((2S,6R)-4-(2-(4-(3-(3-chloro-4-cyanophenyl)- 5,5-dimethyl-4-oxo-2-thioxoimidazolidin-1-yl)-2- ethylphenoxy)ethyl)-2,6-dimethylpiperazin-1- yl)-N-(3-((2,6-dioxopiperidin-3-yl)amino)phen- yl)acetamide |

In certain embodiments, the compound is selected from (S)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide; 2-(S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-methylpiperazin-1-yl)-N-(3-(2,6-dioxopiperidin-3-ylamino)phenyl)acetamide; 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; 2-((S)-4-(2-(4-(3-(4-cyano-3-trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; and N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide or a pharmaceutically acceptable salt, tautomer or isotopologue thereof.

In one embodiment, the compound is 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide or a pharmaceutically acceptable salt, tautomer or isotopologue thereof.

In one embodiment, the compound is 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide.

Piperidine Dione Compounds can be prepared according to the methods described in the Examples provided herein or as described in U.S. Pat. No. 11,149,007, the disclosure of which is incorporated herein by reference in its entirety. Piperidine Dione Compound can also be synthesized according to other methods apparent to those of skill in the art based upon the teaching herein.

### Second Agents

The second agents for use in the methods provided here are selected from a PI3K inhibitor, an AKT inhibitor, a BET inhibitor, JAK inhibitor, and an EZH2 inhibitor. In one embodiment, the second agent is selected from a capivasertib, ipatasertib, AZD8186, 6H-thieno[3,2-f][1,2,4]triazolo[4,3-a][1,4]diazepine-6-acetic acid, tazemetostat, ruxolitinib and sorafenib.

### Methods of Use

In one embodiment, provided herein are methods of treating, preventing, managing, and/or ameliorating an androgen receptor mediated disease, or one or more symptoms or causes thereof, by administering a Piperidine Dione Compound in combination with one or more second agents selected from a PI3K inhibitor, an AKT inhibitor, a BET inhibitor, JAK inhibitor, and an EZH2 inhibitor to a patient having an androgen receptor mediated disease, or one or more symptoms or causes thereof. In one embodiment, provided herein are methods of treating, preventing, managing, and/or ameliorating an androgen receptor mediated disease, or one or more symptoms or causes thereof, by administering a Piperidine Dione Compound in combination with one or more second agents selected from capivasertib, ipatasertib, AZD8186, 6H-thieno[3,2-f][1,2,4]triazolo[4,3-a][1,4]diazepine-6-acetic acid, tazemetostat, ruxolitinib and sorafenib to a patient having an androgen receptor mediated disease, or one or more symptoms or causes thereof.

Provided herein is a Piperidine Dione Compound for use in such methods of treating, preventing, managing, and/or ameliorating an androgen receptor mediated disease, or one or more symptoms or causes thereof, wherein the compound is administered in combination with one or more second agents selected from a PI3K inhibitor, an AKT inhibitor, a BET inhibitor, JAK inhibitor, and an EZH2 inhibitor. In one aspect, provided herein is a Piperidine Dione Compound for use in such methods of treating, preventing, managing, and/or ameliorating an androgen receptor mediated disease, or one or more symptoms or causes thereof, wherein the compound is administered in combination with one or more second agents selected from capivasertib, ipatasertib, AZD8186, 6H-thieno[3,2-f][1,2,4]triazolo[4,3-a][1,4]diazepine-6-acetic acid, tazemetostat, ruxolitinib and sorafenib.

In certain embodiments, the methods and/or combinations provided herein synergistically inhibit proliferation of prostate cancer cells.

In one aspect, provided herein are methods of treating patients who have been previously treated for an androgen receptor mediated disease but are non-responsive to therapy, as well as those who have not previously been treated. Also encompassed are methods of treating patients regardless of patient's age, although some diseases or disorders are more common in certain age groups. Further encompassed are methods of treating patients who have undergone surgery in an attempt to treat the disease or condition at issue, as well as those who have not. Because patients with an androgen receptor mediated disease have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with an androgen receptor mediated disease.

In some embodiments, the methods comprise administering a compound selected from (S)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-methylpiperazin-1-yl)-N-(3-(2,6-dioxopiperidin-3-ylamino)phenyl)acetamide; 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; and N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide or a pharmaceutically acceptable salt, tautomer or isotopologue thereof and a second agent selected from a PI3K inhibitor, an AKT inhibitor, a BET inhibitor, JAK inhibitor, and an EZH2 inhibitor.

In some embodiments, the methods comprise administering 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide or a pharmaceutically acceptable salt, tautomer or isotopologue thereof and a second agent selected from a PI3K inhibitor, an AKT inhibitor, a BET inhibitor, JAK inhibitor, and an EZH2 inhibitor.

In one embodiment, the methods comprise administering 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide and a second agent selected from a PI3K inhibitor, an AKT inhibitor, a BET inhibitor, JAK inhibitor, and an EZH2 inhibitor.

In some embodiments, the methods comprise administering a compound selected from (S)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-methylpiperazin-1-yl)-N-(3-(2,6-dioxopiperidin-3-ylamino)phenyl)acetamide; 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; and N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide or a pharmaceutically acceptable salt, tautomer or isotopologue thereof in combination with one or more second agents selected from capivasertib, ipatasertib, AZD8186, 6H-thieno[3,2-f][1,2,4]triazolo[4,3-a][1,4]diazepine-6-acetic acid, tazemetostat, ruxolitinib and sorafenib.

In some embodiments, the methods comprise administering 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide or a pharmaceutically acceptable salt, tautomer or isotopologue thereof and a second agent selected from capivasertib, ipatasertib, AZD8186, 6H-thieno[3,2-f][1,2,4]triazolo[4,3-a][1,4]diazepine-6-acetic acid, tazemetostat, ruxolitinib and sorafenib.

In one embodiment, the methods comprise administering 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide and a second agent selected from capivasertib, ipatasertib, AZD8186, 6H-thieno[3,2-f][1,2,4]triazolo[4,3-a][1,4]diazepine-6-acetic acid, tazemetostat, ruxolitinib and sorafenib.

In certain embodiments, the methods provided herein comprise administering a Piperidine Dione Compound in combination with capivasertib. In certain embodiments, the methods provided herein comprise administering a compound selected from (S)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-methylpiperazin-1-yl)-N-(3-(2,6-dioxopiperidin-3-ylamino)phenyl)acetamide; 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; and N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide or a pharmaceutically acceptable salt, tautomer or isotopologue thereof in combination with capivasertib. In certain embodiments, the methods provided herein comprise administering 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide or a pharmaceutically acceptable salt, tautomer or isotopologue thereof in combination with capivasertib. In certain embodiments, the methods provided herein comprise administering 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide in combination with capivasertib.

In certain embodiments, the methods provided herein comprise administering a Piperidine Dione Compound in combination with ipatasertib. In certain embodiments, the methods provided herein comprise administering a compound selected from (S)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-methylpiperazin-1-yl)-N-(3-(2,6-dioxopiperidin-3-ylamino)phenyl)acetamide; 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; and N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide or a pharmaceutically acceptable salt, tautomer or isotopologue thereof in combination with ipatasertib. In certain embodiments, the methods provided herein comprise administering 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide or a pharmaceutically acceptable salt, tautomer or isotopologue thereof in combination with ipatasertib. In certain embodiments, the methods provided herein comprise administering 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide in combination with ipatasertib.

In certain embodiments, the methods provided herein comprise administering a Piperidine Dione Compound in combination with AZD8186. In certain embodiments, the methods provided herein comprise administering a compound selected from (S)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-methylpiperazin-1-yl)-N-(3-(2,6-dioxopiperidin-3-ylamino)phenyl)acetamide; 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; and N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide or a pharmaceutically acceptable salt, tautomer or isotopologue thereof in combination with AZD8186. In certain embodiments, the methods provided herein comprise administering 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide or a pharmaceutically acceptable salt, tautomer or isotopologue thereof in combination with AZD8186. In certain embodiments, the methods provided herein comprise administering 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide in combination with AZD8186.

In certain embodiments, the methods provided herein comprise administering a Piperidine Dione Compound in combination with 6H-thieno[3,2-f][1,2,4]triazolo[4,3-a][1,4]diazepine-6-acetic acid. In certain embodiments, the methods provided herein comprise administering a compound selected from (S)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-methylpiperazin-1-yl)-N-(3-(2,6-dioxopiperidin-3-ylamino)phenyl)acetamide; 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; and N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide or a pharmaceutically acceptable salt, tautomer or isotopologue thereof in combination with 6H-thieno[3,2-f][1,2,4]triazolo[4,3-a][1,4]diazepine-6-acetic acid. In certain embodiments, the methods provided herein comprise administering 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide or a pharmaceutically acceptable salt, tautomer or isotopologue thereof in combination with 6H-thieno[3,2-f][1,2,4]triazolo[4,3-a][1,4]diazepine-6-acetic acid. In certain embodiments, the methods provided herein comprise administering 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide in combination with 6H-thieno[3,2-f][1,2,4]triazolo[4,3-a][1,4]diazepine-6-acetic acid.

In certain embodiments, the methods provided herein comprise administering a Piperidine Dione Compound in combination with tazemetostat. In certain embodiments, the methods provided herein comprise administering a compound selected from (S)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-methylpiperazin-1-yl)-N-(3-(2,6-dioxopiperidin-3-ylamino)phenyl)acetamide; 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; and N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide or a pharmaceutically acceptable salt, tautomer or isotopologue thereof in combination with tazemetostat. In certain embodiments, the methods provided herein comprise administering 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide or a pharmaceutically acceptable salt, tautomer or isotopologue thereof in combination with tazemetostat. In certain embodiments, the methods provided herein comprise administering a Piperidine Dione Compound in combination with tazemetostat.

In certain embodiments, the methods provided herein comprise administering a Piperidine Dione Compound in combination with ruxolitinib. In certain embodiments, the methods provided herein comprise administering a compound selected from (S)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-methylpiperazin-1-yl)-N-(3-(2,6-dioxopiperidin-3-ylamino)phenyl)acetamide; 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; and N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide or a pharmaceutically acceptable salt, tautomer or isotopologue thereof in combination with ruxolitinib. In certain embodiments, the methods provided herein comprise administering 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide or a pharmaceutically acceptable salt, tautomer or isotopologue thereof in combination with ruxolitinib. In certain embodiments, the methods provided herein comprise administering 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide in combination with ruxolitinib.

In certain embodiments, the methods provided herein comprise administering a Piperidine Dione Compound in combination with sorafenib. In certain embodiments, the methods provided herein comprise administering a compound selected from (S)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-methylpiperazin-1-yl)-N-(3-(2,6-dioxopiperidin-3-ylamino)phenyl)acetamide; 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide; and N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide or a pharmaceutically acceptable salt, tautomer or isotopologue thereof in combination with sorafenib. In certain embodiments, the methods provided herein comprise administering 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide or a pharmaceutically acceptable salt, tautomer or isotopologue thereof in combination with sorafenib. In certain embodiments, the methods provided herein comprise administering 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide in combination with sorafenib.

In some embodiments, the AR mediated disease is an AR wild-type mediated disease. In other embodiments, the AR mediated disease is the result of AR amplification.

In certain embodiments, the AR mediated disease is prostate cancer. In some such embodiments, the prostate cancer is castration resistant prostate cancer (CRPC). In some such embodiments, the prostate cancer is metastatic castration resistant prostate cancer (mCRPC). In still another embodiment, the prostate cancer is non-metastatic CRPC (nmCRPC). In some embodiments, the prostate cancer is hormone refractory. In some embodiments, the prostate cancer is resistant to treatment with an AR antagonist. For example, the prostate cancer is resistant to treatment with one or more of enzalutamide, bicalutamide, abiraterone, ARN-509, ODM-201, EPI-001, EPI-506, AZD-3514, galeterone, ASC-J9, flutamide, hydroxyflutamide, nilutamide, cyproterone acetate, ketoconazole, or spironolactone.

Provided herein are methods of reducing AR levels relative to a baseline level, the method comprising administering to a subject an effective amount of a Piperidine Dione Compound in combination with one or more second agents selected from a P1³K inhibitor, an AKT inhibitor, a BET inhibitor, JAK inhibitor, and an EZH2 inhibitor. Also provided herein are Piperidine Dione Compounds for use in methods of reducing AR levels in a cell in vivo, ex vivo or in vitro, comprising contacting the cell with an effective amount of a Piperidine Dione Compound in combination with one or more second agents selected from a PI3K inhibitor, an AKT inhibitor, a BET inhibitor, JAK inhibitor, and an EZH2 inhibitor. In one embodiment, the cell is in a patient. In one embodiment, the cell is not in a patient. In one embodiment, provided herein are methods of reducing levels of wild-type AR within a tumor, the method comprising administering a therapeutically effective amount of a Piperidine Dione Compound in combination with one or more second agents selected from a PI3K inhibitor, an AKT inhibitor, a BET inhibitor, JAK inhibitor, and an EZH2 inhibitor, to reduce the level of wild-type AR within the tumor. In one embodiment, provided herein are methods of reducing levels of AR-full length (AR-FL) within a tumor, the method comprising administering a therapeutically effective amount of a Piperidine Dione Compound in combination with one or more second agents selected from a PI3K inhibitor, an AKT inhibitor, a BET inhibitor, JAK inhibitor, and an EZH2 inhibitor, to reduce the level of AR-full length (AR-FL) within the tumor. In some embodiments, the AR levels are reduced compared to the AR levels prior to Piperidine Dione Compound administration. In some embodiments, the AR levels are reduced by 20%, 30%, 40%, 50%, 60%, 70%, 80%, 90%, 95%, or 99% compared to the AR levels prior to administration of Piperidine Dione Compound in combination with one or more second agents selected from a PI3K inhibitor, an AKT inhibitor, a BET inhibitor, JAK inhibitor, and an EZH2 inhibitor.

The dose of a Piperidine Dione Compound to be administered to a subject is rather widely variable and can be subject to the judgment of a health-care practitioner. In general, the Piperidine Dione Compounds can be administered one to four times a day in a dose of about 0.001 mg/kg of a subject's body weight to about 10 mg/kg of a subject's body weight, but the above dosage may be properly varied depending on the age, body weight and medical condition of the subject and the type of administration. In one embodiment, the dose is about 0.001 mg/kg of a subject's body weight to about 5 mg/kg of a subject's body weight, about 0.01 mg/kg of a subject's body weight to about 5 mg/kg of a subject's body weight, about 0.05 mg/kg of a subject's body weight to about 1 mg/kg of a subject's body weight, about 0.1 mg/kg of a subject's body weight to about 0.75 mg/kg of a subject's body weight or about 0.25 mg/kg of a subject's body weight to about 0.5 mg/kg of a subject's body weight. In one embodiment, one dose is given per day. In any given case, the amount of the Piperidine Dione Compound administered will depend on such factors as the solubility of the active component, the formulation used and the route of administration.

In another embodiment, provided herein are methods for the treatment or prevention of an AR mediated disease comprising the administration of about 0.01 mg/day to about 750 mg/day, about 0.1 mg/day to about 375 mg/day, about 0.1 mg/day to about 150 mg/day, about 0.1 mg/day to about 75 mg/day, about 0.1 mg/day to about 50 mg/day, about 0.1 mg/day to about 25 mg/day, or about 0.1 mg/day to about 10 mg/day of a Piperidine Dione Compound to a subject having an AR mediated disease.

A Piperidine Dione Compound can be administered once, twice, three, four or more times daily. In a particular embodiment, doses of 100 mg or less are administered as a once daily dose and doses of more than 100 mg are administered twice daily in an amount equal to one half of the total daily dose.

### Pharmaceutical Compositions and Routes of Administration

A Piperidine Dione Compound provided herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions.

The Piperidine Dione Compounds can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the Piperidine Dione Compounds in the pharmaceutical composition may be at a level that will exercise the desired effect; for example, about 0.005 mg/kg of a subject's body weight to about 10 mg/kg of a subject's body weight in unit dosage for both oral and parenteral administration.

In one embodiment, provided herein are capsules containing a Piperidine Dione Compound without an additional carrier, excipient or vehicle.

In another embodiment, provided herein are compositions comprising an effective amount of a Piperidine Dione Compound and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a Piperidine Dione Compound with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a Piperidine Dione Compound as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the Piperidine Dione Compound can be delayed or prolonged by proper formulation. For example, a slowly soluble pellet of the Piperidine Dione Compound can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the Piperidine Dione Compound in oily or emulsified vehicles that allow it to disperse slowly in the serum.

In certain embodiments, the Piperidine Dione Compound is administered orally. In one embodiment, when administered orally, a Piperidine Dione Compound is administered with a meal and water. In another embodiment, the Piperidine Dione Compound is dispersed in water or juice (e.g., apple juice or orange juice) or any other liquid and administered orally as a solution or a suspension.

The Piperidine Dione Compound can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In another embodiment, provided herein are unit dosage formulations that comprise between about 0.1 mg and 500 mg, about 1 mg and 250 mg, about 1 mg and about 100 mg, about 1 mg and about 50 mg, about 1 mg and about 25 mg, or between about 1 mg and about 10 mg of a Piperidine Dione Compound.

In a particular embodiment, provided herein are unit dosage formulations comprising about 0.1 mg or 100 mg of a Piperidine Dione Compound.

In another embodiment, provided herein are unit dosage formulations that comprise 0.5 mg, 1 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of a Piperidine Dione Compound.

In certain embodiments, tazemetostat may be formulated in suitable dosage unit with pharmaceutically acceptable excipients, carriers, adjuvants and vehicles, appropriate for oral administration. In certain embodiments, tazemetostat is formulated as a tablet for oral administration. In one embodiment, tazemetostat is administered orally in daily dose of 800 mg. In one embodiment, tazemetostat is administered orally twice daily in a dose of 400 mg.

In certain embodiments, ruxolitinib may be formulated in suitable dosage unit with pharmaceutically acceptable excipients, carriers, adjuvants and vehicles, appropriate for oral administration. In certain embodiments, ruxolitinib is formulated as a tablet for oral administration. In one embodiment, ruxolitinib is administered orally in a dose of 5 mg, 10 mg, 15 mg, 20 mg or 25 mg. In one embodiment, ruxolitinib is administered orally twice daily in a dose of 5 mg, 10 mg, 15 mg, 20 mg or 25 mg.

In certain embodiments, sorafenib may be formulated in suitable dosage unit with pharmaceutically acceptable excipients, carriers, adjuvants and vehicles, appropriate for oral administration. In certain embodiments, sorafenib is formulated as a tablet for oral administration. In one embodiment, sorafenib is administered orally in daily dose of 800 mg. In one embodiment, sorafenib is administered orally twice daily in a dose of 400 mg.

### Combination Therapy

In one embodiment, provided herein is a method of treating, preventing, and/or managing an androgen receptor (AR) mediated disease, comprising administering to a patient having an androgen receptor (AR) mediated disease a Piperidine Dione Compound in combination with one or more second agents selected from a PI3K inhibitor, an AKT inhibitor, a BET inhibitor, JAK inhibitor, and an EZH2 inhibitor, and optionally in combination with radiation therapy, blood transfusions, or surgery. In one embodiment, the second agent is selected from capivasertib, ipatasertib, AZD8186, 6H-thieno[3,2-f][1,2,4]triazolo[4,3-a][1,4]diazepine-6-acetic acid, tazemetostat, ruxolitinib and sorafenib.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a patient with a disease or disorder. E.g., “in combination” may include administration as a mixture, simultaneous administration using separate formulations, and consecutive administration in any order. “Consecutive” means that a specific time has passed between the administration of the active agents. For example, “consecutive” may be that more than 10 minutes have passed between the administration of the separate active agents. The time period can then be more than 10 min, more than 30 minutes, more than 1 hour, more than 3 hours, more than 6 hours or more than 12 hours. E.g., a first therapy (e.g., a prophylactic or therapeutic agent such as a formulation of Compound I provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

In one embodiment, administration of a Piperidine Dione Compound provided herein, and one or more second active agents to a patient can occur simultaneously or sequentially by the same or different routes of administration. In one embodiment, administration of a Piperidine Dione Compound provided herein, and one or more second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the androgen receptor (AR) mediated disease being treated.

The route of administration of a Piperidine Dione Compound provided herein, is independent of the route of administration of a second therapy. In one embodiment, a Piperidine Dione Compound provided herein, and a second therapy are administered by the same mode of administration. In another embodiment, a Piperidine Dione Compound provided herein is administered by one mode of administration, whereas the second agent is administered by another mode of administration.

In some embodiments of the methods described herein, the methods additionally comprise administering one or more additional agents selected from an AR antagonist (such as cyproterone acetate, spironolactone, bicalutamide, and enzalutamide), a 5α-reductase inhibitor (such as finasteride and dutasteride), a CYP17A1 inhibitor (such as abiraterone acetate), a gonadotropin-releasing hormone (GnRH) analog (such as leuprorelin and cetrorelix), and an anti-gonadotropin (such as megestrol acetate and medroxyprogesterone acetate).

The methods herein contemplate administration of an effective amount of a compound of Formula I and an effective amount of a second agent to achieve the desired or stated effect. Specific dosage and treatment regimens for any particular subject depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician.

Upon improvement of a subject's condition, a maintenance dose of a compound, composition or combination provided herein may be administered, if necessary. Subsequently, the dosage or frequency of administration, or both, may be reduced, as a function of the symptoms, to a level at which the improved condition is retained when the symptoms have been alleviated to the desired level. Subjects may, however, require intermittent treatment on a long-term basis upon any recurrence of disease symptoms.

### Evaluation of Activity

Standard physiological, pharmacological and biochemical procedures are available for testing the compounds to identify those that possess the desired activity.

Such assays include, for example, cell based assays, including the CTG proliferation assay described in the Example section.

### ENUMERATED EMBODIMENTS

1. A method of treating an androgen receptor mediated disease in a patient having an androgen receptor mediated disease comprising administering to the patient a therapeutically effective amount of a compound and a therapeutically effective amount of a second agent selected from a PI3K inhibitor, an AKT inhibitor, a BET inhibitor, JAK inhibitor, and an EZH2 inhibitor, wherein the compound has formula (I)

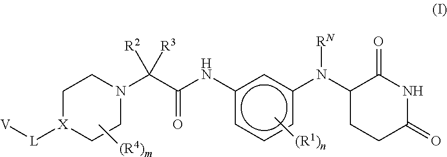

or a pharmaceutically acceptable salt, tautomer, isotopolog, or stereoisomer thereof, wherein
R^{N} is H;
each R¹ is independently selected from halogen, CN, and C₁₋₃ alkyl;
R² and R³ are each independently selected from H, and C₁₋₃ alkyl, or R² and R³ and the carbon to which they are attached form a substituted or unsubstituted C₃₋₆ cycloalkyl;
each R⁴ is independently substituted or unsubstituted C₁₋₃ alkyl, or two R⁴ groups, together with the same carbon atom or adjacent carbon atoms to which they are attached, form a substituted or unsubstituted C₃₋₆ cycloalkyl, or two R⁴ groups together with the non-adjacent carbon atoms to which they are attached form a substituted or unsubstituted 4-7-membered heterocyclyl;
X is N;
L is —O(C₁₋₆ alkyl)- or —(C₁₋₉ alkyl)-;
n is 0-4;
m is 0-8;
   V is

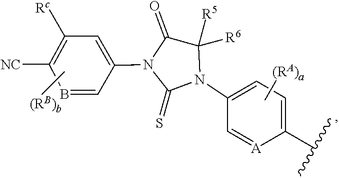

wherein
A is N, CH, or CR^{A};
B is N, CH, or CR^{B};
each R^{A} is independently selected from halogen, substituted or unsubstituted C₁₋₆ alkyl, and substituted or unsubstituted C₃₋₆ cycloalkyl;
each R^{B} is independently selected from halogen, and substituted or unsubstituted C₁₋₆ alkyl;
R^{C} is halogen or CF₃;
R⁵ and R⁶ are C₁₋₃ alkyl, or R⁵ and R⁶, together with the carbon atom to which they are attached, form a substituted or unsubstituted C₃₋₆ cycloalkyl or a 3-6 membered heterocyclyl;
a is 0-3; and
b is 0-2.

2. The method of embodiment 1, wherein L is —O(CH₂)ₚ— or —(CH₂)ₚ—, and p is 1-3.

3. The method of embodiment 1, wherein each R¹ is independently selected from Cl, F, CN, and —CH₃.

4. The method of embodiment 1, wherein n is 0.

5. The method of embodiment 1, wherein n is 1 or 2.

6. The method of embodiment 1, wherein R² and R³ are each independently selected from H and methyl, or wherein R² and R³ and the carbon to which they are attached form an unsubstituted cyclopropyl.

7. The method of embodiment 1, wherein each R⁴ is independently selected from methyl, CF₃, and CH₂OH, or wherein two R⁴ groups, together with the same carbon atom to which they are attached, form an unsubstituted cyclopropyl.

8. The method of embodiment 1, wherein m is 0, 1, 2, 3 or 4.

9. The method of embodiment 1, wherein two R⁴ groups together with the non-adjacent carbon atoms to which they are attached form an unsubstituted 4-7-membered heterocyclyl.

10. The method of embodiment 1, wherein L is O(CH₂)(CH₂)—, —O(CH₂)(CH(CH₃))—, —O(CH₂)(C(CH₃)₂)—, —O(CH(CH₃))(CH₂)—, —O(C(CH₃)₂)(CH₂)—, —O(CH(CH₃))(CH(CH₃))—, —O(CH(CH₃))(C(CH₃)₂)—, —O(C(CH₃)₂)(CH(CH₃))—, —(CH₂)—, —(CH₂)(CH₂)—, —(CH₂)(CH₂)(CH₂)—, —(C(CH₃)₂)(C(CH₃)₂)—, —(CH(CH₃))—, —(C(CH₃)₂)—, —(CH(CH₃))(CH(CH₃))—, —(CH(CH₃))(C(CH₃)₂)—, —(C(CH₃)₂)(CH(CH₃))—, —(C(CH₃)₂)(C(CH₃)₂)—, —(CH₃)(CH₃)(CH(CH₃))—, —(CH₂)(CH(CH₃))(CH₂)—, —(CH(CH₃))(CH₂)(CH₂)—, —(CH₂)(CH₂)(C(CH₂)₂)—, —(CH₂)(C(CH₂)₂)(CH₂)—, —(C(CH₂)₂)(CH₂)(CH₂)—, —(CH₂)(CH(CH₃))(CH(CH₃))—, —(CH(CH₃))(CH(CH₃))(CH(CH₃))—, —(CH(CH₃))(CH(CH₃))(CH₂)—, —(CH(CH₃))(CH₂)(CH(CH₃))—, —(CH₂)(CH(CH₃))(C(CH₃)₂)—, —(CH(CH₃))(CH₂)(C(CH₂)₂)—, —(C(CH₃)₂)(CH₂)(C(CH₃)₂)—, —(CH₂)(C(CH₃)₂)(C(CH₃)₂)—, —(CH₂)(C(CH₃)₂)(CH(CH₃))—, —(CH(CH₃)(C(CH₃)₂)(CH₂)—, —(C(CH₃)₂)(CH₂)(CH(CH₃))—, —(C(CH₃)₂)(CH(CH₃))(CH₂)—, —(C(CH₃)₂)(C(CH₃)₂)(CH₂)—, —(CH(CH₃))(CH(CH₃))(C(CH₃)₂)—, —(CH(CH₃))(C(CH₃)₂)(C(CH₃)₂)—, —(C(CH₃)₂)(CH(CH₃))(C(CH₃)₂)—, —(CH(CH₃))(C(CH₃)₂)(CH(CH₃))—, —(C(CH₃)₂)(C(CH₃)₂)(CH(CH₃))—, —(C(CH₃)₂)(CH(CH₃))(CH(CH₃))—, and —(C(CH₃)₂)(C(CH₃)₂)(C(CH₃)₂)—.

11. The method of embodiment 1, wherein L is —O(CH₂)(CH₂)—, —O(CH₂)(CH(CH₃))—, —O(CH(CH₃))(CH₂)—, or —(CH₂)(CH₂)(CH₂)—.

12. The method of embodiment 1, wherein A is CH.

13. The method of embodiment 1, wherein B is CH.

14. The method of embodiment 1, wherein B is N.

15. The method of embodiment 1, wherein a is 0, 1 or 2.

16. The method of embodiment 1, wherein each R^{A} is independently selected from Cl, Br, F, methyl, ethyl, n-propyl, isopropyl, isobutyl, sec-butyl, CF₃, CF₂CH₃, CH₂CH₂F, CH₂CHF₂, CH₂OH, CH(CH₃)OH, cyclopropyl, cyclobutyl, and cyclopentyl.

17. The method of embodiment 1, wherein b is 0 or 1.

18. The method of embodiment 1, wherein R^{B} is methyl.

19. The method of embodiment 1, wherein R^{C} is CF₃ or Cl.

20. The method of embodiment 1, wherein R⁵ and R⁶ are methyl, or R⁵ and R⁶, together with the carbon atom to which they are attached, form a cyclobutyl, cyclopentyl, cyclohexyl, or tetrahydrofuranyl.

21. A method of any one of embodiments 1-20, wherein the compound is selected from the group consisting of
2-(4-(2-(4-(3-(4-cyano-3-(trifluoromethyl)phenyl)-5,5-dimethyl-4-oxo-2-thioxoimidazolidin-1-yl)-2-fluorophenoxy)ethyl)piperazin-1-yl)-N-(3-(2,6-dioxopiperidin-3-ylamino)phenyl)acetamide,
2-(4-(2-(4-(3-(6-cyano-5-(trifluoromethyl)pyridin-3-yl)-5,5-dimethyl-4-oxo-2-thioxoimidazolidin-1-yl)phenoxy)ethyl)piperazin-1-yl)-N-(3-(2,6-dioxopiperidin-3-ylamino)phenyl)acetamide,
2-(4-(2-(2-chloro-4-(3-(4-cyano-3-(trifluoromethyl)phenyl)-5,5-dimethyl-4-oxo-2-thioxoimidazolidin-1-yl)phenoxy)ethyl)piperazin-1-yl)-N-(3-(2,6-dioxopiperidin-3-ylamino)phenyl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-fluorophenoxy)ethyl)piperazin-1-yl)acetamide,
2-(4-(2-(2-chloro-4-(3-(6-cyano-5-(trifluoromethyl)pyridin-3-yl)-5,5-dimethyl-4-oxo-2-thioxoimidazolidin-1-yl)phenoxy)ethyl)piperazin-1-yl)-N-(3-(2,6-dioxopiperidin-3-ylamino)phenyl)acetamide,
2-(4-(2-(4-(7-(6-cyano-5-(trifluoromethyl)pyridin-3-yl)-8-oxo-6-thioxo-5,7-diazaspiro[3.4]octan-5-yl)-2-fluorophenoxy)ethyl)piperazin-1-yl)-N-(3-(2,6-dioxopiperidin-3-ylamino)phenyl)acetamide,
2-(4-(2-(2-chloro-4-(7-(4-cyano-3-(trifluoromethyl)phenyl)-8-oxo-6-thioxo-5,7-diazaspiro[3.4]octan-5-yl)phenoxy)ethyl)piperazin-1-yl)-N-(3-(2,6-dioxopiperidin-3-ylamino)phenyl)acetamide,
2-(4-(2-(4-(7-(4-cyano-3-(trifluoromethyl)phenyl)-8-oxo-6-thioxo-5,7-diazaspiro[3.4]octan-5-yl)-2-fluorophenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide,
2-(4-(2-(2-bromo-4-(3-(4-cyano-3-(trifluoromethyl)phenyl)-5,5-dimethyl-4-oxo-2-thioxoimidazolidin-1-yl)phenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-(trifluoromethyl)phenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-fluoro-5-methylphenoxy)ethyl)piperazin-1-yl)-N-(3-(2,6-dioxopiperidin-3-ylamino)phenyl)acetamide,
2-(4-(2-(2-chloro-4-(3-(6-cyano-5-(trifluoromethyl)pyridin-3-yl)-5,5-dimethyl-4-oxo-2-thioxoimidazolidin-1-yl)phenoxy)ethyl)piperazin-1-yl)-N-(3-chloro-5-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-fluorophenoxy)ethyl)piperazin-1-yl)-N-(3-(2,6-dioxopiperidin-3-ylamino)-5-methylphenyl)acetamide,
2-(4-(2-(2-chloro-4-(3-(4-cyano-3-(trifluoromethyl)phenyl)-5,5-dimethyl-4-oxo-2-thioxoimidazolidin-1-yl)phenoxy)ethyl)piperazin-1-yl)-N-(3-chloro-5-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-isopropylphenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-isobutylphenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-fluorophenoxy)ethyl)piperazin-1-yl)-N-(3-(2,6-dioxopiperidin-3-ylamino)-5-fluorophenyl)acetamide,
2-(4-(2-(2-chloro-4-(3-(4-cyano-3-(trifluoromethyl)phenyl)-5,5-dimethyl-4-oxo-2-thioxoimidazolidin-1-yl)phenoxy)ethyl)piperazin-1-yl)-N-(2-chloro-5-(2,6-dioxopiperidin-3-ylamino)phenyl)acetamide,
(2S)-2-(4-(2-(2-chloro-4-(3-(4-cyano-3-(trifluoromethyl)phenyl)-5,5-dimethyl-4-oxo-2-thioxoimidazolidin-1-yl)phenoxy)ethyl)piperazin-1-yl)-N-(3-(2,6-dioxopiperidin-3-ylamino)phenyl)propanamide,
2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)phenoxy)ethyl)piperazin-1-yl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)phenoxy)ethyl)piperazin-1-yl)acetamide,
(2R)-2-(4-(2-(2-chloro-4-(3-(4-cyano-3-(trifluoromethyl)phenyl)-5,5-dimethyl-4-oxo-2-thioxoimidazolidin-1-yl)phenoxy)ethyl)piperazin-1-yl)-N-(3-(2,6-dioxopiperidin-3-ylamino)phenyl)propanamide,
2-(4-(2-(4-(3-(6-cyano-5-(trifluoromethyl)pyridin-3-yl)-5,5-dimethyl-4-oxo-2-thioxoimidazolidin-1-yl)-2-fluoro-5-methylphenoxy)ethyl)piperazin-1-yl)-N-(3-(2,6-dioxopiperidin-3-ylamino)phenyl)acetamide,
(S)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide,
(R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide,
2-(4-(2-(2-sec-butyl-4-(3-(4-cyano-3-(trifluoromethyl)phenyl)-5,5-dimethyl-4-oxo-2-thioxoimidazolidin-1-yl)phenoxy)ethyl)piperazin-1-yl)-N-(3-(2,6-dioxopiperidin-3-ylamino)phenyl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)piperazin-1-yl)acetamide,
2-(4-(2-(4-(3-(4-cyano-3-(trifluoromethyl)phenyl)-5,5-dimethyl-4-oxo-2-thioxoimidazolidin-1-yl)-2-cyclobutylphenoxy)ethyl)piperazin-1-yl)-N-(3-(2,6-dioxopiperidin-3-ylamino)phenyl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)acetamide,
2-(4-(2-(4-(3-(6-cyano-5-(trifluoromethyl)pyridin-3-yl)-5,5-dimethyl-4-oxo-2-thioxoimidazolidin-1-yl)-2-cyclopropylphenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-propylphenoxy)ethyl)piperazin-1-yl)-N-(3-(2,6-dioxopiperidin-3-ylamino)phenyl)acetamide,
N-(2-chloro-3-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)acetamide,
2-(4-(2-(4-(3-(6-cyano-5-(trifluoromethyl)pyridin-3-yl)-5,5-dimethyl-4-oxo-2-thioxoimidazolidin-1-yl)-2-cyclobutylphenoxy)ethyl)piperazin-1-yl)-N-(3-(2,6-dioxopiperidin-3-ylamino)phenyl)acetamide,
2-(4-(2-(2-sec-butyl-4-(3-(6-cyano-5-(trifluoromethyl)pyridin-3-yl)-5,5-dimethyl-4-oxo-2-thioxoimidazolidin-1-yl)phenoxy)ethyl)piperazin-1-yl)-N-(3-(2,6-dioxopiperidin-3-ylamino)phenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-propylphenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-cyclopentylphenoxy)ethyl)piperazin-1-yl)-N-(3-(2,6-dioxopiperidin-3-ylamino)phenyl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)acetamide,
N-(2-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)acetamide,
2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(5-(2,6-dioxopiperidin-3-ylamino)-2-fluorophenyl)acetamide,
2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-methylpiperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-methylpiperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide,
2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide,
N-(2-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-(hydroxymethyl)piperazin-1-yl)-N-(3-(2,6-dioxopiperidin-3-ylamino)phenyl)acetamide,
2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-(hydroxymethyl)piperazin-1-yl)-N-(3-(2,6-dioxopiperidin-3-ylamino)phenyl)acetamide,
2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-(hydroxymethyl)piperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-(hydroxymethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-2,4-dioxoimidazolidin-1-yl)-2-ethylphenoxy)ethyl)piperazin-1-yl)-N-(3-(2,6-dioxopiperidin-3-ylamino)phenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-isopropylphenoxy)ethyl)piperazin-1-yl)-N-(3-(2,6-dioxopiperidin-3-ylamino)phenyl)acetamide,
N-(3-chloro-5-(2,6-dioxopiperidin-3-ylamino)phenyl)-2-(4-(2-(4-(3-(4-cyano-3-(trifluoromethyl)phenyl)-4-oxo-2-thioxo-7-oxa-1,3-diazaspiro[4.4]nonan-1-yl)-2-fluorophenoxy)ethyl)piperazin-1-yl)acetamide,
N-(3-chloro-5-(2,6-dioxopiperidin-3-ylamino)phenyl)-2-(4-(2-(4-(3-(4-cyano-3-(trifluoromethyl)phenyl)-4-oxo-2-thioxo-7-oxa-1,3-diazaspiro[4.4]nonan-1-yl)-2-fluorophenoxy)ethyl)piperazin-1-yl)acetamide,
2-(4-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)piperazin-1-yl)-N-(3-(2,6-dioxopiperidin-3-ylamino)phenyl)acetamide,
(2S)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)propanamide,
(2R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)propanamide,
2-(4-(2-(4-(3-(4-cyano-3-(trifluoromethyl)phenyl)-5,5-dimethyl-4-oxo-2-thioxoimidazolidin-1-yl)-2-(1,1-difluoroethyl)phenoxy)ethyl)piperazin-1-yl)-N-(3-(2,6-dioxopiperidin-3-ylamino)phenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)-5-fluorophenyl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)piperazin-1-yl)acetamide,
2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)-5-fluorophenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)-N-(5-(2,6-dioxopiperidin-3-ylamino)-2-fluorophenyl)acetamide,
2-(4-(2-(4-(3-(4-cyano-3-(trifluoromethyl)phenyl)-5,5-dimethyl-4-oxo-2-thioxoimidazolidin-1-yl)-2-fluorophenoxy)ethyl)piperazin-1-yl)-N-(3-cyano-5-(2,6-dioxopiperidin-3-ylamino)phenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-(2,2-difluoroethyl)phenoxy)ethyl)piperazin-1-yl)-N-(5-(2,6-dioxopiperidin-3-ylamino)-2-fluorophenyl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-(2,2-difluoroethyl)phenoxy)ethyl)piperazin-1-yl)acetamide,
2-(4-(2-(4-(3-(6-cyano-5-(trifluoromethyl)pyridin-3-yl)-5,5-dimethyl-4-oxo-2-thioxoimidazolidin-1-yl)-2-(2,2-difluoroethyl)phenoxy)ethyl)piperazin-1-yl)-N-(3-(2,6-dioxopiperidin-3-ylamino)-5-fluorophenyl)acetamide,
N-(3-chloro-5-(2,6-dioxopiperidin-3-ylamino)phenyl)-2-(4-(2-(5-(3-(4-cyano-3-(trifluoromethyl)phenyl)-5,5-dimethyl-4-oxo-2-thioxoimidazolidin-1-yl)-3-ethylpyridin-2-yloxy)ethyl)piperazin-1-yl)acetamide,
2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(5-(2,6-dioxopiperidin-3-ylamino)-2,3-difluorophenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-(2-fluoroethyl)phenoxy)ethyl)piperazin-1-yl)-N-(5-(2,6-dioxopiperidin-3-ylamino)-2-fluorophenyl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-(2-fluoroethyl)phenoxy)ethyl)piperazin-1-yl)acetamide,
2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,2-dimethylpiperazin-1-yl)-N-(3-(2,6-dioxopiperidin-3-ylamino)-5-fluorophenyl)acetamide,
N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide,
2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)-5-fluorophenyl)acetamide,
2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)-N-(5-(2,6-dioxopiperidin-3-ylamino)-2-fluorophenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,2-dimethylpiperazin-1-yl)-N-(3-(2,6-dioxopiperidin-3-ylamino)-5-fluorophenyl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,2-dimethylpiperazin-1-yl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,2-dimethylpiperazin-1-yl)acetamide,
2-(4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)piperazin-1-yl)-N-(3-(2,6-dioxopiperidin-3-ylamino)-5-fluorophenyl)acetamide,
2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)piperazin-1-yl)-N-(5-(2,6-dioxopiperidin-3-ylamino)-2,3-difluorophenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,2-dimethylpiperazin-1-yl)-N-(5-(2,6-dioxopiperidin-3-ylamino)-2,3-difluorophenyl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-cyclopropylphenoxy)ethyl)piperazin-1-yl)acetamide,
N-(3-chloro-5-(2,6-dioxopiperidin-3-ylamino)phenyl)-2-(4-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)piperazin-1-yl)acetamide,
2-(4-(2-(4-(3-(6-cyano-5-(trifluoromethyl)pyridin-3-yl)-5,5-dimethyl-4-oxo-2-thioxoimidazolidin-1-yl)phenoxy)ethyl)piperazin-1-yl)-N-(5-(2,6-dioxopiperidin-3-ylamino)-2-fluorophenyl)acetamide,
N-(3-chloro-5-(2,6-dioxopiperidin-3-ylamino)phenyl)-2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)acetamide,
2-(4-(3-(4-(3-(4-cyano-3-(trifluoromethyl)phenyl)-5,5-dimethyl-4-oxo-2-thioxoimidazolidin-1-yl)-2-ethylphenyl)propyl)piperazin-1-yl)-N-(3-(2,6-dioxopiperidin-3-ylamino)-5-fluorophenyl)acetamide,
N-(3-chloro-5-(2,6-dioxopiperidin-3-ylamino)phenyl)-2-(4-(3-(4-(3-(4-cyano-3-(trifluoromethyl)phenyl)-5,5-dimethyl-4-oxo-2-thioxoimidazolidin-1-yl)-2-ethylphenyl)propyl)piperazin-1-yl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)-5-fluorophenyl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-(hydroxymethyl)phenoxy)ethyl)piperazin-1-yl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-(1-hydroxyethyl)phenoxy)ethyl)piperazin-1-yl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-isopropylphenoxy)ethyl)piperazin-1-yl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-isopropylphenoxy)ethyl)piperazin-1-yl)acetamide,
2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,2-dimethylpiperazin-1-yl)-N-(3-(2,6-dioxopiperidin-3-ylamino)phenyl)acetamide,
2-(4-(2-(4-(3-(4-cyano-2-methyl-3-(trifluoromethyl)phenyl)-5,5-dimethyl-4-oxo-2-thioxoimidazolidin-1-yl)-2-fluorophenoxy)ethyl)piperazin-1-yl)-N-(3-(2,6-dioxopiperidin-3-ylamino)phenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,2-dimethylpiperazin-1-yl)-N-(3-(2,6-dioxopiperidin-3-ylamino)phenyl)acetamide,
2-(7-(2-(4-(3-(4-cyano-3-(trifluoromethyl)phenyl)-5,5-dimethyl-4-oxo-2-thioxoimidazolidin-1-yl)-2-ethylphenoxy)ethyl)-4,7-diazaspiro[2.5]octan-4-yl)-N-(3-(2,6-dioxopiperidin-3-ylamino)phenyl)acetamide,
2-(7-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-4,7-diazaspiro[2.5]octan-4-yl)-N-(3-(2,6-dioxopiperidin-3-ylamino)phenyl)acetamide,
2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)-N-(5-(2,6-dioxopiperidin-3-ylamino)-2,3-difluorophenyl)acetamide,
N-(3-chloro-5-(2,6-dioxopiperidin-3-ylamino)phenyl)-2-(7-(2-(4-(3-(4-cyano-3-(trifluoromethyl)phenyl)-5,5-dimethyl-4-oxo-2-thioxoimidazolidin-1-yl)-2-ethylphenoxy)ethyl)-4,7-diazaspiro[2.5]octan-4-yl)acetamide,
N-(3-chloro-5-(2,6-dioxopiperidin-3-ylamino)phenyl)-2-(7-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-4,7-diazaspiro[2.5]octan-4-yl)acetamide,
N-(3-chloro-5-(2,6-dioxopiperidin-3-ylamino)phenyl)-2-((R)-4-(2-(4-(7-(4-cyano-3-(trifluoromethyl)phenyl)-8-oxo-6-thioxo-5,7-diazaspiro[3.4]octan-5-yl)-2-ethylphenoxy)ethyl)-2-methylpiperazin-1-yl)acetamide,
2-((R)-4-(2-(4-(7-(4-cyano-3-(trifluoromethyl)phenyl)-8-oxo-6-thioxo-5,7-diazaspiro[3.4]octan-5-yl)-2-ethylphenoxy)ethyl)-2-methylpiperazin-1-yl)-N-(3-(2,6-dioxopiperidin-3-ylamino)-5-fluorophenyl)acetamide,
2-((R)-4-(2-(4-(7-(4-cyano-3-(trifluoromethyl)phenyl)-8-oxo-6-thioxo-5,7-diazaspiro[3.4]octan-5-yl)-2-ethylphenoxy)ethyl)-2-methylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((R)-4-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)-2-methylpiperazin-1-yl)acetamide,
2-((R)-4-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)-2-methylpiperazin-1-yl)-N-(3-(2,6-dioxopiperidin-3-ylamino)-5-fluorophenyl)acetamide,
2-((R)-4-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)-2-methylpiperazin-1-yl)-N-(3-(2,6-dioxopiperidin-3-ylamino)phenyl)acetamide,
2-(7-(2-(4-(3-(4-cyano-3-(trifluoromethyl)phenyl)-5,5-dimethyl-4-oxo-2-thioxoimidazolidin-1-yl)-2-ethylphenoxy)ethyl)-4,7-diazaspiro[2.5]octan-4-yl)-N-(3-(2,6-dioxopiperidin-3-ylamino)-5-fluorophenyl)acetamide,
2-(7-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-4,7-diazaspiro[2.5]octan-4-yl)-N-(3-(2,6-dioxopiperidin-3-ylamino)-5-fluorophenyl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-((S)-2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-((R)-2,6-dioxopiperidin-3-ylamino)phenyl)acetamide,
(2R)—N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)propanamide,
(2S)—N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)propanamide,
N-(3-chloro-5-(2,6-dioxopiperidin-3-ylamino)phenyl)-2-((1S,4S)-5-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,5-diazabicyclo[2.2.1]heptan-2-yl)acetamide,
(2S)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)-5-fluorophenyl)propanamide,
(2R)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)-5-fluorophenyl)propanamide,
(2S)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)propanamide,
(2R)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)propanamide,
N-(3-chloro-5-(2,6-dioxopiperidin-3-ylamino)phenyl)-2-((1R,4R)-5-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,5-diazabicyclo[2.2.1]heptan-2-yl)acetamide,
2-((2R,5S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)-N-(3-(2,6-dioxopiperidin-3-ylamino)phenyl)acetamide,
2-((2S,5R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((2R,5S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)acetamide,
N-(3-chloro-5-(2,6-dioxopiperidin-3-ylamino)phenyl)-2-((2S,5R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)acetamide,
2-((2S,5S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((2S,5S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)acetamide,
2-((2R,5R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((2R,5R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)acetamide,
2-((2R,6R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((2R,6R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)acetamide,
N-(3-chloro-5-(2,6-dioxopiperidin-3-ylamino)phenyl)-2-((S)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide,
2-(4-(2-(4-(7-(4-cyano-3-(trifluoromethyl)phenyl)-8-oxo-6-thioxo-5,7-diazaspiro[3.4]octan-5-yl)-2-ethylphenoxy)ethyl)piperazin-1-yl)-N-(3-(2,6-dioxopiperidin-3-ylamino)phenyl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-2-methylpropanamide,
2-(4-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)-2,2-dimethylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((R)-4-(2-(4-(3-(4-cyano-3-(trifluoromethyl)phenyl)-5,5-dimethyl-4-oxo-2-thioxoimidazolidin-1-yl)-2-(2-fluoroethyl)phenoxy)ethyl)-2-methylpiperazin-1-yl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-(2-fluoroethyl)phenoxy)ethyl)piperazin-1-yl)acetamide,
2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)-N-(3-((S)-2,6-dioxopiperidin-3-ylamino)phenyl)acetamide,
2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)-N-(3-((R)-2,6-dioxopiperidin-3-ylamino)phenyl)acetamide,
1-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)cyclopropanecarboxamide,
N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-(2-fluoroethyl)phenoxy)ethyl)-2-methylpiperazin-1-yl)acetamide,
2-(4-(2-(4-(3-(4-cyano-3-(trifluoromethyl)phenyl)-5,5-dimethyl-4-oxo-2-thioxoimidazolidin-1-yl)-2-(2-fluoroethyl)phenoxy)ethyl)piperazin-1-yl)-N-(5-(2,6-dioxopiperidin-3-ylamino)-2-fluorophenyl)acetamide,
N-(3-chloro-5-(2,6-dioxopiperidin-3-ylamino)phenyl)-2-((R)-4-(2-(4-(7-(6-cyano-5-(trifluoromethyl)pyridin-3-yl)-8-oxo-6-thioxo-5,7-diazaspiro[3.4]octan-5-yl)-2-(2-fluoroethyl)phenoxy)ethyl)-2-methylpiperazin-1-yl)acetamide,
N-(3-chloro-5-(2,6-dioxopiperidin-3-ylamino)phenyl)-2-((R)-4-(2-(4-(7-(4-cyano-3-(trifluoromethyl)phenyl)-8-oxo-6-thioxo-5,7-diazaspiro[3.4]octan-5-yl)-2-(2-fluoroethyl)phenoxy)ethyl)-2-methylpiperazin-1-yl)acetamide,
N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide,
N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide,
2-((2S,6S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((2S,6S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)acetamide,
N-(3-chloro-5-(2,6-dioxopiperidin-3-ylamino)phenyl)-2-((R)-4-(2-(4-(3-(4-cyano-3-(trifluoromethyl)phenyl)-5,5-dimethyl-4-oxo-2-thioxoimidazolidin-1-yl)-2-(1,1-difluoroethyl)phenoxy)ethyl)-2-methylpiperazin-1-yl)acetamide,
N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)-2-methylpiperazin-1-yl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)-N-(3-(2,6-dioxopiperidin-3-ylamino)phenyl)acetamide,
2-((2R,6S)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,6-dimethylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((R)-4-(2-(4-(3-(4-cyano-3-(trifluoromethyl)phenyl)-5,5-dimethyl-4-oxo-2-thioxoimidazolidin-1-yl)-2-(2,2-difluoroethyl)phenoxy)ethyl)-2-methylpiperazin-1-yl)acetamide,
2-((R)-4-(2-(4-(3-(4-cyano-3-(trifluoromethyl)phenyl)-5,5-dimethyl-4-oxo-2-thioxoimidazolidin-1-yl)-2-(2,2-difluoroethyl)phenoxy)ethyl)-2-methylpiperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-cyano-5-(2,6-dioxopiperidin-3-ylamino)phenyl)acetamide,
N-(3-cyano-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide,
2-((R)-4-(2-(4-(3-(4-cyano-3-(trifluoromethyl)phenyl)-5,5-dimethyl-4-oxo-2-thioxoimidazolidin-1-yl)-2-(2,2-difluoroethyl)phenoxy)ethyl)-2-methylpiperazin-1-yl)-N-(5-(2,6-dioxopiperidin-3-ylamino)-2-fluorophenyl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-(1,1-difluoroethyl)phenoxy)ethyl)piperazin-1-yl)acetamide,
2-((R)-4-(2-(4-(3-(4-cyano-3-(trifluoromethyl)phenyl)-5,5-dimethyl-4-oxo-2-thioxoimidazolidin-1-yl)-2-(2,2-difluoroethyl)phenoxy)ethyl)-2-methylpiperazin-1-yl)-N-(3-(2,6-dioxopiperidin-3-ylamino)-5-fluorophenyl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-(2,2-difluoroethyl)phenoxy)ethyl)piperazin-1-yl)acetamide,
2-(4-(2-(4-(3-(4-cyano-3-(trifluoromethyl)phenyl)-5,5-dimethyl-4-oxo-2-thioxoimidazolidin-1-yl)-2-(2-fluoroethyl)phenoxy)ethyl)piperazin-1-yl)-N-(3-(2,6-dioxopiperidin-3-ylamino)phenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-(2-fluoroethyl)phenoxy)ethyl)piperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2-methylpiperazin-1-yl)-N-(3-(2,6-dioxopiperidin-3-ylamino)phenyl)acetamide,
2-((R)-4-(2-(4-(3-(6-cyano-5-(trifluoromethyl)pyridin-3-yl)-5,5-dimethyl-4-oxo-2-thioxoimidazolidin-1-yl)-2-isopropylphenoxy)ethyl)-2-methylpiperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-(2-fluoroethyl)phenoxy)ethyl)-2-methylpiperazin-1-yl)-N-(3-(2,6-dioxopiperidin-3-ylamino)phenyl)acetamide,
N-(3-chloro-5-(2,6-dioxopiperidin-3-ylamino)phenyl)-1-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)cyclopropanecarboxamide,
2-((R)-4-(2-(4-(3-(6-cyano-5-(trifluoromethyl)pyridin-3-yl)-5,5-dimethyl-4-oxo-2-thioxoimidazolidin-1-yl)-2-(2-fluoroethyl)phenoxy)ethyl)-2-methylpiperazin-1-yl)-N-(3-(2,6-dioxopiperidin-3-ylamino)phenyl)acetamide,
2-((R)-4-(2-(4-(3-(5-chloro-6-cyanopyridin-3-yl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide,
2-((R)-4-(2-(4-(3-(3-chloro-4-cyanophenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((2R,5R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,5-dimethylpiperazin-1-yl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2,6-dimethylpiperazin-1-yl)-N-(3-(2,6-dioxopiperidin-3-ylamino)phenyl)acetamide,
2-(3-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)-3,8-diazabicyclo[3.2.1]octan-8-yl)-N-(3-(2,6-dioxopiperidin-3-ylamino)phenyl)acetamide,
2-((1S,4S)-5-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)-2,5-diazabicyclo[2.2.2]octan-2-yl)-N-(3-(2,6-dioxopiperidin-3-ylamino)phenyl)acetamide,
N-(3-chloro-5-(2,6-dioxopiperidin-3-ylamino)phenyl)-2-(3-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-3,8-diazabicyclo[3.2.1]octan-8-yl)acetamide,
2-(4-(2-(4-(3-(4-cyano-3-(trifluoromethyl)phenyl)-5,5-dimethyl-4-oxo-2-thioxoimidazolidin-1-yl)-2-(2,2-difluoroethyl)phenoxy)ethyl)piperazin-1-yl)-N-(5-(2,6-dioxopiperidin-3-ylamino)-2-fluorophenyl)acetamide,
N-(3-chloro-5-(2,6-dioxopiperidin-3-ylamino)phenyl)-2-((2R,5R)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2,5-dimethylpiperazin-1-yl)acetamide,
2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-isopropylphenoxy)ethyl)-2,6-dimethylpiperazin-1-yl)-N-(3-(2,6-dioxopiperidin-3-ylamino)phenyl)acetamide,
2-(3-(2-(4-(3-(4-cyano-3-(trifluoromethyl)phenyl)-5,5-dimethyl-4-oxo-2-thioxoimidazolidin-1-yl)-2-ethylphenoxy)ethyl)-3,8-diazabicyclo[3.2.1]octan-8-yl)-N-(3-(2,6-dioxopiperidin-3-ylamino)phenyl)acetamide,
2-(3-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-3,8-diazabicyclo[3.2.1]octan-8-yl)-N-(3-(2,6-dioxopiperidin-3-ylamino)phenyl)acetamide,
N-(3-chloro-5-(2,6-dioxopiperidin-3-ylamino)phenyl)-2-((1S,4S)-5-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,5-diazabicyclo[2.2.2]octan-2-yl)acetamide,
2-((2S,6R)-4-(2-(4-(3-(6-cyano-5-(trifluoromethyl)pyridin-3-yl)-5,5-dimethyl-4-oxo-2-thioxoimidazolidin-1-yl)-2-isopropylphenoxy)ethyl)-2,6-dimethylpiperazin-1-yl)-N-(3-(2,6-dioxopiperidin-3-ylamino)phenyl)acetamide,
2-((2R,5R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)-N-(3-cyano-5-(2,6-dioxopiperidin-3-ylamino)phenyl)acetamide,
2-((1R,4R)-5-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)-2,5-diazabicyclo[2.2.2]octan-2-yl)-N-(3-(2,6-dioxopiperidin-3-ylamino)phenyl)acetamide,
2-((1R,4R)-5-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,5-diazabicyclo[2.2.2]octan-2-yl)-N-(3-(2,6-dioxopiperidin-3-ylamino)phenyl)acetamide,
N-(3-chloro-5-(2,6-dioxopiperidin-3-ylamino)phenyl)-2-((1R,4R)-5-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,5-diazabicyclo[2.2.2]octan-2-yl)acetamide,
N-(3-chloro-5-(2,6-dioxopiperidin-3-ylamino)phenyl)-2-((2R,5R)-4-(2-(4-(3-(6-cyano-5-(trifluoromethyl)pyridin-3-yl)-5,5-dimethyl-4-oxo-2-thioxoimidazolidin-1-yl)-2-isopropylphenoxy)ethyl)-2,5-dimethylpiperazin-1-yl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)-N-(3-cyano-5-(2,6-dioxopiperidin-3-ylamino)phenyl)acetamide,
2-((R)-4-(2-(5-(3-(4-cyano-3-(trifluoromethyl)phenyl)-5,5-dimethyl-4-oxo-2-thioxoimidazolidin-1-yl)-3-ethylpyridin-2-yloxy)ethyl)-2-methylpiperazin-1-yl)-N-(3-(2,6-dioxopiperidin-3-ylamino)phenyl)acetamide,
1-(4-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)piperazin-1-yl)-N-(3-(2,6-dioxopiperidin-3-ylamino)phenyl)cyclopropanecarboxamide,
N-(3-chloro-5-(2,6-dioxopiperidin-3-ylamino)phenyl)-2-((2R,5R)-4-(2-(5-(3-(4-cyano-3-(trifluoromethyl)phenyl)-5,5-dimethyl-4-oxo-2-thioxoimidazolidin-1-yl)-3-ethylpyridin-2-yloxy)ethyl)-2,5-dimethylpiperazin-1-yl)acetamide,
2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)-2,6-dimethylpiperazin-1-yl)-N-(5-(2,6-dioxopiperidin-3-ylamino)-2-fluorophenyl)acetamide,
(2R)—N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)propanamide,
2-((2R,5R)-4-(2-(4-(3-(6-cyano-5-(trifluoromethyl)pyridin-3-yl)-5,5-dimethyl-4-oxo-2-thioxoimidazolidin-1-yl)-2-isopropylphenoxy)ethyl)-2,5-dimethylpiperazin-1-yl)-N-(3-(2,6-dioxopiperidin-3-ylamino)phenyl)acetamide,
2-((2R,5R)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2,5-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2,6-dimethylpiperazin-1-yl)-N-(3-cyano-5-(2,6-dioxopiperidin-3-ylamino)phenyl)acetamide,
N-(3-chloro-5-(2,6-dioxopiperidin-3-ylamino)phenyl)-2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-isopropylphenoxy)ethyl)-2,6-dimethylpiperazin-1-yl)acetamide,
2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-cyclopropylphenoxy)ethyl)-2,6-dimethylpiperazin-1-yl)-N-(3-(2,6-dioxopiperidin-3-ylamino)phenyl)acetamide,
1-(4-(2-(4-(3-(4-cyano-3-(trifluoromethyl)phenyl)-5,5-dimethyl-4-oxo-2-thioxoimidazolidin-1-yl)-2-isopropylphenoxy)ethyl)piperazin-1-yl)-N-(3-(2,6-dioxopiperidin-3-ylamino)phenyl)cyclopropanecarboxamide,
2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-cyclopropylphenoxy)ethyl)-2,6-dimethylpiperazin-1-yl)-N-(5-(2,6-dioxopiperidin-3-ylamino)-2-fluorophenyl)acetamide,
(2R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)propanamide,
2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-(trifluoromethyl)piperazin-1-yl)-N-(3-(2,6-dioxopiperidin-3-ylamino)phenyl)acetamide,
2-((S)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-(trifluoromethyl)piperazin-1-yl)-N-(3-(2,6-dioxopiperidin-3-ylamino)phenyl)acetamide,
2-((2S,6R)-4-(2-(4-(3-(3-chloro-4-cyanophenyl)-5,5-dimethyl-4-oxo-2-thioxoimidazolidin-1-yl)-2-isopropylphenoxy)ethyl)-2,6-dimethylpiperazin-1-yl)-N-(3-(2,6-dioxopiperidin-3-ylamino)phenyl)acetamide,
2-((2R,5R)-4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-2,5-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-2,6-dimethylpiperazin-1-yl)-N-(3-(2,6-dioxopiperidin-3-ylamino)phenyl)acetamide,
2-((2R,5R)-4-(2-(4-(3-(3-chloro-4-cyanophenyl)-5,5-dimethyl-4-oxo-2-thioxoimidazolidin-1-yl)-2-cyclopropylphenoxy)ethyl)-2,5-dimethylpiperazin-1-yl)-N-(3-(2,6-dioxopiperidin-3-ylamino)phenyl)acetamide,
2-((2S,6R)-4-(2-(4-(3-(3-chloro-4-cyanophenyl)-5,5-dimethyl-4-oxo-2-thioxoimidazolidin-1-yl)-2-cyclopropylphenoxy)ethyl)-2,6-dimethylpiperazin-1-yl)-N-(3-(2,6-dioxopiperidin-3-ylamino)phenyl)acetamide,
2-((2R,5R)-4-(2-(4-(3-(3-chloro-4-cyanophenyl)-5,5-dimethyl-4-oxo-2-thioxoimidazolidin-1-yl)-2-isopropylphenoxy)ethyl)-2,5-dimethylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((2S,6R)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2,6-dimethylpiperazin-1-yl)acetamide,
2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-isopropylphenoxy)ethyl)-2,6-dimethylpiperazin-1-yl)-N-(5-(2,6-dioxopiperidin-3-ylamino)-2-fluorophenyl)acetamide,
N-(3-cyano-5-(2,6-dioxopiperidin-3-ylamino)phenyl)-2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-isopropylphenoxy)ethyl)-2,6-dimethylpiperazin-1-yl)acetamide,
2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-isopropylphenoxy)ethyl)-2,6-dimethylpiperazin-1-yl)-N-(3-(2,6-dioxopiperidin-3-ylamino)-5-fluorophenyl)acetamide,
N-(3-cyano-5-(2,6-dioxopiperidin-3-ylamino)phenyl)-2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-cyclopropylphenoxy)ethyl)-2,6-dimethylpiperazin-1-yl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2,6-dimethylpiperazin-1-yl)-N-(3-(2,6-dioxopiperidin-3-ylamino)-5-fluorophenyl)acetamide,
N-(3-chloro-5-(2,6-dioxopiperidin-3-ylamino)phenyl)-2-((2R,5R)-4-(2-(5-(3-(4-cyano-3-(trifluoromethyl)phenyl)-5,5-dimethyl-4-oxo-2-thioxoimidazolidin-1-yl)-3-isopropylpyridin-2-yloxy)ethyl)-2,5-dimethylpiperazin-1-yl)acetamide,
N-(3-chloro-5-(2,6-dioxopiperidin-3-ylamino)phenyl)-2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-cyclopropylphenoxy)ethyl)-2,6-dimethylpiperazin-1-yl)acetamide,
2-((2R,6S)-4-(2-(5-(3-(4-cyano-3-(trifluoromethyl)phenyl)-5,5-dimethyl-4-oxo-2-thioxoimidazolidin-1-yl)-3-ethylpyridin-2-yloxy)ethyl)-2,6-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2,6-dimethylpiperazin-1-yl)-N-(5-(2,6-dioxopiperidin-3-ylamino)-2-fluorophenyl)acetamide,
2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-cyclopropylphenoxy)ethyl)-2,6-dimethylpiperazin-1-yl)-N-(3-(2,6-dioxopiperidin-3-ylamino)-5-fluorophenyl)acetamide,
2-((2S,6R)-4-(2-(4-(3-(3-chloro-4-cyanophenyl)-5,5-dimethyl-4-oxo-2-thioxoimidazolidin-1-yl)-2-cyclopropylphenoxy)ethyl)-2,6-dimethylpiperazin-1-yl)-N-(3-cyano-5-(2,6-dioxopiperidin-3-ylamino)phenyl)acetamide,
2-((2S,6R)-4-(2-(4-(3-(3-chloro-4-cyanophenyl)-5,5-dimethyl-4-oxo-2-thioxoimidazolidin-1-yl)-2-cyclopropylphenoxy)ethyl)-2,6-dimethylpiperazin-1-yl)-N-(3-chloro-5-(2,6-dioxopiperidin-3-ylamino)phenyl)acetamide,
2-((2S,6R)-4-(2-(4-(3-(3-chloro-4-cyanophenyl)-5,5-dimethyl-4-oxo-2-thioxoimidazolidin-1-yl)-2-cyclopropylphenoxy)ethyl)-2,6-dimethylpiperazin-1-yl)-N-(5-(2,6-dioxopiperidin-3-ylamino)-2-fluorophenyl)acetamide,
2-((2S,6R)-4-(2-(4-(3-(3-chloro-4-cyanophenyl)-5,5-dimethyl-4-oxo-2-thioxoimidazolidin-1-yl)-2-isopropylphenoxy)ethyl)-2,6-dimethylpiperazin-1-yl)-N-(5-(2,6-dioxopiperidin-3-ylamino)-2-fluorophenyl)acetamide,
2-((2S,6R)-4-(2-(4-(3-(3-chloro-4-cyanophenyl)-5,5-dimethyl-4-oxo-2-thioxoimidazolidin-1-yl)-2-isopropylphenoxy)ethyl)-2,6-dimethylpiperazin-1-yl)-N-(3-chloro-5-(2,6-dioxopiperidin-3-ylamino)phenyl)acetamide,
2-((2R,6S)-4-(2-(5-(3-(4-cyano-3-(trifluoromethyl)phenyl)-5,5-dimethyl-4-oxo-2-thioxoimidazolidin-1-yl)-3-isopropylpyridin-2-yloxy)ethyl)-2,6-dimethylpiperazin-1-yl)-N-(3-(2,6-dioxopiperidin-3-ylamino)phenyl)acetamide,
2-((2R,6S)-4-(2-(4-(7-(4-cyano-3-(trifluoromethyl)phenyl)-8-oxo-6-thioxo-5,7-diazaspiro[3.4]octan-5-yl)-2-cyclopropylphenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(3-chloro-4-cyanophenyl)-5,5-dimethyl-4-oxo-2-thioxoimidazolidin-1-yl)-2-cyclopropylphenoxy)ethyl)-2,6-dimethylpiperazin-1-yl)-N-(3-(2,6-dioxopiperidin-3-ylamino)-5-fluorophenyl)acetamide,
2-((2S,6R)-4-(2-(5-(7-(4-cyano-3-(trifluoromethyl)phenyl)-8-oxo-6-thioxo-5,7-diazaspiro[3.4]octan-5-yl)-3-isopropylpyridin-2-yloxy)ethyl)-2,6-dimethylpiperazin-1-yl)-N-(3-(2,6-dioxopiperidin-3-ylamino)phenyl)acetamide,
2-((2S,6R)-4-(2-(5-(3-(4-cyano-3-(trifluoromethyl)phenyl)-5,5-dimethyl-4-oxo-2-thioxoimidazolidin-1-yl)-3-cyclopropylpyridin-2-yloxy)ethyl)-2,6-dimethylpiperazin-1-yl)-N-(3-(2,6-dioxopiperidin-3-ylamino)phenyl)acetamide,
2-((2S,6R)-4-(2-(5-(7-(4-cyano-3-(trifluoromethyl)phenyl)-8-oxo-6-thioxo-5,7-diazaspiro[3.4]octan-5-yl)-3-cyclopropylpyridin-2-yloxy)ethyl)-2,6-dimethylpiperazin-1-yl)-N-(3-(2,6-dioxopiperidin-3-ylamino)phenyl)acetamide,
2-((2R,5R)-4-(2-(5-(3-(4-cyano-3-(trifluoromethyl)phenyl)-5,5-dimethyl-4-oxo-2-thioxoimidazolidin-1-yl)-3-isopropylpyridin-2-yloxy)ethyl)-2,5-dimethylpiperazin-1-yl)-N-(3-(2,6-dioxopiperidin-3-ylamino)phenyl)acetamide,
2-((2R,5R)-4-(2-(5-(7-(4-cyano-3-(trifluoromethyl)phenyl)-8-oxo-6-thioxo-5,7-diazaspiro[3.4]octan-5-yl)-3-isopropylpyridin-2-yloxy)ethyl)-2,5-dimethylpiperazin-1-yl)-N-(3-cyano-5-(2,6-dioxopiperidin-3-ylamino)phenyl)acetamide,
2-((2R,5R)-4-(2-(5-(3-(4-cyano-3-(trifluoromethyl)phenyl)-5,5-dimethyl-4-oxo-2-thioxoimidazolidin-1-yl)-3-isopropylpyridin-2-yloxy)ethyl)-2,5-dimethylpiperazin-1-yl)-N-(3-cyano-5-(2,6-dioxopiperidin-3-ylamino)phenyl)acetamide,
2-((S)-4-(2-(4-(3-(6-cyano-5-(trifluoromethyl)pyridin-3-yl)-5,5-dimethyl-4-oxo-2-thioxoimidazolidin-1-yl)-2-cyclopropylphenoxy)ethyl)-2-(trifluoromethyl)piperazin-1-yl)-N-(3-(2,6-dioxopiperidin-3-ylamino)phenyl)acetamide,
2-((2R,5R)-4-(2-(4-(3-(6-cyano-5-(trifluoromethyl)pyridin-3-yl)-5,5-dimethyl-4-oxo-2-thioxoimidazolidin-1-yl)-2-cyclopropylphenoxy)ethyl)-2,5-dimethylpiperazin-1-yl)-N-(3-(2,6-dioxopiperidin-3-ylamino)phenyl)acetamide,
2-((2S,6R)-4-(2-(4-(3-(6-cyano-5-(trifluoromethyl)pyridin-3-yl)-5,5-dimethyl-4-oxo-2-thioxoimidazolidin-1-yl)-2-cyclopropylphenoxy)ethyl)-2,6-dimethylpiperazin-1-yl)-N-(3-(2,6-dioxopiperidin-3-ylamino)phenyl)acetamide,
2-((2S,6R)-4-((S)-1-(4-(3-(4-cyano-3-(trifluoromethyl)phenyl)-5,5-dimethyl-4-oxo-2-thioxoimidazolidin-1-yl)-2-ethylphenoxy)propan-2-yl)-2,6-dimethylpiperazin-1-yl)-N-(3-(2,6-dioxopiperidin-3-ylamino)phenyl)acetamide,
2-((2S,6R)-4-((R)-1-(4-(3-(4-cyano-3-(trifluoromethyl)phenyl)-5,5-dimethyl-4-oxo-2-thioxoimidazolidin-1-yl)-2-ethylphenoxy)propan-2-yl)-2,6-dimethylpiperazin-1-yl)-N-(3-(2,6-dioxopiperidin-3-ylamino)phenyl)acetamide,
2-(3-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-3,8-diazabicyclo[3.2.1]octan-8-yl)-N-(3-(2,6-dioxopiperidin-3-ylamino)phenyl)acetamide,
2-((2S,6R)-4-((R)-2-(4-(3-(6-cyano-5-(trifluoromethyl)pyridin-3-yl)-5,5-dimethyl-4-oxo-2-thioxoimidazolidin-1-yl)-2-ethylphenoxy)propyl)-2,6-dimethylpiperazin-1-yl)-N-(3-(2,6-dioxopiperidin-3-ylamino)phenyl)acetamide,
2-((2S,6R)-4-((S)-2-(4-(3-(6-cyano-5-(trifluoromethyl)pyridin-3-yl)-5,5-dimethyl-4-oxo-2-thioxoimidazolidin-1-yl)-2-ethylphenoxy)propyl)-2,6-dimethylpiperazin-1-yl)-N-(3-(2,6-dioxopiperidin-3-ylamino)phenyl)acetamide,
2-((2S,6R)-4-((R)-2-(4-(3-(4-cyano-3-(trifluoromethyl)phenyl)-5,5-dimethyl-4-oxo-2-thioxoimidazolidin-1-yl)-2-ethylphenoxy)propyl)-2,6-dimethylpiperazin-1-yl)-N-(3-(2,6-dioxopiperidin-3-ylamino)phenyl)acetamide,
2-((2S,6R)-4-((S)-2-(4-(3-(4-cyano-3-(trifluoromethyl)phenyl)-5,5-dimethyl-4-oxo-2-thioxoimidazolidin-1-yl)-2-ethylphenoxy)propyl)-2,6-dimethylpiperazin-1-yl)-N-(3-(2,6-dioxopiperidin-3-ylamino)phenyl)acetamide,
N-(3-cyano-5-(2,6-dioxopiperidin-3-ylamino)phenyl)-2-((2R,5R)-4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)-2,5-dimethylpiperazin-1-yl)acetamide,
2-((2R,5R)-4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)-2,5-dimethylpiperazin-1-yl)-N-(3-(2,6-dioxopiperidin-3-ylamino)phenyl)acetamide,
2-((S)-4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-2-(trifluoromethyl)piperazin-1-yl)-N-(3-(2,6-dioxopiperidin-3-ylamino)phenyl)acetamide,
2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-(trifluoromethyl)piperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-ethyl-5-fluorophenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-ethyl-6-fluorophenoxy)ethyl)-2,6-dimethylpiperazin-1-yl)-N-(3-(2,6-dioxopiperidin-3-ylamino)phenyl)acetamide,
2-((2R,6S)-4-(2-(4-(3-(4-cyano-3-(trifluoromethyl)phenyl)-5,5-dimethyl-4-oxo-2-thioxoimidazolidin-1-yl)-2-ethyl-6-methylphenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(3-chloro-4-cyanophenyl)-5,5-dimethyl-4-oxo-2-thioxoimidazolidin-1-yl)-2-isopropylphenoxy)ethyl)-2,6-dimethylpiperazin-1-yl)-N-(3-cyano-5-(2,6-dioxopiperidin-3-ylamino)phenyl)acetamide,
2-((2S,6R)-4-(2-(4-(3-(3-chloro-4-cyanophenyl)-5,5-dimethyl-4-oxo-2-thioxoimidazolidin-1-yl)-2-isopropylphenoxy)ethyl)-2,6-dimethylpiperazin-1-yl)-N-(3-(2,6-dioxopiperidin-3-ylamino)-5-fluorophenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-(1,1-difluoroethyl)phenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-isopropyl-5-methylphenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-ethyl-5-methylphenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)-2,6-dimethylpiperazin-1-yl)-N-(3-(2,6-dioxopiperidin-3-ylamino)phenyl)acetamide,
2-((S)-4-(2-(4-(3-(6-cyano-5-(trifluoromethyl)pyridin-3-yl)-5,5-dimethyl-4-oxo-2-thioxoimidazolidin-1-yl)-2-isopropylphenoxy)ethyl)-2-(trifluoromethyl)piperazin-1-yl)-N-(3-(2,6-dioxopiperidin-3-ylamino)phenyl)acetamide,
2-((S)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2-(trifluoromethyl)piperazin-1-yl)-N-(3-(2,6-dioxopiperidin-3-ylamino)phenyl)acetamide,
N-(3-cyano-5-(2,6-dioxopiperidin-3-ylamino)phenyl)-2-((2S,6R)-4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)-2,6-dimethylpiperazin-1-yl)acetamide,
2-((2R,6S)-4-(2-(4-(3-(4-cyano-3-(trifluoromethyl)phenyl)-5,5-dimethyl-4-oxo-2-thioxoimidazolidin-1-yl)-2-cyclobutylphenoxy)ethyl)-2,6-dimethylpiperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-ethylpiperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-2-ethylpiperazin-1-yl)-N-(3-(2,6-dioxopiperidin-3-ylamino)phenyl)acetamide,
2-((R)-4-(2-(4-(7-(6-cyano-5-(trifluoromethyl)pyridin-3-yl)-8-oxo-6-thioxo-5,7-diazaspiro[3.4]octan-5-yl)-2-cyclopropylphenoxy)ethyl)-2-ethylpiperazin-1-yl)-N-(3-(2,6-dioxopiperidin-3-ylamino)phenyl)acetamide,
2-((S)-4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-2-ethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-4-oxo-2-thioxo-1,3-diazaspiro[4.5]decan-1-yl)-2-ethylphenoxy)ethyl)-2,6-dimethylpiperazin-1-yl)-N-(3-(2,6-dioxopiperidin-3-ylamino)phenyl)acetamide,
2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-(1,1-difluoroethyl)phenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-4-oxo-2-thioxo-1,3-diazaspiro[4.4]nonan-1-yl)-2-cyclopropylphenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-4-oxo-2-thioxo-1,3-diazaspiro[4.4]nonan-1-yl)-2-ethylphenoxy)ethyl)-2,6-dimethylpiperazin-1-yl)-N-(3-(2,6-dioxopiperidin-3-ylamino)phenyl)acetamide,
2-((2R,6S)-4-(2-(4-(3-(6-cyano-5-(trifluoromethyl)pyridin-3-yl)-5,5-dimethyl-4-oxo-2-thioxoimidazolidin-1-yl)-2-cyclobutylphenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(3-chloro-4-cyanophenyl)-5,5-dimethyl-4-oxo-2-thioxoimidazolidin-1-yl)-2-(1,1-difluoroethyl)phenoxy)ethyl)-2,6-dimethylpiperazin-1-yl)-N-(3-(2,6-dioxopiperidin-3-ylamino)phenyl)acetamide,
2-((2R,6S)-4-(2-(4-(3-(4-cyano-3-(trifluoromethyl)phenyl)-5,5-dimethyl-4-oxo-2-thioxoimidazolidin-1-yl)-2-ethyl-3-methylphenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-ethyl-3-fluorophenoxy)ethyl)-2,6-dimethylpiperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-2-methylpiperazin-1-yl)-N-(3-(2,6-dioxopiperidin-3-ylamino)phenyl)acetamide,
2-((2R,5R)-4-(3-(4-(3-(4-cyano-3-(trifluoromethyl)phenyl)-5,5-dimethyl-4-oxo-2-thioxoimidazolidin-1-yl)-2-ethylphenyl)propyl)-2,5-dimethylpiperazin-1-yl)-N-(3-(2,6-dioxopiperidin-3-ylamino)phenyl)acetamide,
(2R)-2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)propanamide,
(2S)-2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)propanamide,
2-((R)-4-(2-(2-chloro-4-(3-(4-cyano-3-(trifluoromethyl)phenyl)-5,5-dimethyl-4-oxo-2-thioxoimidazolidin-1-yl)phenoxy)ethyl)-2-methylpiperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-fluorophenoxy)ethyl)-2-methylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-fluorophenoxy)ethyl)-2,6-dimethylpiperazin-1-yl)-N-(3-(2,6-dioxopiperidin-3-ylamino)phenyl)acetamide,
2-((S)-4-(3-(4-(3-(4-cyano-3-(trifluoromethyl)phenyl)-5,5-dimethyl-4-oxo-2-thioxoimidazolidin-1-yl)-2-ethylphenyl)propyl)-2-methylpiperazin-1-yl)-N-(3-(2,6-dioxopiperidin-3-ylamino)phenyl)acetamide,
(2R)-2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)propanamide,
(2S)-2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)propanamide,
2-((2S,6R)-4-(2-(2-chloro-4-(3-(4-cyano-3-(trifluoromethyl)phenyl)-5,5-dimethyl-4-oxo-2-thioxoimidazolidin-1-yl)phenoxy)ethyl)-2,6-dimethylpiperazin-1-yl)-N-(3-(2,6-dioxopiperidin-3-ylamino)phenyl)acetamide,
2-((2R,6S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)-N-(3-(2,6-dioxopiperidin-3-ylamino)-5-fluorophenyl)acetamide,
2-((2R,6S)-4-(3-(4-(3-(4-cyano-3-(trifluoromethyl)phenyl)-5,5-dimethyl-4-oxo-2-thioxoimidazolidin-1-yl)-2-ethylphenyl)propyl)-2,6-dimethylpiperazin-1-yl)-N-(3-(2,6-dioxopiperidin-3-ylamino)phenyl)acetamide,
2-((2R,6S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)-N-(3-((S)-2,6-dioxopiperidin-3-ylamino)phenyl)acetamide,
2-((2R,6S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)-N-(3-((R)-2,6-dioxopiperidin-3-ylamino)phenyl)acetamide,
2-((2R,6S)-4-(3-(4-(3-(4-cyano-3-(trifluoromethyl)phenyl)-5,5-dimethyl-4-oxo-2-thioxoimidazolidin-1-yl)-2-fluorophenyl)propyl)-2,6-dimethylpiperazin-1-yl)-N-(3-(2,6-dioxopiperidin-3-ylamino)phenyl)acetamide,
2-((2R,6S)-4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-2,6-dimethylpiperazin-1-yl)-N-(3-((R)-2,6-dioxopiperidin-3-ylamino)phenyl)acetamide,
2-((2R,6S)-4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-2,6-dimethylpiperazin-1-yl)-N-(3-((S)-2,6-dioxopiperidin-3-ylamino)phenyl)acetamide,
2-((2S,6R)-4-(2-(2-chloro-4-(3-(3-chloro-4-cyanophenyl)-5,5-dimethyl-4-oxo-2-thioxoimidazolidin-1-yl)phenoxy)ethyl)-2,6-dimethylpiperazin-1-yl)-N-(3-((2,6-dioxopiperidin-3-yl)amino)phenyl)acetamide, and
2-((2S,6R)-4-(2-(4-(3-(3-chloro-4-cyanophenyl)-5,5-dimethyl-4-oxo-2-thioxoimidazolidin-1-yl)-2-ethylphenoxy)ethyl)-2,6-dimethylpiperazin-1-yl)-N-(3-((2,6-dioxopiperidin-3-yl)amino)phenyl)acetamide, or a pharmaceutically acceptable salt thereof.

22. The method of any one of embodiments 1 to 21, wherein the compound is (S)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide or a pharmaceutically acceptable salt thereof.

23. The method of any one of embodiments 1 to 21, wherein the compound is (S)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide.

24. The method of any one of embodiments 1 to 21, wherein the compound is 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-methylpiperazin-1-yl)-N-(3-(2,6-dioxopiperidin-3-ylamino)phenyl)acetamide or a pharmaceutically acceptable salt thereof.

25. The method of any one of embodiments 1 to 21, wherein the compound is 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-methylpiperazin-1-yl)-N-(3-(2,6-dioxopiperidin-3-ylamino)phenyl)acetamide.

26. The method of any one of embodiments 1 to 21, wherein the compound is 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide or a pharmaceutically acceptable salt thereof.

27. The method of any one of embodiments 1 to 21, wherein the compound is 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide.

28. The method of any one of embodiments 1 to 21, wherein the compound is 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide or a pharmaceutically acceptable salt thereof.

29. The method of any one of embodiments 1 to 21, wherein the compound is 2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide.

30. The method of any one of embodiments 1 to 21, wherein the compound is N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide or a pharmaceutically acceptable salt thereof.

31. The method of any one of embodiments 1 to 21, wherein the compound is N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide.

32. The method of any one of embodiments 1 to 31, wherein the androgen mediated disease is prostate cancer.

33. The method of embodiment 32, wherein the prostate cancer is castration resistant prostate cancer (CRPC).

34. The method of any one of embodiments 1 to 33, wherein the second agent is selected from capivasertib, ipatasertib, AZD8186, 6H-thieno[3,2-f][1,2,4]triazolo[4,3-a][1,4]diazepine-6-acetic acid, tazemetostat, ruxolitinib and sorafenib.

35. The method of any one of embodiments 1 to 33, wherein the AKT inhibitor is selected from capivasertib and ipatasertib.

36. The method of any one of embodiments 1 to 33, wherein the PI3K inhibitor is selected from a PI3Kα inhibitor, a PI3Kβ inhibitor and a PI3Kδ inhibitor.

37. The method of any one of embodiments 1 to 33, wherein the PI3K inhibitor is AZD8186.

38. The method of any one of embodiments 1 to 33, wherein the BET inhibitor is 6H-thieno[3,2-f][1,2,4]triazolo[4,3-a][1,4]diazepine-6-acetic acid.

39. The method of any one of embodiments 1 to 33, wherein the EZH2 inhibitor is tazemetostat.

40. The method of any one of embodiments 1 to 33, wherein the JAK inhibitor is ruxolitinib.

41. The method of any one of embodiments 1 to 33, wherein the RAF kinase inhibitor is sorafenib.

42. The method of any one of embodiments 1 to 41, wherein the therapeutically effective amount of the compound is about 0.01 mg/day to about 750 mg/day, about 0.1 mg/day to about 375 mg/day, about 0.1 mg/day to about 150 mg/day, about 0.1 mg/day to about 75 mg/day, about 0.1 mg/day to about 50 mg/day, about 0.1 mg/day to about 25 mg/day, or about 0.1 mg/day to about 10 mg/day.

43. The method of any one of embodiments 1 to 41, wherein the compound is administered once, twice, three, four or more times daily.

44. The method of any one of embodiments 1 to 41, wherein the compound is administered in a dose of about 100 mg administered in a once daily dose.

45. The method of any one of embodiments 1 to 41, wherein the compound is administered in a dose of about 100 mg administered in a twice daily dose.

46. The method of embodiment 39, wherein tazemetostat is administered in an amount of about 800 mg, 600 mg, 400 mg or 200 mg.

47. The method of embodiment 39, wherein tazemetostat is administered in an amount of about 800 mg, 600 mg, 400 mg or 200 mg twice per day.

48. The method of embodiment 40, wherein ruxolitinib is administered in an amount of about 5 mg, 10 mg, 15 mg, 20 mg, 25 mg.

49. The method of embodiment 40, wherein ruxolitinib is administered in an amount of about 5 mg, 10 mg, 15 mg, 20 mg, 25 mg twice per day.

50. The method of embodiment 41, wherein sorafenib is administered in an amount of about 300 mg or 400 mg.

51. The method of embodiment 41, wherein sorafenib is administered in an amount of about 300 mg or 400 mg twice per day.

### Example 1: CTG Proliferation Assay

This study was undertaken to identify 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide and second agents that can synergistically inhibit proliferation of prostate cancer cell lines in an in-vitro combination screen. Three prostate cancer cell lines LNCAP, VCAP, and 22RV1 were used in this screen. Briefly, LNCaP cells were cultured in RPMI medium supplemented with 10% FBS, 1% penicillin/streptomycin, 10 mM HEPES, 1 mM sodium pyruvate, and 1.5 g/L sodium bicarbonate. LNCaP cells were seeded at a density of 2000 cells/well on 384 black assay plates printed with our different compound combinations of interest printed on the plates. PC3 cells were cultured in F12K medium supplemented with 10% FBS and 1% penicillin/streptomycin. 22RV1 cells were cultured in RPMI1640 medium supplemented with 10% FBS and 1% penicillin/streptomycin. 22RV1 cells were seeded at a density of 2000 cells/well on 384 black assay plates printed with our different compound combinations of interest printed on the plates. VCaP cells were cultured in DMEM medium supplemented with 10% FBS, 1% penicillin/streptomycin 1 mM sodium pyruvate, 1.5 g/L sodium bicarbonate on high bind treated tissue culture flasks (Corning CellBIND) to increase cell surface attachment. VCaP cells were seeded at a density of 3000 cells/well on high-bind 384 black assay plates printed with our different compound combinations of interest printed on the plates. The combination plates seeded with cells were then incubated for 7 days at 37° C. and 5% CO2. Cell viability assays were then performed on the plates using Promega CellTiter-Glo (CTG) Luminescent Cell Viability Assay kit and following the manufacturer's protocol. 20 μL of CTG reagent was added to each well of the 384 well plate, and plates were then incubated for 45 minutes before measuring the fluorescence using PerkinElmer EnVision Plate Reader.

### Statistical Analysis

Synergy is calculated by Bliss and HSA excess scores. Bliss and HSA excess scores are computed by subtracting the area under the experimental response surface to the areas under the corresponding reference model surface. A negative HSA excess score indicates antagonism, while a positive HSA excess indicates additivity and synergy is detected when also the Bliss excess score is positive. Replicates are integrated by fitting individual dose response curves. Statistical analysis is performed by first estimating experimental noise and goodness-of-fit values for each individual combination. These values are used to compute Z-scores for the observed effects. Z-scores are integrated across the combination matrix where the deviation from the model is consistent over adjacent doses and a p-value is computed.

### Results

Combinations with significant synergy scores (p<0.05) across all the three cell lines tested are provided in Table 2. For AKT inhibitor, a synergy score of 8 and 2 were obtained in LNCAP and VCAP cell lines respectively. For panAKT inhibitor, a synergy score of 5.6, 2.1 and 1.5 were obtained in LNCAP, 22RV1 and VCAP cell lines, respectively. For PI3Kβ and PI3Kδ inhibitor, a synergy score of 1.6, 1.5 and 0.39 were obtained in 22RV1, LNCAP, and VCAP cell lines, respectively. For BET inhibitor, a synergy score of 2.8 and 1.3 were obtained in VCAP and LNCAP cell lines, respectively. For EZH2i, a synergy score of 4.1 was obtained in LNCAP. For JAK1/2i, a synergy score of 2 was obtained in VCAP. For Sorafenib, a synergy score of 0.65 was obtained in 22RV1.

### CONCLUSIONS

PIK3K/AKT, BET, JAK1/2, and EZH2 inhibitors have shown significant synergy with Compound I in inhibiting proliferation of prostate cancer cell lines.

TABLE 2

| | | | Synergy | | | | | | |
|---|---|---|---|---|---|---|---|---|---|
| Cell | Second | Synergy | region | Maximum | Synergy | Additivity | Additivity | Antagonism | Antagonism |
| line | agent | region | p-value | synergy | excess | score | p-value | score | p-value |
| LNCaP | AZD5363 | 8.1 | 6.60E−39 | 26 | 9.4 | 16 | 5.30E−107 | 0 | 1 |
| | (Capivasertib, | | | | | | | | |
| | AKT inhibitor) | | | | | | | | |
| VCaP | AZD5363 | 2 | 0.0000084 | 13 | 6.2 | 7.1 | 8.40E−27 | 0 | 0.82 |
| LNCaP | GDC-0068 | 5.6 | 6.60E−17 | 23 | 7.6 | 13 | 2.10E−53 | 0 | 0.96 |
| | (Ipatasertib- | | | | | | | | |
| | pan, Akt | | | | | | | | |
| | inhibitor) | | | | | | | | |
| 22RV1 | GDC-0068 | 2.1 | 1.70E−14 | 23 | 2.4 | 7.5 | 1.80E−40 | 0 | 0.89 |
| VCaP | GDC-0068 | 1.5 | 0.00024 | 14 | 3.8 | 7.5 | 5.70E−24 | 0 | 0.72 |
| 22RV1 | AZD8186 | 1.6 | 1.70E−09 | 24 | 2.4 | 7.6 | 1.20E−45 | 0 | 0.91 |
| | (PI3Kβ | | | | | | | | |
| | and PI3Kδ | | | | | | | | |
| | inhibitor) | | | | | | | | |
| LNCaP | AZD8186 | 1.5 | 0.0017 | 12 | 4.4 | 10 | 2.40E−16 | 0 | 0.87 |
| VCaP | AZD8186 | 0.39 | 0.023 | 11 | 2.9 | 2.1 | 7.40E−07 | 0 | 0.69 |
| VCaP | JQ1 (BET | 2.8 | 0.0000016 | 17 | 4.8 | 10 | 3.90E−24 | 0 | 0.85 |
| | inhibitor) | | | | | | | | |
| LNCaP | JQ1 | 1.3 | 0.0000051 | 17 | 2.4 | 8 | 3.70E−28 | 0 | 0.86 |
| LNCaP | Tazemetostat | 4.1 | 2.10E−16 | 20 | 5.3 | 6.1 | 9.20E−30 | 0 | 0.75 |
| | (EPZ-6438, | | | | | | | | |
| | EZH2 | | | | | | | | |
| | inhibitor) | | | | | | | | |
| VCaP | Ruxolitinib | 2 | 0.0013 | 17 | 8.7 | 5.2 | 2.00E−07 | 0 | 0.76 |
| | (INCB-18424, | | | | | | | | |
| | JAK1/2 | | | | | | | | |
| | inhibitor) | | | | | | | | |
| 22RV1 | Sorafenib | 0.65 | 0.00014 | 20 | 0.17 | 2.6 | 1.20E−10 | 0 | 0.71 |

Embodiments provided herein may be more fully understood by reference to the following examples. These examples are meant to be illustrative of pharmaceutical compositions and dosage forms provided herein, but are not in any way limiting.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

1. A method of treating an androgen receptor mediated disease in a patient having an androgen receptor mediated disease comprising administering to the patient a therapeutically effective amount of a compound and a therapeutically effective amount of a second agent selected from a PI3K inhibitor, an AKT inhibitor, a BET inhibitor, JAK inhibitor, and an EZH2 inhibitor, wherein the compound has formula (I) 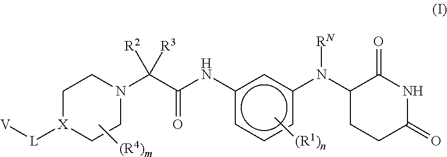
or a pharmaceutically acceptable salt, tautomer, isotopolog, or stereoisomer thereof, wherein
R^{N} is H;
each R¹ is independently selected from halogen, CN, and C₁₋₃ alkyl;
R² and R³ are each independently selected from H, and C₁₋₃ alkyl, or R² and R³ and the carbon to which they are attached form a substituted or unsubstituted C₃₋₆ cycloalkyl;
each R⁴ is independently substituted or unsubstituted C₁₋₃ alkyl, or two R⁴ groups, together with the same carbon atom or adjacent carbon atoms to which they are attached, form a substituted or unsubstituted C₃₋₆ cycloalkyl, or two R⁴ groups together with the non-adjacent carbon atoms to which they are attached form a substituted or unsubstituted 4-7-membered heterocyclyl;
X is N;
L is —O(C₁₋₆ alkyl)- or —(C₁₋₉ alkyl)-;
n is 0-4;
m is 0-8;
V is 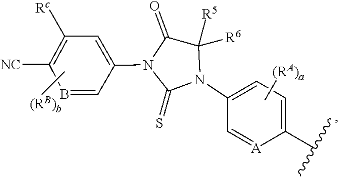
wherein
A is N, CH, or CR^{A};
B is N, CH, or CR^{B};
each R^{A} is independently selected from halogen, substituted or unsubstituted C₁₋₆ alkyl, and substituted or unsubstituted C₃₋₆ cycloalkyl;
each R^{B} is independently selected from halogen, and substituted or unsubstituted C₁₋₆ alkyl;
R^{C} is halogen or CF₃;
R⁵ and R⁶ are C₁₋₃ alkyl, or R⁵ and R⁶, together with the carbon atom to which they are attached, form a substituted or unsubstituted C₃₋₆ cycloalkyl or a 3-6 membered heterocyclyl;
a is 0-3; and
b is 0-2.

2. The method of claim 1, wherein two R⁴ groups together with the non-adjacent carbon atoms to which they are attached form an unsubstituted 4-7-membered heterocyclyl.

3. The method of claim 1, wherein L is —O(CH₂)(CH₂)—, —O(CH₂)(CH(CH₃))—, —O(CH(CH₃))(CH₂)—, or —(CH₂)(CH₂)(CH₂)—.

4. The method of claim 1, wherein each R^{A} is independently selected from Cl, Br, F, methyl, ethyl, n-propyl, isopropyl, isobutyl, sec-butyl, CF₃, CF₂CH₃, CH₂CH₂F, CH₂CHF₂, CH₂OH, CH(CH₃)OH, cyclopropyl, cyclobutyl, and cyclopentyl.

5. The method of claim 1, wherein the compound is selected from the group consisting of
2-(4-(2-(4-(3-(4-cyano-3-(trifluoromethyl)phenyl)-5,5-dimethyl-4-oxo-2-thioxoimidazolidin-1-yl)-2-fluorophenoxy)ethyl)piperazin-1-yl)-N-(3-(2,6-dioxopiperidin-3-ylamino)phenyl)acetamide,
2-(4-(2-(4-(3-(6-cyano-5-(trifluoromethyl)pyridin-3-yl)-5,5-dimethyl-4-oxo-2-thioxoimidazolidin-1-yl)phenoxy)ethyl)piperazin-1-yl)-N-(3-(2,6-dioxopiperidin-3-ylamino)phenyl)acetamide,
2-(4-(2-(2-chloro-4-(3-(4-cyano-3-(trifluoromethyl)phenyl)-5,5-dimethyl-4-oxo-2-thioxoimidazolidin-1-yl)phenoxy)ethyl)piperazin-1-yl)-N-(3-(2,6-dioxopiperidin-3-ylamino)phenyl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-fluorophenoxy)ethyl)piperazin-1-yl)acetamide,
2-(4-(2-(2-chloro-4-(3-(6-cyano-5-(trifluoromethyl)pyridin-3-yl)-5,5-dimethyl-4-oxo-2-thioxoimidazolidin-1-yl)phenoxy)ethyl)piperazin-1-yl)-N-(3-(2,6-dioxopiperidin-3-ylamino)phenyl)acetamide,
2-(4-(2-(4-(7-(6-cyano-5-(trifluoromethyl)pyridin-3-yl)-8-oxo-6-thioxo-5,7-diazaspiro[3.4]octan-5-yl)-2-fluorophenoxy)ethyl)piperazin-1-yl)-N-(3-(2,6-dioxopiperidin-3-ylamino)phenyl)acetamide,
2-(4-(2-(2-chloro-4-(7-(4-cyano-3-(trifluoromethyl)phenyl)-8-oxo-6-thioxo-5,7-diazaspiro[3.4]octan-5-yl)phenoxy)ethyl)piperazin-1-yl)-N-(3-(2,6-dioxopiperidin-3-ylamino)phenyl)acetamide,
2-(4-(2-(4-(7-(4-cyano-3-(trifluoromethyl)phenyl)-8-oxo-6-thioxo-5,7-diazaspiro[3.4]octan-5-yl)-2-fluorophenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide,
2-(4-(2-(2-bromo-4-(3-(4-cyano-3-(trifluoromethyl)phenyl)-5,5-dimethyl-4-oxo-2-thioxoimidazolidin-1-yl)phenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-(trifluoromethyl)phenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-fluoro-5-methylphenoxy)ethyl)piperazin-1-yl)-N-(3-(2,6-dioxopiperidin-3-ylamino)phenyl)acetamide,
2-(4-(2-(2-chloro-4-(3-(6-cyano-5-(trifluoromethyl)pyridin-3-yl)-5,5-dimethyl-4-oxo-2-thioxoimidazolidin-1-yl)phenoxy)ethyl)piperazin-1-yl)-N-(3-chloro-5-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-fluorophenoxy)ethyl)piperazin-1-yl)-N-(3-(2,6-dioxopiperidin-3-ylamino)-5-methylphenyl)acetamide,
2-(4-(2-(2-chloro-4-(3-(4-cyano-3-(trifluoromethyl)phenyl)-5,5-dimethyl-4-oxo-2-thioxoimidazolidin-1-yl)phenoxy)ethyl)piperazin-1-yl)-N-(3-chloro-5-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-isopropylphenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-isobutylphenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-fluorophenoxy)ethyl)piperazin-1-yl)-N-(3-(2,6-dioxopiperidin-3-ylamino)-5-fluorophenyl)acetamide,
2-(4-(2-(2-chloro-4-(3-(4-cyano-3-(trifluoromethyl)phenyl)-5,5-dimethyl-4-oxo-2-thioxoimidazolidin-1-yl)phenoxy)ethyl)piperazin-1-yl)-N-(2-chloro-5-(2,6-dioxopiperidin-3-ylamino)phenyl)acetamide,
(2S)-2-(4-(2-(2-chloro-4-(3-(4-cyano-3-(trifluoromethyl)phenyl)-5,5-dimethyl-4-oxo-2-thioxoimidazolidin-1-yl)phenoxy)ethyl)piperazin-1-yl)-N-(3-(2,6-dioxopiperidin-3-ylamino)phenyl)propanamide,
2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)phenoxy)ethyl)piperazin-1-yl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)phenoxy)ethyl)piperazin-1-yl)acetamide,
(2R)-2-(4-(2-(2-chloro-4-(3-(4-cyano-3-(trifluoromethyl)phenyl)-5,5-dimethyl-4-oxo-2-thioxoimidazolidin-1-yl)phenoxy)ethyl)piperazin-1-yl)-N-(3-(2,6-dioxopiperidin-3-ylamino)phenyl)propanamide,
2-(4-(2-(4-(3-(6-cyano-5-(trifluoromethyl)pyridin-3-yl)-5,5-dimethyl-4-oxo-2-thioxoimidazolidin-1-yl)-2-fluoro-5-methylphenoxy)ethyl)piperazin-1-yl)-N-(3-(2,6-dioxopiperidin-3-ylamino)phenyl)acetamide,
(S)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide,
(R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)acetamide,
2-(4-(2-(2-sec-butyl-4-(3-(4-cyano-3-(trifluoromethyl)phenyl)-5,5-dimethyl-4-oxo-2-thioxoimidazolidin-1-yl)phenoxy)ethyl)piperazin-1-yl)-N-(3-(2,6-dioxopiperidin-3-ylamino)phenyl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)piperazin-1-yl)acetamide,
2-(4-(2-(4-(3-(4-cyano-3-(trifluoromethyl)phenyl)-5,5-dimethyl-4-oxo-2-thioxoimidazolidin-1-yl)-2-cyclobutylphenoxy)ethyl)piperazin-1-yl)-N-(3-(2,6-dioxopiperidin-3-ylamino)phenyl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)acetamide,
2-(4-(2-(4-(3-(6-cyano-5-(trifluoromethyl)pyridin-3-yl)-5,5-dimethyl-4-oxo-2-thioxoimidazolidin-1-yl)-2-cyclopropylphenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-propylphenoxy)ethyl)piperazin-1-yl)-N-(3-(2,6-dioxopiperidin-3-ylamino)phenyl)acetamide,
N-(2-chloro-3-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)acetamide,
2-(4-(2-(4-(3-(6-cyano-5-(trifluoromethyl)pyridin-3-yl)-5,5-dimethyl-4-oxo-2-thioxoimidazolidin-1-yl)-2-cyclobutylphenoxy)ethyl)piperazin-1-yl)-N-(3-(2,6-dioxopiperidin-3-ylamino)phenyl)acetamide,
2-(4-(2-(2-sec-butyl-4-(3-(6-cyano-5-(trifluoromethyl)pyridin-3-yl)-5,5-dimethyl-4-oxo-2-thioxoimidazolidin-1-yl)phenoxy)ethyl)piperazin-1-yl)-N-(3-(2,6-dioxopiperidin-3-ylamino)phenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-propylphenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-cyclopentylphenoxy)ethyl)piperazin-1-yl)-N-(3-(2,6-dioxopiperidin-3-ylamino)phenyl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)acetamide,
N-(2-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)acetamide,
2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(5-(2,6-dioxopiperidin-3-ylamino)-2-fluorophenyl)acetamide,
2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-methylpiperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-methylpiperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide,
2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide,
N-(2-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-(hydroxymethyl)piperazin-1-yl)-N-(3-(2,6-dioxopiperidin-3-ylamino)phenyl)acetamide,
2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-3-(hydroxymethyl)piperazin-1-yl)-N-(3-(2,6-dioxopiperidin-3-ylamino)phenyl)acetamide,
2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-(hydroxymethyl)piperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-(hydroxymethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-2,4-dioxoimidazolidin-1-yl)-2-ethylphenoxy)ethyl)piperazin-1-yl)-N-(3-(2,6-dioxopiperidin-3-ylamino)phenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-isopropylphenoxy)ethyl)piperazin-1-yl)-N-(3-(2,6-dioxopiperidin-3-ylamino)phenyl)acetamide,
N-(3-chloro-5-(2,6-dioxopiperidin-3-ylamino)phenyl)-2-(4-(2-(4-(3-(4-cyano-3-(trifluoromethyl)phenyl)-4-oxo-2-thioxo-7-oxa-1,3-diazaspiro[4.4]nonan-1-yl)-2-fluorophenoxy)ethyl)piperazin-1-yl)acetamide,
N-(3-chloro-5-(2,6-dioxopiperidin-3-ylamino)phenyl)-2-(4-(2-(4-(3-(4-cyano-3-(trifluoromethyl)phenyl)-4-oxo-2-thioxo-7-oxa-1,3-diazaspiro[4.4]nonan-1-yl)-2-fluorophenoxy)ethyl)piperazin-1-yl)acetamide,
2-(4-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)piperazin-1-yl)-N-(3-(2,6-dioxopiperidin-3-ylamino)phenyl)acetamide,
(2S)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)propanamide,
(2R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)propanamide,
2-(4-(2-(4-(3-(4-cyano-3-(trifluoromethyl)phenyl)-5,5-dimethyl-4-oxo-2-thioxoimidazolidin-1-yl)-2-(1,1-difluoroethyl)phenoxy)ethyl)piperazin-1-yl)-N-(3-(2,6-dioxopiperidin-3-ylamino)phenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)piperazin-1-yl)-N-(3-(2,6-dioxopiperidin-3-ylamino)phenyl)acetamide,
2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)-5-fluorophenyl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)piperazin-1-yl)acetamide,
2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)-5-fluorophenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)-N-(5-(2,6-dioxopiperidin-3-ylamino)-2-fluorophenyl)acetamide,
2-(4-(2-(4-(3-(4-cyano-3-(trifluoromethyl)phenyl)-5,5-dimethyl-4-oxo-2-thioxoimidazolidin-1-yl)-2-fluorophenoxy)ethyl)piperazin-1-yl)-N-(3-cyano-5-(2,6-dioxopiperidin-3-ylamino)phenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-(2,2-difluoroethyl)phenoxy)ethyl)piperazin-1-yl)-N-(5-(2,6-dioxopiperidin-3-ylamino)-2-fluorophenyl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-(2,2-difluoroethyl)phenoxy)ethyl)piperazin-1-yl)acetamide,
2-(4-(2-(4-(3-(6-cyano-5-(trifluoromethyl)pyridin-3-yl)-5,5-dimethyl-4-oxo-2-thioxoimidazolidin-1-yl)-2-(2,2-difluoroethyl)phenoxy)ethyl)piperazin-1-yl)-N-(3-(2,6-dioxopiperidin-3-ylamino)-5-fluorophenyl)acetamide,
N-(3-chloro-5-(2,6-dioxopiperidin-3-ylamino)phenyl)-2-(4-(2-(5-(3-(4-cyano-3-(trifluoromethyl)phenyl)-5,5-dimethyl-4-oxo-2-thioxoimidazolidin-1-yl)-3-ethylpyridin-2-yloxy)ethyl)piperazin-1-yl)acetamide,
2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(5-(2,6-dioxopiperidin-3-ylamino)-2,3-difluorophenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-(2-fluoroethyl)phenoxy)ethyl)piperazin-1-yl)-N-(5-(2,6-dioxopiperidin-3-ylamino)-2-fluorophenyl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-(2-fluoroethyl)phenoxy)ethyl)piperazin-1-yl)acetamide,
2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,2-dimethylpiperazin-1-yl)-N-(3-(2,6-dioxopiperidin-3-ylamino)-5-fluorophenyl)acetamide,
N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide,
2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)-5-fluorophenyl)acetamide,
2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)-N-(5-(2,6-dioxopiperidin-3-ylamino)-2-fluorophenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,2-dimethylpiperazin-1-yl)-N-(3-(2,6-dioxopiperidin-3-ylamino)-5-fluorophenyl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,2-dimethylpiperazin-1-yl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,2-dimethylpiperazin-1-yl)acetamide,
2-(4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)piperazin-1-yl)-N-(3-(2,6-dioxopiperidin-3-ylamino)-5-fluorophenyl)acetamide,
2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)piperazin-1-yl)-N-(5-(2,6-dioxopiperidin-3-ylamino)-2,3-difluorophenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,2-dimethylpiperazin-1-yl)-N-(5-(2,6-dioxopiperidin-3-ylamino)-2,3-difluorophenyl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-cyclopropylphenoxy)ethyl)piperazin-1-yl)acetamide,
N-(3-chloro-5-(2,6-dioxopiperidin-3-ylamino)phenyl)-2-(4-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)piperazin-1-yl)acetamide,
2-(4-(2-(4-(3-(6-cyano-5-(trifluoromethyl)pyridin-3-yl)-5,5-dimethyl-4-oxo-2-thioxoimidazolidin-1-yl)phenoxy)ethyl)piperazin-1-yl)-N-(5-(2,6-dioxopiperidin-3-ylamino)-2-fluorophenyl)acetamide,
N-(3-chloro-5-(2,6-dioxopiperidin-3-ylamino)phenyl)-2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)acetamide,
2-(4-(3-(4-(3-(4-cyano-3-(trifluoromethyl)phenyl)-5,5-dimethyl-4-oxo-2-thioxoimidazolidin-1-yl)-2-ethylphenyl)propyl)piperazin-1-yl)-N-(3-(2,6-dioxopiperidin-3-ylamino)-5-fluorophenyl)acetamide,
N-(3-chloro-5-(2,6-dioxopiperidin-3-ylamino)phenyl)-2-(4-(3-(4-(3-(4-cyano-3-(trifluoromethyl)phenyl)-5,5-dimethyl-4-oxo-2-thioxoimidazolidin-1-yl)-2-ethylphenyl)propyl)piperazin-1-yl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)-5-fluorophenyl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-(hydroxymethyl)phenoxy)ethyl)piperazin-1-yl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-(1-hydroxyethyl)phenoxy)ethyl)piperazin-1-yl)acetamide,
N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-isopropylphenoxy)ethyl)piperazin-1-yl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-isopropylphenoxy)ethyl)piperazin-1-yl)acetamide,
2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,2-dimethylpiperazin-1-yl)-N-(3-(2,6-dioxopiperidin-3-ylamino)phenyl)acetamide,
2-(4-(2-(4-(3-(4-cyano-2-methyl-3-(trifluoromethyl)phenyl)-5,5-dimethyl-4-oxo-2-thioxoimidazolidin-1-yl)-2-fluorophenoxy)ethyl)piperazin-1-yl)-N-(3-(2,6-dioxopiperidin-3-ylamino)phenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,2-dimethylpiperazin-1-yl)-N-(3-(2,6-dioxopiperidin-3-ylamino)phenyl)acetamide,
2-(7-(2-(4-(3-(4-cyano-3-(trifluoromethyl)phenyl)-5,5-dimethyl-4-oxo-2-thioxoimidazolidin-1-yl)-2-ethylphenoxy)ethyl)-4,7-diazaspiro[2.5]octan-4-yl)-N-(3-(2,6-dioxopiperidin-3-ylamino)phenyl)acetamide,
2-(7-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-4,7-diazaspiro[2.5]octan-4-yl)-N-(3-(2,6-dioxopiperidin-3-ylamino)phenyl)acetamide,
2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)-N-(5-(2,6-dioxopiperidin-3-ylamino)-2,3-difluorophenyl)acetamide,
N-(3-chloro-5-(2,6-dioxopiperidin-3-ylamino)phenyl)-2-(7-(2-(4-(3-(4-cyano-3-(trifluoromethyl)phenyl)-5,5-dimethyl-4-oxo-2-thioxoimidazolidin-1-yl)-2-ethylphenoxy)ethyl)-4,7-diazaspiro[2.5]octan-4-yl)acetamide,
N-(3-chloro-5-(2,6-dioxopiperidin-3-ylamino)phenyl)-2-(7-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-4,7-diazaspiro[2.5]octan-4-yl)acetamide,
N-(3-chloro-5-(2,6-dioxopiperidin-3-ylamino)phenyl)-2-((R)-4-(2-(4-(7-(4-cyano-3-(trifluoromethyl)phenyl)-8-oxo-6-thioxo-5,7-diazaspiro[3.4]octan-5-yl)-2-ethylphenoxy)ethyl)-2-methylpiperazin-1-yl)acetamide,
2-((R)-4-(2-(4-(7-(4-cyano-3-(trifluoromethyl)phenyl)-8-oxo-6-thioxo-5,7-diazaspiro[3.4]octan-5-yl)-2-ethylphenoxy)ethyl)-2-methylpiperazin-1-yl)-N-(3-(2,6-dioxopiperidin-3-ylamino)-5-fluorophenyl)acetamide,
2-((R)-4-(2-(4-(7-(4-cyano-3-(trifluoromethyl)phenyl)-8-oxo-6-thioxo-5,7-diazaspiro[3.4]octan-5-yl)-2-ethylphenoxy)ethyl)-2-methylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((R)-4-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)-2-methylpiperazin-1-yl)acetamide,
2-((R)-4-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)-2-methylpiperazin-1-yl)-N-(3-(2,6-dioxopiperidin-3-ylamino)-5-fluorophenyl)acetamide,
2-((R)-4-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)-2-methylpiperazin-1-yl)-N-(3-(2,6-dioxopiperidin-3-ylamino)phenyl)acetamide,
2-(7-(2-(4-(3-(4-cyano-3-(trifluoromethyl)phenyl)-5,5-dimethyl-4-oxo-2-thioxoimidazolidin-1-yl)-2-ethylphenoxy)ethyl)-4,7-diazaspiro[2.5]octan-4-yl)-N-(3-(2,6-dioxopiperidin-3-ylamino)-5-fluorophenyl)acetamide,
2-(7-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-4,7-diazaspiro[2.5]octan-4-yl)-N-(3-(2,6-dioxopiperidin-3-ylamino)-5-fluorophenyl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-((S)-2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-((R)-2,6-dioxopiperidin-3-ylamino)phenyl)acetamide,
(2R)—N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)propanamide,
(2S)—N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)propanamide,
N-(3-chloro-5-(2,6-dioxopiperidin-3-ylamino)phenyl)-2-((1S,4S)-5-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,5-diazabicyclo[2.2.1]heptan-2-yl)acetamide,
(2S)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)-5-fluorophenyl)propanamide,
(2R)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)-5-fluorophenyl)propanamide,
(2S)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)propanamide,
(2R)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)propanamide,
N-(3-chloro-5-(2,6-dioxopiperidin-3-ylamino)phenyl)-2-((1R,4R)-5-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,5-diazabicyclo[2.2.1]heptan-2-yl)acetamide,
2-((2R,5S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)-N-(3-(2,6-dioxopiperidin-3-ylamino)phenyl)acetamide,
2-((2S,5R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((2R,5S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)acetamide,
N-(3-chloro-5-(2,6-dioxopiperidin-3-ylamino)phenyl)-2-((2S,5R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)acetamide,
2-((2S,5S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((2S,5S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)acetamide,
2-((2R,5R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((2R,5R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)acetamide,
2-((2R,6R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((2R,6R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)acetamide,
N-(3-chloro-5-(2,6-dioxopiperidin-3-ylamino)phenyl)-2-((S)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide,
2-(4-(2-(4-(7-(4-cyano-3-(trifluoromethyl)phenyl)-8-oxo-6-thioxo-5,7-diazaspiro[3.4]octan-5-yl)-2-ethylphenoxy)ethyl)piperazin-1-yl)-N-(3-(2,6-dioxopiperidin-3-ylamino)phenyl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-2-methylpropanamide,
2-(4-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)-2,2-dimethylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((R)-4-(2-(4-(3-(4-cyano-3-(trifluoromethyl)phenyl)-5,5-dimethyl-4-oxo-2-thioxoimidazolidin-1-yl)-2-(2-fluoroethyl)phenoxy)ethyl)-2-methylpiperazin-1-yl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-(2-fluoroethyl)phenoxy)ethyl)piperazin-1-yl)acetamide,
2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)-N-(3-((S)-2,6-dioxopiperidin-3-ylamino)phenyl)acetamide,
2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)-N-(3-((R)-2,6-dioxopiperidin-3-ylamino)phenyl)acetamide,
1-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)cyclopropanecarboxamide,
N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-(2-fluoroethyl)phenoxy)ethyl)-2-methylpiperazin-1-yl)acetamide,
2-(4-(2-(4-(3-(4-cyano-3-(trifluoromethyl)phenyl)-5,5-dimethyl-4-oxo-2-thioxoimidazolidin-1-yl)-2-(2-fluoroethyl)phenoxy)ethyl)piperazin-1-yl)-N-(5-(2,6-dioxopiperidin-3-ylamino)-2-fluorophenyl)acetamide,
N-(3-chloro-5-(2,6-dioxopiperidin-3-ylamino)phenyl)-2-((R)-4-(2-(4-(7-(6-cyano-5-(trifluoromethyl)pyridin-3-yl)-8-oxo-6-thioxo-5,7-diazaspiro[3.4]octan-5-yl)-2-(2-fluoroethyl)phenoxy)ethyl)-2-methylpiperazin-1-yl)acetamide,
N-(3-chloro-5-(2,6-dioxopiperidin-3-ylamino)phenyl)-2-((R)-4-(2-(4-(7-(4-cyano-3-(trifluoromethyl)phenyl)-8-oxo-6-thioxo-5,7-diazaspiro[3.4]octan-5-yl)-2-(2-fluoroethyl)phenoxy)ethyl)-2-methylpiperazin-1-yl)acetamide,
N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide,
N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide,
2-((2S,6S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((2S,6S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)acetamide,
N-(3-chloro-5-(2,6-dioxopiperidin-3-ylamino)phenyl)-2-((R)-4-(2-(4-(3-(4-cyano-3-(trifluoromethyl)phenyl)-5,5-dimethyl-4-oxo-2-thioxoimidazolidin-1-yl)-2-(1,1-difluoroethyl)phenoxy)ethyl)-2-methylpiperazin-1-yl)acetamide,
N-(3-chloro-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)-2-methylpiperazin-1-yl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)-N-(3-(2,6-dioxopiperidin-3-ylamino)phenyl)acetamide,
2-((2R,6S)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,6-dimethylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((R)-4-(2-(4-(3-(4-cyano-3-(trifluoromethyl)phenyl)-5,5-dimethyl-4-oxo-2-thioxoimidazolidin-1-yl)-2-(2,2-difluoroethyl)phenoxy)ethyl)-2-methylpiperazin-1-yl)acetamide,
2-((R)-4-(2-(4-(3-(4-cyano-3-(trifluoromethyl)phenyl)-5,5-dimethyl-4-oxo-2-thioxoimidazolidin-1-yl)-2-(2,2-difluoroethyl)phenoxy)ethyl)-2-methylpiperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-cyano-5-(2,6-dioxopiperidin-3-ylamino)phenyl)acetamide,
N-(3-cyano-5-(2,6-dioxopiperidin-3-ylamino)phenyl)-2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-methylpiperazin-1-yl)acetamide,
2-((R)-4-(2-(4-(3-(4-cyano-3-(trifluoromethyl)phenyl)-5,5-dimethyl-4-oxo-2-thioxoimidazolidin-1-yl)-2-(2,2-difluoroethyl)phenoxy)ethyl)-2-methylpiperazin-1-yl)-N-(5-(2,6-dioxopiperidin-3-ylamino)-2-fluorophenyl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-(1,1-difluoroethyl)phenoxy)ethyl)piperazin-1-yl)acetamide,
2-((R)-4-(2-(4-(3-(4-cyano-3-(trifluoromethyl)phenyl)-5,5-dimethyl-4-oxo-2-thioxoimidazolidin-1-yl)-2-(2,2-difluoroethyl)phenoxy)ethyl)-2-methylpiperazin-1-yl)-N-(3-(2,6-dioxopiperidin-3-ylamino)-5-fluorophenyl)acetamide,
N-(3-chloro-5-(2,6-dioxopiperidin-3-ylamino)phenyl)-2-(4-(2-(4-(3-(4-cyano-3-(trifluoromethyl)phenyl)-5,5-dimethyl-4-oxo-2-thioxoimidazolidin-1-yl)-2-(2,2-difluoroethyl)phenoxy)ethyl)piperazin-1-yl)acetamide,
2-(4-(2-(4-(3-(4-cyano-3-(trifluoromethyl)phenyl)-5,5-dimethyl-4-oxo-2-thioxoimidazolidin-1-yl)-2-(2-fluoroethyl)phenoxy)ethyl)piperazin-1-yl)-N-(3-(2,6-dioxopiperidin-3-ylamino)phenyl)acetamide,
2-(4-(2-(4-(3-(6-cyano-5-(trifluoromethyl)pyridin-3-yl)-5,5-dimethyl-4-oxo-2-thioxoimidazolidin-1-yl)-2-(2-fluoroethyl)phenoxy)ethyl)piperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2-methylpiperazin-1-yl)-N-(3-(2,6-dioxopiperidin-3-ylamino)phenyl)acetamide,
2-((R)-4-(2-(4-(3-(6-cyano-5-(trifluoromethyl)pyridin-3-yl)-5,5-dimethyl-4-oxo-2-thioxoimidazolidin-1-yl)-2-isopropylphenoxy)ethyl)-2-methylpiperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-(2-fluoroethyl)phenoxy)ethyl)-2-methylpiperazin-1-yl)-N-(3-(2,6-dioxopiperidin-3-ylamino)phenyl)acetamide,
N-(3-chloro-5-(2,6-dioxopiperidin-3-ylamino)phenyl)-1-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)cyclopropanecarboxamide,
2-((R)-4-(2-(4-(3-(6-cyano-5-(trifluoromethyl)pyridin-3-yl)-5,5-dimethyl-4-oxo-2-thioxoimidazolidin-1-yl)-2-(2-fluoroethyl)phenoxy)ethyl)-2-methylpiperazin-1-yl)-N-(3-(2,6-dioxopiperidin-3-ylamino)phenyl)acetamide,
2-((R)-4-(2-(4-(3-(5-chloro-6-cyanopyridin-3-yl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide,
2-((R)-4-(2-(4-(3-(3-chloro-4-cyanophenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((2R,5R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,5-dimethylpiperazin-1-yl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2,6-dimethylpiperazin-1-yl)-N-(3-(2,6-dioxopiperidin-3-ylamino)phenyl)acetamide,
2-(3-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)-3,8-diazabicyclo[3.2.1]octan-8-yl)-N-(3-(2,6-dioxopiperidin-3-ylamino)phenyl)acetamide,
2-((1S,4S)-5-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)-2,5-diazabicyclo[2.2.2]octan-2-yl)-N-(3-(2,6-dioxopiperidin-3-ylamino)phenyl)acetamide,
N-(3-chloro-5-(2,6-dioxopiperidin-3-ylamino)phenyl)-2-(3-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-3,8-diazabicyclo[3.2.1]octan-8-yl)acetamide,
2-(4-(2-(4-(3-(4-cyano-3-(trifluoromethyl)phenyl)-5,5-dimethyl-4-oxo-2-thioxoimidazolidin-1-yl)-2-(2,2-difluoroethyl)phenoxy)ethyl)piperazin-1-yl)-N-(5-(2,6-dioxopiperidin-3-ylamino)-2-fluorophenyl)acetamide,
N-(3-chloro-5-(2,6-dioxopiperidin-3-ylamino)phenyl)-2-((2R,5R)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2,5-dimethylpiperazin-1-yl)acetamide,
2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-isopropylphenoxy)ethyl)-2,6-dimethylpiperazin-1-yl)-N-(3-(2,6-dioxopiperidin-3-ylamino)phenyl)acetamide,
2-(3-(2-(4-(3-(4-cyano-3-(trifluoromethyl)phenyl)-5,5-dimethyl-4-oxo-2-thioxoimidazolidin-1-yl)-2-ethylphenoxy)ethyl)-3,8-diazabicyclo[3.2.1]octan-8-yl)-N-(3-(2,6-dioxopiperidin-3-ylamino)phenyl)acetamide,
2-(3-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-3,8-diazabicyclo[3.2.1]octan-8-yl)-N-(3-(2,6-dioxopiperidin-3-ylamino)phenyl)acetamide,
N-(3-chloro-5-(2,6-dioxopiperidin-3-ylamino)phenyl)-2-((1S,4S)-5-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,5-diazabicyclo[2.2.2]octan-2-yl)acetamide,
2-((2S,6R)-4-(2-(4-(3-(6-cyano-5-(trifluoromethyl)pyridin-3-yl)-5,5-dimethyl-4-oxo-2-thioxoimidazolidin-1-yl)-2-isopropylphenoxy)ethyl)-2,6-dimethylpiperazin-1-yl)-N-(3-(2,6-dioxopiperidin-3-ylamino)phenyl)acetamide,
2-((2R,5R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,5-dimethylpiperazin-1-yl)-N-(3-cyano-5-(2,6-dioxopiperidin-3-ylamino)phenyl)acetamide,
2-((1R,4R)-5-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)-2,5-diazabicyclo[2.2.2]octan-2-yl)-N-(3-(2,6-dioxopiperidin-3-ylamino)phenyl)acetamide,
2-((1R,4R)-5-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,5-diazabicyclo[2.2.2]octan-2-yl)-N-(3-(2,6-dioxopiperidin-3-ylamino)phenyl)acetamide,
N-(3-chloro-5-(2,6-dioxopiperidin-3-ylamino)phenyl)-2-((1R,4R)-5-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2,5-diazabicyclo[2.2.2]octan-2-yl)acetamide,
N-(3-chloro-5-(2,6-dioxopiperidin-3-ylamino)phenyl)-2-((2R,5R)-4-(2-(4-(3-(6-cyano-5-(trifluoromethyl)pyridin-3-yl)-5,5-dimethyl-4-oxo-2-thioxoimidazolidin-1-yl)-2-isopropylphenoxy)ethyl)-2,5-dimethylpiperazin-1-yl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)-N-(3-cyano-5-(2,6-dioxopiperidin-3-ylamino)phenyl)acetamide,
2-((R)-4-(2-(5-(3-(4-cyano-3-(trifluoromethyl)phenyl)-5,5-dimethyl-4-oxo-2-thioxoimidazolidin-1-yl)-3-ethylpyridin-2-yloxy)ethyl)-2-methylpiperazin-1-yl)-N-(3-(2,6-dioxopiperidin-3-ylamino)phenyl)acetamide,
1-(4-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)piperazin-1-yl)-N-(3-(2,6-dioxopiperidin-3-ylamino)phenyl)cyclopropanecarboxamide,
N-(3-chloro-5-(2,6-dioxopiperidin-3-ylamino)phenyl)-2-((2R,5R)-4-(2-(5-(3-(4-cyano-3-(trifluoromethyl)phenyl)-5,5-dimethyl-4-oxo-2-thioxoimidazolidin-1-yl)-3-ethylpyridin-2-yloxy)ethyl)-2,5-dimethylpiperazin-1-yl)acetamide,
2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-ethylphenoxy)ethyl)-2,6-dimethylpiperazin-1-yl)-N-(5-(2,6-dioxopiperidin-3-ylamino)-2-fluorophenyl)acetamide,
(2R)—N-(3-chloro-5-(2,6-dioxopiperidin-3-ylamino)phenyl)-2-(4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)piperazin-1-yl)propanamide,
2-((2R,5R)-4-(2-(4-(3-(6-cyano-5-(trifluoromethyl)pyridin-3-yl)-5,5-dimethyl-4-oxo-2-thioxoimidazolidin-1-yl)-2-isopropylphenoxy)ethyl)-2,5-dimethylpiperazin-1-yl)-N-(3-(2,6-dioxopiperidin-3-ylamino)phenyl)acetamide,
2-((2R,5R)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2,5-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2,6-dimethylpiperazin-1-yl)-N-(3-cyano-5-(2,6-dioxopiperidin-3-ylamino)phenyl)acetamide,
N-(3-chloro-5-(2,6-dioxopiperidin-3-ylamino)phenyl)-2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-isopropylphenoxy)ethyl)-2,6-dimethylpiperazin-1-yl)acetamide,
2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-cyclopropylphenoxy)ethyl)-2,6-dimethylpiperazin-1-yl)-N-(3-(2,6-dioxopiperidin-3-ylamino)phenyl)acetamide,
1-(4-(2-(4-(3-(4-cyano-3-(trifluoromethyl)phenyl)-5,5-dimethyl-4-oxo-2-thioxoimidazolidin-1-yl)-2-isopropylphenoxy)ethyl)piperazin-1-yl)-N-(3-(2,6-dioxopiperidin-3-ylamino)phenyl)cyclopropanecarboxamide,
2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-cyclopropylphenoxy)ethyl)-2,6-dimethylpiperazin-1-yl)-N-(5-(2,6-dioxopiperidin-3-ylamino)-2-fluorophenyl)acetamide,
(2R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)piperazin-1-yl)-N-(3-(2,6-dioxopiperidin-3-ylamino)phenyl)propanamide,
2-((R)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-(trifluoromethyl)piperazin-1-yl)-N-(3-(2,6-dioxopiperidin-3-ylamino)phenyl)acetamide,
2-((S)-4-(2-(4-(3-(6-cyano-5-(trifluoromethyl)pyridin-3-yl)-5,5-dimethyl-4-oxo-2-thioxoimidazolidin-1-yl)-2-ethylphenoxy)ethyl)-2-(trifluoromethyl)piperazin-1-yl)-N-(3-(2,6-dioxopiperidin-3-ylamino)phenyl)acetamide,
2-((2S,6R)-4-(2-(4-(3-(3-chloro-4-cyanophenyl)-5,5-dimethyl-4-oxo-2-thioxoimidazolidin-1-yl)-2-isopropylphenoxy)ethyl)-2,6-dimethylpiperazin-1-yl)-N-(3-(2,6-dioxopiperidin-3-ylamino)phenyl)acetamide,
2-((2R,5R)-4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-2,5-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-2,6-dimethylpiperazin-1-yl)-N-(3-(2,6-dioxopiperidin-3-ylamino)phenyl)acetamide,
2-((2R,5R)-4-(2-(4-(3-(3-chloro-4-cyanophenyl)-5,5-dimethyl-4-oxo-2-thioxoimidazolidin-1-yl)-2-cyclopropylphenoxy)ethyl)-2,5-dimethylpiperazin-1-yl)-N-(3-(2,6-dioxopiperidin-3-ylamino)phenyl)acetamide,
2-((2S,6R)-4-(2-(4-(3-(3-chloro-4-cyanophenyl)-5,5-dimethyl-4-oxo-2-thioxoimidazolidin-1-yl)-2-cyclopropylphenoxy)ethyl)-2,6-dimethylpiperazin-1-yl)-N-(3-(2,6-dioxopiperidin-3-ylamino)phenyl)acetamide,
2-((2R,5R)-4-(2-(4-(3-(3-chloro-4-cyanophenyl)-5,5-dimethyl-4-oxo-2-thioxoimidazolidin-1-yl)-2-isopropylphenoxy)ethyl)-2,5-dimethylpiperazin-1-yl)-N-(3-(2,6-dioxopiperidin-3-ylamino)phenyl)acetamide,
N-(3-chloro-5-(2,6-dioxopiperidin-3-ylamino)phenyl)-2-((2S,6R)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2,6-dimethylpiperazin-1-yl)acetamide,
2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-isopropylphenoxy)ethyl)-2,6-dimethylpiperazin-1-yl)-N-(5-(2,6-dioxopiperidin-3-ylamino)-2-fluorophenyl)acetamide,
N-(3-cyano-5-(2,6-dioxopiperidin-3-ylamino)phenyl)-2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-isopropylphenoxy)ethyl)-2,6-dimethylpiperazin-1-yl)acetamide,
2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-isopropylphenoxy)ethyl)-2,6-dimethylpiperazin-1-yl)-N-(3-(2,6-dioxopiperidin-3-ylamino)-5-fluorophenyl)acetamide,
N-(3-cyano-5-(2,6-dioxopiperidin-3-ylamino)phenyl)-2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-cyclopropylphenoxy)ethyl)-2,6-dimethylpiperazin-1-yl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2,6-dimethylpiperazin-1-yl)-N-(3-(2,6-dioxopiperidin-3-ylamino)-5-fluorophenyl)acetamide,
N-(3-chloro-5-(2,6-dioxopiperidin-3-ylamino)phenyl)-2-((2R,5R)-4-(2-(5-(3-(4-cyano-3-(trifluoromethyl)phenyl)-5,5-dimethyl-4-oxo-2-thioxoimidazolidin-1-yl)-3-isopropylpyridin-2-yloxy)ethyl)-2,5-dimethylpiperazin-1-yl)acetamide,
N-(3-chloro-5-(2,6-dioxopiperidin-3-ylamino)phenyl)-2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-cyclopropylphenoxy)ethyl)-2,6-dimethylpiperazin-1-yl)acetamide,
2-((2R,6S)-4-(2-(5-(3-(4-cyano-3-(trifluoromethyl)phenyl)-5,5-dimethyl-4-oxo-2-thioxoimidazolidin-1-yl)-3-ethylpyridin-2-yloxy)ethyl)-2,6-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2,6-dimethylpiperazin-1-yl)-N-(5-(2,6-dioxopiperidin-3-ylamino)-2-fluorophenyl)acetamide,
2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-cyclopropylphenoxy)ethyl)-2,6-dimethylpiperazin-1-yl)-N-(3-(2,6-dioxopiperidin-3-ylamino)-5-fluorophenyl)acetamide,
2-((2S,6R)-4-(2-(4-(3-(3-chloro-4-cyanophenyl)-5,5-dimethyl-4-oxo-2-thioxoimidazolidin-1-yl)-2-cyclopropylphenoxy)ethyl)-2,6-dimethylpiperazin-1-yl)-N-(3-cyano-5-(2,6-dioxopiperidin-3-ylamino)phenyl)acetamide,
2-((2S,6R)-4-(2-(4-(3-(3-chloro-4-cyanophenyl)-5,5-dimethyl-4-oxo-2-thioxoimidazolidin-1-yl)-2-cyclopropylphenoxy)ethyl)-2,6-dimethylpiperazin-1-yl)-N-(3-chloro-5-(2,6-dioxopiperidin-3-ylamino)phenyl)acetamide,
2-((2S,6R)-4-(2-(4-(3-(3-chloro-4-cyanophenyl)-5,5-dimethyl-4-oxo-2-thioxoimidazolidin-1-yl)-2-cyclopropylphenoxy)ethyl)-2,6-dimethylpiperazin-1-yl)-N-(5-(2,6-dioxopiperidin-3-ylamino)-2-fluorophenyl)acetamide,
2-((2S,6R)-4-(2-(4-(3-(3-chloro-4-cyanophenyl)-5,5-dimethyl-4-oxo-2-thioxoimidazolidin-1-yl)-2-isopropylphenoxy)ethyl)-2,6-dimethylpiperazin-1-yl)-N-(5-(2,6-dioxopiperidin-3-ylamino)-2-fluorophenyl)acetamide,
2-((2S,6R)-4-(2-(4-(3-(3-chloro-4-cyanophenyl)-5,5-dimethyl-4-oxo-2-thioxoimidazolidin-1-yl)-2-isopropylphenoxy)ethyl)-2,6-dimethylpiperazin-1-yl)-N-(3-chloro-5-(2,6-dioxopiperidin-3-ylamino)phenyl)acetamide,
2-((2R,6S)-4-(2-(5-(3-(4-cyano-3-(trifluoromethyl)phenyl)-5,5-dimethyl-4-oxo-2-thioxoimidazolidin-1-yl)-3-isopropylpyridin-2-yloxy)ethyl)-2,6-dimethylpiperazin-1-yl)-N-(3-(2,6-dioxopiperidin-3-ylamino)phenyl)acetamide,
2-((2R,6S)-4-(2-(4-(7-(4-cyano-3-(trifluoromethyl)phenyl)-8-oxo-6-thioxo-5,7-diazaspiro[3.4]octan-5-yl)-2-cyclopropylphenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(3-chloro-4-cyanophenyl)-5,5-dimethyl-4-oxo-2-thioxoimidazolidin-1-yl)-2-cyclopropylphenoxy)ethyl)-2,6-dimethylpiperazin-1-yl)-N-(3-(2,6-dioxopiperidin-3-ylamino)-5-fluorophenyl)acetamide,
2-((2S,6R)-4-(2-(5-(7-(4-cyano-3-(trifluoromethyl)phenyl)-8-oxo-6-thioxo-5,7-diazaspiro[3.4]octan-5-yl)-3-isopropylpyridin-2-yloxy)ethyl)-2,6-dimethylpiperazin-1-yl)-N-(3-(2,6-dioxopiperidin-3-ylamino)phenyl)acetamide,
2-((2S,6R)-4-(2-(5-(3-(4-cyano-3-(trifluoromethyl)phenyl)-5,5-dimethyl-4-oxo-2-thioxoimidazolidin-1-yl)-3-cyclopropylpyridin-2-yloxy)ethyl)-2,6-dimethylpiperazin-1-yl)-N-(3-(2,6-dioxopiperidin-3-ylamino)phenyl)acetamide,
2-((2S,6R)-4-(2-(5-(7-(4-cyano-3-(trifluoromethyl)phenyl)-8-oxo-6-thioxo-5,7-diazaspiro[3.4]octan-5-yl)-3-cyclopropylpyridin-2-yloxy)ethyl)-2,6-dimethylpiperazin-1-yl)-N-(3-(2,6-dioxopiperidin-3-ylamino)phenyl)acetamide,
2-((2R,5R)-4-(2-(5-(3-(4-cyano-3-(trifluoromethyl)phenyl)-5,5-dimethyl-4-oxo-2-thioxoimidazolidin-1-yl)-3-isopropylpyridin-2-yloxy)ethyl)-2,5-dimethylpiperazin-1-yl)-N-(3-(2,6-dioxopiperidin-3-ylamino)phenyl)acetamide,
2-((2R,5R)-4-(2-(5-(7-(4-cyano-3-(trifluoromethyl)phenyl)-8-oxo-6-thioxo-5,7-diazaspiro[3.4]octan-5-yl)-3-isopropylpyridin-2-yloxy)ethyl)-2,5-dimethylpiperazin-1-yl)-N-(3-cyano-5-(2,6-dioxopiperidin-3-ylamino)phenyl)acetamide,
2-((2R,5R)-4-(2-(5-(3-(4-cyano-3-(trifluoromethyl)phenyl)-5,5-dimethyl-4-oxo-2-thioxoimidazolidin-1-yl)-3-isopropylpyridin-2-yloxy)ethyl)-2,5-dimethylpiperazin-1-yl)-N-(3-cyano-5-(2,6-dioxopiperidin-3-ylamino)phenyl)acetamide,
2-((S)-4-(2-(4-(3-(6-cyano-5-(trifluoromethyl)pyridin-3-yl)-5,5-dimethyl-4-oxo-2-thioxoimidazolidin-1-yl)-2-cyclopropylphenoxy)ethyl)-2-(trifluoromethyl)piperazin-1-yl)-N-(3-(2,6-dioxopiperidin-3-ylamino)phenyl)acetamide,
2-((2R,5R)-4-(2-(4-(3-(6-cyano-5-(trifluoromethyl)pyridin-3-yl)-5,5-dimethyl-4-oxo-2-thioxoimidazolidin-1-yl)-2-cyclopropylphenoxy)ethyl)-2,5-dimethylpiperazin-1-yl)-N-(3-(2,6-dioxopiperidin-3-ylamino)phenyl)acetamide,
2-((2S,6R)-4-(2-(4-(3-(6-cyano-5-(trifluoromethyl)pyridin-3-yl)-5,5-dimethyl-4-oxo-2-thioxoimidazolidin-1-yl)-2-cyclopropylphenoxy)ethyl)-2,6-dimethylpiperazin-1-yl)-N-(3-(2,6-dioxopiperidin-3-ylamino)phenyl)acetamide,
2-((2S,6R)-4-((S)-1-(4-(3-(4-cyano-3-(trifluoromethyl)phenyl)-5,5-dimethyl-4-oxo-2-thioxoimidazolidin-1-yl)-2-ethylphenoxy)propan-2-yl)-2,6-dimethylpiperazin-1-yl)-N-(3-(2,6-dioxopiperidin-3-ylamino)phenyl)acetamide,
2-((2S,6R)-4-((R)-1-(4-(3-(4-cyano-3-(trifluoromethyl)phenyl)-5,5-dimethyl-4-oxo-2-thioxoimidazolidin-1-yl)-2-ethylphenoxy)propan-2-yl)-2,6-dimethylpiperazin-1-yl)-N-(3-(2,6-dioxopiperidin-3-ylamino)phenyl)acetamide,
2-(3-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-3,8-diazabicyclo[3.2.1]octan-8-yl)-N-(3-(2,6-dioxopiperidin-3-ylamino)phenyl)acetamide,
2-((2S,6R)-4-((R)-2-(4-(3-(6-cyano-5-(trifluoromethyl)pyridin-3-yl)-5,5-dimethyl-4-oxo-2-thioxoimidazolidin-1-yl)-2-ethylphenoxy)propyl)-2,6-dimethylpiperazin-1-yl)-N-(3-(2,6-dioxopiperidin-3-ylamino)phenyl)acetamide,
2-((2S,6R)-4-((S)-2-(4-(3-(6-cyano-5-(trifluoromethyl)pyridin-3-yl)-5,5-dimethyl-4-oxo-2-thioxoimidazolidin-1-yl)-2-ethylphenoxy)propyl)-2,6-dimethylpiperazin-1-yl)-N-(3-(2,6-dioxopiperidin-3-ylamino)phenyl)acetamide,
2-((2S,6R)-4-((R)-2-(4-(3-(4-cyano-3-(trifluoromethyl)phenyl)-5,5-dimethyl-4-oxo-2-thioxoimidazolidin-1-yl)-2-ethylphenoxy)propyl)-2,6-dimethylpiperazin-1-yl)-N-(3-(2,6-dioxopiperidin-3-ylamino)phenyl)acetamide,
2-((2S,6R)-4-((S)-2-(4-(3-(4-cyano-3-(trifluoromethyl)phenyl)-5,5-dimethyl-4-oxo-2-thioxoimidazolidin-1-yl)-2-ethylphenoxy)propyl)-2,6-dimethylpiperazin-1-yl)-N-(3-(2,6-dioxopiperidin-3-ylamino)phenyl)acetamide,
N-(3-cyano-5-(2,6-dioxopiperidin-3-ylamino)phenyl)-2-((2R,5R)-4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)-2,5-dimethylpiperazin-1-yl)acetamide,
2-((2R,5R)-4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)-2,5-dimethylpiperazin-1-yl)-N-(3-(2,6-dioxopiperidin-3-ylamino)phenyl)acetamide,
2-((S)-4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-2-(trifluoromethyl)piperazin-1-yl)-N-(3-(2,6-dioxopiperidin-3-ylamino)phenyl)acetamide,
2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-(trifluoromethyl)piperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-ethyl-5-fluorophenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-ethyl-6-fluorophenoxy)ethyl)-2,6-dimethylpiperazin-1-yl)-N-(3-(2,6-dioxopiperidin-3-ylamino)phenyl)acetamide,
2-((2R,6S)-4-(2-(4-(3-(4-cyano-3-(trifluoromethyl)phenyl)-5,5-dimethyl-4-oxo-2-thioxoimidazolidin-1-yl)-2-ethyl-6-methylphenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(3-chloro-4-cyanophenyl)-5,5-dimethyl-4-oxo-2-thioxoimidazolidin-1-yl)-2-isopropylphenoxy)ethyl)-2,6-dimethylpiperazin-1-yl)-N-(3-cyano-5-(2,6-dioxopiperidin-3-ylamino)phenyl)acetamide,
2-((2S,6R)-4-(2-(4-(3-(3-chloro-4-cyanophenyl)-5,5-dimethyl-4-oxo-2-thioxoimidazolidin-1-yl)-2-isopropylphenoxy)ethyl)-2,6-dimethylpiperazin-1-yl)-N-(3-(2,6-dioxopiperidin-3-ylamino)-5-fluorophenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-(1,1-difluoroethyl)phenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-isopropyl-5-methylphenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-ethyl-5-methylphenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)-2,6-dimethylpiperazin-1-yl)-N-(3-(2,6-dioxopiperidin-3-ylamino)phenyl)acetamide,
2-((S)-4-(2-(4-(3-(6-cyano-5-(trifluoromethyl)pyridin-3-yl)-5,5-dimethyl-4-oxo-2-thioxoimidazolidin-1-yl)-2-isopropylphenoxy)ethyl)-2-(trifluoromethyl)piperazin-1-yl)-N-(3-(2,6-dioxopiperidin-3-ylamino)phenyl)acetamide,
2-((S)-4-(2-(4-(3-(4-cyano-3-(trifluoromethyl)phenyl)-5,5-dimethyl-4-oxo-2-thioxoimidazolidin-1-yl)-2-isopropylphenoxy)ethyl)-2-(trifluoromethyl)piperazin-1-yl)-N-(3-(2,6-dioxopiperidin-3-ylamino)phenyl)acetamide,
N-(3-cyano-5-(2,6-dioxopiperidin-3-ylamino)phenyl)-2-((2S,6R)-4-(2-(4-(3-(6-cyano-5-(trifluoromethyl)pyridin-3-yl)-5,5-dimethyl-4-oxo-2-thioxoimidazolidin-1-yl)-2-(1,1-difluoroethyl)phenoxy)ethyl)-2,6-dimethylpiperazin-1-yl)acetamide,
2-((2R,6S)-4-(2-(4-(3-(4-cyano-3-(trifluoromethyl)phenyl)-5,5-dimethyl-4-oxo-2-thioxoimidazolidin-1-yl)-2-cyclobutylphenoxy)ethyl)-2,6-dimethylpiperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-ethylpiperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-2-ethylpiperazin-1-yl)-N-(3-(2,6-dioxopiperidin-3-ylamino)phenyl)acetamide,
2-((R)-4-(2-(4-(7-(6-cyano-5-(trifluoromethyl)pyridin-3-yl)-8-oxo-6-thioxo-5,7-diazaspiro[3.4]octan-5-yl)-2-cyclopropylphenoxy)ethyl)-2-ethylpiperazin-1-yl)-N-(3-(2,6-dioxopiperidin-3-ylamino)phenyl)acetamide,
2-((S)-4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-2-ethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-4-oxo-2-thioxo-1,3-diazaspiro[4.5]decan-1-yl)-2-ethylphenoxy)ethyl)-2,6-dimethylpiperazin-1-yl)-N-(3-(2,6-dioxopiperidin-3-ylamino)phenyl)acetamide,
2-((2S,6R)-4-(2-(4-(7-(6-cyano-5-(trifluoromethyl)pyridin-3-yl)-8-oxo-6-thioxo-5,7-diazaspiro[3.4]octan-5-yl)-2-(1,1-difluoroethyl)phenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-4-oxo-2-thioxo-1,3-diazaspiro[4.4]nonan-1-yl)-2-cyclopropylphenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-4-oxo-2-thioxo-1,3-diazaspiro[4.4]nonan-1-yl)-2-ethylphenoxy)ethyl)-2,6-dimethylpiperazin-1-yl)-N-(3-(2,6-dioxopiperidin-3-ylamino)phenyl)acetamide,
2-((2R,6S)-4-(2-(4-(3-(6-cyano-5-(trifluoromethyl)pyridin-3-yl)-5,5-dimethyl-4-oxo-2-thioxoimidazolidin-1-yl)-2-cyclobutylphenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(3-chloro-4-cyanophenyl)-5,5-dimethyl-4-oxo-2-thioxoimidazolidin-1-yl)-2-(1,1-difluoroethyl)phenoxy)ethyl)-2,6-dimethylpiperazin-1-yl)-N-(3-(2,6-dioxopiperidin-3-ylamino)phenyl)acetamide,
2-((2R,6S)-4-(2-(4-(3-(4-cyano-3-(trifluoromethyl)phenyl)-5,5-dimethyl-4-oxo-2-thioxoimidazolidin-1-yl)-2-ethyl-3-methylphenoxy)ethyl)-2,6-dimethylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-ethyl-3-fluorophenoxy)ethyl)-2,6-dimethylpiperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-2-methylpiperazin-1-yl)-N-(3-(2,6-dioxopiperidin-3-ylamino)phenyl)acetamide,
2-((2R,5R)-4-(3-(4-(3-(4-cyano-3-(trifluoromethyl)phenyl)-5,5-dimethyl-4-oxo-2-thioxoimidazolidin-1-yl)-2-ethylphenyl)propyl)-2,5-dimethylpiperazin-1-yl)-N-(3-(2,6-dioxopiperidin-3-ylamino)phenyl)acetamide,
(2R)-2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)propanamide,
(2S)-2-((S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)propanamide,
2-((R)-4-(2-(2-chloro-4-(3-(4-cyano-3-(trifluoromethyl)phenyl)-5,5-dimethyl-4-oxo-2-thioxoimidazolidin-1-yl)phenoxy)ethyl)-2-methylpiperazin-1-yl)-N-(3-(2,6-dioxopiperidin-3-ylamino)phenyl)acetamide,
2-((R)-4-(2-(4-(3-(4-cyano-3-(trifluoromethyl)phenyl)-5,5-dimethyl-4-oxo-2-thioxoimidazolidin-1-yl)-2-fluorophenoxy)ethyl)-2-methylpiperazin-1-yl)-N-(3-(2,6-dioxopiperidin-3-ylamino)phenyl)acetamide,
2-((2S,6R)-4-(2-(4-(3-(4-cyano-3-(trifluoromethyl)phenyl)-5,5-dimethyl-4-oxo-2-thioxoimidazolidin-1-yl)-2-fluorophenoxy)ethyl)-2,6-dimethylpiperazin-1-yl)-N-(3-(2,6-dioxopiperidin-3-ylamino)phenyl)acetamide,
2-((S)-4-(3-(4-(3-(4-cyano-3-(trifluoromethyl)phenyl)-5,5-dimethyl-4-oxo-2-thioxoimidazolidin-1-yl)-2-ethylphenyl)propyl)-2-methylpiperazin-1-yl)-N-(3-(2,6-dioxopiperidin-3-ylamino)phenyl)acetamide,
(2R)-2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)propanamide,
(2S)-2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)propanamide,
2-((2S,6R)-4-(2-(2-chloro-4-(3-(4-cyano-3-(trifluoromethyl)phenyl)-5,5-dimethyl-4-oxo-2-thioxoimidazolidin-1-yl)phenoxy)ethyl)-2,6-dimethylpiperazin-1-yl)-N-(3-(2,6-dioxopiperidin-3-ylamino)phenyl)acetamide,
2-((2R,6S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)-N-(3-(2,6-dioxopiperidin-3-ylamino)-5-fluorophenyl)acetamide,
2-((2R,6S)-4-(3-(4-(3-(4-cyano-3-(trifluoromethyl)phenyl)-5,5-dimethyl-4-oxo-2-thioxoimidazolidin-1-yl)-2-ethylphenyl)propyl)-2,6-dimethylpiperazin-1-yl)-N-(3-(2,6-dioxopiperidin-3-ylamino)phenyl)acetamide,
2-((2R,6S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)-N-(3-((S)-2,6-dioxopiperidin-3-ylamino)phenyl)acetamide,
2-((2R,6S)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2,6-dimethylpiperazin-1-yl)-N-(3-((R)-2,6-dioxopiperidin-3-ylamino)phenyl)acetamide,
2-((2R,6S)-4-(3-(4-(3-(4-cyano-3-(trifluoromethyl)phenyl)-5,5-dimethyl-4-oxo-2-thioxoimidazolidin-1-yl)-2-fluorophenyl)propyl)-2,6-dimethylpiperazin-1-yl)-N-(3-(2,6-dioxopiperidin-3-ylamino)phenyl)acetamide,
2-((2R,6S)-4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-2,6-dimethylpiperazin-1-yl)-N-(3-((R)-2,6-dioxopiperidin-3-ylamino)phenyl)acetamide,
2-((2R,6S)-4-(2-(4-(3-(4-cyano-3-(trifluoromethyl)phenyl)-5,5-dimethyl-4-oxo-2-thioxoimidazolidin-1-yl)-2-cyclopropylphenoxy)ethyl)-2,6-dimethylpiperazin-1-yl)-N-(3-((S)-2,6-dioxopiperidin-3-ylamino)phenyl)acetamide,
2-((2S,6R)-4-(2-(2-chloro-4-(3-(3-chloro-4-cyanophenyl)-5,5-dimethyl-4-oxo-2-thioxoimidazolidin-1-yl)phenoxy)ethyl)-2,6-dimethylpiperazin-1-yl)-N-(3-((2,6-dioxopiperidin-3-yl)amino)phenyl)acetamide, and
2-((2S,6R)-4-(2-(4-(3-(3-chloro-4-cyanophenyl)-5,5-dimethyl-4-oxo-2-thioxoimidazolidin-1-yl)-2-ethylphenoxy)ethyl)-2,6-dimethylpiperazin-1-yl)-N-(3-((2,6-dioxopiperidin-3-yl)amino)phenyl)acetamide, or a pharmaceutically acceptable salt thereof.

6. The method of claim 1, wherein the compound is 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide or a pharmaceutically acceptable salt thereof.

7. The method of claim 1, wherein the compound is 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl)acetamide.

8. The method of claim 1, wherein the androgen mediated disease is prostate cancer.

9. The method of claim 8, wherein the prostate cancer is castration resistant prostate cancer (CRPC).

10. The method claim 1, wherein the second agent is selected from capivasertib, ipatasertib, AZD8186, 6H-thieno[3,2-f][1,2,4]triazolo[4,3-a][1,4]diazepine-6-acetic acid, tazemetostat, ruxolitinib and sorafenib.

11. The method of claim 1, wherein the therapeutically effective amount of the compound is about 0.01 mg/day to about 750 mg/day, about 0.1 mg/day to about 375 mg/day, about 0.1 mg/day to about 150 mg/day, about 0.1 mg/day to about 75 mg/day, about 0.1 mg/day to about 50 mg/day, about 0.1 mg/day to about 25 mg/day, or about 0.1 mg/day to about 10 mg/day.

12. The method of claim 1, wherein the compound is administered once, twice, three, four or more times daily.

13. The method of claim 1, wherein the compound is administered in a dose of about 100 mg administered in a once daily dose.

14. The method of claim 1, wherein the compound is administered in a dose of about 100 mg administered in a twice daily dose.

15. The method of claim 10, wherein the second agent is tazemetostat, which is administered in an amount of about 800 mg, 600 mg, 400 mg or 200 mg.

16. The method of claim 15, wherein tazemetostat is administered in an amount of about 800 mg, 600 mg, 400 mg or 200 mg twice per day.

17. The method of claim 10, wherein the second agent is ruxolitinib, which is administered in an amount of about 5 mg, 10 mg, 15 mg, 20 mg, 25 mg.

18. The method of claim 17, wherein ruxolitinib is administered in an amount of about 5 mg, 10 mg, 15 mg, 20 mg, 25 mg twice per day.

19. The method of claim 10, wherein the second agent is sorafenib, which is administered in an amount of about 300 mg or 400 mg.

20. The method of claim 19, wherein sorafenib is administered in an amount of about 300 mg or 400 mg twice per day.
